SEQUENCE LISTING (3 sequences)

SEQ ID NO: 1 - DNA, 153381 bp, unidentified
  source 1..153381: mol_type = genomic DNA
agaactttttcgtccctctgccccgtcctacttaatgtagtatccggtaatattatatttgaaatatagatatttattatactttaactactatataatgattattattctacacttaatatatagtaatatactaattatctaccttaatgtattagttatttatactaatcgggtactatcaatgatttaccctacatttatatagtattacttatatatctactcttatatggtttccgcttctttagtttcagttcaatttatttactcaaaggtattgctatttaatgctgtacatgctataatgtatatatactaataaaggaagtggcaattatgattacaattaaagatattattgagtttgttgaagagaacggggagtatgaaaaattggagttcttaaccgatatttataagcatacttatacctatactgtagataactatactgtttattatgtaatgattgataaggttttttcctctaacttagtcctatataagaatgatgtaatggtaaataactcctatatccatagcagagctttctgtgctgatggtgagttagatgagctacttaataagtctaatagtgtacttgaggttattgactactattataataagcatatgaaagaagttgctaaagagaatgagaagaagcggtttttaaataaattcatgagtagagataattaataaagaggataataagttatggttagtattagagctgttataaactatattgtagcaaacggagtagaaagagttgaagaatctaaggtatctcatatactagacttgggtaatggttatagtattgaatacattattcccattagaatgtgctattatgagggtattactatacgtaaaagaggctttattcaagagtttaaaggggatgctattgattatttagatgctcacttgaagaaagaaaataacatttacgaagtcattgacttttatgaatcagagtctattaaaaaggagtctaatagacagttagaattagaagaagtaaaaaaatttatgggtagaagagaataaaatattgtaactatatttactcaaaggtattgctatttaataccgtaaatgttataatgtatatagtagcaaggttggtatatatctaggagatattgaaaggctatattgacaatcacattattaattatagatagtataaaggaggaattgtaaatgaattataaagaagtattagaagttattaaaagtaataaaccatgtaaggttaggtttactggtagtactttgttccttgctaataaggaatttaatgcagatactaatagaggtatattacagattggtgtatctagtattaatacagacgactatattaagttacagcagtattgtttagaaagagatgagtataccgtagaaggtgctattttattttaaggagtgattaagtatggattatagagattttattactgattgtattagtggtggttatagtgtgtgtattaaagttactgataatagagttcatgttatctcagaaatggaatcagtatcctatccaaagaaagaaattaatttatataacttaaaatcttacgtttattatatgggtaactttggaagtcctattacaatggaggggttataatgaatagggttaaactattaaaattaatagagaatatggcagatataaataatattgaaattacaagtgtaaacttttatccagaagcactagacattaacaataaagctaagatttgtatgggtagtaattatgtagttcataaagtaaacgagggtggtggtaaaagatgcttgactgtagatgagatgttagatgaagttttagagttacttaaaccttcttttaataagggtgattatatcactaaaggggcacaatccggattacttattaaggtaggggtaaattcttacaatgttctaaatgatgacaatgaaactatctattattatcctatctcgttaaaagagttagaatatatgggatggagaaagagggattagctatgataagttcaagtcatgctaagtacctatcagatatgtataaagatagtgtacatcatgaaactagagatagtattgtagaagatgtattagataatattaatgatggtattattgaagaagctatgagagggaatacaagttatcaatatgttcttagggatttaagagtagataatgaagtagaagatagagttatagaagaacttattatctcagggtatagtgtaaactatgttaataatagcacagagtatccttttctatctacaaataatttagcaaggctagattacttagagattaaatggtaaaataaagggttactctatggtattacattagaatcattctaatctaggattttaatctttaaaccctaggaaaagtagtactaaaacatagaatcccttaggtatgggattctcatttttaaatttatgaaaaagtatatagtatctcctagaggaaagttttaatgtacttaaaatagccgattacttattatcatttaggggtatctaattgataatattggaagatgaccgcttctagtacaagtaaaggagattggtattgtgtttaatagaagaagggtaaaagatattcaaaataatagtaaagaaaaaataaacgtaggacatagtatacagttaaaggaggaacaagaacattatagaagattaaatactgatacttataccatacatgaattagataagattatagaagagcatttaaataatctagaagatatatctgtaaatgacttggagatagattctatttggttaggtactattggtatatacgactacccaatcggtcaaagtatgcaagagcaggtatatagtataagagaacttaagtatatgaaaaaacaatttaagaaactagggtttaaaacacaaatatcaaatagtgatgtaggattcacaccttacgtttatttaagattactatggtatatataaggaggaattatttatgtttaaaaagtcaaaagaaaagaataatgcatatgttaatttagaatatagtagaaatgaagataatcatgaaccacaaaataaagagttagttcagttagttagggacattactgggggttttgttttacctcttatagatggtcatgagtatgtagtattagttcgtggtactaagacattagtatttaatattgtaggtgaaagaatctacctaaatggctcaatgctaggcaatgattttgcttatgctgataataaaaataaccccgtttctattgaagtagttaaattaatttccaaattaatgtaaaggatttccgcttctattattgaagcggttctattatactctattatagtctaattctttcatatttaacgtatacagggttttaaatgtacctaggtatatttatatcaaaaagtactatcgtttgttatacgccgtctatgaagcatatagagggtattatatttcattaagtgtagtgtcagctttcaggataattttcttttctgggtgtatttatacctaaaaagtattcttgttaggtgacttatttatacatatagtatatatactatatgtatacttttataatttttttttattctatgcaactcctattagttacagagttgtagagtaaataattgaaaatagtgattgacattatataaggtatagtctagaatcagtagtagttattactgtataatattggagtgttggttatggttagtagtattataggacttattttaataggaataattagtgtagtattagtaagtgtgttactgtttatgtggtacgtagtaatttatactaaagataaagtattactttcagttgctatattactaacagttattcttggattaattggtgtaggttctgtactcaattactttggactataggaggtgaaactgtggttatatttatattgcatattattacgattattatcctagtattatgtttttgggtacttctttcttctagtagtcctttacatttcttagcatcggtatttgtgtatattactattatcaatatagtaggtatgttagacataagtaagtatactttaaatgtaataggattttttgtaatggctgttgggttattgattcttttaattagaacaaggatatataggtaggaggtaggagttagagttatgattagtagtattatttcaatattgatttgggtaatcctacttatgtgtgcagtagctatggatagtatttcctagaattaaaatttaatgaagataagttgagtaagtagaagcggataaatagaccgcttcttttttgttgacagaggttagggtaagtagtataatgtatgtaggaatattttaggaggttgatggataatggtttctatctataagtatctaaaggattatgacacaacatttgaatatacagttaatccattgcataaagctactatgattggtatatatggtagtaaagaaaagagtagaattgaatatgaaagtattagtgaatttgtaggtaagtttaattttccagatactaataaatcatttaaagcagagttacttgattatggtaaatatttaattgatgatgattatgagatagaaagtgtctctaagttaaaagacttaatctataactatgctaatatagaacaacacaattcttatttaaaactaaaagattaatctaattgataatagaatgataatacgtttcggacgacagaagtaatctacaatgaattatatacctgtcgtccgaaagccaaaaaggagtaaaatagatgaaagttagagtacatttacacaatactaaatataaaactaaaccagatgaagataaagtaggaactattagtaaccaaataagtaaaaaacaaacaagtataaatatagaagatttagctaaagaattatgtaatggtagaacagtattatttggtataatgaatagtaatggtattcgaaataagtttaatgttaatcatcaagaagtagtagcattagacttcgatactgaagaagatatgtctattattaataatgagtttatacaagataatgcttctttctactattatacattcagtcatgaattaaatgagcctagatttagagtagtatttgtattagatgagcctttaaaccattactttgaaattactcaagtatatgaatacttattaaacttatttcctactgcagataagaactgtaaagacccttctaggttattttttggtggtaataactacaaagaaattaattttaataatactatgagtgttaagggtttgattgattatagtttaaaggatacttatatagaggaactacctacttatgaccttattagataccacaaagatgatttagtaaaaatgagaataggtaacacttacaaagcctctgtatacagcagaactaaagctatggaatacttatatagagtagacatgagaaaggtattaggattacctaataaatctaatatacatgatattcttggttatgatagaaatcctagtgttaatgtttggaaaaccgaaagtggagtctggttatatactagaatgtataaagacagagaaagtaaaactaggaatattattcaagttattcagaaacttcgtggtgtcaacatgggtggtgcaattgattatcttatctatatactggggatagactttaatgtaagtgataaggttagaatctttaatgaggaaatagaagagttttcaaatactttattatctgaaaacttaaatcttacctatcctcaagttgcaaagatatttaaagatggtagaagtatgtacgcctcagatgtggttagtattctaaagattatgcaaaataatgttattgaaattgatggagaaattagatttgttacagatttatcttatagagaaattagtaatagaatctatggtcacgaaggtaaaaaagatagaatagcaaatgtagttaacttaatggctacaacagactggattaaaaagttagggtctagtgaaataccagataatatgcttgaatatttactaggacaccaagaaactaagaagaacaagtatagaaaaacagttttagagtataaaggtaagaataaagatacattctttgagatactagaacaacagtgtagagttatggtagaaaataattatacaaccgcttcattatcaagagaagggttagctaacctacttaatgaggataaggctaaagaagtatataatcaaagtgaattccctgaaagtaaagaaatagaatcggatattatacaatacattatgaaagaagttaatgataaaggatactgtttagaaaaagatataataatatattttgataataatagtaagtacagtagaagttatctcgagaaatttatgaagcggtatagaaataagttttctaaagggtatgatttaaaattctcaagattaaataagaatgtcaagaaagagcttggtttgttagatacatttaatagtaatcagacacctaatgcttactacaaatagatttactactttcaattaattactctttacctattgacactcattcctctataccttataattaaactataatcaaataaagggagaagataacttatggaattattaaatagaattgaacaagaaggttttgaaattgaacatattgaagacggggaatatcttattaaagatattaatggtaaggaagcggtacttacgatggcagatgggtatgttattgtcactaataacgatggattagaaactcatatctgtgatagtaaaccttcattaattatcagtgagttattagatgctatgaactatgactttgaaaaagaacagaagaaaaaacagttagccaaagcaagaaaaaataagtttgatgatttattagaccactttgattaatagaatcaattttaaacataattgaggactggttagagggtgtagaaccataggataaatcatttacgataacttaccttgagataagttgagatacgataggatgcaataacttaggatgttaagatacctaagtataagtttaatactatcattgaaaaggagatttctacatgaataaagagcaagcaaagaataagttatacgatgagttactttatacagtacgtgagtatggaggtacagaagcggatttattagagcttattgatagttttagttatttagacgggtgggctccgatattcaagaatccagaaagagaacaacactattcaattgaacaatttcttaaggaacaaaagtctgttttagagaaatacctagataatatagatgtagtatatagtaaagaagtaatcaatgattatgtactgtataggttattcaatgattcccttatcataaaaccagatgaggataagtataatgtaaatattaaagtgagtgataaggagtttattacactttccttattatcctttacggatataacagatattattataggtaataaagataaatttcagcaagtataaattaaatccctagagtgaaataactctaggggtttttcagtaaatatatattaagtattctaatcaaaatagattataggagaaagttaaaaatgactaagtacgaattatctttcagaagtgaaaaatgactaagtagagattaacttttctacccgaaaaagatagtaggtaatagattcctaaatattctgacaatttagacaaaagggagagcggtcacaatgaccgcttccaattacttaatttttttatataactctgataattctttttctaatttttcaattagttttgtgtcattattttcttttgctcttcttaacttcatttcacggattttaatgagtccgcttcccgcagtcaagtttccttcaatattgttatctttcaattggtttaacttgctttttcttccgtctaattttaaaccgaaattacttttcattttcatcaattcctttatattttatttgtagtgatttccactacacttattataatacagtaattgggaataaaagtaaagcattttttaggtaaaattttacacttttaaatagtcggaattgttttcatatttagaacaattcggaattgtctgaaaatttaaacaacttaaataggtcatcaattccattataatataaaaatctacccgtttcaacacttaatttatttaatatgttgtgtattatacatttaatagttcgggtcgtgtttcttctatataatagtaagaaaaagaaatttaaaaaaaattacccaaaagtgtttacaaagttctgggtctatgttattataattatagaaagttaatcaaggaggaattacaatgaaacaagcacaagcaattaaaaaattcatggaattaagaaaagcgctaaaagaagaagggtataatatttcaaaaaaacaattactatcaaacatggaaatatggtatctagatttaaaaattgattcacaaatcgaatggttcattaaaacacctaatgatgttatcattaaagatatggaagaattcggtgacttgtaatatgattaaaattagattaggtaaaaagataatcaaaagcaccgaaaaaactgacaacattctcattagtctaggggcttgcacacttacagcaattatgataaagttaatacaactacaaatgacgttaccacttgaaacgtggttaagataagaaagggattgtttattatgagtaaaaaaatagcacacgatataatgaaagcatacgactttgatgctttcagtgataacgtgtttatgcaatataagaacaaactaagtgatacaaactatcatctagaattgaatgtatttagtgaagattggtttaacgggtcaatcgaattttataagaaaaacaatttcttcaaaaagaatagaaatttaaaaacaggttgggcaacatttaaaaatggttgtacaatcttaaaggtaggtagcacacgctatgttatcttaaatggtgtacttattcaccaaaaagaaatttaaaaaaattgataaaaggggtttacaaaaaccccttaccatgttattattaatatagaagttaggggaacgacacaaacaaaaaaaaactaaaaaaagttttcgaaaagtgttgacaacctaaaaacttcatgataatataaaggtacaaggtaaccaaagcacaacgaaaaaaaaataaaatgtaaaggaattgatttattatgacaaacaaaaaatatttatacgtagaaacgcacacagtacaaggtaaagagattgaagtgtttagaattccaaatgacacaaacggaaacccacgctatgtggtacattttagagacctaggcgttagcctttgggattacgataacatcaataaactatttggctttaagaaatataccgcaaaatggtttggtggcggtgtagtattccaaagtttcaacatagctgagactttagaatacgcattaagtgaagttaaagaagcggttaaatagaccgcttctcacattaaataaaaaaattatataaaaagggattgataactatgaaaatattaatagaaacagaaccaacaaaaacattaaaacaaattaaagaggacacaagatttattaaaaatgaaattgctaatcaacttggattagatagggaacagtttaaattatctaatggcttaatattattgagaggttactccaaagaaaataattgttgggttaatttaactaatgtaataggaaaattcccagagtatttaaaaaatgatttatattatgaatataaattatattgtaatgtagaaactaaataaaaaaattataggggttgtttcttatgttacgtaaggaaattttagatatgctagaggttaattgtgatagctatttatcacaatatacaataagaaaagaattgaatttaaatgaggaattttatgattctactgatttaggtaaattatatatagaaactcgtttaaaatctattgagaaataccttaaaattacctcgttagattatgaaataaatttatatttaaataattattgttatgatagtgataaaatctatttagtgattaatcacgagggaacattgattaatgttacaataggtaaagaacaattcatcaatgatgagctagaattcatttttgatttagttgatgaagaaattaatttagactagaatgtattttctagtcttttcttattattttaaattttcagaaaattcataattgttcctacaaaacgagcctctccacttcctattatacgggaaagctccccgattgtcaatacttttcttcaaaataacttatttaaaaagtttcataaacctattgacttttatatcactatattgtaatatataaagtgtaataaggaagcggaacacttaaaaaaagaaattaaaaaaacttgctaaaagtgttgacattcattttgatacatgctattatagtattgtaaggtaaagaaaaacaaaaaacagaaaaggaattgattaaaatgaataaagaaatagtcaaagaaacagcaatagaatacatcatgatgatgcaaaacgaggtagaagacgaaaacatctcatttgaggaatacaaagaagcggttagttttgagagcgtttatgactacactttatcagaagctaataaagaatgtaagtttctaggaggtaaagtcttagcttccttaattaatgaagtaattggacaatatgcgtagggagcggtttaaccgcttctttttttgcagaccattgtctaaatattctgaaaacttaaacaacttagctaggtcatcaactccattatataataaatgacctaccaaaagcaatggtaaattttaccttttttatttttaaaaaagttagtcaaaagggtttacaaataagaatatacatgttattataattatagaaagttaatcaaaggaggaaaacaatatgagatacgagattggagcattagttaatggagaattcttcatgttctctacattcaagaaagaagaagcggaaaacaaatatcacgaatggtgtgaattgttcggtatagaaaacgtaagcgtggaggtgtcttaaatgttcaacctcttacacaaacaaactaaacgtgaactaaagcaaatgaagcgggctcaaaaaagagcccaaaaagaattaaagaaaattgtaaaaaagtgttgacactctacaatatacgtgttattataattatagaaagaaaaacaaaaaacaaatttgaagggaattgattaaaatgaaaaacacattaaacaacgtaattgaaacattagaaagcaaagggtatgagttcgaatatgatgaaactgttacagtattagaaattacaagtcctagcggtgcttatgaggatatcacaccaaggtttatccgtgacggaaaagttgagagtctttttattattcccgactttttagatgaggatttagggaacgtatgttttgagttttatggttggactttaaacttcacaacagctaaagaactaataacagaaattgaaaatatttttaaataagtgttgacagtcacttgaaaccatgatattattaagatacaaggtaagggaagcggtcaaccgcttccaacctagtaaaaaaagttttaaataactgttgacaacctagcatatagatgatatacttgtattaacaaattaaaagaaaaggaattgatgataatgaaaaattacacagtacaagagttaagagaaatcgtagcagaggttaacgaatatgacgacagtttagaagagtttgactacatggatattggaacacttgatgaaattcttagcggtgtagaacctactgaggttctaagaatggcacactttggagaatttgattggtcagacgattatttcagaatagacatatacggaaacctggaaagcgttaaatattttgaatttgaagagcttgtgaaagataatcacgatgaaattgtaaaacgctacaatgaacttgtgaaaggtggagatattgaaccaatcgaatttatttaatcattgttaaggtatccaccgcttaggtggtgggtatctcatagagtgattaaatttaaaaaaagtattgacactctagaaaatagatggtaatataagagtatagaaaaataaaaaaagaaaaggaattgattattatgaacaacacattaaacactatcgaaatgaacgtcgaatacttaggcactaagtgggtaaagggttgggaacacaacgcctataatgtagcactaacacataatggtgtcacagaggagtttatctggaagcaagggctaggaattcataaggagccctctcttgaaagagttttagaacaccttattaaagagtcttattattatgaagaggacatttacgatatgtatgatgaccctgaaatagcggagaaagttatagaacagttaaaagaagaagaaaataaattaaataatttattttctgaatatgaattagaagagttctactccttttactttgaagattagaattcatagaagcggttgacaaggaggagttaaatggaagatgtaaaccaacttttaaataatggtggggcggttgtatttgatagtacaaatatgaccgactacattattacaaaccaaggaaatatttattctaagagtaagaaaacaggagacctgcacctaagaaagcttagaactgaccgatatggttaccaatcaatagttatcaaaaataagtgctataaaccacatagattagttgcagaaaattttattccaaatcctaataactattcagatgttcatcatgtaaaaaacaaatataataatgatgttaatagtttaaaatgggtatctagtgaaaagaataaagaatacttttataaagataatttcggtcaattaaaaactactattagatataataaaagtttaaatattgtcgaatatgaaagctcatacaatcataagaaagaaaaagaattacttcagaagattgaacagttttctaatgaattacatgatttactaaatgaagcggttaaaattgatgtcaaaatatttaaagataatagttgacaaactaaccgcttcatgttattattaaattgtaagataaaaaaattataggagatgatttaaatgacaaacacactagaaaaagcaatcgatgctctagtaggcgcatcagcagatacaattgaaaaggacgttttatactggttaactggtgaggacgttgatacgctagatgaggctattaacctccttgagaatgtatcatgtgaagcgggagacgtaaaccaccttgtatacacatctgattgtattaattgggtgctagaacacgaggaggctattattgactttcttaatgctaatattgatgacgaggtagactcgttagataatatttcttgggagcaacacaaccaaataaattctattattactggttgggaatctggggacgatagagtaacgtggtttgtagatgattattacgaacacgccaaagagaatgcccgagaggacaacgagaattgggagctaatgacagaggaagagaaagaggaggcaatcagtgaggaaatagactatctagttaaattacacgaaccattagaattaacaagctcagacaaggctaatcttgcatggcttacgttcgagcaaacagcttgggacttagcggaagaattagaggaattaaagaaagttagttagttaggaggactattcctcctcttttttttatagggaattgtctaaatattctgaaaatttaaacttttaaagaatgtggattttttttgtaaaaaactattgcaattctaaacgtaatgtagtaatataatagatgtagaggagataacctcacaaaataaaattttaggagttggtaattatgaaacaatttataatggcggttgaatatgaagacggacgtttaggttacgtccgcatgtacttcgagggctacgagctcttagaggtgcgggaggttgttttaggtcacgaggacatgcctccacgctttgaggttgaggtggacgagatagcggactatctcggatttgcctacctcgaggaagaagaccaatttgggcgcttcttcggtgagtggttggctcttggtgaatcttgtgacgtgcctagtgattgcctcgaggacgaggacagatatttagtagaggaggtggagtaaatgacagagggcaaagcaagagaatacgctgaacgtcttataaacgctagttattatctgtgccctcacgacctagttggaatggctagtaagtgggacgacatagcagacgacattagaataatcggcgttgagtgtttcaaaaaagagtacttaaagggtctagaggttgaaaccttagaggtactcaacagatacgagtaaggcggtcaatagaccgtcttttttatttgactttgtatagtccgtatatagtccatatacgcacgctaaccgcttctgtgtactcggtaatgttattgtacctagatagataaacgtttgttagagcgtctatatgagctgtatatgacatatagaatagtttggttcgttggttgatacagtaaagattgaatgaagaaaaaaaatagaaatataaatagggaattaatatatagtttttatttagataaagataggttatataaaaaatataattaataaaatacgggggtcggtaggaagcaacataatccgtggcacattcacatagatacaccccccacttagggtagtatagatatgtagtgtatatacaaactaatatacagaagtcaacatacctctataaagtcaacattacaaatctatcctgtgggttagaaatgtatagtataaatagataacccttaaatataaagtcaacatacctaaaataaaaaaaagggaaattaaatccctcttctcttaggtattattaacaacctctaattcatgtatagtaatcatatccatcctatagaaatctcttaggtctcctttaatgaacttttgccccttatggtttgtttcctctttataaccttcttctttaatacgtttaattaagttctccttatctatgtatatcttatcttctctaaaatgaaagttatcttcataaggttcacaattatcatgttctacttggtatagtttcattagtcattttactcctcttcataagaccattcaccatattctacattatagtgtgatatatctactaattttctatttattattctatttttgatacatttaaataagtttcaattagaggtaagacttgtgataggtattgctcttcagttaaatctaaactattttcagaccaaataacaaaagaagtacccctaactgctaaaggactatctacatcaacaaattcactaccttctacatcatcctctacttcacagaaagtatcagtattaaactgacgttttatataatcttgttgccattctatatcttcttgactaaatttagattcaggtaagaaagtcgttgaatagaaattacaatttgttaagtctctattatcattagcaaaatctttagctgataaaccaccttcagattttttccaataataaacttcaatacctctatctaatcgttttaatccattagtatatatactatcgttccatacaataggagtaacgttattatcaaataagtgttcagctattgtattaataaagtctatataagcaggttgattattacttgcacctgctgactcatcagcaccaatattaaatctcattttatcttgtttaaatagtgagataatttcactaactaaggtcttaacagcagtataagtatcatcattataccaataatcagctaaagtatcatcaaagtctgattgaatgtttttatcaggatacagctcttttacaatacttaaccatttacctgaatgacctgggacggagaaacatggtgtgatttgtacatctttatcttttgcataattaacaatatcttcaatttcatctttagttaagtaatctttattctgagtggtttgggtttgatttagatattcagagtaaataccatatctattgttatctgaaatatagagttgtaagtgttttccatcattatcatgaatagtatcaataacttttttaatactatctttagataggtatcttctagcaatatctaatagaacctcacgtctatgcttagtcttaaagctaggtacaggcgttttactcgaatcttttgtgtaagatagggtgtaattatcataccatacattaggttgtaggtttctaagtttgatactaatattactagtatcttgtttaagctcgtactcagacccttgtacaagtttatcatttaaaaataaatataaggtcttattttggttcatattaacatctagctcctttagtatttagtaattttaatataacaacattagtattacttaatatgaaatgtagattttaatagtatctaacctagtctaatttatcctcagataataatttcaccattttggttattatgctttacaaaaataagactcatggtgttaaagtcttacaagggttcacttttacaatggctaaatcggtcaaaggtttgtaaatcttctgtgtctacaacaaactgatttcctttcttatagatgctaaccttttctcgtatttgtccatgagtacttgtaatttgtaaccttttttcaatcacatagatagttttcattatttaaactctgttccagccttcattgcataggcgtaatgatttctagcttataaccttttaaatcttgttctatagtatttagatgttcccatacctcaatccaatggactctagaatcatcattataaatatggaagtcttgagggttttctaggtattgttctagctcttcatagtatagaacttcaaaagcggtatcgtcaattcccattaataatcttaatctttccttataccctattttagcattataataagtattttctaattgtctttctaccctattaaatagtgtttgaataccttgtaattcatgcataatacactacctcctaattagttatataattagtataaaggataatagttagtatgtcaataattattctaaagtttcttctatgatttgttttaatgtatagcattcaatttcatctaattcttcaagatactgtaactgatagtagaagttatctacaattttatcaaaggcttttgctttcttttcttgtaaagatagttgttcttttgtcgtaatttcattattagtttcattattcgtttcttctaaggattctttggcgttatccttatttatcgttacttttagcttgttatcatagtaatcgctcttttggtttaagttgtagttatcattgtaatcattccaatctacatagatacattcatgtgtttcatcctcatagataatatctatagctgcgatgtcatgaaaactttctaatctttcatatgcagtagtttctccgtctaagctgtcgaaaattaagtctttcgggttattgataagaatccctaaatggctacaatgttttgttttatttatgtgaaaatcttcttttaaagtgcgttgagagacataggcttccgtaattcctccgatatagaataagccaatatccttaggttctaaacgaataacatcacaattctctaacactaaatctacatactttgtattcataatggctatttctcctttacattaattataatatattcaatgaaataaaaaagtaccttacttaatatctttaattaactctttaatgagttgtaatgtaggttcttggtctacatctacaactaatgtatctaactttgctagtgctttttcttttatatatttattttcttttgagagctctacaagttctataatcttatctaacttatctgataaaatgtggttattttgatttgttatttcacaaaaatcacctaagataaatctatttacataatcagtctctttactaacaccatagatatagcctaacattctactattatcattagttgttcttctagtagattctgaggatgtatatagttctttaattttgtaaaatgtgtcggatatatacttggcatatgtttcgttaaacttcatataaaaattcctttcttggtactataatattaactctatcccctaatactgaattaatatacttaatatttttcactctagttcctccaatttttccatcaatgtcctattccaacttatcaatcttttattttgttcttctaataacttcatttttttaatgttactatttaataaatattgtttatttttatagagtaattcaattaactttaattctttgagtctttcttcctctatatacacttttcccatactatccctactttagtttttttatagtatctattactttccttgtatttctaatatcagcattctttaaattatttaatagaaaatttaatttatttacatagctgtctactgtggaaataaaaacatcttccgtatcattatactctgaacgcatttctaagaatatagttatattgtttttatctgagctgtgaacgtcagttacaatagtgtctaaagggtaagaatttttgtctacactgaatacttcgtatggctcattttttattagattatcatagacaaaatgtccgttatgccttttaatttctatccaagaactgtatttgatgtatctttgctctttaataaaatcttctttagttatttcattaatctcccaaataagcaattcgtcatatacttctaaatcatagtttgattgtatatattctaattcttctaggttagtagaatatccaagtatttcagtattacttgtggtatagtctatatactcatcccaaccacaatgtgtagttgtacactccgtttgctttattagtgcatatagttttgtcataataacctcctccttaaacatattatataataaaaaagaacctgtgtcaacaagttctttaagattaatcctattaacttttttactaaattgattatatactcagtatatccgattacttcttattttctttttcaatcatttccattctgtcatgcaacattctgaaaaactggtatctgtatctatctgttaaagcgtatttatccatgcattccaatgtctcacgatacaattgtttccacatattttcatatcccattattttcacatcccgttatttttattgcctacatactttgggtacacatcaataattatatccatatctgtacctactaaagctcaacatacggtgtcaacattttaatagtggctactaccttatctacgtcatcactaaaaatatactggtcatcttcgtaatgtttgtacccttcttttaacaaagaatattctgcttcgtttagggtgtgaaaaaatgagtcatctgtcgtaaaatctttctcaccaccccagtcataaattctatactcaataatataaaatctcataatattctgtcctttcttgttacaactaactaaccggaattttcggaaggttttttagtaattttctagtaggctttcgagctttcgagtaacttattacaccaatagtaaattttaaattcactgcaagtttaaataaaaaatattatttatcttataatcgttagcaaaagcaacatcaccgttgtaaattggtctgttttgacaccttttctttttattaagcccaatctggcattttgcataaatgtatttgttgtttagcattggttcttttaagtcttttctttgaatcaactggtttagacgtatcctttttctgtgtctgctgttccatcttctttttgtaagctgtgattagttggtcgatagtgtaataatgattggcaattgcaaatggtaaaaataaattgtaaccacaccagcaaaacactaagtcgtttatatcaacaaggaaactatctaaaaccttaaaatcatcaaaatttacattctcgtacattaagaagtcattgaaacttccatcgttaatattgtccaaaattgattctgtgtctttttctacttgattcgcaatactcaatccaaacgctaacaagtctgctagttcatctaactgaacctctaacggtttacctcgtttctttttccaatttttaaacgtttctaatgtgttaaaccattcgaaaaactccgccacataagcaattctgctatcttgtaaattaagcgtcggtattctatcgtcgaattccttttgtatttgtaataactcttgtaattgttctactgttagtgtattagtcattgtttgcgtcctcctgttttaattgttaattaatccaaataatctattaatatcttcatgagtatgcccgtcccattttggtgctctttctacttccttaacattgtaaatatcccaatactctaaatcatagtgatatgtgtaatgtccttgtggagttgtaataccaacaataaacattccgtcaaacattgttccatcatcatgttgtttagatttccatgctttttccttatgtgtatttaaaattactgcgaacaacacagctctatcatgatagagttgaccaaaagtgtggtgaccgtctgaaatttcctttgtactaattatatttttttcctttaattgttgaatttgaatgtttaaattattaatagtaaaaaccttcattttactcgtcgtcctctttatttataagccaatacgtataatctaatccgtaatgttttgcaacatactcagacaggacagcaacgttacgtcttaatttagtgttctgtttacgtaacctcacataatctgaaaaaacagccccacttaaaagaagcgttaacacatataaaactgtcatttactcgtcctcctcaaacttttcttttaattttaaatatgttttgtattcttgttcttttcggtatttttgagacttgttgttgttttttcaataatattatgttcaactaatttgtccataatataaattaagctatcgtattctttttttgattcttcatacctcatagaattcactgttgctactttgtctataccagaaatactatactcgtcccatcgttgcagaacgtcaattaaaaaagaagcaagtttttcaatctcatcaaaaatacagtcatcattttctttgataccatatttactattcattaagtctgaatttatttcactcatagtagtcacattccttcatttttctaattaattcttctgtgttactgttactgccgtgtctttcatatgcataatgactaaactgtgatacaaatactttcatatgatgcttacacaagtcgataggacaatacatctctgcatatacttcaccatttaaatatgttacttcgttaaactctccatctgctttgttaccgcaaatatcacatactacctcttgtacttctttggttacctgtgccataagagccctcctataaaaattttatttaatttatttttctaatttaataaagtaactcattaaaccaatttaccatctttccagataagtgtatgtgtactatcatcgttaacggtgtgtaatgctttaattgtaacttcactatattcatccgaatctaaaagatattttattgacttgtcctttctaatttctaacactgaaatcccatcgaactgatacgttactaatagattcggaataacagttttatccgtaattacttcttctacttcgacggtgaaagtgtcccaatgatagatgggtaccatggtgttgaacgagacgtcgctataaaacttcactgcccttccataatttgatgtgaaagtttcacctttcacattattctctcgagcccattgaattaactgatgtaggttcatttccttcttaactttaattttcaatataaacactccttaagtagttatttcttcttacgttttttaatactatattttatgtttaatgtcttattaacttgcttaagttgaagatatacttctttccagtttactaagtattgataagacatatcttcgtcaatcataaatgatacggggtctttaaaataatcatctagttcattactctctaatacactaaatgggacatcttctaaacccatttttctttgtaaaaacttgaagtaattttcaaaatagtctttctttttaagttcatatttatcaatttttttcttcacttttgtaaatagtttttgtaattttttacgttcttctttcttcatttttttaaacctcctatttgtttatacattaagtatagcaggtaagaatgtataagtcaacacacaagaggaacttttttaatattttatgtttttaaatgattctttaagttctacaaaggtgtagtacttattatgagccttatgcttttgactgatatctacaggctccattgtattaataaataagagtttatcgataatatagtgttaatttccttcttaactttaattttcatcatcgttcctcctcatattcaatctttgatggtaccgcactgttaaaatgtataatgtttccgttttccaacttgatatctattccatcattttcatggtgttcaatcgaaactactttttcgtcaacggtgaaatgcccatacggtgataatcccacttcatacacttcggttttgtagggtaattgtactgagtatgatataatattcatcatcgttcctcctcaacgccctctctccagttttcgactgaaacaatgttgtacataacatcttgtaaattatccaatatttcaccatcttcatcgatataactttcgaattctcgatactctgttgaattgctccccttaaaagtagcgccagaaccatttttaatacattgaaatttaaagtcatatcctgcataatttacgtgacattccaattcgtcattctcatttttatagattttaatcatcatcgttcctcctctaacaattctttgttttcgtaaatattcccaacaacctctaaaagttctattctctcgaataaatcttccacatagacatcccactcaatgattgttttagattcttcaattagaactacaccatattcttcatccatgtcgtcccacacaatatcaccttggtacacctcgacagcatgcttgtcagtcaatcctgtcgactgcataagttcataattaaatatttgatgtaaggaacttgctaatgctactataatttccgacccgttaatttgaatggttagttcatcagggttatacatttcattctttcttttattccacactctaaatttcggtttcatttgaccattctcccatctttccaaataagctccattatgtagtcgtcgtttaatatataaaaggcttttggtaacgtatctgcgccttttacaacttctcctatagcctctcttatcgacgtgttatagtgcgtgtgtataattagtccgtaatcacttacaaacaactcgacaagagttggtatttttgtatcttccttaatttcttcttccactttgacgacgatgaaagtttcgtcaggctgtattaactcagagtaacagagcccttcacggtcaaaatccacttctccaccgtaattccctgtaaaacttctattcttaatatcgttatcccacgcccactgaatcagctcgggaagagtcatttcctttgtaactgtaatatacggcatagttagtcctccttatataattatttattgtaaaagtctacaagaaattttaccgttttattttttaattccttttctactacttccatagtttctttactaattttaatgttatcctcattagatttaattttatcttttacctcttgtattaaaggttctactttattaattaaatcttttttcttttcaacacttacactacttctcttgttatctaatacttcttttgacatatacttaacttttgcaaagtctttatagctaacatttaagttatctaaatcatctaataaatcattataatattctaaatgattatagaatgtgtaaaacttaacaaggtctttaccagttaattcttctttttttagtatattattgatattaccaatgacagaatatgctataggcttaaaattagctctaacataagttaaaaatgtaaagtcatcataaaataaacttaatacctgtgtgttaaatgtagtgtttttagcttgctctagttgagcgcatagattaagaacattatcaaaaccactctttaatatcaaagaaataaatctttccaccgcataataccttgattcttccgtatgtttacttgctttttcattgttcctaaaaatagtatcagataaaggctgtactactaaactcatatagtctttgtccgaatactcgtcagatgttccttggtaagtacttccgaattctattgtagatagtacaaaacttctttctaggttcattatatcaccttaccttatttgttagtataaccttatcttactacacaaatttcttaatgtcaacacataatttataacatgttgtgttattactataaccagtatacattaccttttccattttgtcaacaaaaaaagtatctacaaattaatgtagatacttatactataattatagaattttaggtgtgtacatgaaggataactcatgtagggtttagtagtaagatacaaagcgtctaccacgcaacatacaacatctcttgtatgttattatatacgtactggaaggagttaccagtaggaacctacagtcatatacaaagcctatgccaggcaatatacggtatatctcgtatatcaggtataataccttatagttattatcttgtaacctataacttccgataattagggttttataataactactgtataagtaactataacctataagttttttgtatactagggactataatggactctgtaggattcgaacctacactccaacgcttatgaggcgtttgctttaccattaagctaagagtccataaattatcctgagagagggcttgaacctctaagtaattactttcaacacttaactatatataatgtaacatatttttaataataagtcaagttatattaataactataatatttatccatttcttctttatacatttcttgtagtcctctatttataagatgtttagcttcttctttagttaaacagcttttatcacttacttcttttaaataattatctttttctaactttaagataacctttaattgttcactagtggctttgttaagtttttgtttttctgtatatagccaattgttaaaattcatagagtatcttcctccaattgagcatttgaataatgatataggtctttacaaatactttttgcataacaacgaatagcataattactgtcatcaatgtcttgtaggtacagactatggactaaatcataagcataaaacagtaaatgttgtgtttttttcttaccatctctaacttcaaaaataattgtatctaggtttttaaggtctagtattttaaattccttagttgtattcagatttttagggaatttcatccacccatgctcatacatgatttccaccgcattactgaattgtcttttagctgtgtctttattcataataatcaatccctttcttaatttctatactaagtataccaaataagaaagggattgtcaacaataatattaaattaattagggtgtttctttatattatctttatagcaaaatatacactctaattcataccctttagttgcggaagtgtctaaaaatacccctataaaaatttttctataaatgtatctatatcctcttttgtaatgtcttgttgatgaagtaaagttgtatttacttttgtaagtatcatgtcatacatgtccactaaagaattatactttttaaactcttcataaggtataataacgacattagtctcttttatgactctggctatatagtagtactctaatttatcttctgaagcatgaactgtactattgtggtctattacaccgtgaataagctcactaaataccctattacaaaagtaggtaattaaatcattattcattgattagctcttctttaattaagtgttgtttaagacgttctacatagttacaaatcttttctacttctttatcttcatcatctttacgacctaagcgtactacgtatttaatgatgttaaataacaaagcatctttaaatccttctaaatgttcttctgtaaaactaataacatctacttttgaattattagaatagtgttctggtactgtaatacttggttcaatattatataattctacaagttctttgacatgttgttctagacttttaaaatgtctttcatgttgagatacattagaacaatcctccactattaatgtttttaattgtagtagttctttactctctttacccgtagattgatacttttcaataaattcttttgattccatttcataacttcctttcagaataagttacctatattttaccacatataataaataatgtcaaataaaaaatgaggagctacaagagcccctcaccaaaggaaagattatgatgttatggaatgtatctagttgatacatttataatataacattaatctaatccggtagaaccgaagcctccacggtcactgtgattttcaaatttatctactactactggttcaggatacactacgggagcaattactaattgagcaatctttttacctttttcaattttttgcttagtgcttccgatattatctaccataactttaatttctgaattatatccgaaatctacagttccaaactgaactcgtaagtctgtctttgctgtaatacctgacttaggtcgtacttgagcttcataacctataggtaaattaattgcaatacctgtaggaactaatgctctactaccttcaaagacacatgtttgtgtggtgttatcgtctttcttcttacgtaaagctctaccaatacgttgtaatacttgtcttagagattttctacaaggatttaattcaatatcctctactgcatataggtctagcccgctatcataaatgttagcacgtactggtatttttgcatcattatgtaacaatttaatttctaatttttccatcataagtctccttaagtaaagtctttaatgtcataatcaactagtactacatcttcaaaatagtcggagtgcaatactaaagagttttctaatttgtctctaatctcttgtgtttcatctccattttctaagtttactcttacttttgctttaattgtgacaatataagtctcttccattatttaattattcctttcaataaataatatctctctagtatctccttatatagtttaagcatgtcattatctaaatttttatcttctaaatatataataagtttttccacttcatcttcacttagtacgactaccttttccataacacccctccttacttagttatcataacataaaaaaagaacgctgtcaagcgtcctcttcgtgtacttcatatatagtttttttaattatacttataggtttatatcccttatctaccaatgatagtacacgtttattgttcctaaagttaatagtatttgggaacgataaagcagatgtcacgtcagaagtataatcagtaggattgcctagatagtctgttgctttaatatacatacttccatctgtaaatactataactttttcgttttctacttctgttcttttaataatcataaaatcacctaataacctactattagtttactacctactgttagattaatttctttatttaattcttctatttgtttaacagtagtattatagtcctttgcaatagcttttaaggtatctccctgttcaactatatgtataatagggtacctataaataagacagtagtcaatgctagttaatgatactgatttaatattgttaagttcttgaagtctatcaatacatacaccatatttcttacttaaagtccataatgtttcccctggctttactttgtgatagaatttattatcaactctttttaaatcctttattaaatccactattacgaccccttttttcgtaaaagtcggcttggaagtaacaaattagtattaataacatattaaccagttcagtaataacaatatgtggtgtcgtattagttaataccatactaaccgatagtaaaccaatggcaaaccctataataagatatagccatttgttagttccatcagactgtttagttttatagaactgtataatctggcttatgtaagctagtataatagatagtgttgctactgtttgtgttaattctacataggtaccaaaaatatatagaaataaagaaaagataattaaaaataaaaatataaaccaatctttctttctatacactactacaagtaaacaaacaagtcctaaaaataagttaacacctacagaaataatttggaataatgttgattctgtatgtagtaagttataaaaactaatacctacggtggttataataaggtaccaaaaccacctagatacaccattaatattaccatcttttactagagactttaagccaggaatatacccaatcgtaattaatatagcatacatggaacttaaaattaaaggtaaattactcattatttgaagcacctagtttctctaattcctctaatacctcttcccacgaaatgaaacctttaccgttggttaatgacaaaatcatgccaaatacaaactgattagtgctaaattcagctctatcagggtcattataaggtttaccatgaccttgacgaatatcagaacaataaattaaaacgggcttatctacaacattataagcatctaaaatatcatattgtgttaatgcatctaagtctctaaaatctatatcttcatatttatcaataattttttgtgcttggtgtttcattccaagaataattcctagctccgctatcgtgcctaagccttcgttgagtacatctaggacataaatatctgattcttgcattgctttaaagtcattgtttaagattcgttcagctagtccttcttgtatagcatttgatttatcattaattgacgagtccttatgtgggctatatggagttatcccaacaataccttctaccctatcatgttgtttttctctatactcaatcattgcttgattaagtaaatgacctcctaagtatactactttgtctttaatatagtttgccactatcatatttctcctatcatttaatatatttttgaacataattgctgtattcatttaattcctttctgtctggatacttaataattaataaacccataatacctaaactaataaaaggtaatatggttactaagcctataaaaccaactgtagtgtttagaaagaaaataagtgcatctaccgttacatatgtaagggaaataacaaatgatagaactataaaaataaaaaatttagttaaccaatctccccgttgtacctttttagacaattttttcatataaaaaccccccttgataataatatacaccaaggaggatatagttgtcaagaattattttaattttattaccacttaaaatcttcctaaaaattagataattcacttgttatatgattccaacttttaccagttctaatatttgatatggtggttttacaaacattgtatttactagacagctcggataaagataagtttgaagtgtaaatatctacgacttcctcattagttagttttgaattatgaacatcttgaccttttgggtgttgtattagtcccgtccgtatagcatggtttatattatgttttctattacaccactctaaattctctactctattatcatttttattaccatttatgtgatttacttcttcataattattttcattacttagaaaatatttagctactaatctatgaacatatagtatttttcttccttcagaatgtcgtatagttaaatataaataaccttttaaattagggcttaaaacccttttctctccttttcttcctaatacttcaccttttttatttatccaatacccaggaaatcccataatattatgccaaagttcaccatttatatgtttatacacatgcgtatcattctctataaatttaggtgcttttttgcgttctctacattttctatggcagtttctcatactatcttttctggtttcatagtattctccacatataccacaagtaccaataagaaatgttctattatttcttgtctcttgatgcgtaaattctgttccattctctaattttattttattgtctaaatttttaattatcaacacctcattactaggtatttattctaccattctaattcatctgcaactctactaatcttacctctaaagttaattgtaaattcatgatgtttagcaaaatcttgttttttaaataattctatataaggttcaaatccagatttatttcttttaatatcagattgctctggctgaccttctacaataacagtacagctatcatgcatacgtgttagtgtttttttaagttctccacgagtaaaattctgtgattcagaaattataacagttttctcttgtatattgctacctctaaggaatgtgtgtaatgtttgtgtaacaaaggcttgttctatcttttctttatctatattagggtctaattctagtaatgttttaattacttgcatagggtttaaatttagttcaataagtgcgtcatgtaatggagagaaataatccatagcctttgtatttaagtcacctggacggtgccccactgagttttcttctgcaggactcataataaacataatttcttttcctttatttaaatagtctgcgtaggcacacacaatacttgtataagttttagcgctaccactttttgatgagttcgttaagactagaatatcatcatcaaaaaagtcctccgcaaatgctaactgctcatcactagccttacttaaaaaatcgtggaacacgctatgttgtaacatgttatactttacgttgggataatctcttaacttatattctaactctttagtcgtttttgccatagatacacttaccaactttcacagattttatagtatttcacctattaatataatatttactttagtataccatatatcaagtattaatgcaacaaaaaaactacctaaattaataggtagttaatatattacatcccccaagcacttaatccttgagcttgataggctcttacagcactatcaatttgtccttgtacggttgaagtatctccccaacctggcattgtttggaataatccactagcaccgcttggattttgagcatttacttgtccgttagattctcgagcaatgatatgttcccaagtagatgcgggtactcctgttctatcttccatttcttttgaagcagataaaccagttgcacctgctgtatttccgttacttagttttactgtacctgcttgagtaacgttatcttgggacgtgtgtgcggtagttagttgtgtattattttcattagtatactctacattattatttgtgttatcttgttctaaagatgctaattgagaacttgtattatatacttgaccctctgttccgtaatggtaatcataaccaaaatacccgttataactatagaaatgataagtaaacccatctaatacaaacgagaagtcatagttaccttcttgaataggtgtagttactaagtcctcggaattagtttgagccttatttgctagggctacataatcaatttcatcagcactagctttatcagagatacctccaaacgttaaagttgctcctaaaactaatgatgctaaaattgttttcttcaaaattaaaactcctttatttttagttacatatatagtaacatttttttatttcacaagaataatgtagcatactttagttacaaactaattacttaattattacatattattacactaaactatctttagctagaatactaatagtatcatgggtaaaatcaacttccatatgttctttaaaccctctagatatacctgtataaataactttatctacttttgtgtagtcattatctaagacatatctaacatgattaataaaactatccaaaggtaatcttactgtaccatctttcataaatacaataaggtcacccataggtaaattctctgttagtagatttagttttaccatatctttaatagagaattctactccatctagctcactaatgctaggtacatgtcgtaagttatatgttttattgttgtttgagacatttttagtaaaaaacccaacagcacctttttcttcatagtaaactgccatatagttgccacaattagattctaggtatacatctaattcttctggtttataatcggaagtaacacttaatttttcactttcgttctttaagtattctgaaatttgttttacattaatagtaataacttcttttgtatcataaatataaggtttcataacaatcatccttttcctattgattttttagcaagctcatcagctaaatcattaaatttatcattactatgagctttaacttttttaaattctatttgaatatatttagagtattctcttattctttctatatataattgacttaaccaattcctagcaacccaagaaccttcataccagttaatgagtcctatatagtctatatggataatagaaattttataccctaattgtatagcttcttctattgcataacatacagctagaatttctcctgctacattattaaactttgctagtttttcattagatacattgttagaaattgtgtgtatgattttattatcatctattaacactgctccacaacctacctgttttttcttataatcactactgccatcggtgtatattagtacagtattgctcatatctacttacctttacctgggaagtaaggatatgtacatgcacagtagacattttctataaacttaacgacagctttgttaaagtcaaaacacttatagcaaaattcaagagcttcatcttcactatcagccacaacttttacaataaaatcatctaacttaatttcaaatagatttttcattaaataaccccttatcttagaataaatttaataacttctaatggtgtttctagtgtttctaatatagtataatctgtattctctaatgtctctataaactcctcgatgtttatatctacctcttgtaaatattttataatttcttcatcttctttgtgtgttgtattcataaagtttccatatgataaatcaaaaagattagctaaattaactaaattaaatgcaattttaacattatctaagtcatctaaatgtgggttgctttctataaagttatataagcagtccttagctttttccgatagcattaagtctaataactcacttttttcatctctagttgtctttttacgagttaaaaacacctcgtcaaattccactaacccgtttttatttgtatttagtaacttatctaaagcaccaaacatgacaatcctgtcatcaatatcatctaatttattaataatagaaagtatgtcaaagaagttatcttctcctagtactttataaatatcttgtagttcaaaggtatcaattagttctaatagttcttctaaatcacttggtgataaatatttactcattatcatcaacctcaaataaaatatgacttggtgtttctaagattttttctaaattaattttattaccacttctaggggttttaataccttgctcccaataactaatagtagaataatttactcctaattctttagcaagacttcttaaagtatatcctttttcaattctgattctttgtaattttaacattactatagccttcctttctttcttagtataattaaagtataccgtattatatgttcaatgtcaacattattcttctcttatgtttacaaatttgatgttatccttataggcaattttagacataactaaatcatctgctttatatttagccttaactgtgttactttcaattaccctaataactaaatcttcttctctacctgattgcgtagtgtatttatatgtgactttatatacagaatccttattacctaatacctttttattaaaccactttttaaatttattcatagttaccatttcctttctaaaaataagtataacataaaaaataagagagtgtcaagcactctcttaaatagttatttccatctaactctaccccaatatttttctttaacgatacgctcttttttatctgtaattttacagatagccatatagaagtatcctgcactagggttagtagggtattttagcttaatccaccaatagccatctttcttagtgacgctataaaatggtacccattgattagcataaatccaagaaccactttctactactttaccatctaagcctggtgagcgtctaactttaatagtcctatctgcagtaaaagtacctcccatattccaagtaatttttggttttggtttttctgctttgaagtttggtctgataaaaatcatattagggtcataaggatgtactcgaatagtagcgggttcccaacctttaccaccttgggcattaccatatgtccatcccccacctaaccaattgttttcaatcacaacaatttggttaagggtagcacttaacacccatgctacatgaccatgcccattaccataagctcctggaaataataccatatctccacctttagctaagaagctaggtgtgtttttatatatttttgctttatccttaagtaaactagcgttatcactagcaatattttttgcactttctccttgtaaagagtatccagggaataatttattaaaaccatagtttgctaaatcataacattggtacccataatcgtaatgtttgttatacttcctcccatacctatatatatgtataaaagtataacggctctgtaaaaccttaagcccttatagatttcctataagaccagactatatcatcatttaattaacaaatttcattgtgtactttttcccatctctactcttgacgtattttgttttacctttaaaatactcatatattatattattacctgatacatttagttctctgcatgtctgagacatagatgggtatactttatctgtctcataacagtatattttacgcatacccctaccatctttgtagtacttttcttttaaattgtatctttcagctatattatgaacatgagttgagtctacattatgttttttagatattttatagaaaacaataccttttttaatatcttcaataatactaacagtcttttcatctctatagttaatagatttttctaatatctgagttacttcatgttcttcgatgtcaggtagaatactattatttatagatttagatataaaactatacttacttattgagcaatctagctccacaaaattaatattgttttccatacagtattttctttttatattagtggatttcttagatttttcgaaagaatctttccatttagagcttactcttgcattataatgttgttcaccatgtgcttctatagctatattaagttctggtagatagaaatctatatatcttctttcattaggaactttaaactgatactggtaatcaataccttttacttctaagtaccctatcaaaaacctctcagggtagcttgtaggtttatttttacatttaggacatgagaatccattccaaaacaagttatgagctctaacctgtttctcatatccgcatagtggacaaatacagtctatatatctgtcactacctctactatagttcttagcatcatcaggattcttaaagtatttatgtagatgtttataattatcaagagtatgtttactattcctaggtttaaattttttattcattttgttaattaatgccctccactgcctaccgataggtagttcttagtcgttgaacctttttctatacaagcatatcacactttgtaaaatttgtcaacttatatagaaacttggatgcggatttcccaatctttatcttttttactataccaaacacattactgtctgcccctaactatgtcaccatgttaggttagtagataaagctctaagggggttcccgtcaattcaaagggttttacatggactatattattaatccatcgtaatcaatatatttccctgcggtacctcttaaaaaggagacaaattgacttcgtgtcatctttgctaacatttaatcaatcctcctaattccacttaatttgaccccaatatttttctttaataatacgctcttttttatctgtaattttacatactgcacaatagtaatagtctttactagagcctggttgtcggtacttaaatctaacccaccaaaatccatccttcttaataactttatcaaatggtacccagtcattttttgtatatagccatgaatttttatcaacaattgcaccttttaatcctggtgatttacgtactttaatagttgtgttagggtagaaagtaccacttaaattccaagctgttttagggtctgtgtgttttacctttggagttactgaaccagacttaactgatttactacctttaatatattttgctacaattttatctaagttacttgtagctctaccatatcctgcttgttgtaataaatttcctgggtcttgtttatctgattgaatatcttgatgtcctggcattcttgttttgtagtctattttccagtaatcacaaaggtatgctagtacacgagcaaagttatctaacgattttttagttctttctttatctgaaaagtaacaaccttctacaccaatcgcagcatcgttagcatctaaattataccataaattatcagtaggtgcgttatacattacgtggtaggcaatctcagtcgtaggtatacaaataattgcctctttatcatcaacaaatatatgagcacttgctacataatcccaaccgatgttgtatgtatttctatagtaatctacattgttttgtgcagtagagtttaaatttcctgtatcatgtgcaacaataaatttagggacgccagtagtcatatttttacctggtcgtctagttgcaactggtaatagttggaactttactgttacaccattccaattttcagccatttatcttcctcctagccttttggtgagcttgttttatgttcatctgggacatcatcaataggtgtttcagaatctagtttttccataggttctgcaattgctaaaatatcttcatgttctaattcaccaactacatctggagccataggatgactacctccgtctacaatactattaactttattattctcccaagttaatgattgttctgggtctttagaatcacgtggttcatggtaatctgtttgaacaataatactatctttaatacctttagtattgttatctactaaaatacctaaacctgcaagcaatgttaagatagagccaataatattaacaccttgctctacttgactagaataatctaagccaaaagcacctgttaattgattgataaatactgctaaggcagatacactagctacccaaaacgttttacttttaaaacgggtacttaagttaataccaccaacaactgtaggtgtcttatcttttttagtcaaaataaaaaacgccctttcttgttatattttttttacattactaatataacaagattagacgttttaactatatattaaagttcttttaatgtactaataacttgagacatttgtttattatctaggtcctctagatttgtctctggtaataattctaacttttcccaaagttcatttttattattaattttatcttctatcaatgctttacctactaaactttgagtaaaataaatttgtttttgactagccataataatcatttccttttattaaaatctagccattgtgtactgaaaatcaggagagtaattgaaagaaaggagaaatgtatgaaaaccaattccacaatggctataatattgtatactactacaattaaatatcataacatagccgttctcacacggaactatgtataaagaaatattattaggttacctagttctaataagttctatacgggagcgacctcgtagtccacttatagaagcataagcaatatagggatagggtctattacctaccaactttcacacggtatgaagaattattgtaaagtatgcaagtatttgttagcgttattacttttcttttattgtagatttgtgctacttgtactggtatcattcattatcaaacatgtttccatgctccaactacaaaagtgctttattagtgtaattatataggagaccaccaagttcctacataatgattacacctgacagataccataccttatatccctttatacatagagctgaacaatccacctcgtattcaataatactcaaagggtatttcctaagttctcacgtaggtttaacaccctgtgacattttcacaccttacagccaccacactgttaaatttcagtcactatgttatgatatttaattgtattaagttgtaaatataatataacacacttttaaaattcagtcaagtaaatttaaaaactttttatagtttttttatactagggtaaaaaagtagtaaaaagacctagttgtgtaaggggtcatttatttatatataaagtataacataaataaaaacccgtgtcaagttaatttagtaaattttttgaatttattaatttttgtttttctagttctaaatttgatagttgtttatttaatataattttaaaattagtattgtcagtttcttctacgcagtaagagtacttgcctgccatttcaaatacaatcctagaactttcttctagtaatgttttatcttgagatactaatctagtaagaccaaatttattggactctgttttactaataccattatttaacattctttttctagttcttgttattctagattcaatgttgatacctaggttagagtttaagttcttcgataactctgaccaacagtatgatattgacttaccatacataatacgttcttttttgcatctattagtagcaatacctcttctcatagaaactagctcttttctccattcttccgtatctacatttaggttgtagattacgttattgtttactgagtaagaacccgtttttctgatagaaggtagaacttcttcatatacccaatcttgaaatttgatagattctttctttctacttaagaacacgagtctataaacattaggctcatttatatagtttaatttctgtttaccacctttcgtaagggtatcggtaataacgacccccttatgttgtaaacgattttttgcatcactagtgttagttatccctaaaaccttacaacaatctttgaggttgaaataaacttcacctgtatcttcctttataaaacttctaattccctgtccttcatagtttaaatttaataaattttccatagttatcatttccttatcatttatttggtacttctattctacaacaacattaaagtaaagtcaacacttttttacttattttgtaattcttcttgaagagaatcccaatatttttctgcttgacttctattatcttcaacatgagcagtgactttattacatttgacacaatgcatatgtttaacatgtccttcttctcttctttttgctctttttcttggtaatttaaaacttgaaccacactcttcacatactaaatttgagattacataaacttgctttttcatttttatcaattccttttctatttatttggtactcttagtttatcttatctaattctgaaagtcaatacttttttggtaaatttttgttatattttttgataaaccctttaataccaagggttacaagaagaaaaaaacatgtttggaaaattaacacctctcttaatatatatatatatatattactaataatactaataatactaataatactaatattactaatattactaataatactaataatactaatatatataagggggaccttaattttccaaacatgttaaaaaatagctgtaacccttgatactaaagggtttagtaaaaattgtatataaaaagaccaactttcagttagtcttaataaaacaatttgaaactttcaattctatttgggttaatataaattctaccagatgttaagctattttcagtatagactttaaataatcttgcactagacatataggcatttaaatctgtatcataaactggactatcttcagtaccaattaaaaaatacttattaccacttgccattgttatttcaacatatttaatcaaaacttcttcctcctaagtaaacttattgaatacaaaagataaaatagcaccaagtactaataataagactttctcaactctttcactttttttatcatctttatcttttgtactttcagctaattctttcactttctcatctaatgcctctaattgtatatagataggtgtttgtctttcaacattaactgctaattctttatctaaagtagttacgatactctttagttcatcaacttcattttccagtttcttaaatgtatcactgtcaatataccctttttcttgtatagtattacttattgattcaacttttttaaatagttcatctacattattcgtcaatgtaaatcaccacttaatgataaaccgattcagagtatacatcaaagttatctataaacttatcaataatctgggcataatttttagtaataatttttccataagaagtttttatatcaatttcaaaatctataacaccaaactcttgaaggtctatacggcacttaactttatcagggtcgttacaaggtaagtaaaagtaatcaaaagatataatagagccgttagtcaaaagtctgtttattctatacatgtgaaaaatatccctatctcttttaaaatgagttaaggcatctttaaattcaacttttagaatatctttatgcttagtcaattttataccgaacctcctttattttttaataaaaaaacttgcataaattaaggatttatggtatactaattagagagtaaggtagccaccactattatgaaagtataaagaaaaacacccataaaatttcctagatataggatgtatatacctaataataaaacacagtacataataaaaataataagtacttgatacataatatttaaaccatatatactattattagttaactgacgtgtaactatatttactcctgttagtatgataatgaaagccactaatacaatacatacttcaattatagtcaccaacatattgtcacctattatttctctaatatcattcaaaacttaatataacatagaatgacaacattgtattatattaatatagtgaacaactatgccttagagcaaggggtggaataaaagtttgaaacggaaactagaaaaaatagatagtattgtaaagaggatactgatactaattatatcaattttagtcatattatcaatagtcaatgcactgtcactaatatttgaaatgagacctaatgttgtaatgatttactttatgtttggtgctatgacatcttatgtagttttagtgtattatgataaatttatacaaagaaaagatgacccattacctgactatattttaaaagaagtacaagtaattaaaaacgaagtagatgttctacaagaaatgttatcagaagaacaaggtaccacaaaagaagagattaaagtgagagcaaaggcaagacgaaaggtagaccatgaaaaaacacgaagtaaaagataagcgtagacttattaaaaaaaatggaactaaagtaactcttgttaaaaaaaacgacaaaagaattacaagccctagtcgtatttgttgtatatgtggggaacaattatcaaaagtaaattatagcaatgggagagttcttgcaaaaaaagaccatatccatgttcagtatagtagtcttttgtatttagatatgtgtaagagtgtatctaactgctataagaaccttaaagaaagggggtatcttagtgagtagaacaggacgacttcgtagaaaaatggaaaaaaagaaaatcgaacaagaagataaaactaagtatattcaagatggttttatgagtagtatccaaactttattgttcgattttcaaaacaaagttaatgcagggcaagtagaagtaaaagaccctaatgatttatataaattatttgttatgtattctcaaatgcaacaacttataagtgatgagacggaaggagcaggtgttttacctcaactttctggtaagcaacaaaaagttttcgatgaaataatcactgaaaatagtgaagatggagaaatagacatgaaaaaactatctgagttatcagaagatgacattactaatatgattgcggataaagaaaaggcaatgaataacgataattcagagacattttagaaggagtgatatgtttggatggtaaagagttattaaagattactcaagaaacatttcaaacggaacaagtaacaaaagaacaagtagaccatattattaatatgttaaacccatctacttatatgctgaagtatcatacactccgtggacatcctattacattctctattccaaatagagatagaagtaaagcccaagcgcatagaccttggcaagtccgtaaactacatatgcggactataaaccttttctaaacttggaaactcctaacgtaaagacgaggacaatcaagtgctaaaacaaatgcctagagactaaatttactggtaagcaattaaatggaattgtgtagaacagtataagaggaatttaattccgtaacgaaaaggacgtgtatacctaaagatatacgtatgagatagtccaatcccctaataaatatcgggaaaccgagggtagtaaatgatagtaaatgatactcaccctaataaagcagtaattaaatcgagacaattagggctatcggagatgtcagtaatggagatggtacattttgcggatttacatagctacgcctctgtaaaatgtatgtacacctttcatacccaagaccaagttaaaaaatttgtacaatcaaggttaaacccagtattacaaaaaccttattttaactctattgtagattgggaacaagattcacaaggctttaaaaaactaagaaattctagtatgttctttagaacgagtagtaagccaggagctttagaaggtgttgacgtagattttttagtttttgatgagtatgaacgtgtgcctaaattatcagagtcttctggcttggaggccatgtctagttctccttttaaagtagttcgtagattttctacaccttcagcacctggtataggaatacatagattataccaacaatctgaccaatggtattatgcacatgtttgtcagcactgtggtcatgaaaatgaaatgaagtatgcagattatgacccagatgacttatctaaaagtggaaatttattatgtgtaaaccctgatgggatagacgagatggcaaaaacagtacaagatggtacgtatcaatatgtgtgtcaaaaatgtgggaagccactggatagatggtataatggtgtgttaattctctagcatacctaaaaccttgttaaacgggcatagtaaaataatgtatagaaaaaacaatatactggtaagaaaccctaaatctgactatacacagacagtgggaaccaaccgtgctaagtgttgactttatataaaatatatgatataatgtaattataaagttaactaaatgcctaacgactaaatttctaggtaacttacaaaaaggtgtaagtgagaactagataaaaattacttaacatgaaaacactaccataaaataacagataaggaggggagtacaatgactaaaaagaaaacccatgaagaatttaaaagtgagttagattataaatttaataatgaatttattcttttaagtaattataatggtataaattcacgtattaaagtaagacataaacactgtggatacgaatttgaacctatagctaaatatttattaagggatggtaaatgtaaaagatgttcagataacaagaaaggtaaaaaaaagaagaaaaacacatgaacaatttttaaaagaagtacaagaaattcatggtaataattatcaagttttaactaaatataaaactagtcatgataaagtaaaagttaagcatttatcttgtggttcagagtatactattagaccagattcattattaagaggcagtcaatgtaagaattgtcttaatattaagttatctaaggataacttaaagacaacagaagagtataaaaaagaaatagaatatataactcaaggggaatatactcttataggagaatacacaggagttaaaaacaaggtaaaggttagacataatttatgtggtaatgagtacttagtttatccttttatgttttatcgaggaagaagatgtcctaaatgcaattattctagaggtgagagattagtagaggttgttttgaaatccctaaatattgattttgaacctcagaaaaaatttgaaggattaaaaaatataaacctattatcttatgatttttacttacctgagtataatgtcttgattgagtatcaaggacaacaacattatgaacccgtagagctttttggaggagaagattactttttaaaacaacaaaaaaatgataaaattaaaaaggaattttctaaaaacaacaatatttatttattagaaataccttatactaagtatacttttgatagtgttaaagaggaaatagaagtaatgttaagtaatgtaaagcaagggactactaaccaaaagaagtagtcatgatatagtctaaaccctttaaaatactaggaaactgggggtagtaattgggagatgccattttccaaatagaacaaagaataatgcaggtataagagggtactatattagtcagatgaatgcggtagatacatgccctttaatgtagaaatacattaatagcatccctttaattcatgggacatctctcatttgagacaatcatgagccaagcctagtaataggaaggtgcaacgactattatgtagttccaagtggaacgaaacgggggagacctaagtctcatatgatatggttatgatatagtctgagctatatggtgacatatagaaggataggagtaacgaacctattcgtaacataatgatggatttcagctgaccaactcgtagaaaaagaacttaataccgactctaaacaagcattttacaattatgtactgggtaaataggtagccccttcatatagtaatatatgtcgaagaactctgttaaacaggggaactctaagtgcttaaatgagcatatgacaatcctgtgctaaatctttaatttaagtgttgacaataaataaacttatggtatacttaaattaaggtaaatgccgaacgactaaatttctaggtagctatataaatggatatagtgaaaactagataaaaactaataatttttttaaacttaatatagaagaaaggaggaaatacaagatggcaagaagaatgacaacagaggatttcaaacaaaggataacagcagagacagatggagaatatatagtccttagtgaatatgttaataaccaaactaaggtgttaatacaacataaatccaacaactgtaataatcatcagtattgggtaaaacccaaaggaatactttcaggtacaagatgtcctgcttgtaataataagaaaaaaggcaaaaaacctaagacaactgaacaattcaaaaaagaattaaagggtatagttggagatgaatacacactagtaggtaaatatacaggagtatacaataagattactttactacataatgaatgtggtagagagtgggaagttacccctaatgcttttatttccagtggctcaagatgttggtactgtaatataaaaagacataatctaagtcaaagaaagccacatgaagtatttgttcaagaagttagagacaggtacggtaatgattacaaagtattaggaacttatattaactcaaaaaataaagtactggttagacataaatgcggaacagaatgggaaataaaacctaatcatctattgtcaagagacatgtgtcctacatgtaagatgtctgttggagaaaaacatatagaaaaatatcttaatagtagagatattaagtttgaacctcagaagaagttttctgatttagtatataaaagaaacttgtcttatgacttttacttacctgaattaaacatcttaatagaatataatggagctcaacactacatgccaatagagagttttggaggtaaagaggcatatgaacagcaagttataaaagacaatattaaaagaaagtatgcaaatgaaaatgggtacaatttaattgaagttaagtatactgagaagtactataatactatagcaaaatatttagataaaaaattattagtgtaaagcagagagcccttaacctaaactaagggttatgatatagtctaaaccgtaaataaatactaggaaactagcggtagtaattgatgccttacgaagatttaaaaatgcgtgtattaccagaagatgtgtttgagaacaaatcacagattgctaaagaacaattatttgatagagatagatataagtatatatcatgtagtgttgactggggtaattttcattggtgctgtgtacatggtatgacagaagatggtcatgtagacctaatacgactatttaatgtaaagaaaaatagcagaccagatttagtagaggcagacttagaaagaattatattagagttttctaaatatgaccccgatattattatagctgataatggagattcaggaaataacgttcttaaattaatcaatttctttggtagggaacgagtgtttggatgtacttataaatctagtccacgctcatcaggacaactatatgcacaatttaatgaaaacacaaacaccgtaaccgtagacaaattaatgcataataagaactatatacagggactaaaaacaggtagaataagtgtgtaccaaaaacaagatgaagaacttaaaacatacttaaaacattggcaaaatgttttaatcatggatgaagaagatgagaaaacaggggaaatgtaccaagtaattaagcgtaaaggagacgaccattacgctcagagtgcaacgataggatatattggactcaatcgtattaaagaattacttgaaacatctaaaggaacatcttttgattctacgttcgtttctgctgactataaccaagattcaaataatacattctacttaaatgattaggaatgatacaatgttacaagatattatagataataaagacactgatataaatatcggtgtcttacactttttaacaaatagaacttttacatatgaagaagtagcaaagaataataaacaactaacaaaagactataattttattgttaagtcttatggatttaatgatttctatgacttgtttttatatgtcaataatgaagataactttgtttctaagggtggagataaagacgaatctaagttaaataaagtgaaacgtaaagtcatacgtaatggaaaagaaatagaaatgactgtatatgaagaccctaatgcaggtaataatcaacaacaagataaaaaacccaaagataatgaacaacaacctaaaacaacatcagctataggctctaaatctacaggaactgaagaaggacaaaaagttaatcctaagagactatcaaatacaattagtaagttaggtagtaacatagaccacatagatacttctttagttaatatctctagggaatttacttctacagatggtgatttaataggtaatgccttattcgaggaaacggaagatactattaaactacatggatacataacaaaagaagatgttactggagtaggtgtacgttctattatagaattacttttattaggtataaagaaagaaaagaatatagaagtatatgatatacaattacctgaggcggtacaatatctaaagatgttagggtttactaaaaagaaagattctttttacatgaagaaaaaagatgtattatcatttataggggacttacatgaatttctttagtatcttgattatatttacaatttgttatatactatatgtgttaacactattagtagtatattatagaaaagaaattagatttaaaaaacttttaatgaatctagaaaatttaaaatataagttttatacagaaggggaagatgttgtaatggaaaataagttaacgctagaggaatctatcacacctttaacaaaagaagaaaaagaagatgctattaaagagtttagtcgaattttatgtgaaatgactaataagttgtattctacatataatgtattaaggcaagacccagtagacgaaacacaaaaattagatggtcatttaatggtttttcagagccgtttaggtgattcccttacaggagatttacatgataaactatttagattagcatttgataaacgtttaaatatctttgaagtaaataagcaatacaagaaagatgtgcaggataataaagctattgctctaggtaatgttgttgttacagatacagctattagtaatattttatccagtaatgaattaggttttagtatatcatttatgctaaaagaagattgggaagaaaaagaacgaattagaaaagaagaggaagaaaagttaaacaaagaaaagtaatgatgttatgagatacagtgaaagggaataagtttaatgaataaaaagttagacggtaaagagatatcaactgtaattatcataatattaatagggttctctatattatttaatattttatttttatctcaactagataaagaaaaagaagagaaagataaatacaaaaggttagttaatatttatcagaaagataataaaatagataccaaagataaaaaagaatatgttaaaaggttaaatgatgcagaagaggaacttgataaagtaaagaaggaaactaactataaaggttacaatgataagtctgataaaggtaaagcagagatagataaggaacttagagagtctatttttggtaaacttagagatgatgaaataattgttaaaactaagattgaatacacagataaggttaaatcacctactatagaaatagacaatgagggaataggaacagtgttactaccgattataaaccagaagaatgataataccgaagtaaaagtagttgattctactaacacaccaactactacacctccagtagtaagtgcaccagaggtcagtaatacaccaataccccaacaaccacctagtgtaccagataataacggtaacacagtacctattgcacctagtacacccaatgaacctagtgatggtggaaatttaacaccaaatgttccaattaatccaacaccacccgttgaagaaccgcctattgtgttacctccaccagtagaagaacctataaaacctacggagccagatagacctacagaaccaactcaaccagaagaaccaagtgaccctgttgaaccagaagaacctaacaaccctgataacccaaacaaccccgaggaaccatctcaaccagaagaaccaagtaaccctgataatgacgataacactgatgttgctgaggaaccaagtaatcctagtgaacctaatagtataccgtgggcaccactagaaccacctttgagtatgcctttagttcctttagaacctgcaacaccaataacataattattactaaaaaagcataggtttaatacctatgctttaattatattatattaataagagacgctattatagaaaggatatgtaactattgaactttgatttaaatatactaacttctattttagttttaattataggtacttgtttacttacattagtaataacagaatgcttaggttatacaaaagaacttaaattgttacattatgcaatttatggtatagtaattataggaacttattttttaacagaaatgtttaaaaaaatatctagtataattaaagaagatgttattttatccgttcaaaatacggaaaaatataacggttttaatgacccaatatttatattcattattacaacgattggtgctattattggattagttatttattatacgcataaatatataaaagaaactaacttaaataagaaacggtgatgtagactttgacctatgctaaagataaaaaatgggaagatgcaaagacttttataaaacaacagggtatgaaagataattggatagaaatagtagattattataggcaaataggcggtacgcacgttgctgtatttattgctctagaaaaagaaaaatatatggtactagaagcaactactgataatagagtaatattagtagataaaaacaataatattgttttagaagattataatgtggttatggaatctaagaagatgttttattatatagaacaaccctttaagcataaaatagacttaaatagttctataagcaatattacatataataacacagatacattaacaatgtacaaccataaggatggtgatacggttggctgatatttttaaacgtctaaggttaggtagagattatgaagatataattgatactgtacctatagatgaaggattacaggcaaatattagaaatatagaagaaaagagcaaagaacttaataagtcattatacggtaaacaacaagcatatgcagaaccctttctcgaaatgatggatacaaaccctgaatttagaacaaaaagaagttatatgaaaaattcagataatttacatgatgttctaaaacagtttggtaacaatcctattttaaatgctatcatcttaacaaggtcaaatcaagtagctatgtattgtcagccctcaagatataacgaaagaggtttaggttttgaagtacgtatgcgtgacttatatgaagaacctggaagaaaagaaaaagaagagattaaacgtatagaaaattttatactaaatacaggtaaagataaagatgtggatagagattctttccaatctttttgccgtaaaattgtacgtgatacttatacatatgaccaagtaaactttgaaaaggtattcaataagaaaaatgctacaacaatggataagtttattgccgtagaccctagcactattttttatgctactgataaaaatggtaagataattaagggtggaaagcgttttgttcaggttataaataaaaaggttgtagctagcttcacttctagggaaatggctatgggtattcgtaacccaagaacagaattatctagtagtggttatggattatccgaagtagaaattgctatgaaacaatttattgcctacaataatacggaaacatttaatgatagattcttttcacatggtggaacaacacgaggtatacttcagattaaatcagaacaacagcaatcacaacgtgcattagagaattttaaaagagaatggaagtctagtttttctggtataaacggaagctggcaggtgccagttgtaatggctgatgatattaaatttgttaatatgacaccaaccgcaaatgatatgcagtttgaaaagtggttaacctatttaataaacattatttcagcattatatggtattgaccccgcagagattggtttccctaatagagggggagcaacaggtggaaaaggcggaaacacacttaatgaggtaactaaacagttacaatataaatataaatataaatatataaactgtttgccttgcttatagagtaatctgtaagtaccgagtgttaaatgctaagaactcctaaagactcaataactgaaacagaatcctaaaggataaatggaaaagttacgaaagtagaaaaaatattgagtatggtataaggttaaatcctaagtactaatacaatggatgtttagcagggaaagttctaagggaatgtaattcctatgaaagaccctcaacgactattccgtataggtgacggaagtaaaaccacaagtgattggtggaagaaaaatactcctccttacttaataaggatgaacaaatagtctccgctcgttctgaaaggaagagcttggaattaaccaaggacttagaagtagcgttctaaagttaaagattgttaaaacttataaatatttaaaattttattccaattacctgttgacaattactcaccattatgttatcatagatacataaggaggtgaatataatgttaaaacaggaaaaatataaacgtataaatataagattattaccaacaaaagaacaagaagttaaaatgtggaagtcagtaaataatgtacgaaatgtaaaaaacatatttatagctaggtgttatgagatagacaaagatactgaattttttagtgtagacccattaaagaaagagttgacaaaattattaaaagataaaaagtatatgtatcttaaagagacctcaagacaaacattgaattttgcaataaaagatgttgtagactctttcattaattacagaagaaaaagcactaaaaaaccagtattcaaaaaaaagactactgccactaaatcttttaaagttaggaacggttctaactcatttagaagggtaggttttacattttatttagaaaaaattggtaatattaaattttctaaaaagtactatttaaaaaataaatatatttttgattatatggaaaaagatagatatatacttcctacagtaatctatgatggaaaatattggtttatatctataatgtataggagtgacttaattaattctcaaaaaccaagagaattaaccaatgaaacaataggtatagacttaggtattaaaacattagctacttgttcaaatggtaaatcttataaaaacataaataaatctcaaaaagttaaaaaaatagagaagaaattaaaacgtaaacaaagacaagtaagtcgtaagtacgaaatgaataaacaggataaaaagtttattaaaactaataatatcattaagttagaaaaagaaataaaactcttacacagaaaacttaaaaatattaggaatactcatattcatactatgactaaagagatagtagaacaattgccacaagaaatcgtaatagaagatttgaaagtaagtaacatgatgaaaaataaacatttatctaaacacataagtaagagtaattggcatgaaatacgtagacaattatcttataaatgtgaagatagaggaatattacttacagtagcaaatacttattatccaagttctcaaacttgttcttgttgtggtaatcgtttaactaaacaagataaactatctttatcacaaagaacattcacttgtgaatgtggaactactattgatagagatttaaacgctagttataacttaagagattacagatactccaaatggtatcaaaatcatttagataaacaaactaaaagttctaaatgatatgtaggattccgtaaacccgaatttaagcctttggagtatcatacaaacctgagtagagtaaatgaatatagtaatttactcaaaaagggatacgttgaaaaaggaataaaattttataattatttataagttttaacttacggcagaccctggtaagaagcaacaacaatcacaaaacaaaggtctacaaccattattgagatttattgaagatttaataaacacacatattatatccgaatatagtgataagtatgttttccaatttgttggtggagatactaagtcagaattagataaaattaaaatcttacaagaagaagttaagacatataagaccgttaatgaggcacgtaaagaaaaaggtcttaaacctattgacggtggggacgtgttattagatggttcatttattcaaagtatgtcattaaatacacaaaaagaacagtatgaagatacaaaacaaaaagaacgttttgatatgatacaacaattcttaaattcaccagaagatgaagaagaaccacaacaagagcctactggagataaagttgatggtagggaatctaacgacccaacaaaagtagatagtcctgtaggtacagacggacagattaaagagcaagataatgttaagagtcaggaaggttctaacaaaggtcaaggaacaaaaggaaaaggtaatgaaaaaccttctgattttaaaaaataatttataatagataagagagaaaactaaatttctctcttattttcttgtatgaaaattaattatatgttatagtgtatatataatgttatatatacacttgattgttatattataaacgtaaagtaaataaattaacaataagtgaggtgcacacacctttggaagaaattaagtttaatgcctttttagaagctgatttacaaaagtcagtagatgttaaaactgatgagaaaaattactactttaaaggatacgcaagcacacctagtagggatagacaaggtgaagttattaaccctaaaggtatgtcattagatatatttaagtctgatgggtatgtaaactatgaacaccaaaaagacaaactaattggaatccctacagataattgttacatagacccagataaagggttatatatagaagtgcagttatttaaaggtaataagtatgctgaggaaatggtagaacttgcacagaacttagaaaagtccggcactggtagaaaaattgggttttctattgaaggtatggttaaaaagcgtaatacccaagataataccattattgatgaaatggtagtaactggggtagcattgaccaagttacctgctaacgagcaaagtagagcagagtttgttttaaaatcatttttgactggtacaggaacaacaccagatacacaagtagatgcaggagcattaagaagagaatctttagcttctagtatcactaatcttacatatgtaactaaaattaaagacttaaaagaatttaataatacatggaaccaagtagtagagcaccttgaaaagaataaccttatgggttatgaagaatctgttgtgaccttacaactagcaaaaggtctttcccgtaaagatgcagagttagctgtaatggaaatcaacaaaaaacaattggaggactaatcttaatgagtaaattttcaaatattctagaagaatataataaattacaatcacaagatgttgaaaaatcattagaagaaaataaagatgaagaacctaaagaagatgctactgtagaatcagttactgaagaacaagtatctgagacagaagcaccacaaaaagaagaaccacaacaagtatcagaagaagatgctgaaaaagcacaagaagagtctaagaagctagaatcagaaaaacaagaagaagaaaaagaagtagaaaaatctgttaaagaatctaaagaccccgtagaccataaagatactaagacggaaaataaagacaatgagaaacgcaaaaacaaaaaagaagattctgaagatgaatcagaagaaggcaaagaagaatctaaaaaagataaaaaatctgaaaataaaaaagattctgaaaaagttaaaaaatcagctttatctgatgaagatattgtagaaggatttagtacagtactaaaatctttacaagacttacccaaccaatttgctactaaagatgatgttagtgaaattaaaaaatctctagaagaattacaagatgcttttgctgaaaaagaaaagcaacaagaagaaaaagtagaacgtattaaggaagaagttaataaagaacaagaagataaagaagaagaaacttctgatgaatctgtagaaaaatcagtaacaacttctaacactacacaacaagatgatgttaattatgtttctaaatcagcagttgtagaagaagaagtacaagaagaacaacctgaagtagataagaaagaggttaacactattacacaagaagaccgtgaggctttcatgaataaattcaaatcagaatctcaacgtcgtgataaacctactcgtcagttaaatgatgcatatttagcatatatggatgttcgtaacaatggtgagaatgcaagtccaagttctctaaaaactgttaaagactttattaagtaatacaaagtagttgtgttatattatacatgaaattaaattagtaaaataaagatacataattaaatttataaatgaaaggtgattctactatatgcataaagataataacttgacagcggaacagcataaatacgctgataagttccaagaagaagtaatgaaatcctaccaaacaggatatggcattacacctgatacacaagtagacgcaggcgcattacgccgtgaaatcttagatgaccaaatcacaatgttaacatggacgcaagatgatttaattttctaccgtgaaatctcacgtcgtcctgctcaatcaacagttcaaaaatatgatgtttacttacgtcatggtaatgtaggtcattcacgttttgttcgtgagattggtgtagcaccagtatctgaccctaacatccgtcaaaagactgttgaaatgaaatacgtttctgatactaaaaacttatcaattgcatcaactcttgttaataacattcaagacccaatgcaaatcttaacagaagatgctattgcggttgttgcaaaaacaattgaatgggcttcattctacggtgattcttcattaacagcagacccaacaggtcaaggtttagagtttgatggtttagcaaaattaattgacaaaggtaatgtaattgatgctaaaggtgagccattaacagaaacattattaaaccgttcagcagtattaattggtaaatcatttggtacagcaactgacgcttacatgcctattggtgttcacgctgactttgtaaactctgtattaggtcgtcaaatgcaattaatgcaagataacagtggtaacgtaaatgcaggattcaatgtacaaggtttctattcatctcgtggattcattaagttacatggttctacagtaatggaaaatgaattaattttaaatgaagatttacaacctttaccaaacgcaccacaacctgctaaagtagaagctgaagttgaaactgataaaaaaggtttattcacagatgaacatgaccgtgcagaattaacttataaagtagtagttagctctgatgatgcacaatcagcaccttcagaagcagtaacagctacagttaacaacgctacagatggcgttaagttagagatttctgttaatgctatgtatcaacaacaacctcaattcgtttctatctaccgtcaaggacgtaaaacaggtgacttctacttaatcaaacgagtaggtatgaaagaagtaaacgaagaaggtaaattagtattttatgaccttaacgaaacaattcctgaaacaacagatgtattcgttggtgaaatgtctccacaagtattacacttgtttgagttactcccaatgatgaaattaccattagctcaaattaacgcctctgttacatttgcggtattatggtatggtgcattagcattacgtgctcctaaaaaatgggtacgtattaaaaacgttcgttatatcgtttaatagaatatacaaaaaattaaatataaaattttatagcatgggtcaaattcccatgctttttttattaaatatataaggagacttgatatgttacattactataaacctaatcttaaaattgctactatctatggagaaatcacagtagacgaaaatggaaaagttagtgggttaacaaaaacacaagagaaagattttaaagatattttaggttacacttacacagaagataaaaaaccgcaagcaaagaaagaagaaccaaaagaagaaaataaagaagaagtaaaaacagaacctaaaaagaaaacaacacgacgaactacacgaaaaacaacaactaaaaaagatgaagagtaaggtggtattttatggttaattccatgttcggaggaaatttagacccttatgaaaaatcactaaattatgagtacccttaccacccatcaggcaatcctaaacaaatagatgtaaaagatatagagaacttaactttagcagactatggttggactccagaggctattaaaaaccactacatgtttggtgttaccgtacaaaacccagatacaggtcagcctatggacgactctttttacaatcatgtcttggagatagccatagacaaagctgaaaaagcgttggatattattattctaccagatattaaaaaagagatgcgtgactattatgaaacagaatttaatagttatatgtttgttcatgcttttaataaacctattttacaagtagaaaagttacaattaggttttaatggtagaactatttatgattatcctgctaactggtggaaagtaagtcatgtgggaggacatgtacagttatttcctacagcgttaatgcaaacagggcaatctttagcttacgatgcagtatttaatgggtatccacaactagcaggagtatacccaccgagtggagcaaactttgccccccagatgattcaattagagtatgtagcaggtatgttaccacgtaaacgtgcaggagtaactaaaccatgggaagtaccacctagtttagaacaattagttataaaatatgctttaaaagaaatttatcaagtatggggttacaatgatgttaccctgtgtgtagcgtaagttgcacatttccaaaggttaaatgctatgaatccgtaaagctaactaaactaaaacagaatccgaaaggatagatgggaaagttgtgaaaacagaaaaaattagttagatgctataaggttaaatcctaagtagtaaataatcggtctatagcagggaaagtcctaagttcaaatgaataaggaagaccctcaacgactattccgtataggtgacggaagtaaagcccaaagtgattgtgggaagaaaaatctttgccctatagattagggataacaaatagtctgcacacgtaccgaaaggtagtgcttggaattgtccaagacttatttagtagcgtaaataaggaaacaaagttaattaaataataactttctattgaaattaccacaaatatatgttatagtatacttatagcatatataaggtggtgaaagaaaatgaaaaaatcggaaaataaaagttatagaggtataatactaaaagtaaaccctaccaaagaacaagaagatttaatgtggaaacatgtaaatcattctagatttatttataattatatgttagaaagatattgggaatttttagataatggtaaatacatagaccataaacaaatgctttctatattaaaagatatgaagaatgataataaatatagttttttaaatgaaatatctagaaaaaccttagttggtaaaatagatgatttaagagaaactttaataaagtatcataatcatgaaattagaaaacctaaatttaagtctaagaaaagagaacctaaaaggttccctatcagatatgatagaatgcatttcaataaagacaaacatgttaaagttgaaaagatagggtatttaagaatttcaaatggaacatataaaaataataaatcagtattagagaatgacaatattaaacccttaaatcctaaatgttactatgatggaaaatattggtatatttctttttcaattgaagtagataatcaatacaaaaaagaaaaagaaactactaatgaaactataggtatagacttaggtagcggtaaaaaacacgtaatttgttctaatagaaaaatttatggtaatgttatttattctaaaaaaatgaaaaggttggaaaaaaagttagctgacctacagaaaaaagctagtaataaatatgataaaaattacactaaaactaataatattttaaagatagagtaccaaataactaagatatatagaaaaatgtcgaatattcgtaaaaatcatgctcattctataacaaaagaaattgtggaaagttatcctaaagagattattgtagaggatataagagtacgtgatttgataaaagataaaggatactcaagacaaaaaagaaaacaaatacagttctctaatttctatatgattagacaacagctacaatataaatgtaaagatagaggtattaaatttacagtagcagatacgtattatccttcatcacaaatatgtagtaaatgtggagaaagaaaaagaaactctttaaaattacagttaagtgataagatatttaagtgtaatacttgtgggtatgaagaagatagagacataaatgcaagtgttaatttaaaaaattaccataattcactatgggtaaaagaacaaaaccaataatatgtttgcttttagtagggttccataacccgaaataataatgccttttattttacgaaggaagttaatttaattaacatatggtaatctcattattggcgcaggaatagcaaacaaatctctagaagtagacggtattagtgaaactattggtactacacaatctgctatgtatggtggagcatcagcacagattattcaaattgatgctgatattaaagaattattgtctggattacgttcttattatgggaacaatatggtaggaatttaaggactgattaaatggaaaaaagatatattttaggttcaactaacaatgcagaagtatttaataaatctacacagtactcaacaaatacaggtagtatggaacaagataaaacaaagtatacaactagattagaatttgacactactgatatgattagatttattaatgacagaggtattaaagtcttatgggaaagtgcttatttttgtccttgtttaaataagcaaacagggcaacctagaatggattgccctagatgccatggtaaaggtattgcatatttaccacctaaggaaacaattatgatgattcaatcccaagaaaaaggcactaactatatggatataggactacttgatacaggaacagctattggtactacattacttgaaacaagagtatcttacagagatagatttactgtaccagaagtattaatgcctcaagcatctatacactatgtaagtgaagagcgtatagaaaaaggaatacctatgtactatgatgtaaaagagatagaatacatagccacacaagatggtattattcctaaggatgattataggttagaaaataatagattatttttagataaaaagtataaaggtcataatgttactattaaaatattaatggtacttagatatattgtttcagatattctaaaagagtcaagataccaatatactaaatttaaccagtcaagacctaaatttgagaatctaccacaaaaattactacttaaacgagaagacgttattgttacacctgcaccgtataaggtagaaagtgggataggtaatgatatagaggctaaagtagaagaccctaaagctatacctaataaacctaatattagtaatatgggtggattctttggaggagcgtttgaatcctagtgggtaaacgagtacaagtaaaaggtaatagacccaacttatttaaaggtgaccaaccacgaaaagtaggaagaaaagctattaatgagatgaaagcagagattctagataatttacaggtagtagctcaatcagcttcagatgatgatgtatatattaagcgcatgcccaattacttagaagtaactgaaaagcaattagaaaagcaaggtgttatagaccttaaaaaagcctttgctaaatctagtaaacgtaaacctaaaaaagatggtggttggtacttaattgtacctataagaataaaagcaaaaagccttagtaagaaaacgtaccatgatttacgaaaagcacagatacctagcggagcaaaatcagttactaaagtaacggattatttagatggtagaagaagagcagtaacacatcctgcattaaaaccaaaggctatgagtaggaacacaacaagatttaaaacaaagaataaaaagcagtctgggtatgcaatattcaggacggtatctaatacctcccccgcaaactcatggattctaaatagagataaagtaacgaataaaaacttttctaagactacaattagaaatatagaaaggcttatgaagtggaagctaaaaaacatgaagtaaagtaggtgtattaacaatagctattatttctatggactcttatttacgaaaagaattagagccaagactacgtacagtactacataattgttatattatagatgaagttttaaaagactttgatgaacaggctcgtgattcttttaaagaatcattttgtggtaaggatgcacctaatgaggttactatagggtatgattttcccaagtttaaaaccaaccatggtgcgcactacttaatacaactagatgaaggtagtgaaatgcagaactcactaggcaacgtacaaggttcttatatagaggctaatgatgatactagacgggaattagtcacagcaaaaaaagttaatgatagattagtatttacagtatccaaacctatacatgaagttttatacgtagaagatatagcattttcttcatcggatgaagtacaggttgaggataatacattttcttttaattatgaaggtaatgaagagtatgaaggctataatgctatcattacatatgtagaaaaaactaacaatactaaaggtatcgttaaaggttttacggctgaagaacgttgtattattgtagggttatcatacaatgtagatgttgccagatgcttagatgctattctaaagatgttattaatatctatgagggatagtatagaagaacaacaatccttacaactacagaatctaagttttggtactatggcgcctattatagatgatggagagagtcctatttttggtagaccaactactattaagtacacagtatcacatgatttagactatacaattgcacaagatattaataaacttactttcaaggaaaggaaagataagaatggcaagaaaaagaacaacacctaagcataaacataaagggtatgtccatattgacactttcttggatacagcaaaaactgtatttcatttatcagaagggcagtgtgaaggttttagagcccttatgtctggaaaacattatcaatttcaagaaacagacttcttaccttacttagaagattatttaggtaagaaattagaagtttaaaaaaataaaggagactataatacatggcaaaagatatatttccacgtagaccaattcaacgtccccatgcttcaattgaagtagatacatctggtattggtggctcagctagtaacagtgagaaaattctatgtttaattggtaaagcagagggcggagaacctaatacagtctatcaagtacgaaattatgctcaagctaaatcagtattccgttctggtgaattgttagatgcaattgagttagcttgggggtctaatccacaatatacagcaggtaaaattttagctatgcgtgtagaagatgctaaagcatctcaactagaaaaagggggattacgagtaacgtctaaaatacatggttctgtatctaacgacatccaagtcgcattagaaaagaatacaatcacagattctttacgtttacgagtagttttccaaagagataattatcaagaagtatttgataacctaggtaacatcttctctattaattataaaggtgaaggggactctgctacattttcagtagagaaagataaagaaacacaagaagcaaaacgtttagtgttaaaagtagatgaagaagaagttaaatcttatgatttaaacggtggcgcttattctttcactaatgaaatcattactgacattaatgagttacctgattttgaggctaaattatccccattcggtgataaaaacttagaatctaaaaaattagatgaaatgaaagatgtagacattaaaggtaaagctgtttatgttaaagccgtatttggagatattgaaaaccaaactcaatacaaccagtacgttacatttgaacaattaccggaacaaacatcagaacctagtgatgtggaagtagaagctcatgaagattccgcaattgtatccgctacatctaaacctaaagcaattgagccatttgaattaactaaactttctggaggtactaatggtgagccacctgcatcatggtcgtctaagctagagaaatttaaaaatgaaggcggatattacattgtacctttaactgaccgtcagtcagtacattcagaagttgcaacgttcgttaaaaaccgttccgatgcaggagagcctatgcgtgctattgtgggtggcggtattgcggaaacgaaagagaaattattcggacgacaagctatcttaaataaccctcgtgttgcattagtagctaactctggtaaatttgttatgggtaatggtcgtattttacaagctcctgcttacatggtagctagtgctatcgcaggtttagctagtggattagatattggtgagtcaatcacgtttaaaccattatttgtcaacagcttagacaaagtttacgaatcggaggagttagatgaacttaatgagaatggtattatttctattgagtttgtccgtaaccgtatgactacaatgttccgtattgtagatgatgttacaacattccctgataagaatgacccagttaagtccgaaatggctttaggtgaggcaaatgacttcttagtaagtgaacttaaaatcttacttgaagagcaatatattggtacacgaactatcaatacaagtgcttcacaaattaaagactttgttcaatcttacttaggacgtaagaaacgtgataacgaaattcaagatttcccacctgaagatgttcaagtaattattgaaggtaatgaagcacgaatcagcttaactatcttcccaattagagcgcttaagaagatttcagtatcacttgtttacagacaacaaacattacaagcctaatgttttatctcttatagagggtttaggcttgtaataccctctataaatataaatttatggagagtgaataattaatggcatctcaaaataagcagacagtccatactggtaataccgtaatgcttatgattaaaggtaaacctgttggtagagcgcaatcagcgtctggtacacgttcttacggtacagagggcgtgtatgaaatcggttccatcatgccacaagaacacgtttaccttaagtatgaaggtgaattaacagtagaacgtctacgaatgaaaaaagaaaactttgcaaaattaggatatgcttctcttggtgaagaaatccttaagaaagatattattgacattgttgttattgacaacttgactaaagaagtacttgtgtcatatcacggttaaactaaattgaccctatatttagagataagtatagaaaataccttaaattgctggaaaaccctaaagctagttagactacaacgtaactagaaatggtaagcgtgaatgttgagaaatcagaaaaaattaactggatggtataaggttaaatcctaaatactaagtagtgggtaatcagcatccaagctcctaagtcaatagataaggagaaggttcaacgactataataggtacatcttattaagatgattgtatagtctattccgaaataaagtatcacgaaagtgacggtatataaggctcagcaaataactatgatgaggcatggaatactaacgagattgtaactgaaagtataacttttagctacctttcagcgtcagataaagcctcaagataatatcttactagacctagagtattctaggtctttttttattgacaaactattacttttatgatagttttaattatgaggtgataatacgaacaatagacaatctaaattaaaaggtcaaaaccaatttcattattatggcaagccagcagataaaggttgttataaacgattaaagaaaaggaaaacaagaaatgagcttaaaaaatatttacaaacagaaagtaagaaatacattgacaaatagaaaaacaataggtaaactaagtaaaactagggcaacttggaacattaatccagtaacaagagtaaagaaaggcaagaccaagtatacaagaaaaaagaaacataaaaatgttgacagtgattattagtcattgtataataaggataaaggagctgatacaatatgttttattcagaaagaagaacaacttgcaaggaaaggcaacgtaaaaaacattattcacaaatggcgttacaagaaaagaaagattgtgtttcacttttacaaaatgtttttgagcaatgtaaattctttaagatgacaccacattttaaagaaaaatcacttactaggatagattttgacttacttaaaaacttcatacttcataacaactattcattcttaaatattatagaatacaatgaaacaaaaacacggtacaatattcaaaaacgtattgtactaaaatatcatgagattgtgaaagtaaaaggtattccttgttttcaatatttagttatcacaaataatggtaagttagttactacatattataataagattaatgatgtacatagaactttagacttaacatattatgataaaaatttaaaaataagtgttgacattgaataataaatagattacaataaacatataaacatgaaggagattgattttaatgaatacaaaacaagaagtattagaaagtatgaaaaaagaattagggtgcttacccgtaagtgtaagtatgacaagtaaatatgacgcattacatattcatggtgcattagaaaaatttaaagaactaacagatattggtaataaattaacaaagaaatatttaaatggttcagaaattttaacaattaattaccaagattttgattcgatgcaagactattatttatatttattaagaaactactcaaaaattaaacaatgtttaggtgaaattactgaacattttgaacaacgaattgcatagaaatatattatgtagtataataaaaaatatactttgatgttatattaattatgaataggaaaaaggaaaggaagttacggaattgacagaagaaaataaaaacgtagaagaagaaaaagaggaaacacctaaagaagaatctaaaaagttaacaaaagaagaaattgataaacttaaatataaacaacaagaagaacgtgaagaagttattaataaagttattcgtggtgtaaatgatatttatgaaaaagaatttaaatttgataacttagatgaacctgtaacatttaagattaggtaccctaatgctttagaacaaggtcaaattttaagtgtgcgttcttcttattttaatggtactgatatgtatcaatcacaagagattatctacgcattccatatgttagctacattaaacgtagtaggtattgatgtacctaaagaatttagaaacgctgaggagatttaccgattagagccactattagagctttactatgattgggtggcatggttaaatacctttcgttactagaggtgacgttgaggaggtagggggaatagagcgcctcgtaaaagaaaattactctaggaacttatggactattatgcgtgagtttaatgtactccctacagatgaaaggtttagaaatctaactgaccttcaaatagaatttatattatacaatatggtacaagataataaagaacaagaacttattgctaaaggtaagaaatttgatagtcactttgaagatgatgataggtcttggatggaagctgatgaggaagactttgaagttgtaccagattatattgacttagaagacattaataaacaaatcgaaaatagattatcagcagaagaaaaacaacgtagacaagaacgttttgaatctgatgaccctgacgaaatggatagaacagatttagttttagaagaacaacgtagacgtatagaagaattagatagggaattaggtataacgaatgataaacctgttgaatatagtaaagatacaattggtcaagccttagatgagcttgatgaagatgattggatttaggggtagtagtgtttctactaccctatttttaagtaataggtggtagtagaaatgcttaatgataattataggttagtattatccgctgatagttctgacttagaaaacggacttaaagcaatacaagcatatatggatgcattggagaaaagtaatattgatgctccattagataaatttaatgaaaaattaagacaaattgctaaagaaataaaaaatgttcaaaaaatacaaaaaaatgtagaaggcacatcttttgttaaagctaaggacatgaatgagttagtaaactcatctaaaaaagcacataaagaaattagtgacttacaagacgctttacaaggtgttgctgaaactgataaaataaaagggttcattgacccaaaagtacttaagacattcagtagtttaaataatgcccttaaagaaacaggtaatattgttgatgaagtatcaggcaagactataggtgtagatgctgatattaaacaacgtatgcgtgacatgaaagagtttggtacacaaactaagaaagcaatggaattgcaaaagcagtttgacgatattagcgcacataaaaagaatttatcagctacgcaaagacgggttagtggtactctagatagaactagaagtaccaatagaatgacctacatacaaagtgagaaaatgaaagctgacttgtctatgttgggtagtagtaaacaacgtaagacacaagcggaaaacaaaatgaactcacttagggatgagcaacgtaaagctaggcaacaataccaagacatacaaggtagagcttttaggaatgaaatatctgaatcagagcgtagaaagttatctgcggaaatacaaaacgagattaatataagaagtaaagttattcaaaagtatgaagaactgtctaaagaattagataaaagtataggttcatataataaaaatagtagtgaatacagtggtcgtacagtagaagcacgtagaggtagctttggtagtttagtggctgaccgtgcaccttctataggttcccatgcagttatgggcgctttagcaccacttacagccatgttctttaatggtcgttctatatcacaacaaaatagacctaaaaaaatagctttaggacagcagttaggaactaacattgataatgttggagactacatgaatgaaggtatgaaagaaaaaggtttaggttatgatagaggagctacgctagaaatggcacaaggtgttactagtgctactggtgtaacagaccaaagtaaagtaaaaggtatggtagacgtactagcatcaagctcaagaagtatggggcttaatgatgaagaggggtttaggagcaccctgtccgatattcagcatacaggtgcatataaagactccccaagtctaggtcaatttacagatgctgtgtatggcggaattaaacaagcaggtcttgaagggcgtgctgatgagcaaatcaaagccttaggtcagattagtgaaagtgttggtcgtggtagaaccttaactgacaaagacatgaacaatgtgactgctttccaatcaactttagcaagttctggtagtaaagcactacaaggacaacaaggagcctctgtaatggttggattgaatgaaggtattagaggtgcaaacaacgacccatatctacgtaacatgatgggacttactgataagtatggtggcggtgttgttgggatgtataggtctcaacagcaactagataaaggtatattagacccagaaaacgtaactagtggcttagataggattaaaaatagtggggcttctagagaagaacaagaagctcacgcatgggcttatatgagagagttaaatccagaaatatctacagaagcctctaagaaaatatatgacttaaatgataaaggcaaacttactgaagaagaattacacaaagcaatattagataaccaagaaaaaggtaaagaatctaaagacaaaaatgaaaaagattacaaagagtctcaagcaggaaaagataacagaaatgaagcaaactctaataacacttccgaagatgcctatgatatgactgcggaaataagaaacctttctgggactattacaggactaggtgtaggtgttactgtacttgcgggcgctgtaactggcttagcagtagcagttgctaaatccgcaggtatgatgggagcaggtcaagtatttaagtggggtacaagtaagttccaaaagccaggtactaaaggagtaccaggaaacctgacaggcggtggaactaaaggtgggggattctttagtcgagctaaggacttaggtaaaagagctgtaggttgggattctgaaaaaggatggcaaggtgctaaaggctcatttaacgacccatttaaagaccaagtaggacgagttaaaggtagttttactgatacagctaaaaaagctaaaaatactgattggaaaggcaaagggaaagacctttggggtactactaaaaactttggtaaaaccctttttgagtctggaaaagatgtaggcaagggtagtgtatttagtgatataggtaaaaaagctaaaggactctttacaaatagaggaaataatggtatagttgcaggaggagcttctacaataggagatataggtggactatctagaatgggtggtctactaggaaaaatagctatgccattaggagtagcgggttccctatttaatgtaggtagccagatgaaacaaggttctagtcttaatgattctctagtaggtgaggctagtaactggattgaccctctaggactaggttatggtaagtctattacaggacaatttactgataaagcaatgaaaggtaaatcacttattgggtggaatacacctaaagggtcaggagattttggtttactaggtaaagcatggagctcattattcggtagtaaagatagtgaaactaaagcaggtggcggtggaggctcttcagatactaatgtattgaatggagcaaaagttgatagaaataaaaacaaaaaaatgactgctgaaatgcttagagaaaagaacaataaaacagaaacagaaaatcttaaagtatatcgttcattactagatagagctgaaagagtattacaagatgctaaagggctagataaaggagactctgagggaggttcatctgatggcggttctagtgcctcagacgtagaaggtacgggtaaaaagaaaatctggaacttccttaagaaaaaaggcttatccgatgcacaagtagcaggtattatggggaactttgagcaagaatcaggtatgaacccaacggctactaacccaagttcaggtgcttatggtattgcacagtggttaggtggacgtaaatcaaaccttcaagcatttgctaagtctaaaggtaaaaaggctagtgatttagatgcacaattagactttctatggcacgaaatgcaaggttcagaaaaaggtagcctgggtagatttaataaagctaagacagtagaagaggctactaagtactgggcgcaagatttcgagcgtatgggtgcaggagaagctatgatgggtaaacgtgtttctaatgctaacaaatataagaaagcgtatgccggtggaggtggaggtacttggagtaatccactacaacaatcttcattaagtaatacacctgtatcagcatttattccgaattctcaaagtaataattcttcagttaataataacaatagtattaacattaatattaatgttgaaggttccgaaaaccctaaggatacagctgttgctattgaagaacgattaaacagaactttaagcagtaatatagatatatttacaaatagacacagaagagtataagggtagggtactaatacccttcccttatttttaaaggaagtgacgttttggcacacaaaatgcatagatttagaagacctaaaataagatttgagtttcatacagatgaaaatatttttacattaaggtatgagaatacaaaggaagtgtcgggaaaccaattaggagacaagattataaatttcgaaactaaaaatgcaatggcagacgatagtgcagtatttgtattaacattagcaggagatacttattgggataagttgctaatgataaatgatattgttaaaatatttgttaatcctagtctcgaagatgacacagaagagcaagtactcataggtatgatttcatctgtaaacaatgtaggtagttatggtaataaccaaaaagcgtatagaattactggacaatcattttctaaaccatttatgaagtttggattaggtgttatgcgtgaggttaatgcttttctacctatggcaggttggttagaagatggtgaaggagaaggtaaaattaaatttactggtagttcttcccatgaagttatgaaacagatattagacaggtttgtaccttatatgaagtataaatatcccaacaagcaaggaaagcctatcagtaatttaactgagtattttatgtgggataatttagagagttgggatgaatatgagaatatacgtgatgttactccgtttataaactttgacggtagcttatcagacctaatggactttactaccgcaaaaccctttaatgaaaaattttatagaaacaataaggaacaaggaaaaaccgaattagtattacgacgaacgccttttaacccgacacagtggcgctcattagaatacacggtggtagaaacagaagaccttatagaagaagatgtaggtaaaaatgatgtagagacctattctatatttaacgtaacaaatagtatgatgtataaacaaatagatgctaaagtgttttctaaaccacaatatcataaagaacttgtagatagatacggatatactagactggaagtagaaaatccttttatatttgatgaggataaatctactacggatgaagaaagtaatgataatgatactgggcaagaattaggtacatatgataaagtagtaaaagacctaaacaatatgggaagagccaacatctcccgtaatacagaaaaaatggtttctaaattagctagaacgtatagaaatatacgtaaagataaagctaaaaagatagtagactattatgtagaaaacggaaaagtaccacaagaagaatatgaaaaaatagtaggaagaacagtagaatcagagcaagaacaggataaaagaccagaagctactaaaggttcagttataaatatccttgatggtatatttaaaaaagaagatgactataagaataaagaaaagaaaaataaggctatcggtaaactaagtaaggaatttagatttgggaatagggcaaagtctaaaaagctaatagataagctagataagacaagaggagctaccaaagataaagatattgagtcctttattaaagaattagccggcttgtctaatgtttctgctgacacaggctcagaccctagtgattcccctttgcttatattttctaagatgctttttaactggtatcataataatcctaatttcttttcaggagaaataacagtactagggcacccaaaatatgatttaggtcaaagagtattcgttaaagataaccaacgagatgagatgtgggaattttatgtagaatctgtacaacataaattttcatacaaagaggggtatatcacaattttaggtgtaactagaggattaaaatccgctactataccagaaggaagtaaatacagatttgcaaaaccgtggggagactcttctaactttgaaggaggtcttttaggggaaaaaacatctgatgctttgaaatcacaaggagttgaagagcgtaaaagttctggcagtgattcagagtctagcggagaacaccactcaggagcttctttatctagcctagacaaaaagtataaaggtaaagaattacctactcctagaaacaaaacagacactaattggactgcaccaggaaaccctaacacaggtcagtgggcaagggaatgtacatggtattgttacaatagacgttatgaattggggctaaaagctgttgcgtacggagatgctgcagattggattgcaggagctagaggagcaggtgtatctacaggaaaagaacctaaaagaggagcagttgctgtatgggctagaggagttccaggaggctctgcgcaatacggtcatgtagcttttgttgagaaagttacagatgatggtatttatgtttcagattataactacgcatcacctaagacttatggtgaacgactatataaaaaaggaagtccagaatatagaaatgcacaatacatttatgattaaaggatggttgttatgagaatagagcaaatagtagaactatatatagataatatatatagctatccatacccttatgatgaggaaatgtttaataaaaagaatgcagagatacagccttttgtagataattctctatacaaagcaatacagcgcttaagaccttactatgatgttgtagagtacatgaatatatcaaaaaaagagtatacagtcagggaaatgacggtaggggagtatgaagtggactttaaagtgaataccgtatcaaaaacaaactttaaccattattcagaatctacttttaaagagtctattaaaatacaattaaaccctattaaaattgaaagtttagacagctcagcagaacaatattatgctacttatcaagaactagaggataactacaaatatgaattaaccttaccttataaagtagtagagatactacaaaaaaatgtaggcaataaaagtatacaatatcaatttaaaggtgctcctaaagataatccgtttgatactagtagtatttctttattagattcttacaatatggtatattgggtatataataatgaatatacaaaattaaactacccattagactacaattcgttgttaaatagtgaagtatttaaagatgttagttttacacatagatatatatatgacatagacacattagaagatggggacttactattttttggtaaaaacagtagtatagtaggcgtatatataggggataaaaaatatgtaacaataaaagggaaatttcctagagacacaacatctatattaacatacgatttagaaaaagattgggacacatttaacggtaaaatatttagattaaaggaagagtacttatgatgcaattacaatcgtctttaggaaaaggtttaaaaaagattgattcagatgactttaatatacaagatttacttattggcaaagtaattaaagttaattatagaacacatactgtagaagtaaatgtaaataacaatactttaggaacaaaccccgtgaaaaagggtgaaaacttttctgtaccctatcctagaaagtttattggaacaactcctgaaggtagtacttttggaagtaatgaactaattacacctggctctattgttcttgtgggatttttagactcatcatacaacaatcctattataataagtacatacgggacttctttacaaaataaactgttaagtacatctcacatggagacgggtaactttaaagatgataactattataaatacttcagtgtattgcacaatatatatccatctatgacatatgactttcatgatggtgatggtactatgattaagacctttaatggtaagtcttttttatctataacatctaatgaagaagagagaccgcaagcaacagattatcatgtcggcacagactattatgatttatacccgtcgtactatagtaacaaaaagactaaagaacctagaaatcaaaaagctccaaacgttctttttaagcaccaaggaatgctaactagtaataatgaaccagataatcatatcacaaccttctatatagaccagaatggaactattagattttctgttatggagaaagatttacagaagcgtacgttttttgagattgataatgaaggtaatcaacgcacaacataccaaggggatagcttattgttagacgaggcacaaatctgggtagagtatggtattaattctgaaacaaacaccttttatataaaaaatgacaaacataaactagagtttacagatagaggtttagagataaataataaaccaattttagataacctagataatagtgtagaagatgctattaaaaatttagaagatgttaaaaaaggattagaagaaatcaactatctcttagacggtgtgggtaaagagaatattaaagagcttatagataccacaaaagtagctatagaacaagctaacaaagcagaaagtaccactaaagattttcaatctaatatagatacggtgagtgctaagctagacagcgcaatagagaagtacgatactttcatgaacaagactattaaagactatatagagagtactagcacacaattacaaacaatcaatcagaactatgtatcaattaaaaacattgtagaacctttaaaaggtatatcagaaaaagtaacaacattagagaatacaacaactgcatctagtatttctaagctagttaaagataactatataaatgacaaagatactgtaacaatagagcaagtaaatacatctatacctactattattgtagtacctgagtctttacgaagttctagtacttttcagcaagatttaagtaacttttcagtaagtgttcttgagcaagagtctgaagagcagagaatagcttacattgattcggagttaaaaaccttagaagaaacctctttacataggttagtaggaaaaaaagagtatcctctaatagtaacatataaagataaccaagataatgttaaaacatttaatactacaaactataaattattgtacaataaagagtttattcctttagacaatgttaatgtacaaatatcaaatagtaatataatatccaaatttaaaataggatatggtgtactaaatgagtttatagataataaaagctttacgtcaaaagattggtaagcacctatgcttacctttttttgatgttatattaaaattgaattgatatattaaatagaatatgatataataggttttgaggtgattaaccatgccacaatctgatggaaaaaacacattgatgagaatgagtttaataatcccagacaataatggaaataagcatacatacaggtttaaagtaaacccagaaaattatagtatagaacaccctcaacgttctaatattattaaaacaaaatcagatattatttttgaggactatggttcagatgtaaaaacaataacattttcaggtaccacaggatttaagcaagcaagaaatacaaatggtatagttcaaacaggtaaagaacgtatggatgagttagaagaaagaattgtagaatactctaatatatctaactctggtagcgtatctatatctaaaatggaactatacaattggacagataataagtattataaagtgcacttagcaccacagggattaaaaattgaccgttcagcagataaacctcttctttttaactatgaaatctctttaatggttttaggagatgcaggagagccagatagaagttcccaagtagaccctgaatttggtaatgtagtaaaaccaggggaagaagatgttaaaaatagactaagtaacagtagtagtgatgttataagaaacagggatagaaacaatcaaaaatattcagtttatgacaacactagagaaaacataagaatgaaaaacggtaataacttatcttctaatggtaaagtaatatataatccaagagtaacaactaatggattaagtgggacattagattctacggcacttttaatagggtatggagatgggggaatacaaacatgagacatataatagaacagccatctggtttattaacatacttaaattctattcataatgatacacggtacttgaattttaaccttttagacgaagagattatgtttgtgtctagattttatacaccaagactccaattaagtgagttatctttaagaacattaaaggaaatacaagaagataaaataccaaaaaaagaaagatattttaacactaacacaatacctgaaaaagtaaaaaattcttctttacagttgcagaatcctcgtgtatactatatactacagactattattttagaggtatactctatagtaaactgttttatagagaatccagataacttagtatacttaacagataaagatatggatattatagtaagtaatatagcttatacaatagattacttaactgagtatgaagagtataatagtttagtcttagatttgagacaactagagatgaacctaaataacattaagttacaattacctcttttaaagaaggagatactggcaaatgaggtttaaaaaacatgaggtattttatggtgaaactatgcaagcaatatctcaaaagtactttaacactccaagctactggattgaccttgtagaacataatgatttaagatacccatatattgtagaaacagataatgaaaaaaaagaaaacccagaacacttagttacttatggagatacaattattataccttcggaagaagagttatctaatgtatccttacaggagctaaataaaaaagatacagaaactttggtagaattatcacttgggagagacttaaatattacagatgatgagaagtactttaatgctcatggtagtagtgatgagatattagcttttacagacaatggtagcggggacttagatgtcgttaggggtgtaagtaacataaaacaacagttacaaacacggttattaacaccaaaaggtagtttactactgcacccagaatatggttctgagattcacaatttatttacatacaatactatagaaacagctacacttattgagatggaagtggcacgaactttaaaagtagatacacgagtagagaatgtaaatatcataaactggacgataaaaggcaacgaataccatggggaattcgaggttaatatacgttctgttgagaattctattagatttgttctaagtatggacgattctggtataattgcattattttaatagaaaggaaatttttatggaaacaaggaaatttactaatatattatctaagttaatagataaaacaatgatagctacacataaagttactgactttactcctggttctgctattcgttctttgtatgaggctatagccctagagatagagcaattatatattttaactaaagaaaatatctattggggtattcaagaaggtgttattgagtcttttgattttaaacgtagacatgctagaaaggcatatgggaatgtaacacttaggttttacaaccctattgaggaagattatatcattcctaggggtactacctttacatcaactaatcaagagtacccatatcaatttgaaacattagtagagtatagagtacctaaagattctactgaagctatactagaagtattctctaagcaaacaggagcatcaagtaatattcctgagaacacattaaatgtaatgacaagctctcctcctagcttacgttctgttagtaatgaaacatcattcaatacaggcgcagacttagaatcgttagaagattttaaaaaacgattccaagactttgtagaaactagaggcagagctactaataaatcaattcgatatggtgctatgcaagtacctgatgtagaaggggtttatgtttatgaacaagtaggattagttacagcttttgttcatgatagaaatggtaacctaccagatactgtaaaaaagaatgttgagcaagaactagaggactatagacctagtggtataatgttaagagtagctcctgtaattaaaacgttaactgatattagtgtacgagtgactttatctaataaggacaggatttctgatacattagagcaccatatagaacaagtaattagaacgtacttaaataattttaaagtttctgatgatttaattttgaatgacttaattcaagttattatgaacatagatgatagacttatttatgatgtagagttttctacaatccatggaaacacaataactaaacctgaggaaattattcgagcaggtaacatagatgtaactttaatataggaggggtcttgtgagtacatttttaaaaaatctacatccgttattaagaagaaagaagagtactagagataatcaagaccctaactacgctttaataaaagtgcttaacgaagaaatcaatcaagtagagaaagatgttattaagagtaagttgcaatcatcattaaaaacagctacaggaacctacttagataaatttggggactggttcggtctatatagaaaaaaagatgaggaagatgataaatacagggatagaattatagagtatctcttactgaaacgaggaactaatagttccatagttaaagctattaaagactatctagaaaaagaagatgcatatgtatatgtgtatgaaccttataaaaatatattttatacgaataaatcccacctaaatggtgaggaccatcttatgggttattattatagatttgccgtaattaatataaaaataggtagttactttccaacagagattttagaggttattaataagtttaaacctgcgggggttaaagtatatttaacctatgacggggcatataataatactggaagttatgtaataaaaatgttagagggtcttccaataattacaaactacacacaggtaacaaggctaagtggttttggagacacattttatgggcacttaaatttagcagaaagaaaagaatcggttactatatcttcatctttatttaaagtaaatagaagcaaaattaatagtgaagatgtactttcgggctcaagctcaacaggtagagtattttataattacgcatactattctagacatgattatgtattaaatgataatagcccagtaacatctagagatacgtatagtgctaaagaaatgccactagagtactacgcttataccaaagaattagataattacactgtgaattatactttacctacaacacaaagtgttaagtttatctataatagctttaatttggaggaatatttcttaaagtacaagcctgaagtagtaacaggagataaaaaggaaagtattactaatttcataggagaagcaacctatgactattacatgagtgccttagtacctccagaagaaagtatcatggtagacttacagatatttgattttgtaactaaacaatggaatactataaatacatctagagtatctagtaaagaagttaatataggagggtctattggttatttgactgactatattaacagtatgttagtactctttactagattacgtatccgagattacaaagaagtttccataaaggtcaactatatagacatatcgtttaataatacacaaacagatagatacacaaacaatagatttttaccaaatatttatagggtactatcatccttatctaactataattatatagatgcaactaaattaactttacctacaaatggggatattataagtaaacgtgcttactatccatcacagtatataagaataggtgatgcgtatagtaataatataaaccgtaacttattaaataatactattgtatcatcaggtaactatacaagtcactctaactctactataactaaacaaggtaatgaactaaaagtagagactggtgaaaaccaaacaaaaactgatgtagaactaagcaataatattgtattaacagcaggcagtacatatactctgtcttttatgtacaaatcaaatgacaatactaaacctttagactatacatttctacaagatactacaggaacaaagttacctttagtttttagtaatattaatcatacatcagcctatacaagagtaataaaaacatttactattgatggttcctctgataactacagtcttattataggtaaaaatagttctgatatttttcatataaaagactttaaattagagagaggcagtattgcgacagcctaccaacctaacccatctgatttatatacagtagaccctaataactctatcactatagagttacttaaagaggatagtacacctatagaaagtgaagtttatcaagaaaagtctaaaaacttatttcaaagttacaaagggtcaatagatataagccatgatattactaatacagagatgtttacaacatctgggtttgctagtacgctatattctacggactatttagataagattctagaggaagggaaacgctatacgattcacttcgagcttgaaattgtagatactatagatagctcgtttaaaactttttctgatacgcatggtatatggttttatagtgcgactacacctaaagataatattaacttacgagagcaaataagtaggacatcacctagaaaactagagaaaactgttacttttacagcacctagaataacagaccacagattaataatttattcaggtagatacacaacagatggtacagcatatacccaacctattaaaagtaatacaataaagattactaatctagtgatacaagaaggagaagaatatacagggtactctaagtctagttatgatgaggttaccttcctagataaaacagttaaagttaatggcgctagaacggacataggaaagattaatgtaagttcagtaaataatattcctagagcacggctattatacagcacttatgggggtaattatgacttaataaagaatattagtttatctcaaggaactaatactgtccttaataaatatattgatttatatgggttaagtaccatagactattctgatataacaccaatgtcagatgttatactacaatcaatatggaatatacctattaatactttaaattcacaatcaggaaacttagacggattatataaaggattctttaatgccttgtggcaaacaattactaataatgatgagcgtgtgtatactattgaaactttagacatattgaagaatgctgaaggtggcgtgatggacaacgctacaggggaagtagttacactaacatctacaaatgtcactagcaaaacaactttagatacatactcatatagtaattataaatatcaagagactgtattattgaactctaatataccactttctaataatacattaactttagatacagagtcctatgagaaagacactacattcttaaaaataatttaatagcgtattgacacggttcttgttatgtgttatgataagaaccgtgatatattaatatatgacgttaaaaaaggagataaataaatggctattgcaacgattaattcacatgtagaactagctaaatactatgctagtaaagattttcatttagtactaggaaagacatcaccatggtctaatgaacaaagccctccacaacctaaagacacaacaagtgccttacaagaggttattgggtataagaaagctaaaaaagtagctttagtacgtcctgcacaaccaggaactgatgactctaagcaaaaaatccaatatggtaatagagaatgggtagaagtatcccaagaaaatgctattacagaaggtgctaaatatgtttaccttcaagcagaagtagtaggggacgagcttcctttaggaacatacagacaagtaggttttactatgggcttaaaatcttccacagataaactaaatttattacctagtgaagtacaagatgcgggaacattaattttctatgataataaacaatttcaaaaccgagtagctcaaacaacaattattgagtcgtttattatggaattataagagaggggataattatatggcattaaattttagtggttctccttatttagatagatttgacccaactaagaacagaacaagggttctttttaacccagatagaccattacagcaatcagaattaaatgagattcagtctatccaaacatattatctaaagaactttggtgacgccgtatttaaagatggtgataaggtatccggaatggggtttaccttagatactaataacattttaagagtaaacccaggatatgtttacattaatggtaaagtaagatactatgaaagtaatagttctgttaaacttacaaaagtaggaagagaagaagtaggtattaaattaacggagcgtattgtaactgctgatgaagataacaacctactagaccaaacaagtggagtacctagttatttttctaagggtgctgacagggttcaagaggacatggtacttactgttaatgacccttcaagtgctactatttatgtattccaagacggtgacttatatgtacaagctagtaatcctgagtatgacagaattaataaagtattagctgaacgtacgtatgaagagtcaggctcttataaagtgcaagggtttgaaatttttagtgatgtagagggtaaaggtgaagacacggtacctatcacagttgactcaggtattgcttatgtattaggttatcgtgtgaacaaaccagtatccacacgtcttaacttacaaaaatcttatgacctaggtacggcagaaaatgaatctactgtatataatgactctaataaaataatcaccttagcaaataaacctgttaaacaagtaaaccgtgtaactggacaggtacttgtagaaaaagaagcaatttctagagacaatgtaggagatactgtagattacttagcaaacaatacagcttttgaaattgtacgtgtttggacagaagatagccctgagcatactacaaaagaatataagcaaggcacagattacagactaacaaacggacagtcagtagactggtcgttaggcggagacgagcctaatggaggtactacatactatgtttcgtataagtataatagacgtatggaagagagcaaagattacactttgaaaacaacaggggaaggtgcaaataaatactggtacttagacttcaatactgaaggaactaagccgattgaccagtctgttgtattagtggactataactactacttagcacgtcaagacttgattgtattagataaagatggtaatattaaggcattaaaaggtgaacctaatattttaagaaatgttacacctccacttcaacatgacccatttactcttcaactaggttatgtaacaatattcccagactctgatgctacagaatgttacacacaatctgtagagcgtttatcaatggagtccttacaaaagatgaaaacacgtattgataacctagagtataaccaagcattgaacgcactagatgatagtgctatggaaggtcaaaacccactcacattgcgttcagtgtttagtgatggctttgttagtttagagaaagccgatgtaacacacccagactacggtgttgctatgagtttcgaggattcagaggtgacactacagtatacaactagcgtgaatgaccctaacttactaacaggctcaagtaatattcacaactggggtagaattgtttcagcaccatttactgaggagcgcacaatttaccaaccactagctagtgaaacacttaatgtaaacccatataatataccaaataaacaaggtaaactaaaattaacacctagtgaggataactggattgatgaagagaatgtaacaattaaccagttcggtaaagctaaggtagttaatacacgtagatggtgggttcatccagaggcttcagctaaacgtaatgcaaaatcaagagcagaacaagcaaaaattgaactagataatggattagagtttggtagtacttttgaaaaatggcgtgcaacaactacaggtaccatgactgaagtgggacataaaacattagaagaaatgattgagttcattagacaacgagatgttaagtttgaagtcactggactacatcctaacgacaataacttagtactgtactttgacggtgttagagctcctattacaccagacgcaggatatagaaaaggagctgaagaaggttctattgttacaaatgcaaaaggaacagcaaaaggtaagtttacaattcctgctggtatccgttgtggtaaccgagaagtaacacttaaaaacgaaaactctactagctcaactacatattccgcacatggaagaaagaagattaaccaagagattatcttacgtacacgtgtcacagtaaacttaattgacccattagctcaatcattccagtacgatgaaaacagaatcatcagttctgtaggtttatactttgcatctaaaggggatgacaactctaatgtaacggttcaagtgcgtgaagtaggtaatcaaggtttccctactaaaactatttacggagaacaagtagttaactctagtgatattaaggtatctagtaatgctagtgctgagacaagagtatactttgatgaccctattatggtagaagcaggaaaggaatacgctttagttattcttacagaaaatgatgagtatactatgtgggtaggtacacgcaataagccacgtattgatagaccaacagaaaccatttctggtaatccatatgcacgaggagtattatatagctcatctaacgcaagtgcatggacaattcaccaaaatagtgaccttaaatttggtatttacacagctaaatttaatgagacagcacaaattgagtttgaggaaatgactgatgttaaaggtgacagacttgtattaatgtctacatacttaacaccagagaacacgggttgtacatgggaaatgaaagtaatcactgatgctatggatgagggtacaatattcgatacgcttaaatgggagcctgttggtaactaccaagatatagaaatcgattctgtaattaaaaaagttaagttacgtgcaacatttaactctaataaatatatttcacctctattaagtgtatctgatttaacatttacaacattcttaactaaccttaaaggtagctacattgctagaggcgtagatatgagtgaggcaccattcaatactattagatttagttacgaggcatttctacctcaaggggcaacagtgactcctaagtactctgtagatggtggagtatcatggaaagaatttagtaaaacagcaactaagactaaagccaacaacgaatttactagatatgttattgatgagaaaacaaataaatcatctaaagataaaaaattaaaagttagattggacttagcaactaagaatagtttcttacgtccacgtgtaagacgtctcatggttacaactagagatgaataattttaaaggggattctatccccttttctataaaggaatgatggtgtatgccacaggagtatagggacccttactcacaaacaaaaatatttattccaacgccacaagaaaagagtgtgttggagttacatgacagattatcaaagaaagaaaaactatttgatgaaaaactaaaaaaactagatgaattaattagtaggagtgaacttaactaatggcttttacttttaaacaattaaaaggtacggactatttaaaagatgcttaccctaaaataaatgagttaggtaacaacagtgcctccttgtcacaagatgtagataatatacaaagaatgtttcaaaatatccagacctttaatattactgatagcagaggtagaactcctgaggggtctcagataaatcaagattacgtaagtagctatgttgatgtatatggctctttagtatatccgccagacagcgttaattctacaggttggatttacgctaagattactgcgtcgggtgagggtatgttagtttttttacctgataattcagaaaaactatttacaaaggttaaacataacggctcatggtctgcatggtctagttatagctctgatagagaaattaataaatttattccaggagatatacttaataatggtgattttgattacaagtggggaacaggttctggtacattagaggatgaggtagctaatgtaaactttgggctgtccttgttctatgcaacacaaaacatgagaaattctaagttccctgaacccgttatgggtacaatacagaaaagtgttgaaggtagcggttttaaagctattataaaagctacaggggtttccggcaaagaatatatcataaatatacgtaatggttacagtaaacctgtatctcaaactattacaacaacggaactttggaaaggtagtcaagatttagctactaagagtactgttaggttaaaagacacagttaataattatgattatattgagtattacgttacattttctttaggtggtgcgtatacatttaaggtatcacctcaagtatctacggtatactttcgtgatttaaaccttgtgaataacgattctagtgcgggatttgacttatttgagggcgctgttactgttgatagttctggattatcaattacaccttatatgagtaagaaagtaacaggaaatgcagttcagtatgaccaaactggaaagattacgttacaagccgttactggcatcaaacaaatataaaggaggattagaatgagcagacaaattaattttgcagtagatgggtatctcttactcttattagataagaacgttattacaggatacgttacatatggacaggaggaggagggtgtagctcaagtgttagtacatgaggacttactccctagggacttctttaaagaatttaaagccactaagtataaatattatagttcaccagaagagattattattaacccagattataaagaacctcaacaacaagaagaaagtagtgctgagagtaccccagaacagcaagaaaaggattttaaatcagttgaacatttaatggacgaactacagagtattaaagatatgcaaaacaagattttagagttacttatgggaaagtagggtagtacatggcattaataaattttgaagcaataacagagtctgatacacttaaagcactgacagaggctgtgaataatatagggacagagttatctaaggatggtaacatttttgctaaagtagaaaaactagaagcacaatctaatagtacccctaaatattacctagtgcaagataatggtaaagctaagcaagtgacaagcagaaattcattaagtagtatcactcaaacaggattctactacgtagcctctaatatatctatttacttaccagaactacctcaagaacttagaacagatttgcttttaagtgtatataaaacagaattaggtactgtacagtttgttaatactattacaacagtggaatctagaaagtatcttgcttatcgtgtaaattataatggtatggtaggttcttggaggtttatagactcacggattaataatagaagtactttggctgtaggaggttcttataaagatattattgcaccaggacactacatcgtaaatagtatgaaagaattaccggacaataaatctgatggctatttatccgtaataactaatcaatctgatgagcgttcttatatttacattagtcccgataataaaatgtatcactcagtatctaatcaaaccaaagactttgactgggtaaaagagtttggtattgaagatgtagaagattacttattaaccacggtaaatggtgagtataatcttttattgtatactgatgacggtgtttcttttgagacaagagtaaaagagtacttaaactctttacctagacctaaagccttcacattctatatccagggcggagtagcaggtaatccctcaggaagaactagctgtaggggtatttatatcttcgacgccgtagaccacggtgtatactatgctattgattaccatggtagactattcacgggggctgtgagtaatggggaatttaggaaagtgcgctcagcaccatcatctgatgtattatggactggtgcaaagaacttcaatgatataggtaaggttgagaaccttgatttccctattgctgattatgattatgtaaaggtatttgttacagtaccttctcaaagtaataatacaagtgatattacgtacgatttacaaaacggagctcatgagttttatattagaggacaggcacgagtatctgtgtcaggtactgtagtatcctcaagcactaaatctgacttataccgtgtaggtattgctttcggtaaaaagtcatggcgtattacagatatgtcaaaaactggtactaaaaacgcatatattacccgcattgtaggtgtaaattactaaaactaggattaatttcctagtttttttattgacttaaacaataaagtatgatagaataacccttgtgtatagggtggaaggagcttttatgaagttacgtataaaaaaccttttcacttatgttgaattcgaggaggatgataagtatttaaaggatatatttttaaagcgggtacacacaacaataggagcaagacagcaaggatttcagtttagccctgcatataagagaggaagttgggatggatatattgatttctacgtttatgaagaagataagtttcctacaggactactacatagagtagagctattactaggagagttacaatcaagatataacttccaatattctaagattgatgaacgttctgaaagtttcttagctcctgaagatattgataaagaaataaagctattagataataaaataggacaaattaccttacgtgaatatcaatatcaagctgtgtatgaaagtttggttaattttaatggcgtattaaagattgcgactaatggtggtaaaacagaatgcgccagtggaattatagaccaactattgccacaacttgaaaaaggagaaacagtagcattctttacaggctctacagaaatctttcatcaatctgcggacaggctaaaagagcgcttaaatattcctattggtaaaataggtgcaggtaagtttgatgttaaacaggtaaacgttgtaatgatacctacattgaacgcaaatatgaaagaccctacagaaggattaaaagttacacctaagcaaaatattagtaaaaagatagctaaggaagtattgcctaaatttgaaggtggcaagaatcaaaaaaggtttatggatatgtacttaaaacagtttgaacctaaaactaaagtagaccaaaaggttaaagatgagttaattaatatttacaatacttctaagtcagaccaagaattattattgaaccttagaaaacataatgcaatattccaaaatattgctaaaagtaaaaatgaaaagaaatatgctaaataccatcaaatgcgtgagtttttagattctgttgctgtaatgatagtagatgaagcacatcattccaaatcagattcttggtataacaatttaatgacttgtgataatgctctttatagaatagctttgacaggctctattgatactcaagatgatttacttaatatgagattagaggctctttttggtactataatctctaaagtatcaaacgaatttttaataagtgaagggcattctgctaaacctactataaatacaataccaatcctaactcctaaggacatagaaaaccagaaagaatatagggatgtctatgatatagggataaccaataatgagtttagaaataaacttattgcaaaactagcagaaaaatggtataataaggataaaggtgttttaattattgttaactttattacccatggagagaatatatctgaattattagaagacttaaatatagagcacttctttttacatggggaagtagattcagaactaagacaacaaaagttaaatgatatgagaagtggaaaattaaaagtaatgattgctaccagtcttattgatgagggagtagatatatcaggtatccatgctcttatcttaggtgcgggtgggaaatctctaagacaagttctacagcgtattggtagggctttacgtaagaagaaagatgataatactacacaaatatttgactttgaggatagaacacacaagtttttgtacgagcattttaaacagaggttatctatttatgaggaagaaaagtttgaggtaaaagatgttactagaaagggatagatgagatgagtaagataaagagaagattatatagttatttagagcagtattcattaaagcatgagtatgtagcaacaacaaaaaaagaactagctgaaaaacttaatatctctttatctgctcttacctctaaccttaagaccctagaacatgaaggtaagattgctacttctactaaaagaggtaataaaggtggttttattatcacacttaacttacctaatactgtaaatgaggtagaaggtagcattatagagtcgaataaagattacgctaattacctaaaagagaaagtattccctacctatcaatacaactataaaggtacacgaagaactaaagttgaaatgataaaatataatgctattaaggataaacagagaaaagttatatatgatatgaactattatttagatgggttactataccctgataaagaattgtttaaattatcttatgacccagaaggtttttacaaggcatttattttatataagctctacaatcaatactcagctatgttttgtaagtataagtttgaagagactaaagaagatttttatactgctaaaatgcagtattatatgaattatcctacacctaaaaacttttttagtacaaaagaatttactatattttataagttacagaaattagttaaagaatataatattaacgtatttaagtatatgcaaaatgtatttaagactgtagattatgaatacagaaatggatataagaaaacatacttgccacaacctaactatcttataagtgaaaaatatatagatagatatttaaaatatgtaaaaggtgtacataaaaatgtaaataatactcaaaaacatgtaggtgacttagagttaattgtagatagctctatctttacagaggacttagtcgtacagcaactccatcagctatatacggaaggacttaatattaataaagtagacatggaatctatatttaatgacgctttagatgtagaagatgtaatgctaggattgtctactaatgaaaagcaattaatcttactaaactttaattatgagatagaacaaaaagtaagtcacttacctaatagagaaaaattattaattagtgattatgtaaaacaattaattatcaatgagtatgatagttcctcattttctaataaggctttactctccatgtttatactggagcgtgaatattttgtaggagataagatatttaacacacaagagtctctatttaaatataataactttataggtactctacatactcaaggtaatcaagtagtaggatacacttatttagtccacagaaattttaataaccttatgtacacaatcagaatgtatggagacttaaaagggtatcaaacaaatttaaaagacttatataaagtgttagaagaaaataatctaaaagggttgattccttttacaaagtatggtacacttgactatgacaaattgaaaggaagatatgagaatgagtaaaaagattaaggagattattttacataattcattaaaagaccctaattttgctagagatattctaacacaactacctaagcatttgtttagttcagaatccgaaaagatggggtatatttatactgcaataaaaagactcacatacactgttgataattactcaacagataccgttgctattaaagtagagggtttaatgaacacaaaccaagagtcagaagaagatgttactaatgctttaaaatatttagatgatttatatactgttaaagtagataataaagatgaatctttaaaaacagaaattaataagtatattaaaaaagagttatctaaggaagccttagtaaagtttatttcagaaaataagcaagaagattctgataactttacagaactaatagaacaacttaaacagatagaagttaataatattggtgaaggtgatggggaatttcttgatttctttaatgatgtagagaagaagaaagaattactatctaaactatccgtaaataaatattctactggatacacttcattagacgctcaaatagagggtggaattgcacgtggtgaagtagggttagttattgctccttctggtagaggtaaatctttaatggcatcaaacttagctagaaattatgttgttaatggtttaaacgtattatatattggtttagaggaaaagttagaccgtatggtacttagaactgaacaacaaattttaggtgtagataaatcagcattattaaaagataagttacaattaaatgagcaagtattcgatgctttacaaactaaatataaagagtaccctacactaggtaacttatatatgtctaagcatatgcctggtgaagtgagtcctactaagttagagcaaattattataaacacaatgattcgtaaagatattcaaatcgatgtagtaattattgactatcctaaactaatgcgtaacccttatgagaagtatgcatctgaaagtgagtcaggtggaagattatttgaggatatacgtaggttatcacaacaatataattttgtatgttggacgttagcacaaacaaatagaagcgcttatggtagtgatgtaatcacaagtgaacacgtagagggaagtcgtaagattattaatgctgtagaggtagctttagtattaaaccaaaaagatattgaatttaaaaatgggtttttgcgtctatatttagataaagtacggaatagctctaatacgggtgaaagatttgttcatatgaaagtaaaccctatgaaaatgagaattaaagacgaaacggaagaagaatacaaagagcatttagctttactatccgatgacggtagagaagatacaagtaaattccaaaagaaagaaaataaagtagaccaactaaataattcatttggaggactagaaatttaggtaaggtaaaaacttacctatttttttattttatgtattgacaccttatttgctatactgtatactaatagtatgaacaaaaaggagatgtaaatatggaattaaataaaatttataatgaagattgtttaaaaggaatgaagaaaattcctgataatagtgtagaccttgttgttacagacccaccgtacttagttaactataaaacaaaccatagaaaagacaaatctcataggtttaacaatgtaattttaaatgacgataatgaacaattaattattgattatataaatgaatgttacagaattatgaaacctaacactgctatgtatatgttttgttcttcgagtaaagtagatttttttaaacaacagctagaaaagaaatttaaaataaagaatatgattatatgggttaaaaataatcacacagcgggagatttacaagcatcttttggaagaaaatacgagattatatttttagttaataaggggagaaagaagttagaaggtaaaagaattacagatgtttggttttttgacagagttagtggaaagaaacaaatgcatcaaaaccaaaaaccagtagatttgttagaacaatgtattaataaacatagtgtagaaggtgatgttgtttttgatggatttatcgggggggggagcaccgctattgcttgtataaatactaataggaattacattggttttgaattagacgagggatattataaactagctaaagaaagaatagaaaaccataaaggagaactagatgaaaataaacaggatatgggctatgcctaatagtaaaacttttaaaataaaacctattaaagagcttatcactaattatattgaacataataaaattaatattgacccctttgctaatgctagtatatttaaagactttactattactaatgatatagatactcagtatgacactaattaccacttagatgctttagattttttaaaaacatttaaagacagtgaagtagatttagttttatatgaccctccatactcaccacgacaagtaagtgaagtatataaaaaactaggaaaatcagtaaatatgcaaacaacacaagccagttattggagcaatcaaaagaaagagatagctagaataactaagaaaggtggctatgtaatcacttgtgggtggaacagcggaggtattggaaagtctaatgggtttgagattgttgaaatactattagttcctcatggtggttggcacaatgatactattgtgacagtagaaagaaaagtaaaggagtaatgaaattgaaatttatattttttacagatagtcattttcatttatttacaaattacgcaaaacccgatgaagagtacgtaaatgatagatttagagaacaaattgaggcgttacaaaaagtattcgatttagctaaagaacataatgctaaggttatatttgggggagaccttttccataagagaaatagtgtagacacacgagtatataataaggttttcgaaacctttgctagtaatcaagatgttaaagtttatatggttcgtggaaatcacgatgcggtttctaactctttatacactcattcaagtatagacatatttgagacactacctaatgtcgaagtaacaaaagatttacgaagtgaacctctttctagtaaagttcaattaactatgtgtgcttacggagatgaaacagaggaaattaagagctacattaagagtggttatcaagaaggtaaggttaatattttagtagggcatttaggtgtagagggcagtttaacaggtaaaggtagccatagactagaaggagcctttgggtatcaggacttactaccagagcagtacgattttattttattaggacattatcaccgacgacaatacttaaataataatcctaatcatatgtatggtggttctcttatgcaacagtctttttctgatgagcaagaatcaaatggagtacacttaattgatactgatacattaacatctaaatttattcctattaacacaagaagatttattactttaaatgaagattcagaaaatgtagaggcattactacaatctaataattttattcgttgggtaggaacaccagagcaagctaaggttttagaattagatgatgaattagaaaatgtacaattccaaattaaaaaacagtacactgttgaaaaaagagtagattctgaagtaacagattcaccattacaaatcacagaaaagtatgctaaagagtattacccagaatctgttaatgaaattaaagaatgtttaaaagaagtactataaactattgacaaagcatacctaaaatggtatgcttttagtataggaggagataataatgttatcatataacaatcaggactttagtgacacaaaatttaaagatattttagataaagaacaactcgtagaatctctactaaaaaatcgtaaggttaatacccttgatattagcaacgtaaaaccaaaagtatttgatatgaatagacccgtattacaatttcaaaatgaagataacacattaacaaatatttgcaatgtagataatttagatgatggtattaataagattaagaaaatatttaaagactctaatttagaagatagattacattactttagagtgaacgagcatgaagattaccattggattgactatggctcacatcatacattttttagagtaacaaagagataacatagtatgtagggggatgtaaatatggaactaaataaaatttataacgaagattgtttagaaggtatgaaaaggattcctgataagagtattgacatgattttatgtgatttaccatatgggacaacagcatgtaagtgggatgaaattattcctcttgagccattatgggaacaatacgaaagagtgataaaggatgacggtgcaatagttttaactgcatcacaaccttttacaagtaaccttgtaatgagtaatcctaaattatttcgtgaggaagttatttggttaaaaaacaaaggagcaagcgggctacaagctaagcaaaaacatattaaagtccacgaatcaatcctagtatattctaaaaaaggtaaatatacctacaatcctataaagtgggaagtggaagaaaaacaatttttaacacaaagaaaaacaatgagcatgtatggagaaacaaataatatatatggaagtctaaaacgaaaaagaaaggctgatgatggtactagaaaccctataagtgtcattccctttaaagtaccaataacaggagcaaaaactaaaaaatatacaaaagaagttgatttgagaatacaccctacacaaaaaccagtagcattatttgaatatttaatcaaaacatattctaatgaaggagaaacagtacttgataactgtatgggtagtggtactactgctattgcttgtttaaatactaacagaaattatattggttttgaactagatgaagagtattataaaaaatctttagaaagaatagagaattataaaggagattgttaatggttaggtttaaaaaagtagaaattaaaaactttatggctataaaagaagcaactttagacttagataaccaaggattaatattaatagaagggattaacaaaactaatgattcttttgaggctaatggtgtaggtaaatcaacattagtatcagctatcacatattccctatatggaagaacagaaaaaggattaaaggctgatgaagttgttaataagaaagaaaagaagaatacattagttagactttactttggtataggtgaagataactacattattgaacgatatagaaaagataaagaaaacaaaaataaagtaaagttattctgtaatgataaagaaattacaggttctacaaacgatgttacagataaacaaatactagatttatttggtatcgattttaacacctatttaaatgcaatcgtatacggacaaggtgacatgccgatgttctcacaagctactgacaaaggtaaaaaagaaatcttagagtctattactaaagtagaggtatacaaaaaggcacaagaagtagctaaagaaaaagtaacagaagtaaaggacaaacaaaataaattacaggatagcattcagaaattagaatataaaattgattctattcagtataaaattaactacgagcagaataattataaagaacaattacaacgcttagagtctgttaaggaagataataagaaaaaagaacaacaaaaggcagaggaagaaaaacaattaaaagaacaactatataagttgcctgcatttagatatattgaggacagtttccaacattcagagcaatataataaggtacaacttgctctgcaaaaattagaaaaaaagaaaaaagaattattagatatttacaataatacaaaagtagaattatcagtggaatctacgaacctttcagcagaacaaaacaagttaaatagtttagatacttctgataagtgtcctgtatgtggctcacagattgataataatcataagttaaaagagcaggaaaatattaacagtacaattttatctattaatgaaagaattgaatacctaaacgcacaagaagatttatataaaaaagctattcaacaagtagatactaaagttaatcaattattagaagtagcaaaacaagaagaagaacaagaaaagcaacatgataagttagtttatgcaactaaagagtataataataacattgaatataataagaaatcattagaacagcagttaagtaatttagattttaaatatcaacagcaggatttatcaaacctaactccaacagaacctaatattaaagagtacgaaaatgaattaaaagaaataaacaatactattgacaaaataaagaagaatatattacaattagagaataagaaacatcagtataaagatgcggtggaggcttttagtaacaaaggtttacgttctgttgtcttagacttcattacaccattcttaaatgaaaaggctaatgagtacttacaagtattaagtggttcggacattgaaatagaatttcaaactcaagttaagaatgctaaaggagaacttaaagataaatttgatgtagttgtaaataataagagtggtggtacaacgtataagtctaattcggcaggagaacaaaaacgtattgacctagctattagttttgcaatacaggaccttgtaatgagccagaatgacttacaaacgaacatcgcactctatgatgaatgttttgatggtttagatacgattggttgtgagaatgtagtaagactacttaaagataggctacaaacagtatctactatttttgtaattacacatagtaaagatttaaaacctttatttgagaacacaattactatgattaaagaaaatggagaggcaaggttggtgaactagcataatgaaactaaaaattaaaaataaatttatgggagtactagaagttactaattctatgggtgtaactaatctagatgtaccattaaacaatatacacgagtggtatcctttttctaatgcgtattcttataagtacaatgtaaaaacaaaagaattagtattaaaaagattacgttcatcattaccagtgtcttatggtattagaacctctaaagaatatagtaaagacagagtatgcaacacagttacatggttaaatcatgcagtaaaggacagcaatttatatattatcaacaaagctaaatcttacggacttcctgttattacagaaacctacacacaagaagatgtagattatggctttgcacaactaaatgttattttttcggaactaaagtctttgattattagtcgttacttagaggataaagatagtgactttattactaaatttaatcgtcataatccagagacacagtatcatttagcagttcaggatgcagacgatgctgttaatgcaacctacgatgaattaggtcaaatgtacaagatgctacttctaatgaagaaattaagcaagcattaaaggattgattttatgagattccaagactttttaacgcaagaactaggaaatcctaagataagtaatggagagtaccaatactgctgtcctttctgtggtgaacaaaagtacaagttctatgtaaaagactctttacaatctgataacggactataccactgtaagaaatgtgacgctacgggaaaccctattacatttatgaaagactattattctatatctgcaaaacaggctatagacttgttagaaactaaaaatataacttttgagataagtccattagataactacagtaatgaactatcagagtcagataaacttatacttcatctaaggcataaaagtcataaaaataatactaaagataaaaaaccaccaagactgcccgaaggttttaaatatctaaaagataatttacaaaacaaagagtcttatccttttttatattacttaaaaaatagaggtattacacttaatcagattatagagcataatatagcttatataacaaatggatatttttacaaaacaaatggagaaaaagcaccattaagaaatagtgttatcttttttacatatgataacaatggtaattataaatattggaacacacgtagcatagaacctaatccctatttaaaatctattaatgcaccttccacggaacaggagtatgcaagaaaagattgtatctttaatttcaataacgctagaaaagaaaaagtaatgattattacagagggtgtttttgacgcacttacgttcggtaaatcgggtgttgctacgtttggtaaacaagttacccaatctcaactagaccagattattcagtatacacataaagatactattatctatattatgctagatagtgacgcacttacaaataacattaaattagctagaaagctacatgaacattttacaacgtttgtagtacctcatggtgtggaagatgcaaatgatatgggcaaagaaaaagctgttaaagtacttaaagaaaatagaattttagctacaccagagaacctacaaaattacttattacaacaaaaattaacatttaatgattgatttagtagacaatttatgttacactagtagaagactagtgaaggaggaatatctttggagaaaaaaagtatttataattttgtagatgaatatttaattttagaatctacaaacaataaggaaaaactaaaacaactagaagaagaaggcaatagactcactgctacatatacagatgtagcaaaggcaattaccaacttaacttatggtatgaacgaacaactaactgacgttcatgctatggttacatcattactttctatcttagtagaatctatgtatgaagaaaacgtaattgaccaaaaagtattagataaaattgaagagtatcaaaataaactatatgaaaaggaagatgaataatggaaaaatctgtatcaactcatgctaaggatatttcaactaatgacttaaataaatggttagaagccgtagtaacaaataaagttaatggaaaagaaattggagaaaaggaagctaaagaattacaacgattaggtaaacgttctgtatctttagaggtagctacaagtattgctgattacttagctaagtatgaagtagaaaaccaagtttcacgacttctacaaagtattgatttacttatggttattatggaagatgaactaggtattgatgaagaaaaacgtaataaagctgtagctaaactaaatgagaagtatgaagcctatattaaagaacaacaagaaaaaattaaagagcaacaaaaacaagagtcaaaagaaactgaccgtagtgagaaagtcgtttctatgaaacctaaaaatgagtagtaaaagtaaaggtgatacattcgaaaggaaaacagctaaaacactaggtgagtggtggggttacaagtttaaccgcacaccacagtctggtggtgctagttggggagctcaaaataatgcagtaggggacattgtaacacctcctgatgctgagtttcctttagttatagaatgtaagcatagagaaaattggaacatggataatgtattactcaataacaaggagcctcatacatggtggcaacaagttattagggattcagaacatgtaggtaaagttccttgccttatttttactcgtaatagagcaagggattatgttgcattaccttatatagaatccgtatataataaccttagagataaagattatcccgtcatgagaacaagttttattactactaaccttaaagaagaaaagttttattatgatgttcttgtaactactattgaagggttgacccattttaaaccttgctatattaaatcttgctacaacgaatttaaggtaataccttacaaaacaatatctaaaaaaactgtgaacaaagacgaagatagtttaatagatgatattttagataatatataacaaggaatgattttatgacaaaagaagaacgacctatgattgtatacttttcaggtacaggacaaactaaaagattagtagaaaagattaataatggctcatttgaggtaatgagaatgaaaacaggtaatgaagaaattaaaagaccttatattcttattacccctacttatatgaaaggtgaaattcctaaacaagtacaaaaatttattaataataatcacgcacctaaagaagttattggaacaggaaatcgtcaatggggtcaatatttctgtggtgcaagtaagcaaatagctgaaatgtttaaaattcctttgattgctaaaattgagcaatcaggtcattttaatgaagtaaatatcattaaagaatattttacaacagaatacaaagttagttactagaggtgaaaagatggcaacttatggtaagtggattgaccttaacaacgaggtcactcaactagatgaaaatggaaaaaataaactctataaggataaagaagccttagaagagtatttaaaatatattaaagaaaatacacgtaattttgataatgaagtagagcgtgtgcgtaccttaactaaagaaggtgcttacgataaattatttgataacatcccagatactattatcgaagaaatgacaaagttagcttatagttttaactttcaatttcagagctttatggcatgtcaaaagttctatgaatcttatgcagtaaaacagtatgatggggacgataatcctatttttgtagagaactatgaacagcattgtgtaggtattgctctacatttacattctgatgattatgtacaagcaagaaaactattaaaagcacttattggacagcaataccaaccaagtagccctactgctattaatagtagacgggctaaacgtggagagttaagctcttgttatatctttgtcgtagatgacactacagaatctattaattttgttgttaataatactgtaaatgcaagtaaaaatgcaggaggagtgtccgtagaagcctctaggattagacctaaaggttctagcgtaaacggaaacccaaatgctagtaaaggtgtaattccttttgctaaagctattgaacaatctgtgagttggtttgaccagggtggattaagaaacggctctgctgtcgtatacttaaatattttccatcaagatattcaagacttcttatctgctaaaaagattaacgctagtgataaggtaaggttagatacattatctattggtgttacaattcctaataaatttatggagctagtaaagtctaataaagatttctatacatttgactcctctaacttatacaaagaaacaggaaaacacttagatgaaattaattttaataaagaatacgattctttagttaaaaaccctaacattaaaaagaaaaagttaaatgctcgtgatttaatgactgagattgctaaaacacaattagaatctggttatccatatgtattctatattgataatgcaaatgataatcacccattaaatggtgtcggtaaagtaagagcaagtaatctgtgtaaataaatgcacctttaacgagtaatcgttatcgaaaaatccatctaaaaagggaaagcctaagtctaaatgatacggtgaccctttgctaacctagttataatgcttggtgtgacataccctacacgtgtcgaataactagaaatgcccaacgactatccgtaacctatcttactaagtaagagggataaggttagagccaagtggtacaagtattgagtaataatcatataaatcttgtttaaatcgaaacggtggacttcccaagcggaagaatgatatagtctactctatatagaaatatatagcagtcataaaatgacgagtagtgattaacgaccactactgaatacataggactgagataatgcaggtacaagaagtatcagaaattaatccttatatggaatcacaaaatgataagattggtagagatgttatttgtacattaggttctttaaacttagttaatgtagtagagagtggtggattacccgaagttgtagactcaggtattcgagcattatctgcagtaactgataaaatgtatttaccttttttacctagtattcaaaaagcaaatgatgaactaagagctataggattaggggttctcaacttacacggacttctagctaaaaatttaattagttatggttctaaagatgccttagatttaattaatagtttaatgagtgctattaactactactcattaaaatcaagtatggaactagctaaagaaaataaaccatttaaagattttgaaaagtctgattattacacaggtagatactttgacatgtatattgaaaagtcacatgaacctaaaacaactaaagctaaaaaagttttagagaaagtttacatccctacaacagaagattggaaacaattagctaaagaagtaaaagaacatggtttatataatgcatatcggattgccgttgctccaacagctagtatctcttatgtacaaaatgctactgcaagtacaatgcctgtatcgtctcctattgaaaaccgacaatatggtgatatggaaacttattatcctatgccttatatgtctcctattacacagttcttttacgaaagtgagacagcatacaaggtagataataaacgcattattaacacaagtgcggttattcaaaaacatacagaccaagcagtatctacaattctatttgtagaatcggaaattcccactaataaattagtaagtttatattattatgcgtgggaaaaaggattaaaatcattatactacacacgttcaagaaaattatcagttattgaatgtgtatcatgtagtgtgtagaaaggaaagaatatatgtcactaaaagaacaagtagaattatataacagcaaagataaattaaatgctatcaattggaataaagaagacgaccctatggcagaattgtactggttacaaggtgttcagcaattctggttagagaccgaatttgatgtatcaagagatttaagttcatggaataaactttctgaactagagaaagacacatataaaaaggtcctagcaggattaacaggactagatactaaacaaggtggggaaggtatgaatctaatttcataccatgaaccacgtaataagtatcaagcagtatttgcttttatggggggcatggaagaaatccattaattaattagtagtggatttaaaacgtagttaaacggggaaacccctaacgtaaagacgagggcaatcccgtaggaagaattagtctagtaataactgaggagagaaaataaacagtaagtaaagatggatattatttactagactaatttaatctctaacgactagggaaaggtgaggtttatgtcgagaggacttattccaataaaccctgtaccgtagagccaagctaaagggttagcatcaagtgaaattagcttgtataagactattaaatcgaaaagctacggtttgcagagtaacgactgcattcatgagatagtctgagcgtacatggaaacatgtagaaggataaacaattgcaagtttattcgtaacataactggctagaagctattcatatatttttacaacaatccttaataacaaggaaacaaattacttactagacgaatggtctgaagaagaaccgcatttaacaaaaaaagcacagtatattggatacttctataataaattactacaaaaaagtcctagtactgtagatagatatatggctaaagtagcaagttcatttttagaaagtgctttattctactcaggtttctactatccattgcttttagcaggtcaaggtagaatgacacagtcaggagctattatttacaagattacacaagatgaatcttaccatggttccgcagtaggattaacggcacagtatgattatgagcttttaaccgatgaacaaaaagaatatgtagaccaattaacttatgagttacttgaaattctatacgaaaatgaagtacattattcagaaatgctgtatggtaagttagggttaacggaagatgttattaactatgtacaatataacttcaatagagctttagctaacttaggtaaagaagattactttgaccctaaaccatttaaccctattgttaacaatcaaacaaatgtagatagaatgagacatgtagatttctttagtggtaaagctgattatgaaaaatctaccaatattaaagaccttaaagatgaggactttaaatttgaggataaagaatctgctttagtagacgaatttttataaaggaatgatgttatggaaagactggagaaagacaaagccctagaaattcttaataaaacgagtattgttttacaaaatcatgaagattttaaagtatctgacgtacaagatgcctacggtgctaagtgtgtgacaattggctatgatacagaagattattttcttatttatgataatagaaaactgactctaaattcaaagcctaatgattcggggtttgacaatacagtaactgtaaattcatttgaagatattctaaatacgattggggaagaagattaatatggaagaagttaaaagtttagtagacttaaatgtacaagtaagacaaaagaaagatgctattgcattactaacacaaaataattgcggtaaatgtgaaatcttaaagcaggttatcccacacttaaatttatctaaaccgttatttgttttaaatttagatggcaaggacgctgaccgtgaacaaatcatcaatcattttgacctcatgtctactcccgtacttgtagggtataaagatggggaattagttaagaagtttgaagatgatgttacacctcaacaactacaagaactagaagatttataattttgaattaccgctactttatgttatactaaggtagcgggtttgtttggggtcacaaacaaaaggaatgattaacaatcgcaaagaataaaactttaactatttataatagcgacaggtactttaacatacacactaaaaagaaatccgagctagatgctattagagtactttcttataatgaaaagtcgttagaaaatattatggatgatttagaatccaagtcatctaggtatgttatgagagatgatagacattacttattgtttaacgaaaagtataataatgataagttaatagaaaaaattattaagcatagtggtaaagtaacgtattacacggatactgttgttccttactatgtatttaaagatttagctaaacaccaagattctacagtagtctatcgtatgcgtaaagactttactgataaagaaatagataatgtagctttatcttttatgggtactaaagttgttgtagacatttctgttgtacttcctaatgtaaacccctatgaaattattagacagctacatagtgttaaaaccaatgtagatgaggttcatttaagcttccctaggcttaaagaggtatccgataaacagaaaaagttttatgtctttgatggtgaggcgtatactctgaaaccagaatataaagtagactttgctgatagaatacgggtatctttatctgtatggaagatgtacatttatatattaacagacgacaaagaccatacagacgtacagagtgttattgataaactaaagaagtataggaaagttaagatataatggcaaacagaaaagatattataagaagaatatctgataaaacaggatactacatgcaggatattgaagaaatacttaatgctgaagcagagtctatatacgaacttctaaaagaaggtaacgacagaataaaacaccataagttctatcaaatagaagtagaaactagggaacctaaggaagcatgggatggattaaataaaaggtattttcaaataccagaaaaaaaatatgttaagtttagacctatgtcagaactagagaaggtcataaaagatattaatgaggaagagtaaattcttcctcttttttattgacattcttttatttatgttatatactaaatatgtattaataaggaggaatatatgtatggaagatttaattagttacttttattctatggaaagtaagatgttaaatggatgtaatttaaaaaacattatggtagaagataataaagatattacattgtattttaaggtaaataagaacgtagagcttatttatgtaaatcagtacacggagtacactttactattaactgacgtagaaacaggagatgagtaccttacaactaaagagaagctaggggtctggttacacgaaattactagaaatatgcataatagaaattatcttaaagttgttgatgaattctatagactagattggagagggtaagtatgaaagcattaatattattcgaccatattagagaagagcattactcagtggttaataataaaccacaatttaaactctttagtacaccgaatggtaaaatactaaaaagtattttagataagtttgcagggttaaaacgtacgaaaggtattaagaattacgatattaactacctctataatgctatcccacaacctatctataatgattatggtaagattattaaatataatgatgtaaaacaaagtgaagttaagccttattatgaaagaatgaacaatatcatcatggaaaacaagtacgatattattattccattaggtaaattaggtgttaaatatttattaaatgtaagttctattggtaaggttaggggagtacctactaaagtagacattggtagccatacaacgtgggtactacctacatatagtattgaatatacgaatgtgaataaaaatgcagaacggtatgtagttgctgatttagagctaataggtaagtttattaaagagggtgaagaagcattcaaacctaaaactgtagagtatgaattagtaacagatattgaacgagtaagggaaatatttagtaaagaagtaaaaaatgacaataatgatggtgtggatattaccgcatgggacttagagactaactctttatctccagagaaaaaaggaagtacacctctagtgctatctatgagttggaagaatggtcaaggtgttacaattccactatataaaagtgattttacttgggagaatggacaacaagatattgatgagattctttctatgctaaaagagtgggtagctagtaaagatgatattaaagtgggtcaccacttaaaatatgacataaaattccttatggctactcaaaattttgttaactttgaaagtaatctagatacattagttggatggtacttaactgtatcacaagaaagtactgaatctaaaagactttctgatattacatatatggtgacggatatgggaggttatgataaaccattagaggactttaaagtttggtttattcaagagttgcttaaaaaattatctgacactattaataatagaaagaaagataataagaaggaagctaaaaaaaagtatggtgttaaggttacagaatatgattcatggattgaaagtaaactacaagaatctgttgctttagatgagcatgatgaaaaattacatgtatctttagaagagaaaaaatatattaacttaggactaacaccagaggttttgaatcataatgtgtttatggatgaaggcattcaagaagttttgaaatcgtccaaagagtatatgtcattatcagaaaaaggtaaatcatatgtactaaatactgcattaactttaattaataaatttagaaacgttaaagatgttattaatgaggtggatggtggtaacttcaactatgattggattccactagaacttatgcacccatatgctagtggagatactgatgcttgtagacgtatatactgtaaaattatggataaatttaaacaacaagatagacctaaagcacgagaactatttgaacgtgattaccctagattagatagaaccttagcacgaattgaaagtaatggattatttgctgatttggactacatggaaaagaataatgaggcatataataaggaactagataaagtagaaaatagagtacgaaaccaccctctgattaaagagtttgaagaatctagatatgaattatatttagcaggagtagaggaatttgaaaataatacccctaaagagcgtgataaagaaataacgcaatatagagataaatttaaaaatgggggttggaaatttaaaattagttctggtgtacacaagggagaagtgttatatgatattctaggcatcatgcttccatatgaccaaaactatgtgaaaagtgcatcgtttacaaaaaaaagagaaaatgaaattacttgggaagattacaaaactgactctaaatctttaacatacattttaaagaatatggatttaaaagaagatgtgaaaagtttaatagaagatttactttattatgcttctatacaaacgaaaaggaactcatttactaacaaactacctaatatgataaataaggaaacacgcacattgcatggtagctttagtgcaacaactactagtacagggagattagctagtagcaacccttaaattggggttgtaaaactttgttaactgcggggagaaacttgacaggtcttaactactaacttataatggaaacatatgtaagggcaaacagtaatgtgtttggtatagtaaaaaggttaagaatagagagaatccgcaaccaagcacctgaaagtatataaaagtatggtgaaggctcaacgactaggtgttgagatagtacagtcaatacacacccacgaacgcaaagatattatttctgtggtagggaataataaggagaagcgtatggaagaaatttggaaaacaataaaagagtttgataagtatgaagcaagtaatacaggtaaaataagaaataaggaaacaaagaaggaattaaaacaatggattattaattcaggatattatagtgtagccttagtagatgatttttacgataaaaaacccagaactgtgcatagattaatagctaaaacttttatagataataaggatagaagtaaaaatatagttaatcatattgataataataggctaaataatcatgtagataacttagagtgggtagattataaaggtaactccgcacatgcaaatagacaaggaagaatgaatacgtatagtgctagggagcaacttaaaaaagtatctaataaagcagtgtatcaaaaagatatggaaggtactattattaaagtttgggattcagcaacgcaagctgaaaaagaaacaaacggatactttagtcaatcacaaataagccaagtaatttatggaagatttaaatcacacagaggatataaatgggagtacgttgataaagaatcaaaaagaagtaagcaaatgaaaattgatgtgtttgacttagaaggtaaagtactatatactaagtatagcatgaataaaataatgcaattacttaatatgaataatcataagacattgagagacaagttaagaaaaacggatgactttgtagaatacaaaggatataaatttagaaatagcaaataaacaagaaccctaccacagaaatgatatatgatatagtctgtactgtatagagatatacagttactgtataaacaacagtaaatataatatataaatattgtatattgtacacaagtaaaatttacaaaacatagtgtcccacacatctaacgttaatttgtttgattaccatcaccctataaagcgctctttcgtttcaaggtttaatgatggtgttatccttcaggcggattacagtgccttagagcttagagtgactggtttaatgacaggagaccctgaaatgactgaaacatttttaaatgacggagatttacacaaaaataccgctagtttaatgtatggtaaaccagaaacggaagtaacttctgatgagcgtaagaaagcaaaaaccgtatcattctccttactgtatggtgatgtacccttctcttttgctagtaagaatgatatgccagtggaagaggcagaggaattatttaataagtattatgagcataagcctgctgttagagatgctattaatgaaacacatgagtttgtgaagcaacatgggtatgtagaaaccttacatggctttagacgattcataggaaatgctacaagtagtgataaaaagaaacgtaatgaagcattaagacaatccttcaacgcaaaaatacaaggtttaggttcctggtttacaagtatggctattacttatattgatgactttattcaattaaaaggtatgaagtccaaaattattgccacagtacacgacagtattgtagtagacgcacacccagaagaagtagaggttatggctaaagtagtacttagatgcatggaaaacttaccttttgactttttaatgacggattataaaggcaagactattagatatcctataaaagctgacatggaaattggaagtagttatggggacttagtagaatatgatgaagatgattttaaatctttcagtacaccacaagggtatatcaagtattacttaaatttacaaacacttagtgattactttgaatcagggaagttaactgaggagcagtataaacaaaaagtaaaattagtagagcaagaaaaatataaatatcaacaaatgtcttgacagtagaggtaacttatgttagactttagtaagcaaaggagagatgcttatgcgttttaagaagtacaccaagaaagaatctattgagctcattaaaagtggtaaagtacatattatgtataactatcacacaagcaacccacttacattgtttgatattaaatttagaccttgttttttaaaagataggtctttatgggtagtaaaaaaagaaaatacaaagagaggtagaaagaaatggaaattcaagtagatagtctagattttgatactattactattaaagataagaacaataatgaaaccattctttctatccaagacgagcttaaaatagatgaaattaacttaaatgaatcttttatgactcaatctagtaagtttgcttactgggctaacttactagagattgttcgtaggtatagtgaagctgaggaacgaaagttagaaacaataggagcgcaactcaatttatctattcgtgagtggtataagcagaataaagaaaagccaactaaagatatgattgaggctcgtattatgatagataaacactatcaagagcagttaaaaatagtagaggaatggaagtataaaacaaaacaactacaatatgtagtaaaagcctttgagcaacgtgtaaatgcgctaacacatttaggtgctgagcaacgattaactaataagaataaagacattacaaatccatattcttattaaaaaagtattgacaatacaagtaagatatggtagaataaaaaagaattaaaatttaaggagagattatatgtcatttgcagattttttaagtcaagaacaaaagaaacaagaaggttttaataacgataatgtagagcgatacacacctaagcaaccggtattacgtttaggtaagcaaaaagatggttcacaaaaagagcaagtactagtacgtattttacctgcaacagatggagggtttgcttttgctaaagcattccgtaccactttcattaattacactaaaaagagcggggagcaaacaggttctgggttaacactacctgccgataatggttcatcagtactagacccattcattaatgactggattgctaaaaaggtacaatttagtcgattccctaataaaccatcattacgttactttattcatgttattgagtacgtaaataatggcggtcaattacagccgatgaaagatgaacaaggaaacttaaaagtacaacctatggagatttcaggtactgcatacactaacattattaatgctctaggtgaccctatgttagcacctaagacggaaacagacttaagattcattgctccggataatgcatttattgtcaatatccgtaaagctaaaaaaggtgaaatgcaatggggcgttactgtataccctacggtagaactaggagctttacctaataactggaaagacttaacaagtgacttagataagttagctacaccaacagaagaacaaaaccctaactttgttaacttcttaattaataacgtaaataacactgaattatctacagacaacttcaagtataatcgtgatacaaataagttaggagaagaacctacacctagacaagaaccaacacaagcaccaagtgataatgatattgataatatgatgcctagtaacttagggggtaatgaaccataccaaccatcatctaatccttttgatgtaggtgatttatcagaacccgagccatatgaaatgcctcaaaaaccggtacagcaacaagagcaacctaaacaacaagaacagcataagcaagcacctactcaacaaaataaccaaagtatgccacaagatattaattctatcttaggtgacttagatattgacatttaatcaaaagggcgttatagcccttttattatacaatcataagattttaaggtagaaataaaaggagagattttattggctagagctaaaaaaggacaagaagtagatttatcacaattaaatacaattgatttaggtaaggaaatgggattaacattactttccgatacaaataaagcggatattaaaaacattttacctacaatggttccacaatatgatagaatcttaggtggtggtattcctttaggacgacttaccgaggtttacgggaaaccaggctctgggaagagctctatggcagtacatctatcacgtattacaactaaattaggtgtacttacgatttggattgatgttgagggcacggcggataacagacgtatggaacaactaggggtagatgtttctaaactattttccattcaagcaggagaaggtagacttaaaaatacagtagaattatccgttgaacaagtaggtaaagaattagaatactggattgatacttttaatgaaaaaatcccaggtgttcctattgtttttatctgggattcactaggtaatacaagaacacaaaaagaaattgacggcggtattgatgaaaagcaaattggtattaaggcttccgctacacaaaaggttattaatgctattacacctaaactaaatgaaactaatacaggtgttattgttattaaccaagctcgtgacaacttaaatatgtctaacccatatgatgaccctattaagtctacaggaggtaaggcttttgagcactcagcaagtctacgtattaaaattgaccaaggtagaacattaaaacagaaaaaagaaacatcaggtaaagatgagtattcaggtcatattatgcgtatcactactaaaaagtctaaactatcaagacctaatcaaaaagcggaggcggagctcttatcagactgggatttaccagacggaacattaattaatggtttagacccacaaaatgttgtatatcaagaagcagtagaaaagggcttgattactaaaggtgcttggagaaattatattacacttaatggtgaagaaattaagttacgtgatgtagagtgggttcctagactaaaagaggattacgacctatatttagaattatttaagcgtacttatgtagaaaatttccctaatggatatgcacctttagaaaataaaacagtagatgttacacaactagatgaatatcaagtattagaagattattataacgagctaaaagaaagtaatgaggaacaagcagtagaggaagataataatgaataacttaatcgatgataacattaagaaagttaaaagggcgttagttgatactaactcccttgatattgtaccagagccataccttgctattgctagtaagtttagaaaagtaaaggaaaaaggtgagcctgttattctagaagatgcgggattccctcatacgaattctactattatgtatattgactatgtttctgatagatgggttttaggttactcttatacaaaaacagaaagacaaaatgtaaaaatacctcggactattcattattcagacctctatgttacggataagtctcacaaagttaatgttatttttgaaggggataacccttatgaatagatacgtggtagaccctactataacagatttagaaagagatgtagatacattactagaaaagtacaaaaatttacggtgggcattatatcatcggtatgcaagagcactatctaatgaggctgaaagacaggaactacaagaatacatagatgagcaatttattaagttagttaaagaatataatattagaagtggcgtagattttccaggatatattaaatctaaacttacgttgagagttagaaacagctatgtaaagaaaaataaaaaatataaaaaaactgaacttttaggtaataaagataatacagtagagatgctatcagaacagctagttagtggtgttaatgaaaccgatgtgattagctatgtatttgagggtgtagactttaatgatacacaaaagctcttacttcactatttattagatgatggattttatgaagattcatctataattaggttagtttctgacgaactagcagtccaaagaaaagatgtatctaaagagctagaggaacttagaacctatattaagtttaaaattgatgcgtatcatgaaaactttactaagaacttaatggagcatggtaaagtaaacacagaaaatacaacatgggaactataaaagggtactttaagtaccctttttgttatattaaaattgaatttataaagaagaaaaaggagcttaaactatggctaaaaagaatgtaaatgagctacttaaacaagaaactgtagaagtagcaaataaatatttacaagtagtaacacatagagatggttacacacgtacacacgaaggaaaatactcatacaaagtagtagataagtctggagaattatttatttttccagttcaaacagatggttatggtaaactacaagtgatggtagaatcaccaattgcttacacagatggtaataacatccactttgtagtgaacacattgacagacccatacacacaagcatttattcgtaccgaagagattagtggcttaaataaaggtaaacagcttattcaagcatttttagcttttattgaggaccgtttccgctttggtatttataatatctttgttgtggataaggaagatgctaaagtattagccgatgttgcggacacagatgcagacgaggtggaagaagttaaaggtcgtgcttttgaagatgtaatcactaattatccaacaggtaatgctcgtgaggacgtacgtcacgcagaccaaagcgatggtatgggagatacttcagaaccctcagcacctaaaagtcctactgaggtacaagtagaacctggtgaaacttcagcagatatttcagcacaataaactaatataaaggagtaataaaattggcaactaaatttaatctttacaaagatgatgagcttgttaagagcgtagacaaggaagaaataggtacaaccactattacaattgactctttagaggctaatacaaactatcctgagggtacttacactgtatctttctctaatgaatctggagaatctaaaaaagttaatgttccagaatttaatactaaggctattggacttgcatctattacattagatgtagatactttagaattaaaggttgatgaggaacatcagttagaggttacttttgaacctagtacagcgtctaatcagaaagtatcgtatgaatcagatagtgaacatgcaacagttacatcagatggtttaattcaagcggtttctgcaggtacggcaaacattactgtaacatcagaggacggctcacatacagacactgtagtagttacagttaacgaaaaagttccttcagaacctgttaatgtacaagtagaacctggtgaaacttcagcagatatttcagtagaataggagagtaagtaatggctaaaacattaaaagtatacaaaaaagatagtggtgaggtagtaggacaaaaagaagtaactgaaggtactaccacagtcacaattacaggtcttgaggaaggtactacatatgaggaaggaactttccaagtagctttctctaatgaatctggagagtctaaaaaagtagatgttccagaatttactacaaccaactcagacactatataaaaagttagactaggattaatttcctagtctttttttattgacaacgttttatctttgtgctatgattgtttatgaaattaagaaaggagattttctatggaacttattttattaagaggagcatatgactatttaaaggttaagtatgccatggagaacaaagaaaagtatgttgcggtggcttttgacgatacttctgtagaaacattaaatgtaatgagtatgatgaatgtcattcaagactatctacacatgtatggttatagagtatcagagtatggtaaaatgaatactgatgaaggtgttaaaggttacttatacgaaaggattgaggactaatgcttattatctataataaaaatcatatacagtcaccagaagtttacaaacacttagggtacgttgtagacggagacgagtgtactgtaaaagataatcaagatgccattaaagtaattaaacaagcacaggatagggacattatgttagtaggattcatttatacaaaagacctcgagtgcttaacagaactagataaaaatatttattgggtatctaatgtaggtaccgtacctacattatttaagcacgtaaacaacagtaaaacaaaatctttgttagagcaaacagtagagttattaccagaagagtataatgtttttgattatactaaagaatatatgaagaaagcaactgactactataactttaagagtgatgaatacttagaagaattagacattgataaagaccttattgacatgttagctaaaaagtgctatactaatggagacttatattttatttatgagttagaccctaaacttaagttacctattgcatataatattgcaagaggtaaagataaagctattgtagttatagggtcacaaacagctagtgataatgacatgctctctttctatgtaaaagggtattgtgtaggtgcagtagcaagtatgtttaatgcagagtatgagattggtagtaacacctttactgcatttatccctagtcatattaatgtagtaggtaaaagcctaaaaaaatatatggaggaatacaatgtataagtcaatagaagaagtttatgcaattgtctttggtgttttagctaaagatgaaggtaaaattgttagcagtaaatttaataaaattttaaatgaaattggtattgatagagtctcagttaatgacttaaaagatatttctgagatgattcatgaagatggttatttaaacgggttgaaaaatgatgctattttaggtaaagtatcattaaaagaccttgagagcgtagaaggaaaaaaagtatttaaagataataactatttagatacagtttctacttatgtagtagagaatgagaaacggctatctaatttacgtgagttacgtaagcaccaaaaaaatggtgcttacatggaaatgcttatggacaaccttaaagaacatatggttaaagaactacaaggtaatcctttagtagcacacaagaaaaatttaggttatagtgataatgaagatgagttaattgtactacttagtgatttccacattggtgaattttgcgcctaagtatatagtaatatatactaagtattcttttaattgactgggaggctaaggctaaaaagctaagctaatcagcatccaagatttattctaataattacccctctaagtaccttctaaataaggagaaggatatatggagaagaaaataaatgaaatacctgagtataattatataaatgaccattattatataacagataatggggatataataagttacgctaagaatggcagaagagttatgaaattacatataaataaacacggatactatttatgtaatttaacactacctaagaataagaaaaaaacttttatagtgcatagaatagttgcacatgcttttctatctaaccctcttaataaacctcaagtgaatcatattgatggtaacaaattaaacaacaatgttgaaaatttagagtggtctactaggaaagataatattaatcatgcatggcaaaaacatttaacagataataggtgggctaaaggggaaaacaattatcaatgggaaggaaaccataagaattgtcgttctattgttcaaaaagactcacaaggtaatataattaaagtctacaattccattgccatagctagtagagaaaataaagaattaaagagatataagatagataaagctttaagcaagaaaagtaaaagtaatgagtatgcagggtacttttgggaatactatgaagaataaataaggttcaacgactatccctaggacgaatgtggttaaataaatcgtcaataggagtagggctcaagttaatggagtgggtgagaatcccttaaatcgaaatggagaacccctaattaaaaaatagggtgaagatatagtctcaacttctatggaaacatagagaagttcataagagaactggtaagatagttgcgtgtcttattgaaggttttggcaactgtaactgatattggtaacaaatacaattttgaagtgctacaaaatagaattaatcagtttttagatacgacaattaaagagattgttaatagaaatatatctaatgtaaatgtgtattttgttggtgatttagtggaacatatttttatgcgtgacgttaaccaagcatttgaaacagagttcactatggcagaacaaatatctaaaggtacaagaattcttattgattttttaaatgctatttcagagtatacagtaggtaccttaagattcggtattatcggaggtaatcatgacagaatacaaggtaataagaaccaaaagatttacaacgacaacatagcttatattgtactagattcgttattcttattacaagaacaaggtatcttagagggtatagaaattattgataatcgttcagacgtgtacaatattaaagatactgtagccggtaaaaacattattgtaaatcatggtgatggtctaaaaggtaaaggaaatcatattactaagtttattgaagataaacacattgacttattaattacaggtcacgtacaccatttttcagtaaaacaagaagattttaatagaatgcatattgtagcttcatctcctatgggatataataattatgctaaagagctacagttatcaaaaactaaaccttcacagcaattattatttgtaaataaagaaaataaagatattgatattaaaacagtatttttagattagagggattctatgagtaatttattaataatttgtatagtatatgctgtagtggtaacaattaatgtaatcactaaagttttagaaatatggtcttttgagaagcgtagtgttgctatgggtaaaagcgcatttagtaattttgtttactattatgaaattatcccgtacacagtaggattgattactcttgtaatcatttcttactatagttatatattacctaaccctaatacagtagttcatattgttgtatttacaggattagtagcttttttctactttttagtaggcgttattgcagtatttattatatcagtagtagaggcaaatattgtacgtaagagaactaaagaaaatgagagtcaagacgaataggtcttgactttttttattatgtatgctaaattaataaagaggtgattttttttatggatttttcatttacaggctttgaagatgataacattacaatgcgtatatctgaaggtgtatattattttaaagatgagcctatatactgggtagagcacatcgaagagaattctgatgagtacgtttttgtatatgacatacatgataagaaagaagaaaaaccacaaaaagcatatagagtagaagaaatatctaaaactttaaaaggtggaacttacttaagtaatagaataaaaagccttattcctatcaagaagcaatataaaaaagtatataaagaacccttatttgttgctcatgttattcctagtggaataggtatagacactattactggtaggagtggtaaagggttttttgaacgacaacgagaagaacgtattgcaacgtctaaaggaataagtgtaaaaggcggggactatacaggagtgtttattacactagaaaacgtaaagtggaataaatcgtatattcctctagaaagtgtcatagagtattataagcaaactaaagaaggtaggagtaatgtctgagatgttttatgaacaagatattaaagatttaattagaactaagaaacacttattccttgatgatgaaattacgagtaatatagacgatattaagatatttaatgagaaagtaatatgccaagggaaatgtagaacagattgcttagtactagaccgtaatggtacagttatggggatagagataaaaacagaaagagactctacacaaagattaaataaacaactgtactactatagtctagtctgtaaatatgtttatgtattttgccatgacaaacatgtacctaaggtagaaaagatactcaaagaacataaacatgaccatgtaggaatcattagttatatcaacttcaggaacaaacctacagtaggtaagtacaaacaagcaacaccatcacctttcagaagcccttaccatactttaaatatattatggaagtactctttactaactatgttaagaatcattagagactaccctaaatattataataaaaatgcaatacatacatttaataaaaatggacatcaaaatgagttagctactaagggtacacagtctttaagaatgaagaagcctgctatcattaatcaaattattggttttataggggtagaaaaaacatatcagttgttttgtaggtgtgtgatatatgggtatgataatagatggcaacttattgatgaatcattttttgatgtagttaagaatggggtgattggtatatatgaagaagaaagataaaccaaaaagaatacggcaccaacctgttaactttgctcctacaaacaacctagcaggagcaaaccctacattttttcctaaaaagagacaacgagctaaagagacaaaaggttatgagtaccgtattcagtttattaaaaagtttgagaatgtctcaaattcagatgttagtatgcaaaaaaaatatatattaaatcttattagtcgagacttgcatttacctgaaaaatatattacactaaaacagaaaggtactaaaaaagaaaccataatgtgtacagatagagtatattatgtgtatcgttctaaaaaattaattggtaaatgctcaattagagagcaaagaacatttaaaaatactcatttaatttttgtatttaaactaagaaaaaggaagataaactaaatggaaatgattaataaagaaaccgctattagtatgtctacattacttaaaaaattaaaaaaaggacctactattgtaggtgtaggatatggctccgagctattcccttatcaattattagtagagccttatatagaggtagaggaatctgaggatgggtttttgaaagtgtacaataaagtatttgttccttctcataaagtaacagagtttaataatttcagtattgtattggaaaatgctatgttaggctacactactaaattacaaaagcaaaataaaaatacttttgatattatgacttcttataaggaagatagagatattcacgagtatgctatttctagtgtatttgatagtcctactattactaagtcttcttttacaattagtaaaaagaagtttactgtgtataaacttacagaaacacaagttacatccttagactttaaagactttacacatacactatatttagtaataccggaagatgatggctttcctaaaggtagactaagtaagacagaaacacaacactttcttaacctattctcagatactaaagtgataccagaaacaattaacgctgtatcttcttcaagaagtaatgtaagttttgatatgtacgaacctaatggatttattcttattttcaaagctaacatttatgaattaggctctaaaaaccacaatgtctcttttcacaaagaccttattaaaggtagtactctaggtaataacactattacaatacatttgaagaattcacatgaattacagttattttggtaatatttagtccagttctattatttttatatataacaaaatagttaataaaaataatgacaagtagtgtagtataaaaaagtaccagtaaaatattgaaaaatatttttataatttatattcattttgaaaataattaccatagaaatttactactagatagttaatagtttatttctatggttttttaaaaataaaatttaggaaaaatagtgatatattaagtatgaaataccctttgaaaccttattctaaaagggttatagtagtttaatataaagtgtttcaatattagtttaacttattatattaataaaggaagatacataaatatggctagaaaaaagatgagaaataaaaacgataagactctaaaagttgtacctgagaagaaaagtattcttgtagagttatacgagaataaacttctacgtacaaaagtagataatgcaattgatgaggacctaggttatgactacatcatagaactatgtaaagagtatgggttagaattatctaagtctgctattagtagatataaaactaaacgtagagaagctattcaaaatggttgggacttaggggaatatattgatggaagaaagaaaaataatgtttcttctattgaagataaagaagtagatgtatctagtaaagtaaacccttcacatccatttgaagtagctaagaaaaaaacacaagatatttatgatgatattgaggtccttgataatattatccaagcaggtgctttaggattacaatacgtagatactgtagaccctgctctagctattaaggctatggaaactaaacataaaattacaaatggacagttacaaggaatgagccttattggtatgcgtgagcttttagttaaacaaaacgcccgagataccgcaataactcaagttattctagagtacatcccagaagaaaaacatgaggaagttatggagcgtttagaagaaacagaaaaagaattctatgaaaacttagacctagatgaagaagatatgaagttaaaacaagcattggatagagtaggatactctatttagaataggagaaagtatatggtagaagaaatagtatttaaacagttagaagaggcacaacctattaaagaattaagtacaattattagatatttagaagaaggtaaagttctattggttaaacaagaaaaccaaggagatattattgtaaaacttacaagtaacagtacaaatggacataaaatgacttatattagcagagatattaaagaagagtcgagctactataaaagacattgggcgccttttgatattagcattaatgcttttattgcttttgatgtttacttagcagaagatgttaataagtataattataataataagtttaatattaaggatattgtagaatttgtagcaggagatagtaaggacattgcttatgtgcatgatgtatttactgatggcagtaagtttctatacagcttaacaggggaagaaagattatttgaagagtatgaattacgtaaagttaatcgttgacaaagtatatgagttatggtaaagtatgtatatacttattaaaggagagattataatgattatttacttaaatgaggaagagaaaaatttagtaggaggtaaaaactatactcccctagaagataaatttagaactaagaaagaaaaagatattgtagagatgtatcgtaaaggacatcctgttactgatatttgcaatgtacataatattagtatgggattactatatactgtaattaacaaccataaattaggtaaacgcaatattacgaaaaacctacaaaagaaaatttcacatgttttaaataataaagaaaaactagaaaacttgttgaatgattatggtaagttaacaaataaagatatttaccataagtaccaaattcataaaaacggactatattatatcttagatttatataatattgatagaaaacaaggtactaaagaagaggtattatcagatagtattgtaattgaagagtaaggtgctgatagtatgaattatcaagaaagactaagagaaattagaaaaaatagtacacaaaaaaaagctaggactaacaaaaactacactttaagtgagctagaagtatccctatttgataaagatagtgatgttattagtccttttacgagtgtaactaaagatgatattattaaaggtaatgagttatttattgcttctttatctaatgatataacacttgaagatgggactactttagaaaaaggacaatatattggtaatagtataggtagactagcagatatagagaaactagaggactcaggtatatattctaaaagtctttatgaaaacggacatagaattaacttacttgcagttatcaaaaatcctaagtctgtatatacacaaaaagatttagtattagatgttaaaaataaaactgaggttaaagatataacgactaaaagtgcttttgtaatatctaaacaaaaagtagattatctagagcttgtagatacctactattttgataagctcatagatgatgtccttaaaaattataataacttatctaatcctaataacttacttaagagatatattaaaaataaaaaaactttaagtgagggtaaaagaatgatttattgtcctttactagatgaatgtttagctatagtaggtgactctaatgagtagtaagaagtggttattttatatcatagtattagctgtgttaacaagtgctgtgtttgcgtactcttggacttctatatactatgagaaaaacacgtatgaatctggcgtagtacaagacagtgaagtaaataacacaaagaataatggtgacgtagagtatgttacaacagtaacactaaaaaaagatacggataaaaaagctacctttatgaatgatttacctaacttcttacctggtaggatagataataaaacaatagataagagtttctataaatctagtgattttcgtgaaggagagagatttaagctctacagaatatacacaaaagatagcaatggtgaagtaattcataaatatcgttttgaaaaggaagtagataaataatggctccgctaactatatcaattgtagtaggtatctgtattgtatactatttattggagaaagattaggtcaagaaaacacttgaccttttttatttgtctatatataataatgagtagaggtgatagatgtgggaaatttaaaaacattttatgaggaagtcagacatttggaaaatagaaatgttgctaaagaccaaataatattagaggcaatggagacatatagtagattaagaggaacattaacaaataggcaattaagtattgacatctttttaaattatggtatagtagaatatatgaagtataaaaaacatttacgtaggttacaaaagtgtgttttaaatacatatacggaaggagaataactatgattaaagaaaagttttataacgaattgagttactacagagatggacataaagatttggaaagtatgtttgagttagcattagagtatattaacaagttagaagaagaggaacaagcaatagctgagtatgaagagactatcgatgagaatttagaagatgctattgatacaattgaaagccagttagagattatcaaagatttggttaaatggacgtaatttgatgagtagttttgatgaattcctcaagaaagagattaaaaaagttaaaaaaccgtatagtaaacaaaagaagaaagtagaaacacctatagaagaagtacaagaaaagttagctaaaattagaagtaaatcaaattatatggacttaggtaggaggtataataaatgaaatcatacacaagcaaaagagtatctgctagtattgaggaaatgaagcaacacaaagactttagattattgtttgggtttgatgatgtaactttgtatctacttagtgaacaagaaaatgctaaaacattcccaatggataagaaagaactagataaagtagagaagaaatattctaaagagaagttcttcacatacttataaagaggtgaatttatgttagtagcgttagtaggtatttggtctttactatttatatgtgtagtattaaatatcaaattaaataatattttcgttataggagattacgtagagtttaaggacagggttgtagcaacagtatttttcttattcttaagtttaactatggttattggattattaattatattcaaaggagtatatggacaatggtaaaagagaatattaaagaacttttaggatgtatctgtaattgtgtagattcctttatgccaacaggaaaaagttacgtaatttatactgataataaagtgattgttaagcacaagggtagtattgttgtaattattgaagatgatgtagtatatggtaatgtctaccaactaaaaactggtactatagagaagcaaacattatacactttagttaaagcacttcaatatttagtaagtttagattatcgtgaaagagaagatgtaaatatttaaaaaagtttagggaatctgttgacattcagattcccttttgttatactttaattaagataagaaaaggagctgtttattatgaaaaaagtatttattgcaggaagtttaggtttaatgctattcgtgggtggttgtagtaatcagccaacacaccatcaagaagtaattttagaagacagagacacacaaactgaagaagtaaaagcacaagataataatagtaatcaaacagcagagtttaatggagaggttaatccaaatggtaacggagctccacaacatgttacgaatacacctagtggagataaagaagtaacagagttatttgagaaagattttcaagatattgcagaccaaactgctaccctaagaggctctattgatgggtttataagccctgagacaaatattaaagcaggaacttctgtgttaaaggcaaatagaattgtagaccatgtagatgcactttatgacactacagaagacgagacaattattaaagaacaactacaacaaatggctgacttatataagaaatcagcggacaaccttgagaaatatgaagacactttagatgagcaatacttaaataaaggtaaagaacagtatcagcaagcatatgatttagaaattcaagctaagcaaggtactaatgtaatttaggaggaataaacaatgaaatttgaacgattacaaaaatcaccagaagaactaattatgggctctaaagctgtaacaggctcaacattagtatatgaggataaggtttactataaatcaaagaaagacaaagtagatgtactagcacctttagatatgttttatgatataaagtatgtacttaataatactactgcatctattattttatttagaactttatgtggacaccctaaattaacaaaatttaaaggagattacaaagaagtattatataaaattaacgaagaaaaagtaaaattcaataatttagattataagtatttttacaataaccaagtaaaacttgatttacaagaagagcaggaaaaattaaccaactttctttccctccctttagatgagaaaattcagtacaaatcagcaagacgcagagagcatttacgagattcacatagctatcgaaaaggttcgtataattattggggtattaataaatacgctagggaagagaaggttaaaatccaagaactatatgataatgtagggtctactttatctgatattgtatttgataatttatattataatagtaaaggtcatatatatgacaaagatgatatgacagtattaaagttagtaggaatgtataaaacacagtcgttcaatggcaatagatatggtgttaaagtaaaatactttagagacttagaagctattctaccagaaactaaaattaaagaatcatccttagatactaagactggagacttatatattaaggtagatggtgcttactcgtacagcttctctatgaacatatataaagaagagtatactaaagagtactataaacaaggcttaacttttaaaacattagtaaaggcagttaagatacactatattaaatatagttctatgacaaacaatgtattatcacaacttaacaatgacatagtaaaaagcgttttagtattagatagaattattaattctccttatactgagctatttgacaatgtgtctaactatataactatacctaaaacaaaaaagtggaaactacaggatttattaggtatttctaaacaacaactagaaaaattgaatgaatataataaacatgaacagcgtaattatgttatgatggtagatatattaaaaacatattataaagcactttacacagaagaaaacttaaataagttaaatgataccacatttttaatgttaaaccataatcactctatcgtatatgtacttagagaattgagcaaatactatagtattagtttctcacaattaaaaaagatagtatattatgtagatgtagattcttttgaaagacagtgtattggaaatgttgaatatacttatgttgattatataaaaaactaccaaaggttaattagtgcgggatacagaactttagagagcattaatttcacaccattttctttaaagttagaacatgatattatatctcatgagtatgtaatgttgagagatgatgttgatgactcaatattagaaagtaaatatgactcaacttatgatgtcattacaaagaaaaaatataagtacaatggtgagaatattttatttctccctgcagactccaaagataaactaaagaacgaaggtaagagtttatctcattgtgttggtggatacacatctagaattatagaagataaatgcgtaatattccttgctcgtaaagaaaaagatattaaccagtcatggtttacagttgaaattagaaagactgttaatggtttagtcttagggcaacaacagtctattaataaatataagttgccacctgaattaaaagaacaactagaaaaagatattagaaagattaatgcaaagcatcaggagggattaaaaagagcctcttgacaaaggggcttttataatatataattaggaggaaataaataatggaaagggttacatgggaagatattaaaagattaataatacgcaatggaacactagtaccaccgaatctgcatgtttatacaataggcaactatcaaattaattatcatatattagaagataaaatatgcttatacaaggataatatagaggtagcagtcataaaaggacatgtatattatcatactgcagatttagaggaggttgctaacaaagccttaagattaaacagaggtattacaagtgtaattgactattatgcaaaactatatttctggtatttagaatcagaaaatgaaagaaaaaagcaggaagaagagaaactagaaaagttacaatcttttatgaataaataaggaggtaatataataacagtaattttacttaatatcatagcaatcattgcgcttataggattactaatgatagatgataacatcagtagtagtagtatattatggaaagttttacttattcttaatcttatatgcttaggactaaatatagttgttcgtattagttagtagaggtagtatatactgcaagaataaatataataaggaggaatatgatggtagcacttttgattaacgctatggtaattataatgttgttagtagttgctgatgagcttagaaataatatattgttaatatttttacttattcttaatctggtattgttagttcttaatatcgttgtttctattagttagtaaagctagataaggaggtattgtatgaggtctatggagtattatgaaaataaaatcaaaaagaagattactagaattttcaaaaagcaaataaaaagaaaatacaaagaagaaactaagaaaggtaaacttacaacatttacttacgcagaaattaaaatagaaagcgctcatggaaacctgtatttatttgaggttcataaagaattgttagtagattgctttgttaaagcattagaagaactgaacaccaagtataacatatcttataataccgatacagggtattttacaatagaaaggaaaaagtaaaaagattaataaaaaatataaggagtgtgtatcttaatgaaacacgtagaattaagagaagatgaattaggaaatcaatatattttcttctataagaataatctagagacaaaggatgttgtcccgtgtttagacttaaataaagggactttaggtatggcagaaaagatagacattgaggaagtattcccactattagtaggacatattgacacaaacgatttgaaaaaatatatttagggaagaattttataagcaatctagacttaggtgttgacattaacctaagtcttttgtataattaaagtatagaaaaggaggaattaattatgaataagaacctaaaagcagtactaggagttgttattttactgtttgtcttggtgagttgtgttagaatattgtctacccccgattatgtaggagaatgggaagctatagaagatggatatgcacatgaatgggttgagtttaccaaagataaaatgatagttagtggtaaagaatatgacattgaagcggaagaaaagggagagtcctcacgcattaaagtaaaagatgttaatggagatgtgcaaattgaccttattattgcttttaaagataaggataatgcaactgtgtatgcggaaagtacatttagtgatgcacaggtatttgacatcgtaagaaagtaggttatgttatggactacgtaaaccagaagggaattgtaaaagagagtcaagtcaatttaactaaacatgcttttgagagataccaacttagattcggtaagcatacaagggaacaggctattagttggataacaatggctttaaataacgctacttacttatctaatgctaatggtaggtctacttatgagtatgataatgctagaatagttgtagataaacatatgaatgttattactattatgaaagaagagcttgactggaactctatcaaagatacaagaaaggacatagaagagtttattataaagaagttaagaaaagaagtaaaaccttttttaaaagtagataaagagttacaaatctctatctatgaaaaagaaattaataagttaaagactaataatcctacaactaaagaatatattcaaaaagatattgacgaactactcaataacaagaaaaataacattactaagattcaagggattattagaactgctaaaaagtattacgtaaaaccaaatgatatagtaaaaaacttagaaagctatgaggtgtaggttatggaaattaaattgaatagagaagaaaagggtttgttaggtattaaaactagaaatcatttagatagtagtaaacaacctaagaatggttggagaacaattccattcaatggatattatgaggctcaccctagtgggttaattagaaataagtatagtaagaaacttattaaaggtactcaaacacgtaaatataataagtttactagccaagagattgtatatctattagttcctaaaaaaacaacttattctagagctaaaattattgcaagtacctttccagaaaagattagtcagtctaaaggagatttaagcaatggttatgtttgttttaaagatggggatagaactaattgtaatgtaaaaaacttatttattggtaaagggaaattacgaaatgctgtatataaggtaaataactcttttatgacaaataaggacattgttaacttcgtaaacagtttaactaatgatagtattggtaatagttttaaacatatattagataaaactgagtgtattagggaaagtaaagactatactattattaaaaaaggtaaaagaattagtattcttaagtcagatagcagtttatttagttttgtagtagaaagaaaaccaattgaaagaggtagagtataatgggaactttaattttaattttacaatgggttggattaattttattattaggagtactacaaattttttctattaagagtgtaaatcgtattgatgatataaaatactacttattatcttgttttacattcgttgtaattttcttcctagtagaaagtatggcagggtacacagagtatttaatgtttcttattttatttatcattcttattaatacaatccaaaggaagaaagtaaagtaaggataggtaaaggtaaaaatggtaaataaaaaaacttagacttaggtacctatattaacctaagtcttttgtataattaagatatagataaggaggaattaattatgtatgaacaaatcattaataagtggtttaggttactagttgatggtattcatagatatgatgatacaatccgtattgaagatatttataagttagtagaggatatggattctatagatatagttaatgcttactacatgaaaggcaatacagagtattgggtggcaaattgggcaaatagtaagagtgaaactagcattgaaaatatcttaagaaatagaggactaaagtattcttttaaatcagaaccagagtgtaatgcttacttagttaccttaaataaaaacatagttagaataaaagaatactgtgatataattattttagaaattctaaaggataataacatagtagaaacgattgctgtgaatgattataatagagtttctttatacttagatacattaataaaggaaaagaagatgtttaccaacaaggagattagactatggataaaatgattaaaacattatttaaaaaggactaccctagtgttggtgatagaaatatcaaggatttagccaaggggttcaaagagttcttgttaatagttgatagtatgggtatagacttaaatgacttaagagatatggtggttagacataaagaagaacaaagatttaatggggatattaaagaagaagagatatataagagacttagagacacttttgctgggacatccgctcttacagttacaatattcttagtatttatagctacacactttaaagatactgatgtatatgtattgatagagaaggattaatgatagacaaaaaacaactagaagatttacgtaagagtataaaggaactataatatatataaataaatccttaaaaatccactatatataaagtatatagtggttttgttgtgttgtaaataaactaagtatatatatacataaaatagcaaaaaggtttttattataactattgacatctgctcaaagtacctttatactaatagggaggtggataggaaaatagttataaaccactatagagaaaattataaataagagtaaaaacttctatgtaaaagagtgtacaagaggtaaggatattaaggatacacatagaaggggtaaaaagatataaagataggaacaggtatatagtagataaaagggaaaactgaagtatattaacctagtacagtatacataagtataagggatagagaagataagcagatatatagaagggaaaagaaactaaccatagataaacatattaaaataattaaattaaaactaagtaaaagaaaactaactctatgtaagtatatatataacttaaagtatattaattatatacactgatacgtatagtgataaatacctataactaaaaataacattaaataagtaaagataagtaaactaaccccaattaaaagaaaactaaaactaattaaaagaaaattaaaagaaaactaaaacgaacctcaacctcaaccccagaactttttcgtccctctgccccgtcctacttaatgtagtatccggtaatattatatttgaaatatagatatttattatactttaactactatataatgattattattctacacttaatatatagtaatatactaattatctaccttaatgtattagttatttatactaatcgggtactatcaatgatttaccctacatttatatagtattacttatatatctactcttatatggtttccgcttctttagtttcagttcaatttatttactcaaaggtattgctatttaatgctgtacatgctataatgtatatatactaataaaggaagtggcaattatgattacaattaaagatattattgagtttgttgaagagaacggggagtatgaaaaattggagttcttaaccgatatttataagcatacttatacctatactgtagataactatactgtttattatgtaatgattgataaggttttttcctctaacttagtcctatataagaatgatgtaatggtaaataactcctatatccatagcagagctttctgtgctgatggtgagttagatgagctacttaataagtctaatagtgtacttgaggttattgactactattataataagcatatgaaagaagttgctaaagagaatgagaagaagcggtttttaaataaattcatgagtagagataattaataaagaggataataagttatggttagtattagagctgttataaactatattgtagcaaacggagtagaaagagttgaagaatctaaggtatctcatatactagacttgggtaatggttatagtattgaatacattattcccattagaatgtgctattatgagggtattactatacgtaaaagaggctttattcaagagtttaaaggggatgctattgattatttagatgctcacttgaagaaagaaaataacatttacgaagtcattgacttttatgaatcagagtctattaaaaaggagtctaatagacagttagaattagaagaagtaaaaaaatttatgggtagaagagaataaaatattgtaactatatttactcaaaggtattgctatttaataccgtaaatgttataatgtatatagtagcaaggttggtatatatctaggagatattgaaaggctatattgacaatcacattattaattatagatagtataaaggaggaattgtaaatgaattataaagaagtattagaagttattaaaagtaataaaccatgtaaggttaggtttactggtagtactttgttccttgctaataaggaatttaatgcagatactaatagaggtatattacagattggtgtatctagtattaatacagacgactatattaagttacagcagtattgtttagaaagagatgagtataccgtagaaggtgctattttattttaaggagtgattaagtatggattatagagattttattactgattgtattagtggtggttatagtgtgtgtattaaagttactgataatagagttcatgttatctcagaaatggaatcagtatcctatccaaagaaagaaattaatttatataacttaaaatcttacgtttattatatgggtaactttggaagtcctattacaatggaggggttataatgaatagggttaaactattaaaattaatagagaatatggcagatataaataatattgaaattacaagtgtaaacttttatccagaagcactagacattaacaataaagctaagatttgtatgggtagtaattatgtagttcataaagtaaacgagggtggtggtaaaagatgcttgactgtagatgagatgttagatgaagttttagagttacttaaaccttcttttaataagggtgattatatcactaaaggggcacaatccggattacttattaaggtaggggtaaattcttacaatgttctaaatgatgacaatgaaactatctattattatcctatctcgttaaaagagttagaatatatgggatggagaaagagggattagctatgataagttcaagtcatgctaagtacctatcagatatgtataaagatagtgtacatcatgaaactagagatagtattgtagaagatgtattagataatattaatgatggtattattgaagaagctatgagagggaatacaagttatcaatatgttcttagggatttaagagtagataatgaagtagaagatagagttatagaagaacttattatctcagggtatagtgtaaactatgttaataatagcacagagtatccttttctatctacaaataatttagcaaggctagattacttagagattaaatggtaaaataaagggttactctatggtattacattagaatcattctaatctaggattttaatctttaaaccctaggaaaagtagtactaaaacatagaatcccttaggtatgggattctcatttttaaatttatgaaaaagtatatagtatctcctagaggaaagttttaatgtacttaaaatagccgattacttattatcatttaggggtatctaattgataatattggaagatgaccgcttctagtacaagtaaaggagattggtattgtgtttaatagaagaagggtaaaagatattcaaaataatagtaaagaaaaaataaacgtaggacatagtatacagttaaaggaggaacaagaacattatagaagattaaatactgatacttataccatacatgaattagataagattatagaagagcatttaaataatctagaagatatatctgtaaatgacttggagatagattctatttggttaggtactattggtatatacgactacccaatcggtcaaagtatgcaagagcaggtatatagtataagagaacttaagtatatgaaaaaacaatttaagaaactagggtttaaaacacaaatatcaaatagtgatgtaggattcacaccttacgtttatttaagattactatggtatatataaggaggaattatttatgtttaaaaagtcaaaagaaaagaataatgcatatgttaatttagaatatagtagaaatgaagataatcatgaaccacaaaataaagagttagttcagttagttagggacattactgggggttttgttttacctcttatagatggtcatgagtatgtagtattagttcgtggtactaagacattagtatttaatattgtaggtgaaagaatctacctaaatggctcaatgctaggcaatgattttgcttatgctgataataaaaataaccccgtttctattgaagtagttaaattaatttccaaattaatgtaaaggatttccgcttctattattgaagcggttctattatactctattatagtctaattctttcatatttaacgtatacagggttttaaatgtacctaggtatatttatatcaaaaagtactatcgtttgttatacgccgtctatgaagcatatagagggtattatatttcattaagtgtagtgtcagctttcaggataattttcttttctgggtgtatttatacctaaaaagtattcttgttaggtgacttatttatacatatagtatatatactatatgtatacttttataatttttttttattctatgcaactcctattagttacagagttgtagagtaaataattgaaaatagtgattgacattatataaggtatagtctagaatcagtagtagttattactgtataatattggagtgttggttatggttagtagtattataggacttattttaataggaataattagtgtagtattagtaagtgtgttactgtttatgtggtacgtagtaatttatactaaagataaagtattactttcagttgctatattactaacagttattcttggattaattggtgtaggttctgtactcaattactttggactataggaggtgaaactgtggttatatttatattgcatattattacgattattatcctagtattatgtttttgggtacttctttcttctagtagtcctttacatttcttagcatcggtatttgtgtatattactattatcaatatagtaggtatgttagacataagtaagtatactttaaatgtaataggattttttgtaatggctgttgggttattgattcttttaattagaacaaggatatataggtaggaggtaggagttagagttatgattagtagtattatttcaatattgatttgggtaatcctacttatgtgtgcagtagctatggatagtatttcctagaattaaaatttaatgaagataagttgagtaagtagaagcggataaatagaccgcttcttttttgttgacagaggttagggtaagtagtataatgtatgtaggaatattttaggaggttgatggataatggtttctatctataagtatctaaaggattatgacacaacatttgaatatacagttaatccattgcataaagctactatgattggtatatatggtagtaaagaaaagagtagaattgaatatgaaagtattagtgaatttgtaggtaagtttaattttccagatactaataaatcatttaaagcagagttacttgattatggtaaatatttaattgatgatgattatgagatagaaagtgtctctaagttaaaagacttaatctataactatgctaatatagaacaacacaattcttatttaaaactaaaagattaatctaattgataatagaatgataatacgtttcggacgacagaagtaatctacaatgaattatatacctgtcgtccgaaagccaaaaaggagtaaaatagatgaaagttagagtacatttacacaatactaaatataaaactaaaccagatgaagataaagtaggaactattagtaaccaaataagtaaaaaacaaacaagtataaatatagaagatttagctaaagaattatgtaatggtagaacagtattatttggtataatgaatagtaatggtattcgaaataagtttaatgttaatcatcaagaagtagtagcattagacttcgatactgaagaagatatgtctattattaataatgagtttatacaagataatgcttctttctactattatacattcagtcatgaattaaatgagcctagatttagagtagtatttgtattagatgagcctttaaaccattactttgaaattactcaagtatatgaatacttattaaacttatttcctactgcagataagaactgtaaagacccttctaggttattttttggtggtaataactacaaagaaattaattttaataatactatgagtgttaagggtttgattgattatagtttaaaggatacttatatagaggaactacctacttatgaccttattagataccacaaagatgatttagtaaaaatgagaataggtaacacttacaaagcctctgtatacagcagaactaaagctatggaatacttatatagagtagacatgagaaaggtattaggattacctaataaatctaatatacatgatattcttggttatgatagaaatcctagtgttaatgtttggaaaaccgaaagtggagtctggttatatactagaatgtataaagacagagaaagtaaaactaggaatattattcaagttattcagaaacttcgtggtgtcaacatgggtggtgcaattgattatcttatctatatactggggatagactttaatgtaagtgataaggttagaatctttaatgaggaaatagaagagttttcaaatactttattatctgaaaacttaaatcttacctatcctcaagttgcaaagatatttaaagatggtagaagtatgtacgcctcagatgtggttagtattctaaagattatgcaaaataatgttattgaaattgatggagaaattagatttgttacagatttatcttatagagaaattagtaatagaatctatggtcacgaaggtaaaaaagatagaatagcaaatgtagttaacttaatggctacaacagactggattaaaaagttagggtctagtgaaataccagataatatgcttgaatatttactaggacaccaagaaactaagaagaacaagtatagaaaaacagttttagagtataaaggtaagaataaagatacattctttgagatactagaacaacagtgtagagttatggtagaaaataattatacaaccgcttcattatcaagagaagggttagctaacctacttaatgaggataaggctaaagaagtatataatcaaagtgaattccctgaaagtaaagaaatagaatcggatattatacaatacattatgaaagaagttaatgataaaggatactgtttagaaaaagatataataatatattttgataataatagtaagtacagtagaagttatctcgagaaatttatgaagcggtatagaaataagttttctaaagggtatgatttaaaattctcaagattaaataagaatgtcaagaaagagcttggtttgttagatacatttaatagtaatcagacacctaatgcttactacaaatagatttactactttcaattaattactctttacctattgacactcattcctctataccttataattaaactataatcaaataaagggagaagataacttatggaattattaaatagaattgaacaagaaggttttgaaattgaacatattgaagacggggaatatcttattaaagatattaatggtaaggaagcggtacttacgatggcagatgggtatgttattgtcactaataacgatggattagaaactcatatctgtgatagtaaaccttcattaattatcagtgagttattagatgctatgaactatgactttgaaaaagaacagaagaaaaaacagttagccaaagcaagaaaaaataagtttgatgatttattagaccactttgattaatagaatcaattttaaacataattgaggactggttagagggtgtagaaccataggataaatcatttacgataacttaccttgagataagttgagatacgataggatgcaataacttaggatgttaagatacctaagtataagtttaatactatcattgaaaaggagatttctacatgaataaagagcaagcaaagaataagttatacgatgagttactttatacagtacgtgagtatggaggtacagaagcggatttattagagcttattgatagttttagttatttagacgggtgggctccgatattcaagaatccagaaagagaacaacactattcaattgaacaatttcttaaggaacaaaagtctgttttagagaaatacctagataatatagatgtagtatatagtaaagaagtaatcaatgattatgtactgtataggttattcaatgattcccttatcataaaaccagatgaggataagtataatgtaaatattaaagtgagtgataaggagtttattacactttccttattatcctttacggatataacagatattattataggtaataaagataaatttcagcaagtataaattaaatccctagagtgaaataactctaggggtttttcagtaaatatatattaagtattctaatcaaaatagattataggagaaagttaaaaatgactaagtacgaattatctttcagaagtgaaaaatgactaagtagagattaacttttctacccgaaaaagatagtaggtaatagattcctaaatattctgacaatttagacaaaagggagagcggtcacaatgaccgcttccaattacttaatttttttatataactctgataattctttttctaatttttcaattagttttgtgtcattattttcttttgctcttcttaacttcatttcacggattttaatgagtccgcttcccgcagtcaagtttccttcaatattgttatctttcaattggtttaacttgctttttcttccgtctaattttaaaccgaaattacttttcattttcatcaattcctttatattttatttgtagtgatttccactacacttattataatacagtaattgggaataaaagtaaagcattttttaggtaaaattttacacttttaaatagtcggaattgttttcatatttagaacaattcggaattgtctgaaaatttaaacaacttaaataggtcatcaattccattataatataaaaatctacccgtttcaacacttaatttatttaatatgttgtgtattatacatttaatagttcgggtcgtgtttcttctatataatagtaagaaaaagaaatttaaaaaaaattacccaaaagtgtttacaaagttctgggtctatgttattataattatagaaagttaatcaaggaggaattacaatgaaacaagcacaagcaattaaaaaattcatggaattaagaaaagcgctaaaagaagaagggtataatatttcaaaaaaacaattactatcaaacatggaaatatggtatctagatttaaaaattgattcacaaatcgaatggttcattaaaacacctaatgatgttatcattaaagatatggaagaattcggtgacttgtaatatgattaaaattagattaggtaaaaagataatcaaaagcaccgaaaaaactgacaacattctcattagtctaggggcttgcacacttacagcaattatgataaagttaatacaactacaaatgacgttaccacttgaaacgtggttaagataagaaagggattgtttattatgagtaaaaaaatagcacacgatataatgaaagcatacgactttgatgctttcagtgataacgtgtttatgcaatataagaacaaactaagtgatacaaactatcatctagaattgaatgtatttagtgaagattggtttaacgggtcaatcgaattttataagaaaaacaatttcttcaaaaagaatagaaatttaaaaacaggttgggcaacatttaaaaatggttgtacaatcttaaaggtaggtagcacacgctatgttatcttaaatggtgtacttattcaccaaaaagaaatttaaaaaaattgataaaaggggtttacaaaaaccccttaccatgttattattaatatagaagttaggggaacgacacaaacaaaaaaaaactaaaaaaagttttcgaaaagtgttgacaacctaaaaacttcatgataatataaaggtacaaggtaaccaaagcacaacgaaaaaaaaataaaatgtaaaggaattgatttattatgacaaacaaaaaatatttatacgtagaaacgcacacagtacaaggtaaagagattgaagtgtttagaattccaaatgacacaaacggaaacccacgctatgtggtacattttagagacctaggcgttagcctttgggattacgataacatcaataaactatttggctttaagaaatataccgcaaaatggtttggtggcggtgtagtattccaaagtttcaacatagctgagactttagaatacgcattaagtgaagttaaagaagcggttaaatagaccgcttctcacattaaataaaaaaattatataaaaagggattgataactatgaaaatattaatagaaacagaaccaacaaaaacattaaaacaaattaaagaggacacaagatttattaaaaatgaaattgctaatcaacttggattagatagggaacagtttaaattatctaatggcttaatattattgagaggttactccaaagaaaataattgttgggttaatttaactaatgtaataggaaaattcccagagtatttaaaaaatgatttatattatgaatataaattatattgtaatgtagaaactaaataaaaaaattataggggttgtttcttatgttacgtaaggaaattttagatatgctagaggttaattgtgatagctatttatcacaatatacaataagaaaagaattgaatttaaatgaggaattttatgattctactgatttaggtaaattatatatagaaactcgtttaaaatctattgagaaataccttaaaattacctcgttagattatgaaataaatttatatttaaataattattgttatgatagtgataaaatctatttagtgattaatcacgagggaacattgattaatgttacaataggtaaagaacaattcatcaatgatgagctagaattcatttttgatttagttgatgaagaaattaatttagactagaatgtattttctagtcttttcttattattttaaattttcagaaaattcataattgttcctacaaaacgagcctctccacttcctattatacgggaaagctccccgattgtcaatacttttcttcaaaataacttatttaaaaagtttcataaacctattgacttttatatcactatattgtaatatataaagtgtaataaggaagcggaacacttaaaaaaagaaattaaaaaaacttgctaaaagtgttgacattcattttgatacatgctattatagtattgtaaggtaaagaaaaacaaaaaacagaaaaggaattgattaaaatgaataaagaaatagtcaaagaaacagcaatagaatacatcatgatgatgcaaaacgaggtagaagacgaaaacatctcatttgaggaatacaaagaagcggttagttttgagagcgtttatgactacactttatcagaagctaataaagaatgtaagtttctaggaggtaaagtcttagcttccttaattaatgaagtaattggacaatatgcgtagggagcggtttaaccgcttctttttttgcagaccattgtctaaatattctgaaaacttaaacaacttagctaggtcatcaactccattatataataaatgacctaccaaaagcaatggtaaattttaccttttttatttttaaaaaagttagtcaaaagggtttacaaataagaatatacatgttattataattatagaaagttaatcaaaggaggaaaacaatatgagatacgagattggagcattagttaatggagaattcttcatgttctctacattcaagaaagaagaagcggaaaacaaatatcacgaatggtgtgaattgttcggtatagaaaacgtaagcgtggaggtgtcttaaatgttcaacctcttacacaaacaaactaaacgtgaactaaagcaaatgaagcgggctcaaaaaagagcccaaaaagaattaaagaaaattgtaaaaaagtgttgacactctacaatatacgtgttattataattatagaaagaaaaacaaaaaacaaatttgaagggaattgattaaaatgaaaaacacattaaacaacgtaattgaaacattagaaagcaaagggtatgagttcgaatatgatgaaactgttacagtattagaaattacaagtcctagcggtgcttatgaggatatcacaccaaggtttatccgtgacggaaaagttgagagtctttttattattcccgactttttagatgaggatttagggaacgtatgttttgagttttatggttggactttaaacttcacaacagctaaagaactaataacagaaattgaaaatatttttaaataagtgttgacagtcacttgaaaccatgatattattaagatacaaggtaagggaagcggtcaaccgcttccaacctagtaaaaaaagttttaaataactgttgacaacctagcatatagatgatatacttgtattaacaaattaaaagaaaaggaattgatgataatgaaaaattacacagtacaagagttaagagaaatcgtagcagaggttaacgaatatgacgacagtttagaagagtttgactacatggatattggaacacttgatgaaattcttagcggtgtagaacctactgaggttctaagaatggcacactttggagaatttgattggtcagacgattatttcagaatagacatatacggaaacctggaaagcgttaaatattttgaatttgaagagcttgtgaaagataatcacgatgaaattgtaaaacgctacaatgaacttgtgaaaggtggagatattgaaccaatcgaatttatttaatcattgttaaggtatccaccgcttaggtggtgggtatctcatagagtgattaaatttaaaaaaagtattgacactctagaaaatagatggtaatataagagtatagaaaaataaaaaaagaaaaggaattgattattatgaacaacacattaaacactatcgaaatgaacgtcgaatacttaggcactaagtgggtaaagggttgggaacacaacgcctataatgtagcactaacacataatggtgtcacagaggagtttatctggaagcaagggctaggaattcataaggagccctctcttgaaagagttttagaacaccttattaaagagtcttattattatgaagaggacatttacgatatgtatgatgaccctgaaatagcggagaaagttatagaacagttaaaagaagaagaaaataaattaaataatttattttctgaatatgaattagaagagttctactccttttactttgaagattagaattcatagaagcggttgacaaggaggagttaaatggaagatgtaaaccaacttttaaataatggtggggcggttgtatttgatagtacaaatatgaccgactacattattacaaaccaaggaaatatttattctaagagtaagaaaacaggagacctgcacctaagaaagcttagaactgaccgatatggttaccaatcaatagttatcaaaaataagtgctataaaccacatagattagttgcagaaaattttattccaaatcctaataactattcagatgttcatcatgtaaaaaacaaatataataatgatgttaatagtttaaaatgggtatctagtgaaaagaataaagaatacttttataaagataatttcggtcaattaaaaactactattagatataataaaagtttaaatattgtcgaatatgaaagctcatacaatcataagaaagaaaaagaattacttcagaagattgaacagttttctaatgaattacatgatttactaaatgaagcggttaaaattgatgtcaaaatatttaaagataatagttgacaaactaaccgcttcatgttattattaaattgtaagataaaaaaattataggagatgatttaaatgacaaacacactagaaaaagcaatcgatgctctagtaggcgcatcagcagatacaattgaaaaggacgttttatactggttaactggtgaggacgttgatacgctagatgaggctattaacctccttgagaatgtatcatgtgaagcgggagacgtaaaccaccttgtatacacatctgattgtattaattgggtgctagaacacgaggaggctattattgactttcttaatgctaatattgatgacgaggtagactcgttagataatatttcttgggagcaacacaaccaaataaattctattattactggttgggaatctggggacgatagagtaacgtggtttgtagatgattattacgaacacgccaaagagaatgcccgagaggacaacgagaattgggagctaatgacagaggaagagaaagaggaggcaatcagtgaggaaatagactatctagttaaattacacgaaccattagaattaacaagctcagacaaggctaatcttgcatggcttacgttcgagcaaacagcttgggacttagcggaagaattagaggaattaaagaaagttagttagttaggaggactattcctcctcttttttttatagggaattgtctaaatattctgaaaatttaaacttttaaagaatgtggattttttttgtaaaaaactattgcaattctaaacgtaatgtagtaatataatagatgtagaggagataacctcacaaaataaaattttaggagttggtaattatgaaacaatttataatggcggttgaatatgaagacggacgtttaggttacgtccgcatgtacttcgagggctacgagctcttagaggtgcgggaggttgttttaggtcacgaggacatgcctccacgctttgaggttgaggtggacgagatagcggactatctcggatttgcctacctcgaggaagaagaccaatttgggcgcttcttcggtgagtggttggctcttggtgaatcttgtgacgtgcctagtgattgcctcgaggacgaggacagatatttagtagaggaggtggagtaaatgacagagggcaaagcaagagaatacgctgaacgtcttataaacgctagttattatctgtgccctcacgacctagttggaatggctagtaagtgggacgacatagcagacgacattagaataatcggcgttgagtgtttcaaaaaagagtacttaaagggtctagaggttgaaaccttagaggtactcaacagatacgagtaaggcggtcaatagaccgtcttttttatttgactttgtatagtccgtatatagtccatatacgcacgctaaccgcttctgtgtactcggtaatgttattgtacctagatagataaacgtttgttagagcgtctatatgagctgtatatgacatatagaatagtttggttcgttggttgatacagtaaagattgaatgaagaaaaaaaatagaaatataaatagggaattaatatatagtttttatttagataaagataggttatataaaaaatataattaataaaatacgggggtcggtaggaagcaacataatccgtggcacattcacatagatacaccccccactt

SEQ ID NO: 2 - DNA, 92077 bp, unidentified
  source 1..92077: mol_type = genomic DNA
ttctcagatggacgtttttacttcttatgatacttttacagtgagtgaaagaggggatgctgtctttttattgtagacagttttttgaattttcagaaaggggggtactattcagaatattcagactattcagaattttctgacaatttaaaaaaataaaaaatatccccccgtattcagaatcttcagagggtttagaatattcagaaaactttaaaatcattatcccgtcacaaaatacatttattcccattgtcctcattcactcatagaacgcacacatcgaacgatagcacctcatagacctatcacaccagacagacctcacacaagccacaagatagctcatatacgctatatgccatatacatggttatactatggttataggttgttaggttatatggttgtatatccttatcatgtgtatctatctatcatatcaatcaatagatgaatacaatcatatcaatctaatcaatagatgaatcaataacataacattatagatactaatctatctgtatctatctatatctatctatcacatcttaatactcttacactatcctatcactcacacactcacagacgaacacagaacgcacacacaagcatacaacacatcatagatacataactatctatatactgttatcgttccttatatcctttctgtgtgtcctatataacatatgaacacatgtacatactatcgtgtgacctatcctctatctatccacaccatgaccacatacacataccgatacacatctacctgttacctatcacacatatacatacacatcagactcatagcttaacgctcatatcttgctcacatcaaacgctttaccctttacctatacaatactactatatgaccttacatgtccttatacgtcgtctgtgtgccttacatacactaatgaacatatgaacacatattcatatatggatataggtacagtatacagtgatagactgtacctgtgttaaggtatctatatctataatcatacagtgctttaatgtagtagctatctgttaccacctcacactatatggctacgatacttataagcacctcccttatattacacacgtatataatatataccacgacaaaaaagactatccacattgtgaacagtcctctttattctctactctatttaattgaatacatctatatagttggtaccaattttaaaccctaacgcattagatacatctatcagtgccacatcgtctatattattatatctgagtcttgtagccattcttaagtattccattagtaccatacgttctttatcgtcatatagtccacttaattcattgttgatatgttctataacaattactttcatatcatgtaaaaagttcccccaatcagtatctacaaccagtgggatattatccccgttatctattttgtacatcacgtttattttaaccttattatctaatgttaaattaatcatataatatatccccaatcattcgctaacgtcataagtgtttcggcttttgtgttgtcatttaataattttttaatatcatcttgccacgctttaaccacatttaaaggatagtcctcattgcttaatttaaattcaatatcatgaatgatttgatatcttacatagttttcaaacgttacgatattattactttcaaaaattagttcattgtctgttgttattaattggtatttgctcattttataaccacctttttattattttctaacttgtctataattttaaaccataatacttctaaagtcaaacattattttaaatatttttaaaaagttttcaaaggctttttaaagcctttgatttaacttatatttacatatatttgattaccgatagtaaacacatcataacagttgagataatcctctaacacgtcaaacattgtcatctgttcaaaggtttctaggtcgtctgtatcttcatgttgagtatatagagtacttaaccaatcaatataatataagtagtgttgtaacgcccccacaaactcattgacgtctaatgtattaggtgtaaatgagtttgttgccccattatataaacctctggtaaatgtttgataatctccctgttctacaagttcttgaaagtcctcttctgactcataaacgttagtgttcaaatagtcctccatatccttatatatcgtcactttattaaataattcttgtttttgtttaatgttcatttttgacactcctctttttatagtatttaaacgtctaattcatcaatcatatttataagtgtttgcccgtttggtctatcaatttctctaacttcccaaaaatcattaattataaaaaatctcaaatattccgttcttattttttctaattctgtaaactcttttttgttgtcttttaaatcttttactcttacacctattgagcactttctatttaatgttatcaagcccgtttcatgactgtatgatactttgtaccctaaattgattaatgcgcttaaaacatcatctaatgtgttcattttatcccctcctaatcttgaaatgtgctttcaatatttcttaattcatcaatcgtaaaatcataagcgccaacaataaatacattcaattgactttctgtgtaatcggtaaaagctaaaaatgtaaatatcatagcgttaacctctgttactagttgcaccattcttataggctcactagttgcaaaatcgttttctgataattctttaatctcctccatgctcggctttatcatatctcttaattttcttaattgatttaatttgttcattttaaaatctcctttttgttttttatttatagtagttaacaaccgaatttattaatttgtcaaaatcgttataatattcatcaattacattataagcattttttaaatctatatcattattttcaagaaattctatatcggcacataatacgatttcaaattcatcaagtgttaaacctaaaatatttcttacatctttataactacttgttttttttacgctttcggcaatatttttaatatcttctacaatgttatttaaattaaaatgtgcttgtttaaactcttttgttattttcataatttatcaatccttttcgtttttttgttatttcttaattatcattatacgcctattataatcaaagtcaacaattatttttaatcttcatagatgaataagtttttacttggtaaatatctaaagtttggctccattcctaaacaatatacaagcgtgtttaaactgtcacggtctgagttgctttctccatagtatgaaacaatttctttattgttcaaatcaatctctattgtttgtgttccccaatgtctcattattaatttattaccgtcaatttcaattgcatatttttcttggaggtctgaaaaaccgtaaccctcaaagtaacgaccattcaagattgcttgaccttttttctcggctttgtccaataagttctcaatagttttgataaatttcattttatatcattcctttttattttattagagttttaatcactctataagctacctaccgtaataggtagcccaacaatgattaaattattctgtttcttgtttttttaaataaaattctagcattctttcagagtcatttctcaatcttctcaagtcatccacgcttgccaggatatcattcaactcatgaatttcggcgttgtcgataaggtctaacattctgtaaatctctttcgtaaagtattcaatacgttctttgtacatttctgttgtgtcttgatattttttcattttttattccacctttttggttttttgtttgccttgcttactacacttataatactacaggataactatattaattacaagcattattttacgattttattatagtcggaatattctgaaaatttaaaacataagaaaaacgccgtcatgtgggcgtttatctgtaatattttatactaatcatcataaaaagattttaatatttctgttcctaaatcttctaatacctctatcatttcgtctttataaacatatgcccaacggttatcaagcttaaaacttacctctttatcaatatcctttacacttacttttaagctaccgtatatggtcgctttctcttctgctagtcctatgatatctattatatctatatcttcccaataaccattgtagcgcccacataactcactttgaccgatttcacattcaaataattcattttctattaataaaaatgtttcactcttttttttcattttagaattctcctatcatattaatttcttttaaatcatcgtataattcatgtgctttatcttcgaacgctatccaaaccaaatttgatatttttgttaaatctgaaatgtttacgtctctttcgttcaattctaaattacctaatatctctgtttcatggtttttaaaatatgccataatatcaacataatatattaagtggtttacttctccattgttacaaccataatttacaatatcttctaaattcatttgtaagtcataatcattgttattgttttctagtaagtaattaataacatcttgttttatccccgtatttactgttttcatttcttttaatactgtttctaaagttttcatttttataaccacccttattttgaattttaaggctatttatagcccgtttattaatttctaatataaataccctagcgaccgtttaaagtcgctatatggtatcttaaaatacgtcgttgtttattttatagtctttaaagtaatctaattgatttaatttaaagttttctaaccatttataagcttttctaacacttcttatttctctacaatattctattaagtattcttcaatttcatataaagcattatctaactcaatataaagattttctaaatcttcgtcatcttgtaaatcatagtttaaataattttctaaatcataataggcatgttctaaactgtcactattaaaaatcgtttcaacttctgattcaattccataagctagagtatcaaaaatattattatcttctttcatgtcgtatatatcaaaatcataaatattttgaccgtgaaaataaaatgatgatgttcgaccaatgtattcagtgtttaaatcattttcatctaaccactctaaaaacatgtcgtactgaaattggcaaaagtcctcaaatacatttaatgatttttcatcatcaccaatataataagcaacatcatgtaattttaaatcagtgtaacttaaatcttgtgtatgttcttgtagttttttaaattgtttgtttagtttctcaatctgtggtttatattcgttgtaaatttctgtaaagttattcatttttatcatcctttttgattttattagagttttaatcactctataagcaagccgagtagtcggcaagcctaacaatgattaaataacatcttcaatttctttcttttctacaagttcaaaataacgttctacaatctctttagcgcctaatttagtttgaacatctacatgataaccagtaaagctttcaagccaaccgtaaacattccctttaaaataatcatcatagatattaaagttgccaccttgtaccattcttaaaagttctgacggctccatatcataaaataattcatctaactcttccatatcgtaaaaattataatcctccaaactaccgttccaattgttaacttcgaaaaccatttctttcaactcttccactgtatattctgtatattttttcatttttaaaatctcctttttatgtgttttttgttttaaccgtaaatcattctgaatgctatccatattgtacaaccaaatgcgacaagtccatatctgtataatttttctttcatttttatacctcttctgaatattcctcaaaatagtctaataattcttgtctgtagtcctcttctgtcatgctttctaggttgccatatccgtccaatccaataaagtcatctgatggactatagtcgccatatagagccatgttgactacttcttgatgtgtaagttcttgcatttctaaatcagaccaaaataccgattcttcatatttcaaaatattttcatatgcaccgttcgtattttcgtttgcctccactaacatttcgtgtaattccattgtgcttaattcgtttttattcattttcaaaactcctttttgttttttgtttttctaacttgtctataatactacactattttaaaatcaatgtcaaacactttttaaagttttttaatttttataatgtaatcgcctttgtaagtaagtttgtagcgtgttccttttttaaaattacccgtattctcatagattctatactgttgttttttacttaaattgcgtttgtcttcttgatgttttgttattctgatttcttttctgtttacgcccacaccgtaacttatgaaagttgattgtgtttttattatttgtgatttctctttagttggtagacggtcaaagtctgtagcctgtgcattccctcccgtaaacaacattgtgccgattgataaagctataatagtttttttcattttgtagccacctttttttaatttttgataaatctataaactacattgttttattgcaatgtagtctaacatctatcaaagattgttacacctcataattaaatttatatgcatctaattcatctaaccattcaaaccatttgtaagtatcattgtcccaatcttcaaatcctaattgttcatatgttccacttaaagtagtgaacaaatgaccgattgtctcgctatcaattcttacaataatatttttaccctctttgtgaaattcgtaagagtgacctctaaaggtttgtgaaattgttatacccttgtttagtccgtccattagttctattaatcttgtgttgtcttcattaaagattatcattgttttttaccaccttttgatttttttgaattccttaattatcattatacactaatataaatcaatttcaagttaaaatatcatttcatagtcttttattagtccctgttctttagtccaatatctgatataattttcttgtttcttgtcatatacatataattctaatgtgtccgactcaaaaccattgattgatgaaattctgaaagtgtcatgatgtttcaagcatatgttcatatcgtcatcgtatctcttttcaattacgttttttacttctgtctcaactctgaaattttgtaacttcttaattgttcttttatccattatttaaaccaccctttatatgattgtgtgtagtctgtaataactacctgttacatcgttatcattcaataatgcctgtaatagtcctttttcagttctgtaagcttttataactgtttcacgttcaaatacttgtctttctaattgttcaagcgttgtatcacgctcaatacttacatacgaacgtaacacgtcaaaatattttatatcttctctttcttcgtcttcatgttgttgatagtttacaattgtaacgtttgcccaatcattgcccgttacttgcaaatgtgctttaacttgcgttagaacgtcttgtaattcttgatttgtataattgtttagatttttaagtttcatgatatgaccacctttttggttttttgtttgccttgcttactacacttataatactacaagatagtaaaagtaattgcaagcataagtttttaagttttttacaaaaaacgcttgaaaccgttgggggagtaaggataaaaaaatttaaacttttttatttaaacgtgtttcttctatataaagacaaaaaacaggtttttaaattgtcagaatatttagttttattacaaaacattacaaactatcacagattttaaaaaccatgagaaactataaacagttaaaaaagagaaaaaagaacattgacaaataaatatttattgttctcaagttttttaaatattgtaaaatattcagaaaattataaacaattcagaatattttgaagttttcagaaaattcagaatattttataaaatagggaaaattctgaaaaaataaaaattcagaaaattcagaatattgaaaattcggggtcgaatttttttcgggtcagaatcacatttctcatgataagaaaaatctcaaaaatcgtgggggcgaaattttttcaccattttcacatttttctcatgataacaaaaaattgaaaaatcgtgggggaataataaaaataaaaaataaataaaaaaatattgacaaaaataaaaatttatgataaatttaaaacataaaaaatatttaaagaataaaaggagaactaaaaatggaattaaaacataaaataatcattaaagaagagtattattcagaacaagatgctgtttcttatttgattcaatcaatagaaaatagattaaattatttaattaagcaattggagaaagatgaaaatattatcatattgaacatatatagtaatacagacactaaagaccattatcttgatgacaaaatagtattagaaagaattgttgaatatgttgatattgatgaaatagaacaatctgattatcaattagtagaaatgtcagaccagtattatctttatgataaagattcaagagtgctagatttaaaagatgaagaaggagagtggatgtaatggaattaaataatgttaatgagtttgatatgaataaagttaaagagatggttagactttctaatttagaagatatggattcatatgatgatgatactaaaattacggatttctttaaaagagtgtttttagatgacgaatcaatgggtgatggtatgattgctgtaaaagaaatggtagataaatttattggggaattagtatcagagggagaatattctcaagaagattcatcagaaggaaaaatatcaatatataaactatatcctttttatggtttagaagatagaataacagttaagataatttatttaagtattaaatatttttacaaaaatcaattagaagcatcatataagatatatcaaaaacctaactatataagaatatgcttaacttttgatgaagagtaaaaagtgagagatataaataatcatttccctccatatagaggggattgattaacaatctttaaaaagtacagatatttatataagagttttttttggggatagggggcttgaaaagtgacaattttttatatggagttttttgtataatggagggggtcaaaaagtacaggtttttgtataatgatttatatgtatagggaggggatataaaataatagttgacaatatatattaagatatgttatacttattttaacaacataaaggagattttaatatgccagtatataaacatgtgttgataggtttaaatggagagaaatattatttttactcatcttttaaacgattttccgttagtaaagaggaagagttattagttgagaatgataaacatgaagaagttttaataaatactagagacataaaagagatattaggacaagatttttatttaaaagatacttttaaattggaggggtaataaattatgaaaattaaaacaaagaaagaaatgacattgccagagttgattgagtggataacagaaaataatataacttatggacaatatatgccaaatctagctgaaaacaccagatttattgcagtaaatggttatggagaaatgagaatatatgggttcataagaccagatgaaattttctcagttgaaattgaagaaccaatcacagaggatacagtgattcctaagctgatggttaaatttaaatcaccaagcagataccaagttttttcgaatgattatataaacggttttgaagttgaagaagggaatatagaagccttctacatcatgaacgaagacctaacaatgacacttatctggactaaagagaaaggactgattgagtagatggaatttaaagcaggagatagagtgcatgttactatttttcacggtgaaaaagtagatttctatgcaacattagaagatataggaagtacagtagcgtgtttaactgattgcactaataaaaattttaattatagagttatatgtttacctaacaatgagcattttggtaaagataatgaagactactacaccctagcaactgacgaagaagatacagaggagcaggaatggaggataaataattgcctaaagtaaattatgaaaaagcatggtacgaattaaaagaaagaaaaatacaagaatatattaaattacatggggaagatggattaataccatggtctaagaaaaaagatttaggaattgaattatccgacatggataatatcgatggctcaaatgattttggtaatttattatatgatttaggaatcgttaagaaaacggagcgtgaatgattatggcatatgagtatgagaaaaatgtattggctgaaataaaagagttaggtgccagatataaatctccttctatctatgtaatggcaggagaggttgaagaagtgtatcgtaaagcaaaagtgttagataaagttgtgaatattgtcgaagataaagaagatttttatagtaacgaatttatcgaagcagtggtaagcgcagttgatgaatttttagaatacgaattggaggacaaataaatgaacaaagttgtaataattgatgggcgtaattcataccatttacaattaaagataaacaaattcatatatgacaaaaaaatcatatctataattccttattataatacatcaacaggtgaacattatgctatgatttattataaggaagaggataaataataggggactaagaaatgactatcaaattaaaagattatattaaaggtaacactaaattatcagaagatgatatattaaaaggtttaaaaagaataatgtttaaaggagataacaatatgagtataacatatggaaataaatatacagtaagaataaaagattatcattcaggtgctataattacagattctcataataataaagaaatattgattaatgagttagaattattagctttaagtgggaaaatgcatatcatggcagtacaaaattatggaacaccattagatgaaaattctgttgacaaagcaatagattatgtgttagacttaatgagtaaatcataataggaggaatagaatattggcgattcaaggaaagaattttacagctagtttagatggcgttacagctaatgatttagcaaagaaattagacagagataaaagtgaatatagagatagattgaaacatgttgaagatatacttgataatacagaattttttaatatatattatgaagattattttaatccaaatataagtatgaatgaatatatagcatctgaatctaatatcagtgtgttattagagaattatgccagttacctcctaaattccgaacattgcgaagaagaggtactatataaaatcaaaaagggtgaattactacctaaattagacatgtccgaaaatccacctcttgaaagcgttgggggacaaggggttcagaggtcgctttctcaagaaccatctataaaatatgaattttatacagatgagaacaattttagaaaagcattgaatagagatatgtctatagagggaatgtctagcaatggagcaacagaacatgttattgatttcttattaaacataggctctaactttaaaaagagtaaagaccaaactataacatctaaagatttaaatcgcacagattttcttggttctgttttaagagattataaaacatacttagagtttttaagaaacataccaaaagattcacctaattataaaaggtttgttgtagacagaattaaggggtctattatgcaggatatgatagatgctaaggatggtttgcttagagttcatggatataatttaagatacttcagtgaatcaacagaaccatattatgatgctattaacttaggagaccctgagcaattagttggttttaaagatgacaatcaacctaaaggagaaataacagttaaaggattactttacatgaaattcaatggagattttcaaaatgattttcaatgtattctatatgatttagaaaaaattattgagagaactcccttgacagaaaagcaaagaagtgttatacttatggttagagacggatatagtaattttgatattgctcaagaattatctatagataagtccaacattaaaacaaaatatatagaaccttctgctaaaaaaatagctgaacaagtaaggaaagagaggttatctttatgttagatatgactactttaagagcgttaaggggtaaagagtttgtagttactactaatgttgaattaaaaggaactgtggatgttgttgtatgtgccttaaacgaggaagatgctatggatttagcacaggatatggtaactattgaagatgctatagaatcagaaaatttaaaattagatgttgactctgaaggagctgtatgttacaatggataaagagttattatttaaaagcatacacgatatctataactcaacagagtatagagaatggtgttatgaatatcatttcgtgccatttcatgtaacaactataatggcttatttaaaaacaaaaggagaagatttataatggaaaataaagtatttttattgtctgcaccatggtgtagcaagtgtcaacaagttaaaccaatgttacacagtaaggtagaaggtgtagaagaaattaatattgatgaagacccatcaattcctactgaattaggtgtaatgagtattcctactgtagtagataatacgggggaagaacctattgtttatacaggacaggctaaagtgatggaatttatttctaaaaactaataaataatttttaaataatggttgacagccactgcattatcatgttatacttaacaagtaataaaaacaaaacaaaaaaacattaggagagattaattattatggcacaattaactaaattaaaaaacgaagtagaaatttttggtagagtgaaagatattaacttagaagaaacaacaaatagaaaaggcgtagattgtattaaaggttctgttactatcttagttagagaatcaaaagagaaagtacatgatattactgtagatgtttactctaataaatttaaaaaagatggtacagagaatggattatttaaaggatataaaacagtaaaagatacgtttgtttctattaaagaatcaggttcagagaaagaagcaacatttgttaaagtttcaggagagcttaaacacagagagtttattactgaacaaggaaaacatgttaagtataacaatgtaaaagcaaaattctttaatcatatcgaaccagaaaacattaagagtgaagttggtaaaaaaggaccaagagctgtggcagtagttgaaacagttattcaatcaattaaggataaattagatactgatggtcttccaagtggagatttagttgttagtggctttaatgtagatttctttggtaaagaacctaatcctaatttctctccagtaactcctatgtttgacatggtagttaaagagggtattgcagatgcattccaagatatgtatgaagaaaaggatactgctaagttaacattaaaaattaacaactatgcagaagatgctgacgaagaagatgaagaacctgaaatggaaacaggttttggtgaacaagaagatattggaccaactaagaaaaactttgtaaacaatgttgagattattggtggagctccagctatggttaacggtaaagaatacgatgacgagcagattgctttagtggatgagattagaaaagaagagttagctagtttagaagatggatatgtaccatcaacaacaagcgaagctaatgttggattcggaacagatgagccaactaaaaaagaaaacttatcagatgacgacctagatgtagaagatgttgattttgacttttaattagaggggtaacaccctcttttaattaatttttaaggagagatatttattatggataataaatttttagagaaaaaaagagaactggttaaagaagaacttgcctatcttgataatagaaaatttaaaatattagaggaaaaatacaagattaatagacaaatagaaaaattagaacttgaaagagatgttttacaaacagaaaataaagtaatacagagaagattatctcaattatcagaagaagggtatttatatgtagcttctgatgaacataaaaaagattcaaatacagattatgaaattcaatttgatatttttgaataaaaagaaaaaggagaaattaaaattatggcaaaaattacattagacgatattatgaacttagaaggtaacaaagtaccgacagatgcaaaatcatatagtacgtttatttatgctaatccaaagacaggtaaatcaacatttgtaaatgatttatatggagagaaagtattattcttagccacagaaagacgtcatgatgcaatctctggtgcaatggtaatcaatattgatagttgggctgagtttttacaagccatgaaactattaaaaaaaccagaagtacaagaaaagtttgatgcaatttgtattgatacagttacaagactggaaagttactgtgagaaatatgtactacaaaacttaaatgttgatgatttatcggatgtagcatggggtggaggattctctgcttataataaagagattgagaatggcttatcattgattgaaaaatcaggatatactcccgtattcatctcacatgctaaaacagagacagtaaaagtattaaaatctgaagcatctaaagaacaagaaagtgtgaagggtgctaaattagttaaagaaaaagatggtaaagaatatgtggaatatcaaaaacaaattccagatattaaaacaaaattctttaatatgattaatagaattgcagaccatgtgttatatctaggtattacagttgactctaacggttccgaaactagaagaatcttctacagagacacaccagaacactttgcaggtgcttcgtttgcacatattcaagactattcagagttatccccagaagcataccaagaagcagtagagaaagctttatctaaagaaggtaaatcacatgttgaagatagaaaacgtgaacaatcaaaaggtcctcagtatgactttgatgaattaatgttagagttaaataaagtaggtaaagaactgcaagacaacggtaagaaagacgaaagaaatgctgttattgagagagtattaggtcatggtaaaaaagccaaagacttagataaatctcaagtagaagtattatctgtattagtaaatgaattaaaagaattataataatacccccataaagggggtatactattaaaaggagatataatctatgaaagagaataacagaatagtttcagaaagtaaattaaatattgtggtacaagcattaaaaaatgctctgttcaatgaatttgagtatgtgtcagtagatggagacatggatgacggtatctcagtgacagtatctaataatgataatattgatgttattgaatatatagattttgaagacattgattacatgatgacctctaaacacaacgcaaggaagttctacagagatttagtaacacgtcttaaaatacagcatattaaaatagcagaattaaaagacaactttactactccaacgaacctagaaaatattaaaaagaccgattttttattgatgttagaacaagatttacaagatattttaaacgataaatatgttggaaaaaattatgacagtaaacgtcatagagcgcttggtaaaacaacttctttagcctatctatcaatgaaatatgacattcctattatattaagtaattcaacaataaaaaaaatagtagaaagagatttcccagaagcaagactaatagatttttctaagtttgaacgtaaagtattaaaagaagatattgttcttgttgatgaattaaaccctgatgaattctatgaggttcaacaaacaggagtaaaattcttaggttttgttcgttactaagatttttaaagatttaaaatatatctagtaggtgagcattatggaaaaattaaaagcatttttaagtagtgtcggtatatcttatgattataaagaaatgattatgaaatatgagacagaaaaagaattaaaactatggatagataaaggtatgtttataaaagtacgttccctttacgattttccttacggttatagaatggatataggcttctgctcggaagattctgtattgggatatatccaatgtggatactactataaagatgaagaaaagattttgattaatgatgttgcacattactacaacaagcacacaggggattgatttccctgtgttttacatattggaaaggagatattaaattggctactacaaatcttgcacaaaggacttttaagaacacagaaaaagcattaagattttttaaaggtcaaagaaatgaacataatactcaaagaaaaataaaaaagaatgttgatgaactattcaaaatatctaaagaagatattaaaacagccagtgaacaaggaagatttaactgtgttgtttattataaaaagtttaacaaagaggaaagaggatataataggggagagatgattgacgttgctaaattattagatgaggaaggttttaaaacaacctatgaagaatcattagacggtgttatgcttattgtgtcatgggaccatagaccattataaaaaggagagttttttatgaagttaaattgggattgtttaagagaaataaaaacaacagcagagagttctaaaaattttcttgaattcagaaacaagagtttacctaaagaactacaaagtgtattattatcagatgatatggtaaatattatagattatatttttgaaaaaagtaatgaattagctttttctcatccgtcttttagtaatagtcacagcgcattttttaaaaaatatggtgttatatatcatatttatcaaggtggaagtggattatctataattgctaatgttagtagtgctaatattggtaaaaaagaactaaaaggatattcaaaagaagaattcttaagagattatgatatttattattactatgatgtaaatgataataaggtaagatgtttttggaatgatttaccatatataataacagaggaggagcaacaccatgattttagtttactacagtagaacaaacaatgtaaaaagatttgtgaataaagtagggatagatattgaaattcaaactatagatgagtatgatggattttctccttatattctaataactccaacatataattttggtaaagttccagaagaagttgataagtttttagaaacaaatgctgtaaacatggtgtctgttatctctagtggtaataagaactggggagattctttgttcggtagagcaggagatattatatcaagaaattatacagttcctttagttcataaatttgagctaagtggaaataaaaaagatgtagaaaaggttgacaagtatatagaatctatggtatactaagtatacaacattaagaaagggtagagtattagatgacaaaaaagaagacacatattgaattaaataatgacatgatgaagacagatgagaatggtcagttacagtttgataaagataaggaagctgtaaaagcattctttgttgattatgtcaacatgaacatggcttggttccatgatttagaagaaaagattaattaccttgtagagaatgattattatgaggaagatttaatttttaaatactctatgaaagacattaaaaagatttttaaacgtgcatatagttataagtttagatttccttcttatatgtctgcacaaaaattctatgaacaatacgctttaaaaacgtatgatggagaacatttcctagagcgttatgaagatagaatgagtattgttgcaatgttctttgctaatggggatactgattcagcaattaattatgtagacttattaatgacacagcagtatgtacctgcaacacctacaattctaaatgcaggaattaaaaaaggtggagaaatggtttcatgtttcttacttgaaactggagatagtttaaatgatattaatatgatgaatagtacagctagacagttaagtaagcttggtggaggtatatcaataaatctttctaagacacgtgcatctggagagtctttaatgggtaatgaaaataaaacaatgggtgtagtacctattatgaagaaccttgaccaatcatttagacatattaaccaaggtggtaaacgtcaaggaagtggagcaacttatttaaatgtgttccatgctgatatatatgagtttttagatgttaaaaaaattagtgcagatgatgacttccgttgtaaaacattatcaattggggttgttattccagataaaatgattgagctggctagaaaaaatgagcctatgtacttattctatcctaagaacctgtatgacactcatggcgtatacattgatgaaattgatatggatgaatggtatgacgtattaattaatgacccagatattagaaagaaaaaaattgacccaagacacttactagaacgtatttctattactcaaattgaatcaggctatccttacattatgtttgaggacaatgtgaacaacgaccacgctttaagaaatctaggtaaagttaaattctcaaacttatgttctgaaattttacaatacagtgatgtatcagagtatactgattatgacgaaaaagataatatcggacaagatatttcttgtaacttaggctcattaaacattgtaaatgtaatgaaacaaaaagatatcgagaaaactgttagactatctgtagatgctttaacaagagtatcagatgaaacagatattaaaaatgcccctgctgtaagaaaagcaaacaatgaaatgcattcagttggattaggagcaatgaatctacacggattctttgcacaaaatggtataccttatgaatctgaaacagcaattgattttgtagatactttctttagagtagttaatttctggactatttaccaatcaacagaaatctcaaaagaaagaggagaaacttttaaaggatttgttggttctgattatcatacaggagaatatttcaatagatacgatgataattttgaaataaaaacagataaggttaaaactttatttgaagacatctttgttccaactaaagaggattgggatttgcttgctgaaaaagttaaaaatgatggtatgtaccattcgtatttaagtgctattgctcctacaggaagtatttcttatattcagtctgcaacagcatctgttatgccaatcgtagaaagaatagaagatagaacttatggtaatagtaaaacttactatcctatgcctgagttaagtcctaaaacatggttcctatataaagaagcttttgatatggatatgtttaaagtagtagatttgattgcaacaatacagaagcatgttcatcaaggtattagttttactttatttgttaaagatgatattgatactagacaattaagtaaaatatatttatatgcacaccataaagggattaaaacattatactacacaagacaaaaagatacagctaatgcagaatgtatttcatgtgcagtctaacttaggaaaggaaaaagataataaatggataataaaaacaaacttgtagaatttataaataaattagaattagataaagatatattaacagcaggaaattggagcattaaagatgataattattctgaaatgttcttactacagaacttgaaacaattctggttgccagaagaagtgtcactctcaaatgatactcttagttggaaaaaattatcagaagaagaaagaagagcatactctaaagtattagctggtcttacattacttgatactattcaaggagatgtgggtatgccatctattgcaaaacacgtacattctcatcaaaagaaagcagtattgtcctttatgggtggtatggagaacgctgttcatgctcgttcttattctaatatattcctaactttagaatcatcttctcaaatagaggaactatttaattgggttgaagataataagcatcttcaaaacaaagcatcaaaaattaaaaaatattatgataatatagaagatacattgtctttattaaaagccatcgtggcttctgtagctttagaatcatttctattctactcaggatttttctatcctttatatatggcaggacaaggtaagttagtgggagcaggagagattatcaacctaatcttacgagatgaatctatccatggcgtatatggtggtatgattttccaagaatattcagagatgttaacagatgaacaaaaggaagaactaaaagtatttactgaaaaactattcacagagttaatagataacgagatttcttatacaagagaagtatatgatgaagttggtttatctatagatgttatagattttgttaagtataatgcaaacaaagccatgaacaatttaggttttgaaggaatctttgagcatggagaaattaactctattgttctcaatggattaagtacagaaacaaaaacacatgatttcttcagtgttaaaggaaacggatataagaaagcgacagtagtacctattcaagatgaagattttatattcgaataaatttataaagaccccttgacgggtcttttcttttatgttatactttatttatattaacaaaggaggaaataatatgatagaactaaaagaattaaaaagagatttagaagaattattggatgaaaaagatattatattatgttatacactagatgatgaagaagaagttgctattagtacagaagatttttatattgatataaaaggattcgataaaactagattcacagcaatcgtaaattcattagatagtggagatatttcagaattaaaatttaaatacttatcagatttttgttctgtttttaaatcaactattctaaattttcaggtcagaaaaaaaccctgtcaatatagagctgtagaaagtgatttaaaaaattttaggatatataacacctaaaaaagatttacgcttcaatatggctttgatagacaccttagaatggattctgtgtcaaagtagtcaaaaaaactactataaaatgtggattttatatatgaaaaggagatattatattgattataacaagagagtgggctatgccaaatagtcgaacatttaaaattaaaccaataaaaaatattatagaaagataccaagaacagttgtcagataattctttagttttagaccctttctcaaacaataatatggtaatagatagagatagattaagcttaaaaactaatgatatagaccctcaatatgatacagactattctttagatgctttagattttttaaaattatttgataatgaatctgttgatatggtattatacgaccccccttactctcctcgtcaggtaagtgaagtatataaaaaattagatatgtctgttaatatgcaaacaacacaatctagttactggggaaatcaaaaaaaagaaatatctaggatagttaaaaaaggtggatatgtaatgacttttgggtggaatagtggaggaataggtaaaactaaaggttttgaaatagttgagatattgatggtagctcatggtggctggcataacgataccatagttactattgaaagaaagatatgaaaaggagataattataatggataataaaaaattagaacaaattaaagaaagacttttgtataagagagttgtagaatggaacgaagattatattatgttagatgatggcactaaggtttatgtatattgttcagaatatgattgttgcgcaggagcaggtggttatttcaaaaatgtaaaactagatgcagttattaccgatgtgacatatgatattaaacaagacagagaatggaacaaagattcagatactagagaaagtgaagctgtgttaaaattattccataatcaaaatgtaattgcacaaaatatggtaaatgcagatgcaggtaatggtggcttttactatagtgtggcatcattggttattggagattttgaaacagatattttagatgcttaaaaaggagatactataatgaaaaaagtttattatgttatagaaattggagaagaatattttaaatcaagaaactttttagatagatttgataccgtaaaagatatagaagatgctacaacgtttgataattacttcgatgccagagatttagcaaccagtattaatggtaagataataagattgaaatcaaatgttgacaagtaatatattattatgttatacttgttttacactttagaaaaggagattatgatatggataatggagctaaggtttttggaggttgtttacttattgtatttgcgacagtggtaggtattggagtaactgttgtattacctgcttatattatatcgcaagtattaaatggatttaatatttatataagcacatggaacacttcatttttattgattgcattatatattattttttatgtagtaggttcattcttttcagggggtcgtaaataatgataattattaaagattggtttaaatatgataatggctcagaagtatttattgctaaagacgaagaagaaaataaaatatataagtttgtaaaatgtactaataaggttagtaaaaatattcttataggaaaaataatggaacatagtgaacattatttaacacctagcataatctttgctaacacacatgaaactgtatacttactagaatactataatggaaggaactttatctcagacggagaaacacatttatatgaagatgatatgatagaagcacataaagatgctgttaaggcagggttaaatccttcatttggctatgatgatgttatgattgacgataaagataatcttattgtggtaaacctatctaagtttaaattaggtgaatcttatgactagaaaaaattgggttattaatggaacatctatagacacagaagatattttttgtattataatatacgcttataatggaagacaattttattttgacgtatcatctaatactcaagaagaactacaagaacttgaagcagatttaaatgtattaaagttaaatgatagttttataggaaagtatggaaaaagaagagttagcatttcagaagtatctgcttatgaaattgaatatgaatgattttattgacagcatatcatttatatggtatgctgtatacatatagttagaggagatgaattaaatggcaagacttgttaaatgttatggtacatgtggggaaaagcatagttcagatgaaatgagaaaactatctggaaaaaatttctgtcccaaatgttatgacatagaaaaaacaaatagagataatagagatgctgtatatcgtgttatagctcatgcttatgatttacaaatgcctacaacattaatgaaaaaacatgtcaaagatatgcatgaaaattatggatggtcttacaaacgtatattggttttagcagattatgtctttaataaaaagaaaattaagatgctagatatgaaatatggtgttaaacatttagaaaattatcatcaagaaatgaatgaatattatctaaaaagaaaaagacaaaaagagaaagctaaacagataactttaaagagaaagactatcgtaataaataaaagttttaatgacaacaatagatataaagattcaaaaatagtaaatatggaggatttataattatgagtaaagatataaacttatctaacctaaaccctgttagggaaatatataatgttataggaagtgtttgtcaagacactgaattattaaaaggacacaatattcatctgaaagagacagactttgctcaaagaattcacagaattatttttagtgccatgtctaatattatatataatacatctggagataaagttaaaacagttagtggggtagatatagataattatttatcatcctatccaaaacaatataagatatggaatgatgagaaaggtattgaatatgtaaaacactgtatagaaaaaagtaacccacaaacatttaatcaaagctatgatagaattaaaaagatggcaatacttagaaaatatctattagagggcttcgatatcagtgagatgtatgactataaatccgaagacttctcggaaaaagaacaaatgatgaaagacttagataaaaaaactaaagaagaaatatttgaatattttaccttaaaaaacttaaagattaaagatgattttaatatagaaacaggaagtgtagaatacaacgcaggtgaaggtgttaacgatttactagcaaggctcaaaaaaggtgaaaaagattacggatatccttttgaaaatggttattataattatttatttagaggaatgagaaaaggtaagttcttactgcgttcagccaactcaggtggaggtaagacacgtaatgcattagcagacatgtgtagtgttgccattagtgagataagaaagaaaaacaagaaaggtcaatgggaatgggttaataacggagaaccagtacctagtttatttatttctacagagttagacatggatgaattacaaacaatactgttagcatatataacaggtattaatgaaagagttatacttgatggagactttgatgagaatatggaaaaagacttaagaaaagcaggtcaaatactagaagattctccttttaaagtagttaagatagaggatttttctatcatggatattgaagaaattattgaaagacatgtattagatagaaatgttcaatttgtttgttttgattatatacaaaacgttgctaaattatctcgttcagagaaagaaagatttggggaagctagaagggaagaccagttattcctagaaatgtctaaagcattaaaaggtatggcagaaaagtataacatttttattgagtctggaacacaattaaacagaaactcaaaagatatggaaagaagagattcagactctttacgtggtggttcagcaacagcagataagatagaccacggtgttcaagtatatcaaattaaagataaagataaacagaacattaagcatatacttgaagataaaggatttagacatgaaccaaactatgctcactttatatataagaaccgtgcaggaagacaagaatgtattgtttggactaatatggacctgggaactataagagaagaagttctttttgttacagattatgattataatttaattgatgatgtagtagatttacattttgaatttggtcaatattcagaaaagaaagaacatattgatattgataagataggaaaaagtgttgaggtagtagaagatatggacttttaggaggtagcttaatatggatgcaaacacattaaaaaaacaactgtctgagaacgacctattaagcatacttgaacatataggtgcagaaccttacttagaaggtaatgctatagtatgtagaactgtctgtcacggaggacataaacacaaactttattactatatagaatcatgtacatttcattgttttactgaccattgtggtaatatggatatatatggattgataggtaaagttttagataaagagttttttgattcttatatgtttgtattatcttattttggttatgagtatgatgggaatgaaattgattacagtgaaaagttagatatgtcattctttaaaaaattcgaaactaaaaaagtagaaactaaattacctaaaataaaagaaaatattcttaatttattctataatatgtatcatataagttggataaaagatggtattagtaaaaggtcaatgaataaatttaatattaagttaaatataacagacgaacagatagttatacctcattatgatgtggaaaataatcttgtaggggtaagatgtagaaatttaaagcaagatgtcgttgaaagaggaatgaagtacatgcctataagacacgggggggtggtatataatcatccaacaggaagcgttttatatggattaaatataaataaaaaagatataaataaaacaggtaaagtaatactgtttgaatcagagaagtctgtgttacaattagacacaatgtatcctgaatgtaatattggcgtttgtgtatcaggttcatcattttctgataggcagttagaaatattaaaagagatggatatagaagaagttattataggatttgataaagaatatgatgacataggagatgaatctgaaaaatattatgcgaaaaaaatacaggacaccgtggcatctaaattaaaagcttattattctgtaagtgttttatgggacactaaaggcttattagagaaacaagatagtcctacagacaaaggtaaagatgtatttgacacattgatgaaggagagaataagtatatgaatattattcaaagagaacagaagtatgattgcattataaaagatacattagcaaataataatatagacggaagtaacatggtctttcatgtaaatcctacagataaatatgtgactagagagactgccttcttggcaaacgatggagtaaaaaattctatgttggtgctatcagatactatcaaagataatgagaagataagtatattagtagatagtgatatggatggatactctagtgcgacattaatgtataggtttattagagatgatttagaacataaaaacgttagtcatattatacatgatgataaaaaacacggtttaacagacagtatcatggaaaaagtattggaagataagccaggcttattaattataccagatgctgcttccaatgatgtatctaatttagaagccttatctaaagaatcaatttctgttatagtaatagaccatcatgatattgataaaaaattagataatgatattcctgtatatatttttaacaatcaaacaatgagaaatgttaataaagaattaacaggtgttggtatggtatatctatattgtaaaacatactgttatctttatgatattgatgtgaatattgataagtatttagacttagtatctttaggtcaaactggagatatgtctgatttagcagataaagaattaagatattatataactaaaggtttaagtaatataaataatagattcatcaaagaaacattaaagaaaaagaataaaccagaagaaccttcttcaagagatttatctttcagtattatttctatggtaaactctgtaactagggttggaacaataaaagagaaagaattattgtttgaagcatttataaatgattcagaagaaatgcaagaaatagaggtaagaaagaaaaacaaaaagactggtaaaatggatactattaaaactgaaatgacagtggaagaagttgcattaaaagaatcagaaaaagtaaagaatagacaggatagacttaaaagagatttaagcaatgaaataaaacaaaaacttgattatacccaacctatattaataggtattatagaagaaaatgacaaaggttctgttaatggtgttatagccaatgcattgctttctgagaacaataaacctgttctagtattaactagaggagaggatagtttttatggctcagcaagaggatatgaaccatatatagaaagctttaaggattggtgtttggaaacaggattgtttacacttgcacaaggtcatgcaaatgcgtttggtgttattataccagaggaaaatatgcctaaattaagaagtattatatctcaaatggaaactgttaaagatattgatattgtagtggataaaatttatagcaaaccagaaccatatgatattgaaaaaatagataagcaactaaatgtttttggtggttctgttcctatacctttattagcatatgaaaatgtaaagtttaatattgcctgtgtcaaaacaagaggttctgttcttactttatttgacaatggtttagagtttgtttcttatggtactagaggtactatcaaagaagagatagaatctaatttgacaaataatacttttagagttaatataataggagaacctagtatgaatgactggggaaatacaaaaagacctcaagttgtcattaaaaaaattgaaatattaccaaaagagacagatgattttgatgatgacttatactatttttaaggaggaaaattaattatgaatgtagatatttatacagatggaggagccagaaataatggtaacaaaaataatagtttgtctgcatgggcttacactatgagatttggagaacataccaaagaagattctggaatttttttaaatggaaccaatcaaatgatggaaattaaggcagttattgagggtttaaaagcgatgaaaagaacagatatccctgtggtagttcattctgactcagcctatgtttgtaactgtattaaagaccgttggtatgtaggatggatgaaaaatggatggagaaacgcaaaaggaaaagaagttgctaatcaagatttatggaaacagctacttaaagaagtagagagatttaaccatattacttttaacaaagtaaaagggcattcgactaatgaaggaaataatagagcagatgaattagtaaacatgaaaatggatgaacatactaaataaggagaggatattatgtctaaatataatacgccaatgaaacatgaagatgatattatcatttctgcattagaagctagtatgttatatctacctcacaatgttttaataccctgtgataaatatggttctcaaaaagatggatatacatggctattctacaaggaagggaaattaaaaggttttccttttgaggaagatgttaaagatattccaagtgttagtaattattatttaataaaatagactaaaaccccttgacagaagtcttggggttttgttatacttaatttaagatattgaaaaggagaatttttatgaaactttatgataagcaaaaagaaattgtagatattatagaaaaccaaatattaaatgaaaaagacctaaaaggtaaaatgtatttatctggagaaatgggatgtggtaagacttatatgggagcttacctagccaacaaactatcaaaagattatcctactattgttatttctcctagattaaatatcaataagtgggactctttattagagaacgcctataaaataaaaagaaaagaaaaatataaagaaagacctattactttattatctttagaaaatttaaatatgtggattcaaaatcaaaatacaggtttacagccctttgaaaaagatatgttagttattgtagatgaagtacatttatgcgacaatacaaaactggaaggttttaaaatgttacaccataaattaactagaaattctaaaggaatttatttaacaggaacaataatggagggtgaaagaagtaaaatagcatctattataaaaacaactcatccatatttttctgaattatttaatatatcaaatgaaataagacgtaattatcctagttttataagaaatgtttggtcttatatatcagtagctgtgtctttagaagatattcaagcactatctgataatagagaggaaataaaacaggagatggctcctataacacctttaccattagataaggaacatgagttattcatggaggttgtagaaagctctcttagagatttggatgtaaacccaaaaagagttatgagcttatcttctagttatttagataacccaagtaaagacttgacttataaatcaatgaagagggtttctaaaaaagatttaatgacaggtatttattctaaattagcattacctttgaagaatatagatattaaaaatacatctaaatttaaaagacttctaggcttgattaatgaatcaaaagatgatagaatattattatatgttaatgagcatgatttaatattaagtttacaatcatctttaaaagaagaaggtatcgagtcattcactttagaggatgtagaagaagttaattattctgagcatataaataaagcatttagtaaatataaggtgggaatagttaacccttctaaagtaaatgtcggtatagatatacacgctgagcaattagtatggtatcaattaatgcctaaaatagataaaatgatacaagctcaaagaagagtatgtagattgagctctaaaaataaatctttagttaccttatttgtctatgatacattaaatgagaaggctagagcagaggaattatctaatgcaacaaagaataatgctgtaacttatggtgttaaacaggaagatgcgttggcacagttaacaggaattatattagatggtataaattaggaggaaaacaattatgaatatgtgtgacttaaatttccaatcagaagtaacaaaaatgatagagctagagaaaattataaatgaagatatagttataccaactccaatcaaagaagtaaatataagtgatttaagaaaaaataaatccgaagaatacgaaaacataccttgtagcagtggtgtttctaggaatgttaaatattctataaaggatggatattatttagatacaggaatagatttacacactgataattcaaaagaattaaaagacattgtaagacacagcttcagtgtgttagctaagaatgttaccaatattggaagcatagaaataaaaggtataaaaaggtcattaaatatagatataatatttaataaaaatctaaatgagtttaagttgttttttactttaggaagacaagattatatgttaacttatgactctaaagacatagataatgttttaaaatatatattatctcttaaattagatgaggacttaacagaatcaagagttgctagaagcttacgtgctctttggatgtatcaactaaaagaagaacttaaagaaacatcaaaacaaaaccatattatagaatcagaaactttgaaagaaataatagactctataacaatggacttaacagacactagagaaagtaatattagaataacatacagggaattatttgagttctttaaggaattgagaaacgaacatattacaaaaaatacaagtaaagtccaggaatggatttcatttaatgataaaatagatataaataaatacttgacttcacatgttaactatgataatattaaaaatataaagaaagaagatagtgaagaaactagaaaaattatttctcaactaaaatcagatgtgtatcttaacagaaaaattaaaaacatctacaaaataaatgataaaaaagaaagtgataaatctatttttacaggagtacatggaacacctaataagagcttgttctctatattgttaaatggattaaaaacatcaacagaattaagagaagagaacaatgagagtttttcatatactggttctggtttaggagatggaatatattttgcacagttgaatcaatgttctaagtcagcaaattatacacgttcttctagtaattcgtcatatattataatagcagatgtagaatatgatgtagactcaaagctagatgttttttcatatgattcatctattgagacaaaaaaatattctttaatccatggtaagaaagtgggttcaagaaatttagatgagattgtagcaccttttagcgacaaggttaatattagatatattgtagaattagataattaaaaggagaaaaaatatggaatataaaatttttgatggtcaaataatggacttaatgaaaatgttaagttcaggtaataaagtatcatactctaaaggatttttaagttataattttttaattaaatcagatactgtaatgactttaaaattatataaacgaatgctaaaaaagtttaaagcaaactatgatgatttatcatcaacaaaagtattattgttaaaaataactgtagataatactgtatacatcatgaaagattctaggcatgaagcaataatattgtcttactatggaactaaaaacaacagttctttaattatggataaagacccagaagaagatattgtggttagttcacatacaggtcataattttagagaatatgaagatgttatgtcattaaaaaacctatttgaatatgataaaaaagaaaaatatgtaagaattaaagcaacacctatcaaaaaacatcttgggttggttgtaaataattttcataggtatgatgatattttaagagattatttgggtttaccttctgaaaattttaattataaaagtatggatattaaattaaaagaacaagaagagcccagttttaaccaacctagactaacaagacaaatgtctttacttggagaaatggtttaccattcttttgaaaaacaagatagggatgaaagtaaaaatttatctaaaaaatattcttttaaaggggataaaaatagaattatcattaataaaactccaccaggaaatttcaaggtattgggtcatcaaaaagatgttcaggattttattgattctggggattttcatgatatgtatgatgaaagatttttgatatgtttaaatattttttatttagaaaacggtttagttttattagttcttagaagtcaaaccttaaactcattaaatgatatgagaaatgaatacaggcaagaatattatcaaaataaaggtatgataatatataagaataaaatatctaagatagtagatatgggggatataagttgccataaatattctagtattttaacagacaaagaaaaaattaatcatacagaatttagaaagttacttgaaaaaatttaaaaagaggtataatatatgaataagatgattataatgggaggagacataagttttgcaaagagtggatttcacattgcaaaaatagataaaaaaggtattagtatggttcattcagaactatttattactgataaaaacttgtctcctattgaaagaataaaccagacaattgaaagaatggcacagttacaaaagaaacataaggtagatttaattattaaagaaggtgctattataggtcgttccagtacaggactgaatgttattaaaacacatggagctgtagaatatttttgttttgataatctaatccctattgaagacatgcataatgccacattaaaagctttctgtagaaaaatagtaaatgataaaacatatgataagaaaaaagtagttgccaaagcaatggaaatgtattataataaagatatagaagatttctatacacctagaggcaggttgttagatgatgtggcagattccgtaggaattactgttgcatggtatgaaaaaaattttaacaaggaagtgttgggaggatgaaatatattaaaaatagttggaactggtttaaagaaatcatgcacccatatagatatgtattttatgtagcagacgttctattggttacagggttaataattatattattaaaggaagtgttgggaagatgataccaaaagtcatagcatgggataaagtcaataaaaaaatgatagaagatgttgccagtattcattttaaatcagaaatgatagtaacaagaaacttgtgtttttatccgtttaaagatattgaactcttacaatcaactggactattagacaagaacggaaaagaaatatttgaaggatatattattgataagggttatagcaatatagatatagtagacagaattggttacgtgaaatttggtattggcgatgattcagacggttatcttaatcaagagtggttaggttggacaactaacaacgatgattctttattagatgtacatgaacattgtactgtgttaggaaacaaatttgagaatccagaattattaggggaggactaattatgaatagactaaaatttagaatatgggataaagaagagaaaaagtttgttgattggaatgtatatttcaatctaacaaattcagaaatcgttacggaattgtggtatggatatgaaattcaaattgaagaacctcttcaagattcaaaatatattgtgcaacaatcaacaggacttaaagatactaatggagaagaagtatttgagggagatattgttaaatttaaaaatccttgtgataaaagattctctacagtaggtattattgtatggcgtgaggacaaagcttgttttgggatagacatggaaggaacaacagaacagtttgaactttatcgtattacagcagaaaattatttaactgtaataggaaacaaatttaaacatttatatttactacgcaattaaacaaataccataagtacaaggttttacttgtgcaaatacccacatatgcaagtgacgtgcaagtaagacagtatttactaggggagcttatggcttacctaaccattaaggaggacacacaatgaaagtatacctaattgtacttcctcttgtgcttgctttattattatatgatttatataaacaagcttctgttcaagatgaggtagataataattaaaaactattgacactctttaaatagggtgttatagtatacatataggaggatgaaatatatggaattagaaattcatgaaagaaatgatgatggtttaatacttacgtcttttgcaggagcttcttatggctacttatgggagacagaagtagaagctagaatgtttaaaaagctatgcgaatctggatttgaattagaagagtcagcaggtattgttaaatttgcaacagaggaaatgagatataatacggatttatattatgaaatagcaggattatctttattaaaaaatgttcaaaaagggttgacaagatattagcaacatgttattataaatatataaacaaataggaggaattaaatatgagtatggttggactattaagagatatcacaaatgaactaacagacagtggcattgaagtaaataagattgatttattaggaaatgtaatacagattgaaggagttattactattgaaaattcagaaaaagatgacactaaagtatatgattataagttaactactggtaattctatagaacgtcttgtttggagagtagatgctgtagagattattattagagaacttaaaaaagttaagaattttaaatcaggaagttttgtaagtaagcatggtgttaaatatatgcttataccccataactctttattaaataatttctatttattaaatatggagacacatgaagtagaggatgtattttatacacaatctgaattatatagtgagtttaatctagtaaaggaggataattaaaataatcattttagcaattattgcaattgtagttacattctatgtattttggtcagtagtagcaggtttatttgtagtagaagtgggtaaagagattaagaaagatgtggataagaacctaagttctactacaaatagtaagcttgatgagaagttaaaaaagaaaggattagaaccatttatcccccaaactaaaaatgaacaaagaagatttaatgaatacactattagttggggaagtaaggtcgcagcaacaagaaaaatgcacgcatacagaaaaaaacatggtattaaataggaggagaattatgaaagagatagaggtaggagatgtattttatttaaaaggaaagcctgaaaaattttatattgtgtcaaatatagaagctcatttatctacagatatttttgagaaagatatgacaatagttactttaaataataatgagacaggcgatgaaatagtaatgtctttaagtgaattttggtatatgtttgaacaggcacctaaagaagagacagtggataaagtggtttatatgggaggacatctacttaatcaagcaatggttgagtatagagataaacaacacagtgaagtagaaggaattatcggagttactccgtacagcccacatcaagacaagtctatcaatgacaaatccaatgcagtacaagaaggtttggctgaacgtattttaaaaaacgactttaaagcaatgcaggaatcagacgtgtttgttttcgatatcttaaatgaaggtttaggaactatagctgaactaggaattgtattaggtatgaaacatcaagctaaaattactatagataaattagacgaagcaattgaaaattatggatttttggttgaaaattcagaagacgaactgtacgaaaaatcaaaatccttattagataaaccagtattctgttattgctcagatattcgtcaaggacatggcgagccttacaatgaccctgatagagctgaatttagcacaaatcaatttgtatatggtatggtgttagaattgacaaatggagaaggttttatttcttgggaagaagttaaagaaagattaaagaacatttaaaaaaatgttgacaatggatattaaagtatgctattataaatatatcaaataaaaggagaaagataattatgagattaaaatcagtagaagaaaatggtaacgttacagtaatcacagggagctttttgggaggaaatttaaacttaactgttcataacaaagagggagaacttattcaagagttttctggcactgtttctgaactaaaagaaatggaaaaaatgagtcaagatgaatcaagagaacattgctatggctatggtgacagttctgaaaaaagagtctttgatgtcgaggaaacagaagaagaaagagaaatgttctcagatgataaaccagaccctgaacctgaagatttagaatttggtgataaagttgcattaatcgatgattttcagacatttgtatatgagcaaggagatatcttaacagtaattaaaccttctgaaatttttgataataattatgaagttattggtaataatggaagaagtcagataatcagtagaaatgcattaaaacctttaggtgaaaaatctaaattctatgtgctaaatgaacgaggagaaagagtatcttcttcattgaaaaaaggaacaattgttaaagtattagatgaaattacttttagtacagctactacggattctactggacttaaacaatatttcggaccagaggaatttgagggtgttatagtagatgagaatggatttgatgatgatatcgaagaaatttttgagtttgcaaaagaaaatgatattaggcttactattaacaacttagatgaagtaatagaacaaaaagaaaaaggaattgaaaaaattaaattagaaccaaacactttatactacttaaaagaaaaagtatatgatactttcggagactctgttggacctggtttagttacatttgatgatagattcggtggatatcttgaccagtatgatgaagcaccagtttttatagacagtcattactttgataaagataggatttttaaagtaagacgtcgtgatattgttaaagtagtaggtaattactattctggtaacggggtaataggaaagactggttttttacttaaaagtggtaggtcaacaggcaataaattggtaaaaggtaaatctttagatggaccatatgtaaacttagcagaggtaaaaccagtatcagataaaaaagcacagaaattctttgaaaaagaaattgctaaaactagaacaaaaggatgattaattatggaattcgataataatgattttgtaatgaaactccctcatagctttatcttaactagagaacattctggtgctaatgaatgttatacgttatactctaaagagagagttaaagtagtaaagacacaagaaatggttgattcatggaaattctgtgggtattactcaactatagaaaaaggtttaaaagcctttatgaagaaagcacctaataagaaattaaaaggtgatgtaaaagttcaagatgttgttgaatttttagaagaattaaaaaaagaaattgatttataaggagaaataatatggaatttattgtaggattattagtaagtattatatttatattagcttatggagcacaaataaaaacattatttcaatcaaaggatttatctggagtttctttatggttctgggtgttcattgcgacacccgttaccttcacttttatccatttagtaagctctggagctgaatggcacgtcagcattccacagttaatcaacgcagtgctaggtttattcatcttaagcttcgtatcttttatgaataaaaaccttctaaaagttttattgttttggttgatgatgatagtaattatattgtctcctacagtattttccattccttatgatataatacaaaatgtatcaactattatgattctggttgcttatattatacagatattcaaatttaaaaaactagaaacatcagcaggaacagacatattattgtacttattctttgctttaggaatgactatacttttaacacaaatgattttaacacatgttccaaatacagttataataacagaagctacaaatataattttattattaacatgtgcgtttcttagtgcaaaatataaaaacaaacaataaaatatgcttgacagtcgctatttttgatggtatactaataatatatctaagaaatagcgattttttataccttaaaaataaacccttagcttatagagcgtttaaagagcctataattttttctttgatgtatttgacctagaatcaattaagtctcttatatcgctttatattaagcttatatgtacacaataggcacagggtactaaaaatttaaaatattgctataaaatcatcattttatatataaattaggaggaatttagaatggcagacgtaattaagacattggaaaataaaatggaacatagaggtgtattctttttagttaaagtagaatatatagtcacagacacagaccatgatttacaaattgttatttataaatataatgatgtgaaaacaggtgctaaggaattagttgtaaaaggactatatggtaaagacctacttccatttgagcaattgtatgatagagatagaagattttcatcagaggagtttaaagattatctgattagtggcattaaagaattattaaatgatattgtaaactcagagaataataaaacagaaataattaaagcattagaagaggtggatgaatcatgggaataaattttaatagacaacatggtggtactactaagataccaataggagataatatattattagagctcttatatgcagacctaagaaaagtttgtgcgtatgatgaatgtcctatagaatactctaatttaggttgctatggagtgtttactgtaagtattgtaacccaagattctaaaattaaaagaaaatggaaaaaacaaactttaaaaatagaagatggggaccatgtaggaagaggagactttagggatttagttaaagtgaaaaataaaataaaagaaatagtagaccaattaaaaacacatgaatgttttgatattcatggttgggatgagagaagatttaaaatatataaacatttcttacacaaagatgaatttattttagataggatgtttgaaacatctgatactatttatctattaagtgaagaaggttttcaaaaaacatttaaaaaaatatttactttaaatgaaaataatgttatgattataaatgagaaagaagttaagaaaaatatcgaagactggagaatgatgtcaaaatgttaaaggaaatgttttcaatccataatcatacagacgtaagtaattatcgtttaagagatgcaataaatacacctgaaaggttaattgattatgccatggagcttggtctccctggtatagcgataactgaccatgaaactctttcaagccatataaaagcttctaaatatgtagaaggtaaagattttaaattaggatttggtaatgagatatatctggtagataaagaaacaattaataatgctagagagaacaatataaaaactcaattccaccacttcttactacttgctaagaataaaaaaggatatgaatttttaaaaagacaatcctctaaggcatggcagaacaactttttctttagaggtctggaaagagttccaacatattatgatgaactagaagaacttatgaaagactataaaggagatgttatagcatcatctgcttgtttaggaggtcctattcctcaagcaatcctcaaatatcatgagacaagagatagtaaagataaacaagaaataatgagactcattaaatggtgtgttaaagtatttggtaaggatgatttttatttagagttacaaccctcaaagaatgaagagcaactaattgtaaataaagtgttacttcaattaggtaaacagttagatatcaaatgtattgtaactacagatgcacattatttaaacaaaaaacaagcaaaagcacatgaaatatatcttaaatcttcaaacggagaacgtgaggtagaatctttctatgctactacatatgtaatgaatagagaggagctactagaattctttgacgaagatattttagatgaattaattaaaaacactcatttaattatggataagatagaacctattacatttaaacaacaaacacaagtaccagatatagatattcctttatatcaaccgaacagaatatttgagaaatattatgacaaatatgaatatattaataaatatgcttattctggaagagaaatggatagatactatttacatttattaggagaaggttttgttaagcgtggtcaagaattcaatgaagaaaacttagaacgtattaatgttgagcttgaacaactatggattatttctgaaaagttaggacaaccattatcatcttattttgttcttacacaagatgtaataaatatgatgtgggaagttagcttagtgggagtatcacgtggttctgcatcatgttattatactaactacttattagatattgtacaaatcaatcctattacttataagcttccttattggagatttttaagtaaagaacgagcaaccttacctgatatcgacatagattctgaagcttctaaacgagaagagattatggaattagctaaagaacgttatggtcatgataagatattaaacagttgtacattcactactgagggtcctaaatcaactgttattacagcttgcagaggtataggactgtcagtagatgaacaacacaatattgctaacttaatacctcaagacggtgctcagctatggtctgtttcagactgtttctttggaaacgagaaagaaggtcgtaaacctgtagctaagtttattcaaaaggttaatgaacatgagaacttaaaagaaactatgttaacaatagaaggtattgtaagtggtcgttctcaacatgcaagctcagttattttctatccacattcatatttagatgtaaactctctaatgaaaactaaaaaaggacttgaggttactcagtttaatgcagacgacggagagtacgcaggagaacttaaacttgatttcttgtctatatctgcattgggtcgtataagagaagccatggaactgcttattaaagacggtaagattgaatggcaaggtaatttaaaagacacatatgataaatacttacacccagacgtattagacattgaatctaaaggaatgtttaaaatgctttctaatggagatatctttgatgctttccaaatgtcttctctggttgcacgtactgcaatgagtaagattcatccagaaacatttgatgaggtagctatcactaacactcttattagacttcaaacagacggagaacaacctgtagataagtttgttagatataagaaagatattagtcaatggtatgatgatatgaacaagtatggcttaaataaagaacaagtaaaactaatggagagacatttactatctcgtactggtatttgtgacacacaagaaattattatggaaattatcatggataaagatattggagattcatcgttagaatttgctaataaataccgtaaagcaattgctaaaaagaatcctgtagccattgaagagtgtgaaaatatattcagagaacagattaagagtaaaggacatcatgtcgattttgccaactatattatagatgaacagattggtcttagtagaaagtattcgttctctttacctcacgtagcaggttatacacttattctatttattgaaatgaatattgcctacgtctatggtttagagtattggaaaacagcttgcttaactattaatagtggtcttgaaggagatatatctaaaggtgcagactttggagaggtttctactgctgtaaacagtatgaaagaagatattttaccaccagatattaatgaatctcaacttaaatttgtagctaaagatggtaagatactatatgcattaaagcctattattggtttaggtattgacaatatagaagaaataattaataatagaccatacagttcattcgatgacttctatgagaaatgtgtaatcaatggtggtctaacagagaagaaaactatcatattaataaaatctggtgctttcgataacctacatccagacagaagacaattaatggttaattttgtgaacaaagttgtatctaataaagataagttgactatggttcaattacctaaagttatacatttactacctaaagaactagaagagttaatgtctttatataaatttagaaacttaattataggtaagaataaagttaagatgaataaagatatagagaaagagtttattgataaatattctaaagatgtggcttttaaatttagaaatgatgtattggaaatagatgaaaaatctatgaataaatactataataaaaaaatggaaccagttaaagagtggttaaaaacagaagagcctgtaaaagaatacaatattaatcagaaaagagaatattggaaagagaattgtgctggtagtatagcagaatgggaaatggaaacaatattgtttaatactaatgagtacgctatagatttagaacaggttaaagaaagatatgatattagtgatttctcagaattaaaatctaatcctgtaaaaagctataacaaaagagggttcccaaacttcgaaatatctgctatagtaggggtagttgtagactggaataacctcaagaagtatatatctgtattaacgcctaatgatggagtaattatagttaaattgcctagaaataagtatagttattatagccaaaaattagaaggcgaccctagttggtatgaaagaggtaataaactaatattattaggctataagaaaggtaaccattttcaggtaaaaggtgattatatatataagaatcctgttattaaaatacatggtcatagaaaatataattatcaaaataaaaaacttgaataataacgcttgactctttatgagttaagtgttatacttttattacagtaaaacataaaggagaaaatatatggaattaaataagatacacaatatagactgttttgagggaatggctttattagatgataattcaatagattaccattttacttctccaccatacggtcgtaaaagaaacgataaatataagttttatgatgatacagtagattggtttgagttgattaataacacaataaaggaatctattagaatattaaaagatgatggttttttatttttaaatatacaaaaaaattactataacaaaaaagactattataagatactgggtctatggtctgactatattgtagagacaatagtatggggaaagaacaatccaatgcctgcaagtggtcttaacataactaactcttatgaagtttttattgtaatatcgccaagtaaaaaacctttaaaatctaaaaatacttatacaaagaatatgtttattacaaatgttaatagtaataataaatataagaaagaacataaagctgtaatgaatttagaagcaagtgattatatttttagtaattttatagaaaaagatgctcttgtgttagactgctttgctggagtaggtacaacatttgtgtcttgtttaaaaaataataatagttttattggatatgaactttcagaagagtatgctgaaataggtaataaaagaattgaggacatgaaagtagtagttgacaataactaatctatatattatacttaaaacataaaattaaaggagatgtcttttatgaatataaaagttaatatcaacaaaacagaagaaaagttattagtaagacatggggatttaatagagacaatatatggggacagaggtataattattaaaactaattatgatgcagaaaatagagatgtgtttaatgtattgatgatacacacagataaagattataactctgccgatttatgttttagaattcctgtttcccttgaaaaaattggtgaaaaaactaagaaagttttaatgtatcataatgaatataatattaaactggaggaaaattaattatgagtattttttcaaactatttaaaatcaaaaaagaaatatgagaaaatggaaacaccagaacaaaaaatggaggagaatattaatatgaaacaagagactttagaaatgggcgtattagttatgggacaaaaaggaacagaggtattagttcctcaaattaaagaaaatgaagaactcagagtagaaagaaatgttcaggttttattagatgccttagatacgatttcatttgataaaaacgatgaagaaattttagtgtttgatagtaaagcttttgattctatctattcagacattccggaagaaggagtattcacattaggtaacgatatctatgcagagcaagatttgttagaattcctagatagtaaatggaatcaatatggctctgtacaaagacgtggagacatggaagaggataagaattataagcaagttattccatctatgattataaaacaaggagataaatacttcacttacacacgcttagaaggtggaggggagtctcgtttacatggtaagagctcaatcacagtaggaggacatgctaacgatgttgaggattactggaactttgaacatttaatggctgttaacgctaagcgtgaattagaagaagaggtttatattttagactcaaataatgaagagattgataatcacttaagattaactaaaaacatgtctgttaaaggattaatctacaatgaaaaaacagatgtagatgctgttcatattggtttattaactatgattgagattccttctgactgggatgttaaagttaaagagacagacgtgctagaaggtaagttcagaacagtagaagaaattaaagaattagacttagagaattggactgcaagtgctttaagtgttctgcaataaaagtattgacagagattaagttctctgttgtactttatgtatacttaaaaaggagaggtttttatgaagttagaagataaggtgttggaaagaattgattcccttggaggtaaattagataatattagtcaacatgcatgggaatccttggtaaaatatcagattatttatggtgttgtagaccttatagtaggtattgtagttatagcattaactatatttctatggaaaatgtttattattcaatataaaaaggcgagtaatagagatgattcccatagtttactatttgaatatagagatgacttgtcaattatggggtttctttatgtaatggctgtagggctattaacaatagttgctcttatatatctagtatatggtatacctatggatattttaaagatatcaaaccccgaaatatttgctgtaaaagatttaattgaacaagttaaatcaggaagttaaaacagaaaaataaataaaaaacttttaggaatcctattgacagggttccttttttagtatatacttatattataaagttaattaaaagaatcattttataaaggagatgaaggaaatgggaaccgaatataagtatcaagtaggggaagtagtaaatgaaacattgaagatagtagagcagattagagtgaatgacagtaagaagaaaaaagatgggacaaattacaccaaaaaaggttatatagtacagtcaactgtctaccctaatgcaccagtatatgaagtaagtgaatatagccttaaatcaggtaaaggttgcgcatacataagaggtttgcgtgtgtttgaaggtaatagtttatggtcattgattgagtatagaaaatatattattaatgaagaagaagctaaatcaattactcctaagtcaaataaaaaaattaaattaagatgtgataattgtagtaatgaaaagatgatgagagtaagtggtttagttaatcaaggcttagcatgtagcacatgctcatctaacactccataccctgaactattattcaatgcatataaccaagttaaaggtttaaaattcacacctcaacaaagatttagaaattttacagggtatatttttgattttgtaaattatgaaactagaacaattgttgaggtacatggtgaacaacattatgatgaagacaacaagtggtacaagagaacacataaatcagatatcgctaaacgtgaatactgtaagaaaaatggttggaattttgttgagttggattgtagtaaaagtacatttgaatttattaaaaatcaaattgaattaaatgaagctttacctaatattactaacaaagatgaaaaattaatgttaaaaattattaaacagaacaaacgatacccgataaaaaaaatcatcaaaatgtataatgaaggattatctactactcaaattggagaagagtttagagtggctcatacaactattagtcgtatcttaagacgtaataatattaaattacgcaacggtgaaaaacaagtaagatgtatcaatactaatcaaatatttagtagcgtaaaagaagcagaaaaagttttaaacattaaaaatatctcaggtgcttgtagagctaaaatcaaatcagcaggaaaacaccctatcacaggagaacctttaagatgggaatatgttgacaatgaatagaaagtattatatacttagtaaaacacacattttataggaggagttaactatgaatatagtattatacattacaataattgtttttatcatgttaggtgtaggactttataaatttagacaagacgattttgacaggtttataggtgcgtgtgtagtagtatcaggagttattttgtttagtatccatttagttttacaagtatttgtataaaaggatatagtttaaacatagaatttataggaggaattataatgaaaaatatggaagagttattaagtcttattacaaaaggtatgagtgaaaaagatattgagctaactcacgtagatttatatgaacaagcagtaggtatcaataataaggttaaggtttgtctaggaaatcgtggtaacgatacatggtttttagttcatagaacggatagaacagatgcacgtgataggttgcgagtatattctaataaggatatggtagatagagtgctggagttactcacaggggatgattggttgccaggagatgatattgaagcatatggtgaatggtttactttagtagtagaagaaaataatcactatacagtgtcaaataatgagtattgttcttatagattgttggatgagaattataatatatccgatagaatgacattaaactacctaaaaagtaactataccaatgttagcttagaaaaaaggttaaaataataaggaggatatgtattatgaaaatgatgtcagttgaggaattaatgaacttttggtggtataaagcctatgagattgcaaaagaagataacccaaatgtaaaagaaaagagagcactattcatagaagcttatacatcttacttgttctatgaagaagcatatgaaaaacatagtgacgaagctataagagaagcagaatatagattaggaaaaagtgaagattaaaggaggagttattatggagacatatgaagtattagcaagattaacagaaaaatttaacaatgagaagatggatattgggaatataagtattacaaaaacaaacaacaatgaacgtattacaaatgatataaccgttgattatagaattaaagtaatatatgttgataatgatgaattaattttttatcaaattagactagatggtaaatcagtttatgacacaagcgatttagatatggctatcgaagcagtagttaacttattatttgatatgaaagaatttaatataggggatattgttgagcgatatagacaatattttatacttgtaccaccaccaggagaaatagacggtaaaaaatttatgctatttagtttagaccatcatgcgttagtagagtcatctgtgctcaataaaaaagaattatataatgatggctatgtaaaaatagataaggaggattattatggataaggaaacatgggaattattaataaagtcttttaagaaggattataattcaaatagaaaacccagttatttagttattttaaatggcatagaatatgaattaatatatgataaaccttatagtagtttttctgaagaaatatcaatccgaaataaacaatctggttctttgataggaagtatgaataaggattcttttaatggcagttccagtttagatacaattcaatataaagaaaatgacactttaatcaatattatagatggaatggaatattataataataaaagagaagaagaacgtaaagcagagattaaaagaagagagaaaaaattagatagttttttatatggagatggataaaaatgtttaaatatgtaattgacttttttaaaatgtttttttgtttattaatccccttgacaattatagtgttaatagggtatactatatatagtgttagtataaatatttttaatatagacaataacactgttcaatttgtaatatctttgtatatattattagttgttttaagtggaatatttactatagtaaaggagattaaattatgaaattagaaactaagtatggtgtattagaaggtacagtagaagagtttaaggaattattggaaggtaaagtgaatagaagttggttatttactagcaaaggctataagattgtcaataaggacacagaagatattcctaaaggtacagttctaaaacatataatagcaaatcagtatgaagacacattgggcgaggtatatataatacatgatgaacaccttgatgattacaatatgtcttgggttgatggtaagtttaagtcatttaaggataaaaaactcttacttgttcagaaagaggagtataaagtagttaaagaggatatctactctaagtatggtaataaaattcatgagaaaggtaaagtattacttgaaacagaatataaccatgtagattctttaggtaacaaatatcgtttaaatcctgatacaatagaaaataagttagaaggaatttatggtgatagtgtaggatttcattttgaggaacatagagctaacaataaattatataaggtaactatataaagaaaggaaataggttatgattatgggaaatttattatttattcaaatattgtttttacttttacaggttgttctaggtagtttatcattcaaattagatagtccatttctattgtatgcttctcatttatgttgtgtagtagttatatatctaatgtttgtaattatattaagttaggagatttttattatgatatatgttttagtaggtcaaacaaactcaggtaaaagcacattagcaagggatttagttactgccacaggaattaaacagataattacttatactaccagaccaatgagacagggagaacgtgatggtatagattatcactttataacaaatcaagaaatggattctggaggattctttggaagaagagatttttatactgtatacagagaggagccttactcttatgcaatgaaagaggatgattttgttttacttggagatgctattattatattagaccctaaaggagtaagagaacttaaagaccatataggttatgataatacaacgatagtttatatcaacgctagtgaagaactgataaaagaaagaggaagtaatagaggagaccaagtagatgaaatcattagaagactagatgtcgatagagactacttcatctctgctaaatattttgctgatattgtacttgacccagaaaaggatgatatgttaaactatatgtatgacaaaataagagggagacgttaatatgttttataatgttgatgatttatcagaaagagaaaaattggaaaatgatatcaatgggttttttaaagtatttaacaaagagtataaacgtaaagataaaaaagtagaaaatacagcatttatagtattattaaaatatgtctttgatttaatagaaagaattgataaactagataagaaaggtaatagatggtttaaatttaaaagtaaacgaaaactacaagatgctggtgttttattagccaatgctgaaaaatgggagatgttctaattatgaaacgggttattaaacggggacatatttactcagtaattttaatggcaatattagcaggttctgttataggatattcgaggtatagggaattacctccacaagtatctgaactatttatggctataggtgcaatgatgattcttttcattgtactttttatagctatatatttcatatatctcttgacatcttttatttttggtgttataataactatagtattaaaaataaaaaacagaagaaaggatgatattagtgagtaaaaaatacactcctaaagaaatgttagctgtccttcacagggaaagtggtagtaaattatcttttaataattatatcacagaacttagaaaaaaatatgctctaaaatatgggtatgttatgagtgcagttgattatgaggatatcttaatgtgcttgataaagaaaggtattgtttcaccaaacataatatttaaactaaaaacagtagaagaagaggagaaagataataatgaatgattttttaattatgttgttaatttttgctttattgttagtattatcgtatttttcaatgagatatgtacattctagtaaatacaacacactaaacattattgtgtgtatgtttgttggtatttcagcaattaggttatcgcagtatatagatgtcttgccaactactataatggtattgtattttattcctttaatcttattaaaaacaagaaatggtcgtaaataatggaatataaagcatacgattcttacaatgacactatgtatttgaatgctcaatggattgacagtggtggatggaatttatctaagatgataaggaataagtggtttaaagtatcacattctatcggactatatgataagaatgaggttatggtttttactaatgacatagtggtcataaataacataccatatattgtatcagaagaacctcataatggagtatacacagcttataatatgaaggatattaaagactttatccaaatgaacacagtacagtctaataatattgaagttttaggaagtaaatttgaagtagaaaacaaatataagtgatatgtataggggctaaatgccccttatttttatgcaaatattgaatgtaattgttctttacctttatctttaaggtatgattctgttgtttctacagatgaatggttggctaactctttaagatgtttaacatctatatttttaacacccatagcttcaaggttgtaatgtgtgccattctcataattctctaaactagagtgtctaaatgagtgagaatttaaagatatttcttcttcatatttttcttctaagattttacggaaagatacagcccaagaatataatgtttgactttgtagaggttttgtgataccgtcttccacatgtatccatagactatctacatcatcttctccacgttgctcaaagtatttttctgcaatctttttagttctatctttgtatagtaattggaatctttttccacgcttacctaatacttcatttgtcattgaactctctaagaatccatgtttttgtacttgatgtatctcattacgtctacctgcactatcataactaagagatacatatagagctctttgatattttttctgactcattaaagtatctattaagtaggtaacttgctcatctgataagaaaacaatatctctaacttcttgtttctctattcctttaatagaagacattatattgatttcatagtcctcatattcatcatcatcatctacacagaattgtaaaagatttcttaaactacattgcattctgttaatcctagctgaagaacaaccagtatcttgcatatgtaggaagaatctcctaaagtcccttttcttaagctctaatatattgtttttgtttaaattctctgaactccaacaataaaaacctttaatatcatatacatattgattaatagttccttctgagcgacctttagatttcatttccatgatataatcatctaatattaatttattttcttttgatactttttcttcgtaaaccgattttttaaaaaacacgtttcttttttccatgattaatgctcctttataatgtttattatgtatatatcccaccagtgagagttttataaggagagttggtgggattatgtatagaaaggggagatgtcccctttcttattatttccagactattttaccaaatctggctttttctttcttaagtttctcttccttatctgttatttttgctatagcgcaataaaaatccccagctttagggttagtcggataacggaagcgacaccaccaataaccatcttttttagttactgatacgaattcaatccattggtttttataaatccaagaacctttatctactactttgccttttaatccaggtgcttttctaactttaatagtcgaactagctgtaaacttacctttccagttccatgtaatctttttcttgttgcttgctggtgttcccccgattgctttaccatggataccttctgcaattttctttgtgaaatgctgtaagttctttttgatatagtccatatctttctttgatgtgatgaatcctagctcaactaatcgataatttatattcatatcgtaagaaacgttcacatttaataagtctgttctagtagttatacctctgatagttcctacagtagaccttaaagctttatcaatacccttatctatactgtcagcagcatacttacttttaataacatgaccaccactagcgcttggactagctgaatctaaatgaaactctattacggcatctggtttatgttttgattttacccaatataaaccataatcttttttgtttcctagtctataaccatatgcagtatcttgatacatatcttgatttttaccataaattaccacagtatgacctgcttgttttaggtattttgcgacattatcaacgatatttttacggataaaatcacgttcattagtgccatttcctactgcgccagggtcattatatccatgtcctgctacaagtacaatcttcttaccttttgcaggttttttagtagaaggtttagctactggttttggttttactttatcaattattgtttcctttttgtaatgaggtctaataaaccacataggaaaatcgtaagtgtggactctacgagtagcaacttcccaacctaaaccatagttgtcattcatacccccacctaaccaattctgctctatgacggttatatagtttaagtctgctttttctactactgcaacatgaccatgaccaccaccatagttaccattaaatacaacaacatctccaggctttgctaaaaagtttttagtgttctcatatactgttgcatatcctttgtgattatggtctataaaaggaatctcttttgcgctatatcctttaaaggctcttcctgtaacatagtaccaataatagtttgctaaatcaaaacattgcgccccaaaaacaccgtcaaaatcccactgcttaccaaccaatgttttaagataagcgacagcttgtttctgtgttttcataatgatttttctcctttcttatatatttttatctttttcgtcgtcttctggctctataaacacttcttctttaggttcttctacgttctcatctaggatttcagagtcatctgcgatttctgtttctgccattacgggaattgagccatcaccatattttttcttttggcttggtttaacatattctaatgcgtttttcccatctttaacaccttttgtacttgggtcattgacagcattatatacagatactactaccattgcgataacatatgggtttgatactgcttgaccaattaaatcagtaagaactttccaacttgtaacatctttaccagtcacaccaaaatatgttccgattggtactgcgattgccaagaaaatttggaaccagaattgtggattgtttgctcttaccttccagttaatacctaaaaaattacctttttccattaaaacgtcctcctattatcttatatataaatcaggatagttagtaaagaaaccatctaccccgtaacctattgctttattcatgtcttcttttgtgtttacagtccatggatgaactaccagacctttagaatgcgcttcatctacagatgcttttgtaacattattaaagtttggtcctataccattagctattgattttgcagtatctaattctgttgtagtcattgttgatgtaagtttaactaagaagatatcagagaattgatttctaatattgactagagattcgtctgcaaaagattgaataataatttgttttggttttgaaccaataccaataagatgtttttcttttaatattcttaaaagctctgcatccatattagcgttgaatggttttttagtttcaatatagtaatttactttcttaccaaaatgattaaccacttcttctaaagtaggtatttgtctacccttagtttttgttcttaatgcttttatttctgctaatgtcatattagcgatggttcctgtaccatttgttgttctgtctacggtagaatcatgcatacaaattaatttaccatcttttgtaatttgtaaatctatttctatataatcagcaccttggctaactcccagctcataagattctatagtatgttcatcttcatatccagatgcacccctatgagcgattaggtaaaaaaattatcaactacttgctcatctaattctacaacttctattgacatccaagtatatgggtctgctctaaagtctatattttctcctgcttcgtgtctgacttctagctcaaaataatcgccttcatttactaaaatagctcctgagcttgcttgaattgcagaagttgattctgctgttcttaaaacataaggcaatcccggtatatattgtccatttttctttattctgagaagtcttgcgcctgtagaatttgaagcccataatacgttggctgccactttaaccattgtaacacccttaggtactgttagtcttgtaggattagtcgggtcccagaatcctgatgtattatatagtgttccttcccaaggtacgtataatgttgttttagttgctaaagatatattattatcaagctttgtcattgcgcccataaatttaggtctaggtactaaacctgggacatctttttcttttacaactttttcccaaccttggtctacgcctttatggatattcttatgataaatatctcctgtatagcttaatacagcttttatttgttttacatcatcataagcagttgttacatctacattccaataacttcctgatgaatcgcttcccactggagcatttttcattttccacatttgataaaaccctgacggtaatgttgtagggtctacgtcggatattctgattctatctccattattttctgttagtttaaatctttgtaacgtgtaatcatttatatcaaatgatactttttcccaatcgtaccatgtattataaaaacgtttttgccacacttgattggaattataaggtctaaatataatacgttttaaaccgccattttctctttcatatacttctaaaaaccctgctgtacttgttgcacctattcctgaaatcggagttgtagtagtataataaaaacctggtgtcaaattatgcattgcctctaaattgtcgcctaacgaaagaatttttgattgaccattatctgctgttagtttatatttttgccaattatcagtattaggtatttctccagggtcacctttatcacctttgtcgcccttaggtccttgtgctccagtagcacctttagcgcctgttgcaccttttaatccttgaataccctgcggtccttgttcacccttttcaccttgaggaccttgttctccttggaaacctttaggacctctaaaatcttctttattttcattgacatattcttgcaactctaatgccacatcatctttgaatccatcccccaataattctatggcattctcttgtattattctacgaactgcatcatcaactatattaacagaaacttccttttctactgctccttctatgccactatccccaatagtgaaataaaaatctgcaacgtgtatagacttgtctttattttttaatatgatttttgcattaacttgaccaacatgtttaattacattttccggtatatcatattttattaaacctatagttgggtttgccattgttacttcttcatctttaaagatagaaccatcttccataaataaatctaaataaggaactaaatcaattttagtaaaatcgatatttctattattatcagttatcttaattcttatagaagctgtacctttatcttctgaatagaaaagaattccgtggtttcctcttttaatagtaccagtctgtatatttacttctaaatctttatttttatacggatacatacattaacctcaattctattattttattatttaataacaggaatatcgataccgtcatgacgggcttcaatacccctgttttatatataaaatttccattttatacatggttatttaattataagtggcgccaagacagatatgagtcccccactaccagctactactgttataatagttgtaactaactttaacatatctgttttcttggctgacgtttcagattctattctccccaaagtatcctcgttaacatccattctatgctgtagctttatcacatctatatccctttgccttataattgttgataattctttaacacttccatttaaatccctaacactagaatttaaagttcttgtttctctgttatttaagtctaaactactttcaagttttcctagacgagtattaatcttctctttgtcttcggtcatcttctcatttatctttcttacttcatcactgattctggaatttattctatggtcagatttttctaagtctaactttgtagcatagtttagctccttcttctcttccacagcgtagaacctcctatataaccacataagaataacattccaaagtgaactatattctgacctggagtaattaagttaatcgcattttccatacctgccattcctaaaaaagcataaggtacactacctattaatcccccaaacattaatagagtatagaaagtattgctcacctcatgtttaaaaatgaagaaggcagacatcattaataatataccagataataagaatagtgctccccaaaaatggagagagagaagattgtttaatgattcatataactctgaatcgtttgttgcatttaggttaaaagtccaatatcccccacgtattgatgtactaaacccaaaacagaaggttactataaataatattttctctataggagatagtccttcttcgtaagttttcataaacatcaaatcctatctgagtgatttaaatatcttcactctgttctttttcttcttcaattaaccaaggaaaactatttcctgttattttataaaattctgtctctgtgagacatccaacatgataatagtcagcaatatctttgtcgctgtaatatcctttatcatagctttttttaataaagtcatacttcgggcatcgtatctccattgtcttcctccttaggaatatcttcttttgattttatttcattcactatagattctaaaatttccactctatcagaagtttttttcattgaaaattgcattaaagtattttctaaaattatttgagcaagagtttcttctgtttcattcttaccttcttctgggatgatttctggtggctttccaatactaggagggtcagccccaacccatcttcctaaatcttcgctgtagtatatagggtcatataaactactaggaggaatctccctgatatagttatcagggatttctactccagattcaaataaggaaggtagtccgttttctttattgtaagcttgtatatacatgtacaaaccaatcctttcttattaatctattacccaagtaaattctccaataatgtaatgacctggtagccaactcgtatcatttgtattttggaagaatattaagttaccttctttacttaccaataatgctccagggtgtaatgaaacaggcatacgtggaacaaaatgttgttctactggaacgtactcagcaggtattgaaccaaacgcaacttgagtagttaagttgttgacattaactcttaaatgtgctattctagtaccattaacttttcttattctatattgacattttaaataagaatcatctgtagtgttacgtttagtagcatttcctttagtttcccaatcaatccatcctgtgtcattgtttaactggttgtattcatcctcgttttgaacttttttccagccagtaaattgaccatctggaaattgagtattagcagaatgtacggttgctctatactcatcatttctggcattggcaattaatttcaattgcttacgtttatttccagattctgttacatctatctctgcaatatatccaccaccatttgggtctaatggagcattaacagacaaagcatcagcaggaatagaagcttcgtatttaccaccaggtaatttaaggatatcattatatatatttccttcttgtcctaacgtacctaaccacttacgtgtattagtatctgggttagttaacacccaatcagaccaagtggggtcattcgttcttcttgtcatatatactaaattagaagtatatggctggaatgttaaaatagcatatccattagacctaaatgttacatcaataaatccactcacaccgtttccattagggctgtttacaggtgtggttacatatgctttttcagtttttgtgaaataattatctaaagtcgtaactgataaatcaggtgagctaattaatcttacaaaaccatcatcgcctgtaattttagatttttgccattcagtagtatcaaacttatttaaggcttcaacatctgtttttatcttattggctttttcatctaattcagtgatagattggtcttttaagtaagtcaacgcttgactataatcaattgcatcgttttctaattgattaataacttgtaaagttctatcattcaatgctttcataccacttgttaaagtggcttccatttgtgttacataatcactaccgttttcaagaccttcattgatagcattaatttgttccataatactttctctaaattctatgaaagttctaatttcgtttaatttatcaattgctggaattgtattaataacactgtctttaatatcaaatgtgaattcagcttgtgttacaatatcttctttagaaggattattaacatcatgtacttctatatatatttgacctctccatttacctggtctagctaaaaccttatcatctacagttacttttaattgaccgttcattgggtctacatactcaacttcttgtacgatatattcatcgcctttcatgagtattaataaagtagatgcgttctctgcactaaccattaacggtaagttatttctagtaatattaaatactaagtcagcagtacctaaatcagtagaaaagaaggctagattagtgtctagcctattatgaaaattagcacttgtttctaaattaacatgtcctcttttattatacattaaatatttctcctttctttttatacatcaaaccactgtgaccatttaccttctgcaaaggttctacgtttcattctatagttctgtgacctagcaaatggaatagcagtttggaatgctgttgaaacatctttaaagtctgctttactaacattataatgagtaacaatagaactagaatctttataaggagagcctttagcatctcttggaacatgataagttccaccaaatgtaacactgtttatatctgttactgattctggagcactataatttctagcaagttctttattattatgctgtaatgaatctggtgttattctaaatattgtacgtgataattcataagaccagaatctccaataatcagttccttgatataatttgagcattgggtttcctttgtcgtctccagtaataaaaccagagtaaccatcaaaaccagttaaccaaaactctttagagcttgttctattgaacttataaagtttaccgtatatgtcttgcactcttgcattaactctaccaatatcttcatatgctttttggatatctaaatctctaatttgtctttcatccctaccaacatacaagaagtctaatactggtctatcagcagtaaaatcttcatcttgtgctttgtttaatttagtagaacgtaacacagaagatgtagaacctccaccatctaaattatatgcaaacttaatatctccataataatcaaataatacttcagctacttctttaagagtcattccttcctcaatgaatgagcttgtattgttctcacgtccatcacaactaaagaaaattaagtctttattaggtagttgaccaataacttgtcttggatgttttacaactgtattagggctataatctccatccttgacaataactttattgtctgatatgataggaccaaaggctgttaaagcattagtaacaccttgagataataatgtttgagcattaacattaggggggtaacttgttaaagtattatcttctgatatagctaaagtccatctgtctttaacataatcagtgtcaagactatctataatacgaccattatatatttgacgaccatgtaatttcaattgactaccactacctgtactagcattagatacaaatgtagcattatgtcttcttgagaattcaactggcgtaatatgttctggtttatctgggttagttccttctagtccacgtttaagttttattaaattaccatcactatctaagtgtggaatatgagttatatagtaagttgtattaaacttacgaccagtataagtcttaatctcattataataagcactgttttggatttgtttttcgtgtttctctgataatgttttagcaacttttgccattccaccaaagaactcaaagtctgctttcaatctatcttgtgcaagttcatgtaagttcccttgtgtatcaacacgcatatctttaagttctgctgcactgttgtctgatttacctattacttgtgatttaacttgatttcttatagttgttaattctttttcaactgtacttgttttaccatgtgcaatctgtttagaattgtgagcattttcttcttctgttttatgcttttctagttcatattgagctagtcttaatcttctctcagcttctttcatattaccagacattatgtgtctattctggctatttaaagctataggaatatttgattctaaagtcatcttttttcctccttattattcaacccaagtatgttgagcatatatccaatttgtatcaggggcagctgcttggtctgctttgtctaaataaacaaatacttgaccagatgctctcatctcaaccattacaggttgtttaccactaccagaacgtgaatagaatacttgtgttgtattcataaatccttttggtaaaagagccacttgctgtgtgtgtagaaactttctaaggtttactctaatcatttttattctaagttggtttccattaccgaatttatatggatttaaaactatttctcttataccacatttaaaaccgtctgtggcatataagccatttatagtaacaccattgcctgcatcataatcgtatggtagccatccagagtctgttatatagtcacctataaggtaagataactgagttaacccttctgtatactcttcaaatccatctatggcatccatgtgtgtttttgcatagaaaggttgaccttcttcatcttggaattgttttatttctacacctaactcaaccgtttcctcttctggaatgtctgcattagttgcttttgtataaatatcatcaattgtactagccatttattacacactccctactgaaactgctgtaatcctattgtctgattgaacagcttttcctgttttacttgattttaatatattactatttattgcttgttgtatatcaactatatcttttttagcattactgaattctacttccacatgcttatttaatataggatgatattctgttaattttactatttttaattctgtactaaagcccaatggttcatgttttaagtatacagtgtttctttcatttaaaacatcttctccattataagttgcagaaaattctacaataggttcatcattaacttcttttattactttttcctgtaaatcttttaaatcttttgcgttatcctcgtagattgtttttgctttcttacttggaaactttttgaaattaggagacttgtattcatatttataatgatataaatcttcaccttttaatacagcagtaagatttaaaataactgctttttcagttcctataaacccagtaggcttatggtctttgtagtttatgttcgggtctggtcctatgaatactgctttaaacttatgtcttcctttagataagtttttagctacagtaatcttatctgttgtcatagttctgtaccaaccactagctgttaagaatttattatcatctaaatagaattcccacataccacctttaggtcctcttttgaagttgaagtctaatgtttcgtttccccataaacagtcaacatttacttcataagaatctccttttgtttctgttctccatgttccagttttagtgaacttaccatttaatgtaagagatggagttttgattgggttgtaatttttagtttcagatttagttttcttcttaccatagcctataacccatgttcttaaatcagaagtatttattgtagatgaaacaccctctgagtttaatgtttgtctaataacaacaccagattctttataaaacctatctggctcataaatataaattgtcttattgtcgccaaatattatatactcaaatatttctgcatgtttcactaaatactcaatcccattttcatctcctaaagcttcaggtatttctaaagcttctggaaaatctccaactattttatatttatatcctagaggattgtttctaaaaccaaactctagcacttgttttaaagtatatttaggcacaggagcattatcagatatatctgcatttaattgttcattttctaaatctctatctatgaaatgattctgaaattcatacataatatggtgggctactatttctttttttatgctaatattatcaaaacttaaagataaggattttataacataaagttgacccttgtaattgattgttccttcattctgaatattgtcaaatatgtctttatttcttaatgttttttcaataacaaaagaaactgttcttgtactatttatttcatattcgtatttgaaactttcaaagtctacgtctgttaaaacttctgatatcttaccatcgaaagtatctagcactaaatcaaagttactcattttatccctccattattatctatatataaagttaaaatcatattttatttcagaaacaactaatccgtctcctgataattcaatgtcattaaatcctttatcaagaattatatatcccttatctgtatctttccctatccttttattttttatatatggatacgttttattaagaagtattgttccttctttttttaatttatcccccaaataattgaactctgttttattagttttatttaacatcctaaatccattaggagctgttactgtcatataaatatctaaattatggtcaagtaaaggatttataatatcactagaagcattataaattttaaacttgttcttatcatgaacatatttaatatccgtatcagatattaagccactttcatattgccaaaagttagacattaagttataatcatctgtagagtacaatgattctgcatatcctttataacaattaaaggttacagtaaatgttgcatcaccatgatttattacttcccattctatttctgctttattgacagcatacttgaaatcaggcatatcagagtgagatacataatataactctctactatgaacgatacttttaattgtctgtgagaatagatttaaatcagccatatcattacctgaataatagaattttaaaaccaaagaaaaaggagcgaaattaatcgctcctggtattacaccatctcttccttgaatttctgaatcattattcactgtagatacggatggaatttcccactcgacaaagtttagacctttaatactttcagtcagtgacatgtttccacttggcttatatatattaacccatctactcttagtcaattaaattcctcctattttatcggagactttccttgaaagtatccttttttattaaattggtttgcttgattattccccacataatctgaaatagattctccatctacaatcaatctcttatcttctacgattcttgttagattaccaatagcaagtattagcttatctaatttttcatcgtttaagtttccaccatcactaatacttatagaaccatcaggtcctacccccatcatactcttagcttggtctattaattgcattgctctagtcttacgagtcaatggaataatcatttcttttctgtttccttctcctgctctaacaagttggtctttttcaataatacctccacggtagaatcttctaggaccacttggagaccaaccaccattaggattaaattgagttctccaatatttgttattaaagaaggctaataattggtcataaccagacttgatgttagtatgacctttaactgcgtagtttctgaaagtttgtggaatatactgtagtaaaccttgtgcagggttaccatttctactattgacgtcatcaacaccagattgtattgctccagcattaccacttgactcagcgttaataagtgatataatattatttatatcagagttagaaacttttgctttcatttgactagcagctcttacgatatcatctctccacgcagaagccttcttgttctgacttccacctttaagactcttaagccatttttctgggtttatagcagtgtcattgcttggtctatctcctcgcattaattggaaatgtaggtgaccagaacccttagtatagttaccagagttaccagataaagctatcttttgaccagctttaatcatttgaccagtttttgctatctgtttagatagatgcatgtaccagttccattcatttgcacctgttttaatttctacagagtgacctccaccgtagtcatcaaatacacgagctttaccacctttaacagcataaactggagtacctggtgtcataccaaagtccataccatagtgtttaccaccattaaatcctagaccaccagtataattaccgaatcgttgccaaatgtcgtgtttgaatagccatgaagcatcacctgaaccaccatcagtagactcgaataactcagttacttttttgataagtttaggtattaaattctcatatgcttttctaactaatgtaccagtaccacctggaacaccgtcaaagttgacacccatcttctccataattttatgaacaagttgtttaggtttgtcaatccaatccatgacttccccgatacctttacccagataagatgctcctgttttaacagacgcttcaagaccttcttcaactgtttccatagttctttgagctttcttagtaataccgtgtgcagagttgtatgctttaccaccgtattctcctgcttttttacctacgttcccagtcatttcttctaatagacttttctttttacctggtgtaccagatttgaattgaggtattaatccttgtgcttgccatgattgagtttctgcaccacttactacagcatctcctctatcaagggatgtgataacatctttaccttgaggtacttcgatacgtttaccttgacgaataataagttcttgatgaccatttctacctttaccgtttcctctacctttatcattaaccgtagctaatgttggtcttctcaactgacctttagagttagttgaaggtttcccttttctagtaccagtagagaatttatcagtatcaatagggtcaatcaatttagaaccagtaatacctttagaaatcttattgattccaccaatcattaggtttaaacctgatacagcacttttagctacagatttacccattgaagatgctttgtcagccatccattttttggctccatcaatatatccaccaatcttatcaatccactcttttgcaccattgtacatagattcaaatcttccaactgtgtgatttctaatagagttagtcattgttcgcatattattcttgatagattcccaagttgaaacaattttattctttattttcgttactgtatttctaaggaagttccatatcgtattccagatatttctaatagtattccaaacagatgagaatatagaagacgttttgtttcttatgctatcccaagtagttcgtatcttgttccaaatccaagtagttgtatttctaattttagaccagatagaattccatatatttacaatataatttcttatattagaatatatagcaatcgttttgtttttaatccaatcccatgttgtacgtactttattccatatccaagtggtagtattttttatcttactccaaatagcattccacacattggcaatgaaattacgaacagtagtatatacagaaattgtcttattcttaacccaatcccaagaatttttaaccttattccaaatccatgtagtcgttgagcttattttaccccaaatagcattccatgtattaaataagaaattctttacgattgtataagttccaattgttttattcttaatccagtcccaaactaacttgattttgttccatatccaagtagttgtaacagatatcttgttccatatccagttccagatatttctcataaagttccatatagaagtccatactgtgaatgtcagttgcttagattggttccaaccagtagctattgcctgacctatgcttaaaccaacgttaacaaaccattgtttagttgtagtccaactattgcttaacatagttacaaatgagccccaccagtttccaatagttgatgtaaacgtagtccatccttgcttaatggcattacccattttaagacctatgttgacaaaccattgtctagtagattcccaagcagtacctagtttttgtgtaaggataccccaactaacttgtgtttctgatagtcctttattccaccaagaacctatcttactacccaatgattggaatcctttaccaatccagtctactcctttattacctaagctagtgaatgtttctacatacccattccaccaaccagttaaagtatttttaatagagtctcctgaaccccaatatttcataaacgcagaagagaaatcattccacattttttgagcaccagtataagtttcaccctgcttgttcatttctttccagatacctttattacctttttctacagatttctggacttcttttgttttcttattagctgtatctactgcatcgttatagtcagactctaagtctaatactatagagcgatgttcgtttttattaatagaaccgtcagctaaagctctgtcagcttccttgataccttcatctcttatcttggcagcttctttaattgctttcttctcagctttagcactagctttcaatgctttactgatttcttgattagacagtccagaagtattagcttccatacgtgatagaatagcttgttgttcttctgcaccttttgcaacatactcgatagtcattttatttctttgttctaacttagattttagaagcttggcttctttatctgttagcttaccatcagtatactgcttttcttctaattctgcaatatctttattgagctttctcattttttgttctttttcggtggcagtctcttctgacttcttgattatgttttgtttttcttcttcagagattgacacactgttttctaatgcttttcttaggttagtatctatttctttattacgtttcttaacagcattaatagattctattgcaccttgtttaatactatctgttaactctttctgtgattccttagtaagtttaccaccatttattttaatcttttcaatagctctaatactaccttcagaaaaatcaacgtatttttgaagagcttctcttgttcctttagaaacacctttacctaaagcatcacttgtatcagtcgctttcttattaccctgtgccatagcatcgaaaacatttttagtagtttctttaagagaattccactctttaattctttctttacctactttagtgtttttccatgaatcgtaccattgtttaccaaattctttggctttattaccaaggtctcccatccatttagctagattcttaatacctgtgattaatcctccaccgatacctttagcaaagactttaactatttcaactgcaccattaacaccatttctaaaccaatcaacttctttataggctttaataagtccagatgctaataatgttaaaccagtaatcagtaaacctataggtccagtcataaatcttaatcctgacaatgccagtttcaatacttttactaaaccaccaaagccacctttaacccaatttaatggattaaccatagctaagaatcctttacctaatccaccaacagcaacttttgtagctcctactggatttttaatagctgtaactgctttaccaaatactttagctcctaatcctccagctgctcctgctgctgcaaaaccttccatagctgtagctgctgcacctaatactgcaacaaaaccagacaatgcgaatgctactggtgctataattacaggaatacctacaagtgctactactaatagtttaacactatctggagcttttgacaaggctctcattacaggtgttaaagctttcattaatgctattaacgcaggtgctaaagccgaaccaatagaacgacctaaagcaaatagttcgtttttaaatagttgaatttgagaacctaaagttttataacgttcagccgcttcatcagataaagctgttccctctttccaacctttattggcatgttcacgtgcatctgctaactgtttgttaccgttagcaagacgtaatacagtatctgtttcacgtaattctttaatacctaaatcaccaagtaattctttatggttttcaccttggtctactgctttcttgacacctttttcaaacaacgtaattgcttcataagggtctttcttccaagtttcagcaaactctttaccagatattcctgccacgtcagcaaatccttgtaattgcttacctccagacattacagcagtgttaatcttagacattaccttactcattgctgagccaccagcttcggcacggatacctacaccagccatagcagcagacaatgctacaatatctttctcagctaatcctactgttgtacctgtaccagaaagacgtaacataaagttagatatttcatcttcctgtgcagccatgttattcattttgttatcgtaaaggctttttatcctctacttcttatagtttcctataaggtcggcgtacattttcaaccacatgggttgtcggacactcttgggagaattattactctcttagcgttcatctcctacgctctacggtgtcgagtgatgttctcgattacctcggtattagtatatcatttagacttaaccttcaccgattttatccgatattaaccaaccaattgcttggatggtgggcaataacatttacccaaatttacaatagtagaaccaagtttttcaaagttcttagtgctttgtttcattcctgttacgttggcaaaacgagccatagctgttgaagcatcttcaatagacatgttcgttgctacacccatttgagttgcaaccttagtgaaaccttctaagttcttagtacctttaatacctaattgagcagctagacccatagtctctgctatattagtagctgtttcaggtatttgtttagacattttcataatagagtcatctaattgtttaaattcttgttcagttgctccattaactgtcttacgaacttgagcgaaagcttcttcccattgtactgttgctctaacagaaccaccaatagctctagccattggataagctactgtagttgccactgcaccaattgacattagtgagttacgaacttcgattacagctgctttattacgttttaaggcttgtaaatttttaccaaaacctgtttgtaataatctttgtttattagctgctacttgtaactgactacccatacctttaaggctttgagtatattcattttgagtagcttttaatgtgtttaactttgtaatctggtctctaacttcattagaatgagcaccaaaacgagctttcaatttacctaactgagcatactcagcttgacgtagttgattaatcgtcttaatttgaccttgtaaccctttttgtttagcttgtaaagctttaacctcattaccttgtgctttataaagattaacactagactttaaatgtgctgacgtagattttaaagtagactctaaatttctagcagatacattcagcttagcattagcatctctattctgtttcatttggttctcatatctagccatagaacctgtaactctattcatttgttgtacgatgttagagtattgaaccgtttcagttttattctgcttttctagttctctttttctgttagctagagactgatactctttctcttgttgcttcatgattctttgaagaactttaaattcttctcctaatcttttggcttcgtttcctgaacctttgaatacattgtttactgctttcatttcatcacgtaggttacgagaaccacgtctaattgctgttaaactttgtgtaaattgtgcagtatccatatccattctgataatcatattaccataaggtttgcctgccttgaccattaaatcactcccctgtaattttataacactatatgttgtgtctaaaaacacaaaaatggcttaataaacactatatattgtatgtatgtatactataaaatctaacttttatgtataaaaaaagacaaaaaaataaagagggaatataacaaccttttacagttactatatccccccagatattatttttataatagtccgttcttagccaagaatgattccatacttgtttccaaaggaccaaccttttctttgttgttctgtcttgctttcttcttacgtttcttagcattcaagatgtcaccaatctcttctggtgatagattgtctaaactctccatagtccaaccttcctttatcaaaggctcatacattgaaatgtaatcatagtaagcttcctcagcagacaaggtttctactttccctttttaacgtcttctggaactgtttcgtcgtttccaactacttcatttagaatcttagttaaagtattagagaattcagttgatggtactccatcccaaattgcatcttctgtaacttctttagcatcgaataaatcgtttgcgatataagctaccatgatatccatttgttggatttcatctactttaggatttccttcttcatctacagcttcaaagtttttgaatacttctaaagcctttcttaatttacgtgaacttacgaatccttgtttgtgtacttttgtttcctctgtttttgggtcaaataattctagtttaatcatattgtttttctctccttaaattttaatatgtatataagtgacttatttaagccactcttctgtttcttttatgttttctaaatatttatatgatgcataatctttactaacatagtcatggttcttattaaaaggcgttgtgttattatatccacccttaaagaataagtagtcttgtcctgctactcctgcattatgaaatatcttattttcgtaccatttatcaatagggtctgtagcccaacaaaatgataactctttagatacatgtggtataatattaaacaatggcatgttccatattgttgcattcatttctactgcccacttttgaaaattagtatctatatctttaatatatttccataatttaatactgtcttcatatacttttttaaaatatccagctacaggctctgatataatatattgagcacctattgaactgttattcatgttatatatgtcttctacagaaatacctattatattagccatttcttgtaatatctctttacctttttcacattgtatgatatagtcatagttgagataaccattacaatcagaaccacaccaatgagaataatttgtttctattttagaataatctggaatttctctaaatataacatctgaatccatatagaaataagtttcattttctcttaagatatcctcttcaagatacctgtacataagatatggttttagacttggtatatattcaaaatcaaatctttcatcttcatacacatgaacattcactcctgtctttaaaatatttttaactacagagtcatcatgtttagaaaatagtattacaatatcttctttatttatgccattttttaataagttatgaatacaaacttgtgtctcccattcaaaccttaaaacagcaggttgtgccataatatatttcataatatctatctccttttaaatatgtataacctccctataatatagggagatgtttcaggtactactaatcttgactaaccttctggtggtgttggaacatcttcaatcgttgttgttgtagtcgtagtcggctgtgtcgtcgtagtggtagttgtactagacgtcgtagtcgttgtagtcggttgagtagttgtcgttgttgttgtcttaggtttagttttaaatgcaggaacatctacaatttcagaaataacttcattctcgtctttttcttcattgtaagatacgaaaccacaccttaactcacctttagcatattgcgtatctggttgtacagctacatcggctttaaattcaccgttaatctttccagaatatcccacaatcgcatctgactgattccatacaacaaataaagtatctgctttattttctacatttgacattaaattgtcctccttttaatttccccttaatcagcaaaaatagtagctgtttctgattcagctactactcttacattttggggatttctagggagtagttgttgaagttgttgttgtagttgttccctcgctaatgttttggaataaagcttccatcttagcacgtttgtcttcacctttagcagtagctacagtgttaccgttagcatctgcaacgaatgaacctgtcatttcttctgcttctggttcagcctgttcttcttcattagttgacattgtttgttcatctaatgcgaatttacctttaagtaatgcaaagaatactggctctccacttaaagcgtttgattctaataatacacctgcataaggaggttgtgtttcagaaccaatgaatacgataccgtcttcattttctacacgacctaataagtctgattgtagttcttgtgtcatatctaatactgagatactagcttctactgaacctgtacctttctgtgctacataataagcaatgtttgaagcataaactttaactggttctgctgaaagaccagaaatactagcttcaaccataccacctttattagctttaccttctacaacatgtgtttttactactttatcatcttcgtcgtaaataaagtatgttcctcttttgaatccaataactgttgccatgtttgttttcctccattttaataagttttatttctatagttgtactgccttgtgattcctatgacaccatcataatcagggtcaaagaaatcataacctgtagaaaagtaaaatccttcttcttctaatatgtcatcaactctttgtataatatcttcaaagtcaacatctgtatcatcatcaaagaaatattgaatctgtactctttctttggtcatccataatgtattgttagcatgaacattaggagtgcttagtacatgttgtatcttaaccattggaaaatgttctattacagaattcttttctggtatcttatacaagtacacaagctttgggtcaatgactccatcccattcacgtctaataatcttttgtaattttcttaatatttgtgtactaatcataatagtttgtcaaactccctttccatttggtcttgaactttcctgtatgctgtatcgaaactgttttgcaataatttatcaaagaagaactgaccattcatattgacagtaccattattgacaaatattgcacgccatgcagtgtcttttccgtaacctatttcatatgttccttctacagtcttatggaacttaacatcatctttcatatgtgttgtttcataaggtttgtttgctccatatttacctaatttcctaggaggtccaacaggtgtgactttctctagctcatctgctaaatacttagcaccttcttttaaagctctatctgcatgtgttttgctttgtttctctaactgatacatacgtttttggaagtttctgtcatcaaatgtgaatgaagttttcattagaataaatcctcaagaataaccatatcgaattgtctcttgtctaaatctggtacataagctgttatttcatgctttctaccattaacttcaacaatccaatcattttgaacatcatatttttgttgacttcttattatgattgttgctctggatttatgacctgtacccatagcttcttgtatttcactaatcctagcctgtctaatattacaccaacaagaaaacttaaatacttcttctattcttatagtgcctctttttgtttcaacttcttcttttgcatagaatttacatcttctattcaatttacctgcatcattaactagcattgccatgactattcacctcttttttctttttggttatagtcaacttcatcaattggggcaacaaatgcagtagacttagcattacttaagtcctcatctcctgtgatagaagtataggtatctataatctcagaacgatagaataatcctagttccttatcatataatatattacttgtatctccaccaactgaaacagaaagtcttccagtgtttctacctactgcacctaattttataaatctatgtgggtttaataggtctttaaatgtataatccatttctttaacatttgaacgtggtgtcataatcgttcttgaatcataccattgagccattaaattaagtctagctaaatcaaactcttctggataaccaccttcaaaatctgagaactttctattcaaataagtttccacaaaagacttagaagctctaatcatttgtttgataaacctatcttcttctggttctccatagtcatctccacgtaaatagtttcttacgtattccagagttaactcttcatttccttccatttaatcacttcctttatccatctccacaaatctaacccagtcagattcatagtcgtcaatatttttcattaagtcatcgtgataaaagggatttaatatatcgactattccgtcatcataaagcattacacctaattcaaaatcaaggtctctttctttatttctgcgtactgtatcatttgtcttgtcaagtatagtatcttctaaaaacctagctcttaacattattaatcacctctaatatatgtatgtaaagaggggattgacccctctaaaatcttatgttattatttaggttgagcaacaactaatgcttcagcattaatgatgttaccatccatgtaaccgtctaacacaagtaattgtgtaccgtttaatgattgttgagtatctgctgatacatgtttaagagagaaacctttcttaaccatgatagcgtaagctgcattgaaatcaccaaagatgataccgtctttgtcttctaaagcttctgttacgaaaatatcaaaacctagtaatttagtacctaatttaccattgatgtttcctgattgaagtaagaagtctccgttaccgtcttttaatttagcgatagcgttaaaacgagcacgtgacatatagaacgctgcgttttgtaagtaagctgggtgtacagagttaatgatatcaactaagtcatcaccagttacagtagcttttaaatctactttgtttaatccttctgaagctaaaccttctgtagaaagtacaccaacgaatccacgttcttgaccttcacctttgaagattgatttttcaatagctttagctgcacgacgagctaataaatcagaagagtaagatagtaaatctacgcctgagtcgtttaataattgttgagttaatgttactgctgcaccaacacgtttttggttaagtgatacagaaccgaatttcaaacgaatagatggaacttcttcgttttcaccaacgaaaccagcttggtcatcagtattttctttagcaatctttaaagtaccagccattgatgggaacttacgagattgtgcgaatactggagcgttctctgataatgataccatgatttctccccaaatattctcaggaattaatgttgaaccatcttcttgagtgttaacgtactgagcacgtttctctgggtttttaccacgaataaatccttctaagtttgaacgtacttccatttcttgtttatctgccatttctctttcctccactttttttcctttgtcgcttaattcacgaatttcttcatcaatagtagcgatttccgagtttaatttatcgaatttgttgttttcatcttctgttaagtttcttgtttcacctggtttaatagtagcatcatttactaactcgtccatttgtgttaacaagttgttacgttgttctgttaattctttgatatttttagccattattttttacctccacgtaattcagctacacgtctttgagcttcttcaatagaaactgaacgagtaccttcaccttcaacctctgcttgagtagtagttgtagtagttgtagcttttgttgttgtagtagttgtcgttggtttagctgtagttgttgtcgtagtagttggttctgattcatggttttgtgtaccaggtaaatcatctgggtctgaaccatcaaattgagctaaacgacctgattgcgattctggttcgttagttgttacattatcgtcacctgcatgaggtacttttgtatcatggttatatgaacctttatcatcaatttccttatctccaaaatatttagataagctttcatctgcaccaattgctttaataattgcttgacctagtgattctgcttgcttagcagtttgttgtcctgctgcatagttatctttttgtactaatacatcattaccgtcaacatctttacctttagttccacgctcttcacgtaatgaacgaacttcttgtgttaattcggtaatagcattatacaaatcatttgtactgcgttgttccatgtttgtttcctcctcgatattatctggcacttcttcgtctgacatatcaatgccacgactagatatgcttgattgagcatatgctggattacgaactgctgaaacctcgtataattctagttccgatatagtacgttcactcaatccttgtgtcactgatttccaagtatctttaattgctctgaaaccaaaggacatatttgtaatcaatccagaacgtatcatctcataccaatctcgtcctgtacttgtattaattatcttagccctcattaataaacctttgtcatcttctataagttccaaagttccattttttgtagatgcaagcactgttgaagagtcgtgctcagctaagaaatcaatatctcctgtggcttcggataaggctctttgaaaagcgcctttagatatcttttcgacaaatcttttagtcatacctaactcttgacttaatgtttctgtctcatttacatatccagatactatcatatcaccctcatcattcatatgggcattttgcattcttacttccatgcgttgattcatatgatatatcttcctttctatttattgatgtttaaccatctgatttcttgttttttactggaatctctttatcttctgattcagcatctacatcttgtgcgttttcttctttaggtatattatcagcagaacctttagcttgtgctaatgattctgggtctttaggatttatcttgattcccatgttaggaataatcatatctcctgttttcttatcatagaatacgtttgccaaactataattcatgaaatcatctaataatgtttcataaccatgtctagttctagcttcgtttccacttatgattccacctttaagagcaacatttgtagcttcgaatttttctttttcagtagtagccaatacttcacgaacatcaaattcaaagtgatatccttcttttttctcatcttctaataataatgatttatctaaagaactttctattgcactaattattggttttaatgtatactgtaagaagtgtaagttattctgttcgttacttgcatatttgttagcatctgcattaatcattgattctggcaggttaaatatacgagctatgtcagataaaacatactttctacttgaacttaaatctaattcattagggtttaaagatactggcttgtattctaatccctcttctaaaataactgttttacctgcattttctccaccttgataaacagtagcccaactctcttttaagttttcaaaagagtcatcagatagtttatgtggtgagcttaatgtagctaaaggtaaagcaccattttgtaatattgaattattatatttttgttgttcaatagctaaaagaagtaaatctgtacttaagtttaatataccttctgattgaacaccatcatcagagttctttagtatgatgattaattcgtaagggtcaaattcttttgttttaccacctgcatcatatttaatgatagcatcttctataaaaccatctcttaaatatttgtcaaccattatacgattggcttttaagtgatgtatacctgtaacatcatttcttactttttctggatataaataagaattaccataaaataagtaatccataacaattctttttttaagagtagttgcattcataagattgttaggttctttatttaataagaaagctctattatcatctactgtttctatagaacctttaggtccttttttagttaaattaatatttaatctagctatagtagaagctattaattcagatgctgccataactgctgaaatagataaagcatcttcttcgtctatttttgtagaaccaccaaagtgagttccagtaatagataatcctttcattctaccagatggtgaataatccaccatattaggtgcttgttttcttctattttttataaaaccatttaaaacattgtcaattactgccaagttaggtttacctccaatcttataatactcttaaacctcgtttagcgtaaacattgtttaatcccatttcttctatttctttatgccataagaacacagcatctgctgttgagaataacatgtctatttttcctgagcttttctttttgtttacatacatatctaagttattactttctacctgcctagcgttcttgaagtgtatctccaataaatcattcttttcataatggaaattaccttttaaaacatgttcttttattgttttaaatgtagaatgtagatttaatggtgcttgtaccacttcaatagcttcatatcctgcttcttctgaaatcctacgcatagatgatatggcttggtatttatcataaccgatacctttaatttttacatcgtattttttttcaaggctcataataaaatcttctacaaacctgtaatcaattactctatctccacaaaaataagcccatccattatctctcataaattgataatcaacacgctctttttgtgtcttgtctgctacaccatcttctggtataaaactccaagatttagtataatacttacctttttcgtaatcataatgaaccattgatactgctgtattatctgttgtcaatgataaatcgacaccaacatatacttccttacctctccaatcaaaataatcaatcttatttggctgtaagtcatcaatgttcatataagcttctgagacatcgccatcaacgaatatattcatatgtttggttaagaagttacttcttgttcctggcatctcaatagcagtgtttcgtaattttactaagtaatcaaagttctctttaactgtttctataagagggtttgcttcaataagtgatttatctgtcatccactctttagggtcatctggtttatataataaagagaataatgcatcatcttctattataccatctaaaaccttttctgcatactctacttcttgtgtcattggattatccgttgactgataagctgtagaaattaaaattcccgtacgattcaccatgttcatctggctcgattgcatggcttgtattgggtaggaatctctaagagctccaacttcatctgctacgtacacatttgctttccttccatccatacggttgttacttgtagccaaagcaatcattttactttttttaagctcacatcttatttcagattttaccaatttaaagtattttgatatagctggagattcttgaatagttttttctatttcttgtttaacgatagatgataattctctgtcaggtgcaactgaataaaattcagaatactcatcttccattaaaagaagtattgtaaaaattaatccaacaaggaaagatttaccacttttccttgcaatcaatagaactgatttttcgtaccttcttttttctggattatttttgtgtttccaacaaagagcatttatgataaaataccattggaaaggagcgagtgcttctctcgttggtttacccttcattacaccatcagccatattaattatacctgttaaattgtcaataacctttatagcatcaacatctaagaaatatttactatcttcatcttccaagtctctaagaaacttttcacattctttaataatatacttacctgcaattatttctctgtttacaacttgttgtgcatattgataagatggatgctcgataatataattatcaatcatttaatctaccacctttatgtataccctcactatcggatttgcaccaataaccactgtcttattgccagcgctctacttgttgagctaagtgaaggtcattcccatacgtatgtatagcgagttatttttaatgtaggtcacctcgctcaactactcatcgttgtttgcatcttgaggtaacgaaccaaaaccccacgccttattatactgtaggtccaaacagtcgctaagcattaagcaaagggtaattaatccttcactttatcgagtttcctcttcagcaaagattccactctcaatcatcctcagatgagacatatatgtatagctgacctaacaggactcgaacctgtaactttcgcattaacagtgcgacactctaccattgagttataagtcaataataaaaacacacacctaaaacccataactttagcatgtgtataaagcaacaaggttatttccccaccttattgcaagtttttggaaatatttaaaataggataatggcttgtgatggatttgcaccaccgaacccgaaggaacagatttacagtctgtcgtgtttgtcttatcttcactaacaagccttaatacggagtatataggattcgaacctatgcatgggtttccccacctactcaggtagcaaccgagccccttaatcctcttgggtaatactccttaaatggtaaacggatagtataggatttgaacctatgaacgctctcacgttgtcggttttcaagaccgatgcaataatccagactctgccaactatccaaaatagaggagagggtttatgaagtcctctccaggagaaattaattatgaatatttaagttgtgagacaacaactctatgcctattgtaagattcgaactcacgaccccttcattacaagtgaagtgctcttccaactgagctaaacaggcttaataataaagtaaaagctctacattatttcctaggcttatgtagggcttaaacttgatgagatttaacctttaaaacttatctcatccttaacaataagtggctaacttaaagtcaagtctttttctaaacgtttaaggtatatttccccatataaaaagtaggaaaatatattataaaataactattttatgtatagtatgggtctaaaaagtttttgaactttaactgagaacagcccatacactaaaaactcagtaatattatgtatataccctagtggttagctagggagttactaagtaaccatcttagtaagtggatgtctacgaacatggataactcgacacctgcttcgggtctctcaatattctaaaatggcgttgagctcgccccacgacctccgaaacgttttgtaatatgtaactccttttaacctgcaactcaggagcgtggtaatttaccgtacgtacaaattccagtttttgtatactcaggacatttttgattttcaactaaggaatttttatttatagtcaatatatccctttgacttgatactatttaatttaaatttattgttatgtatccccttgagcaattaaacgcaaggggtacttctgtgccatcacttatatagaaaacctaaaacattataagctggacttttaaaaagatgtttattttgtcaacatatgtaaaaagattatcactatccatggttgataagttattacacccactatcatagggttagttgattcagggacctaattatgtattgctcacaccccttgaggaatgagcttaatcactatgttaaaagataaaagtttatgtatccctagcctgttccaaggctaggtgtgaggtggtgcgtatgaatcttaatcctagaaaggtagcgaaatccctcgtcggatagttcttttataattgaatagtagttatactatcaatatcattttcattaaatacaataaatttctgtcctgctcttccaccatatcttccactagctgcataatcatctgcaccaattaaacttggattaacaataaccattgttctaccaaagtctttttggaagtcatgatgtgtatgtcctgagataatgtaatctggaacataacctaataactgaggtaaacgataagcactgtttgcttgtctatcaaagttaccatgagtaaggatgaattttttacctactatttctgtttcaatcaatccatcttgacatcctatcatatttacattatcatatttattaaatgacaattctaaatatttaagaataagtttctcgaagttttcttctacccctgcaacctcatttttatttggagttacacgaccatggttaccaataacgttataatattcaatttgaatccccatgtcagcaaactctttaatgaagtcagtaagtaaatcagatgctgtaataatctgttgaataacatcttcttctgattgaatcttagtagatacatgaataagaccatgaaccatatcacccaagttagcaatatgtagtttatcaagtccttcatattttactttttctaaagtcttctgtttaacttttccaatacgttgtttagcaatatctaaatcaaagttattgaatcttcctttatatactgcacctaaatgccagtcacttaaaacaaccatgccttcattattacttgtattttgtaaagtaacaggcatatgtgcaggttcaagctcttctaaagcttcttctaaataatctgataagtgttcaagtcttgcttgtcttctaattatagcactgaactctcttttttggtctctcattttaactttttctttttgtagttctaacttttcttctttaatttcttctaaaatctcttgttcggaatgactaataacgccttctccaaacatttctttaaattgaacaaatccttcatatggtcttctccacttagcttcattataatcatttccagaaacctcgtttaaaagtaatccaatatctttaaaagttaatcctaataaatctttatcttgataaagacgaatataatattctttgaatgtctcatctttcctttttaaatagtttgtcataattattctccttttatttaaagcttatgtaaacctgcctagaaacctaaactcgacagaaagtttttcgttgggggaacaggtctttatatagtgttatttatttaaacctagtgctttcaatactgggtcatcttctttttctttttgttctacgtttaattctgctaatgcagctcttgaactaggactcattcctaattgattagatagcctattaaatctatctaagtaagctaatttaacatctacagcaggattctttttaactatatcattactatttctatctttttgctcaactactacccctttttgctgaataataatatctgcttctctcattcttgatagacagtcagccgtttgttctactaaaggaatatctaaattagataataacccactgacttctaattcttctgtaatgtatttatagtattctttagctaaatcatctaaatggtctggaacatctgtaactttctctgagttacctttaattttttcttcttgctcaaccattttttctagctcacctttagtatgatgttttgttccgtctttcattttaagagaagctggtttgtttgctggcataaccatacctccttaaagtttaactttataaacgttatcattaaatctttttatttcaaaatctaaatgctcttgtgttcctaatgaaacgttacagaatttacataacgtaattaggttatcaggttcaaacataagttctggatatttgttacgtggtttaatatgatgaacttgtaaattactagatactatcttacctagttgtacataacatctattacacattccaccgtctctttttattatcatagacctagtgcttttccattgtttactgtttagcatttttttaagttctttattactttcatagtattttttactatattcatttctattttgtttacgacactcacatacttctccatcaacaattttattgctacaataaggacatatcttatacagagtagtcatcgttcagcacaactccgtcttcgtcttgtatatttccacaatgatggcattcgtatattagttctccgtcgtaagagtctattatgtcaattgcacttccacaatttccacattctttcatttttatcatcctatacatattatattacatgtataaaaaataatttccattatatacctctacaatataagaggtactgggacgacaactttgcgtagtcttttataaagttttttgaatatttaaataattgtaatataactgtaataattctttaatataactgtaacctacagtagtacctttttatgttatagtatgtatgagcaaaaaagtttaaattaattttaaatttatgttgacaagcatagacaatctatgttatagtataattaacaattaaatacaaagattcaagacggacattgttatcaccaccgtctataaataagaaaggtgaacggtgtgacgagccatttactaaatcatatagaaaattcgttcgtaaggcttagttgagctgtgcattttgtgcaatacagttcccttcgatagacttctttaaccgggtagaagtcaataaataaaacaacggtggttgtaaccaaagcttgaggtttatttgttatacttatttttagtattgaacaaataagttttaccacagagcaacttggtcgggtcgtttgacagagatgatttggtatctcttagagttcattatgtaatatctttatggcttaattaataaagattctatattatagttttaatgaattattaagttatatataagggaaaatataatctcaagaaaacaataattaataataagaaaaacacaatgaaaaatttgaaaaattttacattgacattcaaaaatgaatttttgaatgaaagaacttaagatataataaaaaggagaatataatatgaataaacaaaaagttaatggtattaaaatggtagttaaatttaaagaatttaaaaatagcaatggtaaagttgactactatgtagatgaagactttatacatagtaaaattagaataagtgaaaaaaatattaataaggcttacaaaaataaaagaaataaaacaacggtaataagatatgaagataaactatactttagtgaacaaggatttaaagatttgattaatatttgtcataagagttatagaaaagatactgctgatttattacttggaaaatataaaaataaaaacagaagaaatgctgacactgtaactcaacaatcagttgaaggagctatcaatagaaatagacctcctctatctatcaatattttaaggcttgataatgaaatattcttaatagctaatgagctggttgagagtgttaattgtccctcaagagacttcatgaaatcgctagataaaaagacattctttgataaaactaattacaaaactctaagagaaaagaaacgatataaaactatagcaatgaataaagaaggatttttacaaatatgtagagaacttaaactcaaagaagaacaatatgaaaaagttttaaaaatttacgaataatatattgacacattcttttctatgtaatatactatatgtataccaaacgaaaggaatgaacaaaatgaacaaatatattattactaaccatgctgtagatagatacaaagagagaactacagtaaatactaaggaagttaagaaaagcataatgagagaccttaaagctcttaaaaacaaaagaattattaatgtaggagacaaacaatatgtcttctacaaacattcaaaagaatacatccttcaaaagaaaggtgataattacatattactaactgtaattaaacataagagaggagataaagaaaaagccattgataaaagactaagagagaaagcagaatacgaagaaacaatggatgcttttaaaaattataaaaaatagtattgacttattgtaaaactaatggtactatatatgtataaacaaaaaggagattttaaattatgagcaagttagaaaatttatattatgtattagagacaaatgatgtacagaaagcttttgacatagctaatatggaagaagattcttatatggatgttaatcaaggttgtaggttaattattagcaggaaagataatcatacgtttttaattgatgtaactaaagaaggttttaaaaataatagagttactaaaccagaattaaaagccatcagagaatatctttctaaatttaatagagatggagatattcttcctaaaataacagaaaatggtgtaacattgtctttagacagagagtttgatttgaattttgatattgaatatggagaaattcataacattccagaaaatactacttataaaacaatggcatatgctaaaatgattggttattctattgttaatatattaaaagaccatgttatggtacttaatcatcaagataaagaaaataaagaagcctggtttaaggcattttaaggagaatctatatggaaattaaagaaaaaatggaattatcaactaatgtatggcaggttatcggtattaatggtaaatctcagcaagaatctgtagtgtttgacactaaagaaaatgcacaaaagtatttaaatgaacaaaaacacttattaacagcagtagagcctattaaagtaagtgtaatgacactaaaagaggattaattatgagtatattattaattatattgtctagtctaataatttctggaacttttctgataattatatttaaaaaaatgcaagaaaatgatatgattattatgttttggaatatatcattaagtattctatgtctaatattttcttctatatattattctatagctgttttaattatatatattataattttatcatttttactctaataccatgtttattcatggtattttttattatttatgttgacaaccattacatatatatgttatactttaattaagaaatcaataagaggagaattattaaaatgaaattatataacatagatgaattagtaaaatcaggattagaagatggagtttttgttccatatgataaagaaacaaatgaatttttaaatgtagatatacaggaaatatctttatctaatggaaacgtagaagttttaagtggagacaatagagaataccattgtttagcagaactatgttctatgctaggaaggtataattatgtttttatgaaaaaagagaataggggaagaggaaatgaagaattttagtgtgtttttagaggaattaaacggtgtctttattgaacaagggggaacaatttttaaaattgtcgggtgggaatatatcgaaccagggtactatagagtcatcatcaaatcgaatgcaggagtaatagaactaagagttagagataaggaattgaaagaaaacaatattcttttccatttatatccttatagctataaaagagaagttgcacttgatattatctcaagaagggctatgtatctttataaaatgaaaaagaaaagagatagtatacctggagaatttattgatgtggataaaaatgtgggtaatataaatatgactaaaaaagaaatcgatagtattatagaaagaatgtctaaagagtaaggagatttttatgaaaagtaattttttagtattgactatgggtatattgctgatactagcaagtattgtgacagcaatgtatcaagactggggtcaatcaggtagtcttttaatagatgcttttttgttaataatactttctatatatataaagagtaataacgaaaaagataacttgataaaaagacagcaagatgatttaagaagctctaagaaattggtagacatgatagcagaagcacaaaaaaagaacgaagatgatgcgaccgacaccaatgtcggtagcaaacaagatgaaaaagaagatggaggtaaaaaatgagtatattgaccattgcactatctatcattttacttctacttgtaatgtatattgtaatgattgtaatatctttaaaattaatgacatcatttatacaatggaagtacattaaattaatgaacggaaacatagagtttgcatctaagaaactacaatcggattttgaaatatttacaaatactacaagtggtagagatttaattaaaaataaatttaaagacgtagaataatcccttattaattaaagggattttttaggaggatgagtattatggagatggaagttacaataggagaaattatagaaaatataagtttagaaaatattaatgtggaatcacttataggatacgctgacaatgaaatagtagatattatttttactggtataaacgatgcattcggatatcaaaaaaaatcatcagaacttggatttgatattcataaaaaagttaaaatgtctactgtagattattatgttgaaagtattattgataaaataagaaacgatattgtgatttatcaatcaggaaatagaaaaatagttttaaagaaaaaaattaaagatttggagatgaatatttatggatggaaatagaatttatttagtttcagaggaaatacattatgaaggttctattggttatcatatatgtaaagattttaatgaagttgtttctttatttgagaaacttaaaatagactatgaagaaaatggggaatatttcagtaaagatagtaaatgggaatctgattttgtgaattttgactatcttactgttttatatagagaccttgatgagccaggtacattaactaaggaattcgaagagaatgtattagaatttaaagataaaataatacagaagaatcatcaacagtctgcgttaggtgctgttctaagtgctaagattggattaaataatgttaagtcatacgatattgtctatgacaaaaactatatgattacagacattaatattagtttaaacacaagagaccaagcaattataaataatacacatagagaagttgctcaacactttgcgacgaacttatatggtattgaaataaatattcttaaaccatcaaaataatgattgacagcctagtgtatctcatggtatactaggtttataatataattaggaggaattagtatggattttgcagatttaaataattttaatttaataaaaaaacagtttgtaagtgtactacaaaataaatatggtgttaataagaacattgtgtttaagtatatcaataaaaacaatgttgatgtatatttggaagatgatttaattataggtattaattatcatataatggattccactatggtacaggaattgttagatattggtattacaaactataagaatctttatacatcaaagaaatattttctttcactttttaaatattctactggtaatgattttaattttcaaaaatctattgtaaaaatattagaagaagttggattcgaaagtaataaagaaactgatgtggtaaacaatgctcaggagattatagagattactaaaaaagcaaaagaagaaaatattaaaaaagacatagatgaattagagtctcttatattggaaaaagctagagatggtagagaaggactaatgttaagattatcaaaacttaagtcgaatatccttgaagaagaatacaacaatgaaatatttaaacattttagaaataaaggttttaagtgttctatccataatggtgtttctgataggtatataaatattacatggggagattaatttctccctcaatacacaaaaaataattaaggaaaggtgttgacaggtaatgaaatcagtagtatacttaatgtacaagttaaaagagcaggaaaaactcgctgtaattgtatctgtgttctctcagaaaacacaaaaaagagtaagatgtct

SEQ ID NO: 3 - DNA, 148313 bp, unidentified
  source 1..148313: mol_type = genomic DNA
ggaattcttttacctctctcactcagcctattacttattaccgacttccctaactacttattctatagttataatattcatttattatacaatacttaaactatagtattctactgttaatctatgctgaagcggtcttaatctatggttattatataataatcttatataatggtacattaatctagtatattacattagaatcattctaatctaggattttaatctttagaccctaggaaaagtggtactaaaatataaaaccctataggtatgggattcttatttttaaaattactaaaaagtattaggttttccctagggcaaagttttaatgtacttaaaatagtaagtagctacttatcatttagggttctataattgagaatattgagagataatccgcttcaattgtaattaattgttgacaactatgaagcgggtatgctataattaggtatagtcaaatttaggagatgaaatagatgattgatatatacttaggagaaggttataataaagaatacttgtctaaagcactcagattaatcaatgaccatgctcctagggagttaagttatgattttaataatgtagaagcggatgttaatattcacacaatgttatatgttaaacctgaagatagatttatatataaggatatatcctatgacttcccgggtgatttaattatttgtatagttgatgatgatgctattgtataccaccaaggtgagcagatttcaggtattagtattttaagaatactagaagagatattttaaggaggataagtaatcatgataggaataacaatattaattacgataatgagtatatcaactatctctatgtatatttattttttagtagacttgattcagtcaatcagatataatagttttgataaggtaattaacgtcataacatttgtacttatgacagttataatagcatcaggtattttagctatacttggaatatagagctcatttaagaagcggttaagtagttagaggggatttgtcctaaaatagtataccgcttctatatggaaggctgagaggtcttagaattgaaaggagagatataatgattcatatatttttaactgatagttatgataataaagttttaaatactgtactcagatatattaatactactagtgatagagagcttagttacttaatgggtaaaggtgaagcggatgtatgtatagaaaagggagtatttagtaatatagaagatgttaaaattgactctgagtttattgatagaggtaacttatgtatacttataaatgaagatggattagtatgtagttactacagaggagaatcatgtaatgttggttcctttgtaaaggagaggttataatgatagaaattaggttaactgaagattataatgacttgagtcttaaggcattactaaaacgtattaaaagggtagctcctagggaattaacttatggtttagaagcggatatggatactacagatgttaatattggagattcagttccttctagaggtttatatgtagagtactcagaacgttttactagggacttatggataattgtacacccttcaggttatgatgcttattatcaaggagagaaatatggtggagagtctttagatgagattatacatgatatgttttatgattatgcagacccttttgacttagattattagaaaggagagattataatgatagagatataccttagtgaaaattatgataagaatttactaaaagcagaattaaaatggattaaagagaccgcttcaagagaactaacttatgatattaataggaaacctggattggatgtttatgttaatccctataggtgtactaaagacgaagttgaagaatggagtacacttcctccatttgaagatgatatacttgtatttatagcggagacgtggatacatgaatatcttaagggtgaatcaataggtgtagatagtatggaagagtatgtaaaggagatgtaactaatgtttaaggtatattatacagtctaccatagaggtagtatgaaaactattaaggataagctagatagaagtagtttaatatacttcttgtatgatacttggtataaagatattagtaacgtattccctaatcactataataaagagtttgggagtaagagtgatgatatagatatagataaacttattgaagcggttaatgaggaaggtatattacttatcaatagaggtaattatgttacaataagagaatggtaggataggataaacttaggatagaaaataatttaggatgagttacaataggataggataggatagggggttaagttaggatggatactttaacatacactattattcataaagaatctgatagggtaatagctagcggtttaaatgagacagaaactatgaacttagttcaaaggatgataaatactaatctagttactgatatatcattagatgattatatacgcagaccacatggaaagatagatgtagtcaatttactagtagatattagaagacaaggcgtatttgatttcaatcacatttggcacgtaggataggagggataggatgatagttatatatacagatgtttctaaggattatttaaaagacgagttcttaccttggcttaatgaaagggatagatacttagaatactataaagatgaattacctgaggatatagattcctcttatattgtatcagttgtatactgtaaggatatggaaggtctattagaaagaaaagacattgttcttgataatagttataatgaacctgtagctttattaggtgttcctgagttttttggtaattatagtaattatttctattatagaggagaaagtattagtaaacatgacctaggagaaattgttaggttaaaagcttggcaacgtatgggtggggattgactaagtagctctccctaatttcactaagtagctccctaggaattgcctaagtagctcggtatgattttaccctaagtagctccctctgttttctactagtttattttaaccgcttcaggtgtctatatatatatagacggttggaataatatcagaccgcaaaaataaatacactaggatattattcccagtgtattatataatttttttatagaatatttataacattgtattcaaattcatttacttcatgttgtgatttaattaaatttttaattaatccgttttgtgttttatactcttttattagtttttcattttctataattaaattattaaattcttcttttgttgtttcctcatctacataaaatttactttcatatatttcataatattttttatctgttccgccatctaaatcatctgatatttgataatttttgaatataatttcttttgtttctaattcatttactaataattgtgattttgcatattgtaatacatcttcattgtcccacattggaatatagtttattttcatttaaatcaaatccttttcttataatttttttatataatatttgtagaagcggttggggtttgtcccttgccttactacactttatatattacagtatagttattcagaagtcaatacttttgagtaactttttttaaattcttttttcttctatataatagtagtttttagccctaaaaatgtttttaaaagaatttgcattttcttattgactttattatcatatggtagtaatataaaggtacagcaagggaacagcaacaagatattagaattatataaaaaaattatttaatttgagatgatttaaatggatgtaaaagaaattgcaaatactataatggagttgtggcaaatggacggctacagatgtgcagaacctccattatatgaaagcacactaaaccacacacgcacacacacggcgttaattgtttctattaatggaaactatgacacagtgcagatgttccgcaaaacgcctataatgagcatgagagggcaaagccaaccggctagcatgttagttaatgtgattgacgatgtaattataatcgtatatgaaaatgtagtgtacggagttcaaaacaaagaaataaaatttattgaagaaatttaaaaataggggttgcaatcctcaagcatctatagtaatataataggtgtaggggatagcaacacacctcaaaaaactttttaaaaaagttaaagaaaagtgttgacaccttacaagatacatgttattattaagataacaaataagacaagccacctagcaaataacgaaattaaataaaaaaattatagaataggatttgattattatgacaaacaaaaattacttatacgaagaaactcacacagtacaagggcaagacattacggctttcagaattccaaatgacgcaaacggcaacccacgttatgtagtgaatttcagggacttaggtatcggattatgggactatgacaacatcaataaactttacggatttaataaatatcgtgccaaatggtttggcggtggtgtagtattccaaagctataatatagaagatacattaaattttgcactagataatgttaaagaaatagaagcggttaagaattaaaaccgcttctgaattaaataaaaaatttatataaaaaggatatgataatatgaaatttaaaatagaaaaaaataatagtgatataaaaactttatggaatttagcaaaaaatggatatatgagctatcaaactgtacacaatatatttaaaaatgaatcagatgaatttattatatttaacagtaaacaaacttataataaatttatgaaattaagatataatagaagtgcaatacaataatataaaaaaaattatacaattccctaggattagatttctagggatttttatttattttaatttatataaaaaaattatttaataaataagttagtgtaaaattgactattgacaaggttgtattttttatggtataatgaagtgaagaccttttttagtataaaaaaattattatataaaaaatttatattaaatgattttagaaacgctctttcccggaacctcttctcttatatagcggacacgtaggctccttaccgctttcttactatacttatagtatactatagaaaaaagaaaaggtcaacccttttctttaatcttttttatatttttttataattcttctaacggttatttcacttatattatattcctttgctaacataggttgagtgtactttctaggcttgtatttgcttgctatggcttttctttcatcttctgttaatgcataacccctattatacctattgaaagtattgtctttatggtgccttttattgtgttctgacgggctaatacattctaggttatcaatacaattattttgtttgttaccgtctatatggtgtacatggtttgatttaataaaatcattgccaaaatattcatatacaagtctatgcaccatgtgttttttattattgacccttacggttaaatatccgtcacggtcttgatgtactttactcaacctatcatttatttttaccctacctaaattagaaacataaaaattatatttattaaagtattctttttttataggtttccaaatttcattaattttattttcaaccatttttttactcctccttttttggtatcacttccattatataataattcggtcttaatgtcaatagataaatgtaaaaaagttttttaaattaatttcattaaatctattgactttaatatcattatagtttaatataaaggtataccaaatgaaagggattgaacaaaatgataaaattcaaatggaaaaacaaaacaattaaatcaactcaaaaaacggataacattctattacttattataggtggtttagttgcaacaatcacacctaaacttgtaaactggtttttactactacaagataatataaatatttttttaagataactattgacaacctagaaacaacatgttaatattaagataacaaataaatcaataaaggaaatgataaaaatgaaaaaaatcacaacaactttaaacttaatcggcatgaaaaataatgaaaggtttacagaagagttaaaaaactaccgtcaagatgttactttcttgaaagcaaataaaattgtaaaatattcaaaataaggcttgacaacttaaacactacatgttattattaaggtacaaggtaagggaagcggtcaaccgcttccaacctaaataaaaaaagtttaaaaaaaactattgacagtcacttgaaaccatgatattattaagataacaaaaaacaaacagaaaaggaattgattataatgaaatttatcaaaactatcgaaaacttattaactaaagcagaaaacaaagggcaagcaattttaaacggtcgttattatgacggatatagaaacggtgagcttgaggaaaaatatgcaatcgaaattgatggcaacaaattaattatgcgtcattggggtacacaaacaattgagattgacttaagcataaatgaaattgtttcatactatggtgaaagcaattcagaccgtgacagtttaaacacacttgtatattgcttaggaattgcgccaaactttagatacttaccaagcaaagacttgttcatttacgaaaattaattaaataaagggcttgactttcaagccctaccatgttattattaaattgtaaggtaatcaagcacaacgacaaaataaactgaaaaggaattgatacaaatgaaattaatcaatagagataatgaaatcgtaattagcatagcaacacttgagagtgtaaaacaagccctaatttgggagtacatcgaccacttagataataacatcctagacaaagaaatacatgaccaggaagcggttgttattacttcagacactttgcaatcactcaaatttgcggacactatggaagaactagaagaatatgtaaacgacatcggttggaaattagtttagaaaaggtattgacatcctaacatatagatggtaatataagagtatagaaaaaataaaaaaagaaaaggatttgattattatgacaaatacaatacaagcatttttacaaggacaagaagcaagcacagttaaggacgtagcaactcatggagtacaaagcggagcaattggcaaattaatctacacatcagacgtagtaaacttctttgatagttacgagcaggacattgaagcggtcatcactgaatacattgaagaggttacaggacaacaatattatgacttattgaactatgagcttatgagagacctcgagaattatgcaaatgtagaatttgaagacgaagacgaatataataacattcaatttgacctagcagaaaacattgcttctgatgaggttgaaggatttgaagacatggacgaagcagaccgggcggaagcaatctatgaggctatggatgatgttgaattagaactacaagaaactgacaaggttcaatatgttaatctagcggttgagattgtagctcaaagaatggcactatagaaagcacacagagaagcttaaccgcttctctaatacaattaatcaggagatgttgaagatgaatacaagacgggtaaacagagcgttaaacgaagcagttagattattagatgaacaaatagcagatactcaaaagactatgcaggagttgaataaacaactagagaagcaaataaaggctaagcaagagctaatggtattagttgatgttatgaatggtgatgatgagtaatgaacattagagaggttcacaatgtcgttaagagtgctaagagcaaactcctgcaggagcaggctcacccaacggataacctcatagagcagtacatcaatgatgagctacacagacgcacacagagaagcggaacaatacagatgaacaataatactacttcatatagtaatagcccatatggtagcttagaagagcttagagaagcttatgacctatcgtcattatctactggtgagattaaagaactaatacaaacatttgtttaaattattttatcaaaacgctttacaatcttttagtttgtatgatataatgaacttaacaaattaaaagaaaaggaaatgatgaacatgacaaacttacaagaaagaaaacaagaattgaaaacgttactatttaacttagctttagagaagaacaaagcaactgatgagacactgcttagcgtattagagcaagcacatcaagaggtaggaaatcaattaagaaaagtaagaaaagaaatagaaattttagtagaagaaaaagaaagggaattttggaatgatatcgaatttaatggattagactaagagggaataaaatccctcttttatttttatcctattatataatttttttatattatacgggggcaggggtaaaatgccactcaatgggggtgggtctatatacccctatggtctacccaggtacttattttttggggaaaattatgaaaataaatatttaaaagtcaacacctaaaatatagaacgtaagtcaacaccctatattaaaagtcaacaatttatagtacaaatagagaacctctaaatataaagtcaacatatctaaaataagaaagagggaaattaaatccctcctcttaggtattattaacaacctctaattcatgtatagtaatcatatccatcccatagaaatctcttgggtctcctttaatgaactcttgctctcctctatggtttgtttcctctttataaccttcttctttaatacgtttaattaagttctccttatctgtatatatcttatcttctctaaaatggaagttatcttcataaggttcacaattatcatgttctacttggtatagtttcattagtcattttcctcctcttcgtaagaccatgtaccatattctgcattatagtgtgctgtatctgcgccttcatcttcatattctacactataccatgcatcctcctctgtttctgcatctatatatcttacctcttctgtagtaatagtacgttttactttgtatctcttcaactgtttttactcctttatattttcctctagtatttgttttaatgtctgacagtctttttggtctagagtatcccaactctctaaattttgtaattggtatagtaactcatttacaatttcatcgaaggcttctgcttttttatatacttcttctagttctgtttctgctaattttctattccttttaatttgttctgctttagctactaagatatgggctttattcatttcctctataataaagtttttatagttttccattattatttatccctcctattttctatccgttgttttatctcttctctattgcggtggtgctccttactcatttctttacgttccttatttgttaaccttatcctataaacaaggtagttaatgtataagataccggctgaccatagtagcaagaatgatattaaataagtccatgagatactaatttctatcattgtgagtcctccttatattctttatagctcttaatggctattttacaaatacctctatttacagcaacaaatactataaatgataatagtgttataactgctcttacatcccctgtaaaaggtaatgattgaaagagcaaatagttttctaaaacactaatagctgtaatagtagttagatataatatagataataagtaatcctttaattttagtttaacaaatggttttttgtgctcacctgttcttacaataccataaagtattatgaaccacataacaggtactaactgtataataaaatcattgtctacatttaatgcatgtagagcgtaaataataactgcaggaatacctataatgaatgctagaaatacataaaatataattaacattataggaagggctacaagaaaacctagaccttgttttgaatactctaatgtgtttttacctaggaacttaaaaaatgttttattcatcttcttcctccttggaattactttctgtaattgtaatttctaacatattattgtaataatcattcttttgattgatattatagttatcattgtattcattaaagtctacataaatatattcatttgcgtcattttcataaataatatctatagctgtaatatctgaatatgctgtaatcatttcataagcgttcgtattatcaggataagcaaaaccaacttgaggtatttccataggcttatcaataagaataccgaaataagtacagtgacgtgttcgacttatacttgaagtccctttatatgtaccatagtaatctataccttctgtaatacctgatatatggaacctgcttacgtctttaggctctaatcttacaacatcgcaattctctaatactaaatcaatatatttgatattcattttaactctccttttatattaataattttttccattctttatcaacctttttaagttcttttttattatagtccccgtctttagttactacagtgttccattggaacttttgtaataagctaaaattatttataatccatatattacttttactataatacatattgtcttcaaatcttatatctttttctataaaatatttatatattttatatcttctttcatctgcacctgatattttaataatttcattagtatttaattgagtggataactggaagataacatcttttactttcaataggtctttaacattacctctgcctacatggtcattatagcaatcatatttaacttttttcttttgttttctatcattaactacaatgaatatattatatacgatataagctttaaaatgggtataggtagtaggtgcttctgaatcatcacattcttttcttaggtctgtacattgtatttttaatgtaatattatttgatatgttaactacagtagagccctcatgttttttattaagatttatcttatccattttataattacctacttattgtagatacaatgtactcgaacatcttccattactttgcctaatagattctgacctttccagttactttgctctaatattttagggtcatttgctttaagacctactccccatattttatcataaggtgaagcttctacaaaatctttacgtacatctgtgtctaatattctttgctttaggtgtgtagtcataaatttatctttaactacttctaccataatattatatcttactttattccactgttcttcattaaaattacgaactttacgacctaaacttttagcatggtttgggttcttagcatttagtatttcacctgctatttgaaagtcattaaagtatcttgctttgcgccacataaaggcttgctctgagttattaaatgttcttccttggtgtttaaactttatagggtagaaattagaataaatatcctctttaccccaaaacataatatattcccttgtttctctcataatatttctcctttaattccatagtgatggtaatacaattttaaaattatctaatattttactttgtacctgttcaagctcatcatatttatccatatcaaaatcatccatttctttatgataatattttattaagcttaaaatatgttttatcatatctatttgtgttctttctttgccgtctacatctacaaaagtatggtattccatatccacatgattactactctctataaatgcatttaggtcagcgtatagctgaataaaaaaggacatgtcataattccaatacttaggttcatttctacctagttttttcttcattttcttatattttttattcttttttatcccaaaaacttctttttcaaagtcattcaatttaagacctttaaaatattttttcttcatttcttaacctccaatttaataaatggaaaatcaatgtttctaaatactgcgccaacatcacacattaacatgtctccattaatttctacttctccactgtctgtaggggtgtgaccacatacataggtaaaaccatcttttctaggttgaaagtctcttgaccatattaattggtcaattgtttgttcttctacaggtttccaactaaccccacctgaatgagagaatatatacttgtcttctttatagtactttctacaattaaccataagtattttaaattttctatagtcgtctgattctttaagtttctttagttcacttttaataaaatcataattatttcttagattttcctctacactactatattttaaagttactgtactcacaccgtaagagttaagtgtttctatacaatatcttgagagccattcaatatcatagatacttaatcggtctacgttttccataatattataaaactcatcatcatggttccctaacagagttactacattatcatcattagacattaaatcaaatatatagttaacaacatcttttgaccttttacctctatctacataatctcctaaaaatactattgtttcttcaggttttctttcattatttattttatccataattgttaataatttttggtattctccgtgaatatcgggaacaacgtatatagccatctaatctcctccttattgtatataactatcttaccatacttagtaaaaaaagtcaataaaaaaacacctattaaattaataggtgtttatcatttaatgttattttaaagtatcattaccatgtgctaattttttatcatctattgcatggtcattataaatatatttaacctctatatactggtcttcacttttcagtgcatctactatagaagcattattagttattgagcttgttctagggtaagtaaatttttgaccgtcagataaaataatagtaacatcaacttcaaagttaacaggtagtctgtatccataatcttccaaataattaataaagttattaagagaaaatggtttatacttgccatctaaggtatagtcaatatattcatttaatgcattagtaagttctgattctgttaactccattgtatcataatctttttcgttatagaatactacaacattaggttgttctatactagaatctccgtctttatacttagatataaaaaatccaatatttcctttatgctctaaataatctgctttcataattttaagtacttcttctgctataggttttgctaatagtgttacccattcacctttttctgcgtcataaacactaggtagtacgtttaccatcatttaaatctcctcttcttaatttattggtttaaaccacaatttactcttatcacttggttctgtttcactaactacgaaagagttagaatcaatgtttaaagtattaaaaacaatttcttgtttgtcttcattactttttgttgtaaattcgggaacatctgttaatatagactctttaccattaatagtccatgatattttaaaagacccttggctatacactgtattcggtgtcagtttttcaattataattttagcggatgcacctgtaattttttctgaagattttaataatttacctttggaatcatataagtttaatgttctctccacaaattttatctcctttactatattttgtacaattaatataacaaaaaaacacctattagtttaaataggtgtccgacagagctcccgtacttagattacggttaataatattttacgacaactatatgagaccctctgtcgttgaaactcttgtcactgcgttattccacaagatattttagaaggtagcttgtggaagaagattgtttttaaaggtacaattagcgtttttaagcctattcgatacccaggacactatgtccgtactaactattacgtcaataaaggttctacggtctcaattacctactctttattgttaaaactaaaattaagcttgagtgctctagaagccaaaatcaattaattaactatagatacggaatggaggggcactaccatccggagtctacggtcagatacaaagcctctgccgggcaacatacggtatctctcgtacatcaggttgactagacctttagagtttttcactccttctcttataaccagtaacttaagagaaataggttttacttagtagatatgaaacaataaatccacatacaatattaaatcatagtcaagtgattgcacatatgtctaacacctataagttttttgctagcctggtatatggactctgcaggattcgaacctacagtcaaaccgttatgagcggttggctttacctttaagctaagagtcctagaaatatcctgagagaggactcgaacctcaacgactaggtagctacatctagccaatgccattactcaggattgctagtaacgctaaatagaattataacgttaccgtagaccttttctacgcttggtagataggtaaaatataatgatttcaaagtacccatatagttaggctcttattctcattataaggttaaaaaggctaactgtgtttagcattatataagaggctttagttaactactatactaataatataccataaattatacttaatgtcaagttaatttatcaattgaatctataatttttgatgtgctacgtatatctgcttctttactatgtttaaggagatattttaatttcattaaaaaagaatttttttctttttctataatatcttctttatcatcatattctgaaaacataatgaattctatacctatactatttctattatgtgaaaacatatttatagaaaaaggtgaatcaaaatttttatcatctttattaatactaaagtcttcagtaacctgtaagtcatttatttcagatatttcaaagtaaccattaactcttttaagttcaatataactattgtatctaaagtaacgttgttcttctattaacttctcttttgttatataaggatattcatttatgaatataggattacttgttccatagttatctctaatatattctgcatcctctagggaatcagtataacctaaaatttcataacttgttgtatacactgtatcttcttcccacaagtcatagtccatttcctctatttcttcttctaatatataaatttttttcatatattactcccaaaccccgataagatttttaagcttagctataacctcttcttctgtttgataagaaaatacccctgtaatatgttcatagttacctacaatttcataatcttgtgtaccatgtttatctactaagtatgagttattcataacatttaaactatcttctgagtaactaaaatttatgttatagtctactaaaaaattaataatatttttcatttacataacctctcctatcggatattgtcctagcattcttgttccattttcattataaaaagtatattctactacaataatattcatcatatctacatatatagcttctatatacggtgtaatattttcctcttcttgtatgtgtttacctatgatatcatataataattctgagtgtattcgtttatctctcattatagacctccgtaaggaattctacagttttgtctttcaaagatttttctactaattccatagcatctttatagtgtttgatattagattcattagacttaagtttatcttttacttcttgaattaggggctctactttattaaccaaatcttttttcttttcaatacttacattgcttctcttattgtctaatacttcttttggcatatatttaacttttgcaaagtctttatagctaacatttaagttatctaaatcatctaataaatcattatagtattctaaatgattatagaatgtataaaacttaacaaggtctttaccagttaattctcctttttttagtatattattaatattaccgataacagaatatgctataggcttaaaattagctctaacataagttaaaaatataaaatcatcataaaataaatctaaaacagttttattgaatctagtatttttagcttgctctaattgagcacataaattaagaacattatcaaacccactttttagaactaaagagataaatctttctactgcatagtatcttgatacttctgtatgcttacttgctttttcattattcctaaatatagtatctgataaaggttgaacaactaaactcatgtaatctttatctgaatgctcatctgatgttccttgataagtacttccaaattctattgttgataataagaaacttttttctaagttcattataacatcctccttttatttgttatttaaataataacatatattgataataatgtcaatacttatatatcttcttctgtatcaacttcatcttgtttatacttaaagtgttcatagactttaaatagtataatccctagtgttattaatcctaaaatatatttcatagcaatcctccttaataaccatgtttagttacccaccctgctaaagcatccatagctatatcatattcttcttcatttttaattcttataattttctctatttcttcctttgcttgcttagaactaataaaatcaatatcagtatcctctaggttagttaattctaagttttctctaataaaattcttttgacttggtgttatagaattaactcttacattttcgtgatttagaaattggtagaagtccatattactcatcctttttaacgtattctgccatatcttttaaaatacttagtacatactctaaatctctatattggtcatctaacgaacctataatagcatatggtgtcatatcccaggcatgtgcacagtcaaaccctaatactctcttaccctcatagtcataatcatcgtaagtgatacctctatgggcacgtctttctaaggagtcatattctttttcattgatatctgaaggtaaagttatatatccatttagatgaccagtttcagggtgtctcttaacagttagtttaactcctttataataaatatcaagacttaaatcttctcctagaatattgttttctttttctactttttccataatgtattgaggtgcttttttaaacataattagtcatctcctttttatttatatctttactatacactattttttatattttgtcaacaaaaaaaggctactaattaaagtagcctaaatattaattatttagcgttataagaccaacgccaataaccatttttctgtgagaactcaaagtgaaaaccatcatagtcaaattcaatattatagtctccatcttgaagtggttttgaatttagtacaggactattactctttgccaattctgctagaaactcatgatttactttttccatagggtttattcctcctaattattcttacagtactaatatatcataggtctttttctaagtcatttttaaaagtttcctcgtaagaactagcgtaagtaacctcataacccactacgttagtatatcctacatataatgacttataattagattttatcttaatatcttctgattgttctagcttatttaagacttcacctaaatcatctgaagaatagtgttcattatctattgttattgttttaccttgggtatagatatcaatttcttgtatcatcatttcatccttttgattattcattatttgattataagtttctaaatcatcaatgttatctgtatctgaaccttttactaaccattctcctctcttcttaaggaggtcatcaaacttctcatgctctttaattatcttttctacctcacttggtattaacacagccctagcatagtttatatgccacatagacatattatcaataagataattaaccattcttataatctctttttcatttgccatataccaacctccttatatctattactaatataagagaaaagcagacttattaaaagtctgcttctgtacctaattctaatcttctatttttcatatgaggaatcatttttttatctcctgttaatagagataattctctagctttttctttagataatgttagtagtccattataattatctactttactattatattgtctgactaagtactctagttcatcttctatacctgctagttctcctgatttaactccaagtaactttctatacatgtcataatcttcagaaagactttctactttgtttttagatacagaatcataaactgcttgtaaattaccttcttcaataagtttaaaattatattcaccaatgattaattctttttcagaagagtcaagggtaactaaaccacttgtattacctgtaaagtcacctttataatctacaacaattccttcagttattttatctcctaattcaatagttccatcttcattttctttaaatttatgagcatcataaacttctactttatcacctaatctcaaatcttgagttaagttatgtttaccaataattctatccattacttaacctctcctttattaatagggtcttgtgttaagaacatttctaagttctcttttgtaataggtaaccaaaaatatttactttccggaattgtaactgtatagaagtcttcatcattattaactttgatgttaacatctgtaaactcatcttgcattaaccaatgggttacagttaagttatatgacccatcactaacatatcctaaatcaatatcatgtctaaaagccaaatcttctaaatgttctaataaatcgttcttttcattatgtttttcttcttctgtattatttttaattgggttaattaattctgtacaaacaatatcatacaattcaccatctgtaacctcatagttcttttcaattaatacatcttgtattttattgattgaatttgtaactactttcccatattcttcttctgtaaacttacatttatctaaatcaacatctgtaattaattctgcaatccatttatttaaaattgatactgccattgttcgagaaataatactatcgtataccatatttatttaatctccttatttaggtgaatgtggtcttctaatgaaaaatcaaaaggcgctacaccatttcttttattatttgtttcttttttaagtataacataagttagtgaaaaagtcaagatagttactacaaccattgataaaagtttaatcaggtttttcatagttactctaactccttaagtttattttttactttctctttatcgtacttataatctttactagagttttcatttttttctttctcttcttcattaagttctctatactgagcttcttctacctcttgttctttattatcgttatcttcttctgctttttgaatttctacattcttactactaccatttaccttttttctaaaaagaaaccaaagtattaataaaatgatgagtaaaataacaatgctcaatacaacagcccaaatattattagccattacaacctacctccgaatagtttttttacagctcttaagttttcagatgaatcgttatttatatcaattcctacgctagaatcaaaaattacagcattatcaagtatatgctctgttaatttattaccataactacttttacttaccacactaccataaccatgattagttaggtcaaccatatcaggttcaacttctagtactctaaaagatattctacgtaagaatgaaggatttactaagtaaaaggaagatttaaaaacatttaatctttgataagaatgttttatattaacaacaaaccctgttaacttatcttcatatcctgaatttgataacttacctaagtaaaggtttatactatatccttttgtttctaatgtttgaatagcacttaacattatagcccctctgtaagcaagattttcagggtcttccatccaactaatactagaattataaaatacatcaataactttcttctctgctttaactctttgctgagacatcatagaattaggtaacccttttatagcattaggtacgtgaggttgatacccttccggagctacgacaggttttctttttactgacttatccattctaaataatgcatctgtcatttttttaagtttaactaccatatcatatgactctctatcacccttaaccattaagttataggcttcttgaaaactatgagtccctgtaaaatcatagctacctgtatctgatgaattatctctacctgaaactctattcttttttaaagcagaaaagaaatcaggtagaccatcatatttaattacatttaattctgagttatctattaatcgtctacccattgattttcctcctattctaatcctaatttatccataattgtatcaaagtccattgaatcttttgatgtactatcagattttctaggttcctgcttaggctcttgttgcatacctaaaagctttcttgttgcttctgtgtatctgttaccctcaggtaaagagctaataaattgattaatctcatctttcggtacagatttaaagataatactttctacaacaaactcatcttccattactccatctaatttactaccattaataattgcacgcattgagaatacataaggtaatcctttttcatcattctcatgtcttaattgttgtacaaagttaactaggtcttcattgcttgataactgatgttccaccttagtatcatagtcaaattcaacttgagcaaagcggtctaatgtagctccgtctaattgttgtctacctacataaatatggtctgctcctgttcccatagtattacctgctgacacaactctgaaatcttcatgagctgttacacgtccaatagggaagtcaaagtatttatttgcaatagctgaattaagaattaatagtacttcaggaatagatgcatccatttcatctaagaagaataacccaccttttgtaaatgctttatagaattgggtttcatgaaacttaccatttgcatcaataaatcctgttaatttaaattcttgagtaattgcattactgaaataaaaatctaaatctagagcttctgctacttgttctaatacatggttcttacctgaacctgctccaccttttaaaaatactggaatattttggttaactaactttagtatatcttggtatctataatggaagattcctgagatatctttaattgttttttccttcttgttgtaattcaattttaactggtaaattactaagttgttcttctacatattcttcaatttgttttttaacgtcagtaataataatttctctactctcagttcctgctttctcaacaattgcatctacaattgcttgttcatacggattagagtttttctctcctagtttgtctgccaagtcttttgttgtttccatttgttgttctaccaatctctctaatctttcaatagtatcttgctttgccatatttatcattctcctttgatttgttatacatttattatattacaagtatttgtatttgtcaacaactttctaaaactttttttagttgttaataaaaaaaaataccttacacctataacttaacatagggtaaggtaattgtcaacacttttattaaaaatacattaatttaaaaaaatcatcaatatctttagtttcatgtgtatccatatcatacataaacatacaattatatgtatgattattcattatttctaacatgttatgcatagaagttgcattattgaattcctctaaatcaatagttaccgtaagttcttgaccttcataaagtatgtttgctatataatatttcctaacaccttccattgttccatgagaagtttcattatgattaagtacttctacacctagtgaaggtaaatattctgaaaagtaatatttacagaaatatataaaattgtctgttcttttagacacgagtactatctccgtactttatatttctttctaatcgtacataatatgttttaattttttgtacttctttatctactgcatcctttcttcctaaccttgtagtatattttacaatattaaatatcatagaatcaacaaagccatcataagaaaaatgttcttctagaaaagaaataacatccttactacctttatagtgttcaggtaaatgtgcatctacttgtatattataataatcttctaaaagacctatactttcaccaagactagataaagcgtaacctaaatcatttgaatcattagaccattctttagatactgatagtgcatcttctataattgttacttttaatttatctaaataatcttctacttgagcttgtgttttcataaattcttttgcgttcatgtaataccctcctaaattatataaaaaaaaacaccctgcttggctacaagcaaggtgaaaaaggaaagatattatggaagtgtactatctaagtacacctcataatataacagttttccttgctagttattacttattttttaaggtcttcttctttgacaaacactccattaataagcttaccttttctgtcttttatctcatcataagccatatcaatacactcttcaatatctatatctaactgtaaacatagtactgttaatactacaaaaatatccccaacactatctcttgttacatggtcattacttttagcaatacctgaagctaattctcctgcttcttctaataactttaacatttgaccttcaggtttacctgtttgtaaatttctatcttttgcccattgtttaataagttctactttttccattattctatatctcctttaatttctgtatctttgataattaggttatcagagtcacttgttacatttaaattatcttcaactaattcatgtagattattagtaatatcttcttcatacctataacctacacgaacataagctttaactctgatatctatattaacataatcttcttggaatttttccatttctaacttcctttattatatcatattatgatactattgtcaattaatctgagtagtttcctttagcaagttgatactttttgtgtaattcttcatataattctctcataccttcatagtttctcatatcatcttccaagaaactaaggtaatctaataatacttttacatcctcaggttctaaagttataactggttttaccattaggcaacctccttaaattcttctttatttattttcttgatatctttttctaatgcttcttttaattcattaggtaatttataggcatcaattgattgttgttgacctaatacataaccattatctgtaatacgtatttccactgtaaaccatgaattatctaaatcttcttctaatcttgctaataatattaaacaactattttttagaattctgttagcatacccaccaacacaatgagataacattttaccttcatctttaagtttacttacagtatctgcaggaaggaattttacttttctaccatcttttaatttataagttttatcaattattttttctaatttattatcatatttagctttaagttctgcatcatctaattgttgttgtatagattgtttctcatctgtaactatatcatgttctagttttagtgaaaatggtgttaaactaacactctctaatgttctataaccttctcttattaatattgataaatcatgtaagtaatctaagtaatagttatctagtgcatatcctgttatacgttgtctgtcttgagcatctacatctaaatagtgcgtcatctttttgtaattagcaaaagatatagataatatttgattcacaataggtttaacttttaaagcatctgtaacatctcttgaatctctaaccattaaaaaggtatcgtcaaacaattggtgtaaattaacttcattgtgtaaatgattatagtgcttatagagaatattagcaaatcttaagtaattaccttgctcaaatttatttaaagttagtaactttttataagtctgttttgtaagattgaaggcttcatgaactttccatttaggttttttaggtatatggaatagtaatgcatttctttcaaataacccaaattcttctaagttatttattttatcaatatttttaacaatatctgttaaagttattaagtaattagaagttgaattttctcctataaaaatcctatatttatctcctctataatttatatgaccataaacatctgtattatcaggacaccaactagaaaaatcaaaattatggtgctctaatgtttgttctattatctttattataattcctctagttaagttaggttgtgcatagttttttaaaataacatttaataaaacagataaagttaattcattcttatattcacttttactaatatcatctttatataggtttaaagttatttctttattaacaagactatctgttaagaaaaccttaacttctcctgttttaacgtcaaatgaacttttattttctaaaacccatttgttacccatattatgtttatctctaatatgtttaactttaagaccaaaagatgaatagttttcagtacttggatgcatgtaccaaacacggctatataattcatctgtcatagcactatagtactcactagaacctttttctatatcttcattcataacaataatagatgactttataagaccatatttaccttggtctagtacatccataatatcattatttaaactatctactacttttttatattcttctaattgtctagattcattcctccataaatggtcattaccttctagttctttaattttttcttcaacataaccttttgattgtatacgtcttctactcttatcaccatatacaggaaaatcttttctttcttctctattagattcaatatactctttgtaacttcttcctttattatcatcaactacaccttcaactaatttttcaactgtttcataagggttaccttcaaagtttgttacttctttattaccacatagggctaaaaataaatgtatttctgtggctgtatcaaaactaaatatattatgaatatctctaaataattctttagaacctaagttaattatattatttttctttttcttaagaaatacatcttcttctcctatatagatacatcctttattaaccttaggtaaattaataatttcttgttctgttaatcctttttgtttataagttattgccatttaaaatcactccttatttgttatgtactaatcataccatagtaaataatatttgtcaacaaaaaaagaagaactttttaaagtccttctaaatgagtttcgtatataactttttgaattttatttaatggttctaaatctaaattcataataagttttttatactttcttgaatttttaaaattgatagtatttggcatagcaagagcttcatcaacatctttagtatagcttacaacatctgaatagatatctacttcttttacatatagaccttgagttaaactcctaaatactacctcattatgtgctataacttcttctttcttttctatgctcatttataaacctcctggtctactctacacaaacaagtacgtattctaaattagttaaagaaactgatttaatattgtttaattcttgtaatttcttaatttccacatcatagttcttacttatagtccataatgtctctcctgctcttactttgtgataatatttatttccctctttgataaggtcattcaatattacctacccccttgagtaataattagcttgtagataacatataagtataagaacaaagtttacaaattcagtagctataatatgaacataggtatgtgttaaaaccatacttacaattaatgaagctaatcctaatccaataataagaaatagaaatctatttgttccttctgcacttttagttttataaaaggttgttatctgagttacatacgcaaggataatagtaatagttgctacagtttgtgttaaggctgtaaagtcacttaataaaaatagtaacagtgagaacacaataataaaaggtatagagaaatagtccttttttctatatgaagctactaataagcaaacaatacctaaggttaaattaagacctacagatactatttgaaatactgaagcatcagttaataataagttgtaaaaacttatacctactgtagctacaattaaataccaaaaatagttactaactcccttgacactttctgctttaactaatgctactaaacctggtatatatcctactgtaattaatatagcatataatatacttaagtaatgtgataaattatccatcttgttcccctaatttctctaatctattaataacttcttcccatgaaataaatccttctccgtctgttagttctaaaaccataccatacacaaattggttcgtactaaattcagctctgtcagggtcattgtatggtttaccatgtccttgtctaatatccgagcagtagattaatacgggtttatttacaatgttttcaagtttatctactattaattcttcttcagtatttaatgacgtttcaaacttatttgtaaaataattaaaatactcttctccattatcatatatatggttaattgtttcttctgcttgatgtttcatacctaataaaataccgagttctgcaattgttcctaatccttcattaaggatatcaaatacaaaaatatctgattcttgcatagccttaaagtcattagttaaaatacgttctgctagcttagtttgttctgcattagctttatcatttattgacttatctttgtgagggctataaggagttactcctacaatgccatctacttctttatgttgtttatctctgtaatctaccatagcttcatttaggatatgtccacccatataaattactttgtctttaattttattacccatctatagtatctcctttttcttctaaaatttctcttaaaatatgtggcatttttttcttaatttgtttatctactattttcagtatattttctttttcttcttccataatatcatcaacaaagttttgacctacttgtttcataattagaccgaagttttctaattctaaatcatcttcagataatctatcttcttctatagccctaaaaatcattttttccattcttgctcttgtaatggcataatctgccactgactcgttcttttttacttctgttttcattttttgacgactaaattctttaaactcattagatactaatttaaagtagtcatcatattctgatttaccatctaagtatttaattactataccttcacccttatcaggtttaactgtcatgtcagattttcctactaattcttgaatttcttcaggttttaaatcatttaagtagtgagatggtttagatactagcaaagttttaactgtttttaaccctaaatgatgtgcaattacattcatgtcttctactgataaataaacttcattttctttatcataaacatcaaatacataaaaattgttgtaaaattcttctttgtactgaatcttatgtttgactaaccattcaccaaaaataatgtatttttctaaggctgatacgtacgtatttcttacatttatattttcatgtacccaatcataaaaaccatttaaagtttcattctcatttaatttttttctacgtgaaaaacatactaattcaccattttctactgtgaagcttgcattacttccatctaatttttcttgtacaactagacctctttctttaaatttatctagtacaatacctttattttttactttagtatacgatttcattaattatcctcctttgaattatgtactatagaaaacaaaataagacttacgcttgctaaaaatgctaatactactaaaccaggtaaatttaaaactgttgataagaataatgatattgcacttataacataaactagaccgcttagaaataaagttaataatacaattgttataagtttcaccaaccaattgttattaataaataccttagctaaataattcataaaaaaatcctccttagttattatagaataattataccataactaaggggatttgtcaacatattattttaccatttaaaattatctgcatattgtgcaagcttagagcggaaattaactgtaaaattatgaaatactgctccttcataatttttaaagtattccatataatctccaaaacctgatttactttcgttctttaaatctatttgtttaaaattaccttctactattacagtagaattttttgtatgaacccttgtaagaacttttttaagttcactacgtttaaagttctgtgcttcatttataattatagtagcatctcttagatttccacctcttaggaatagatgggatatttgagatacccaacaatctcctagtttatcttctttaacattatcttccatcattaacatttcagttatttgttgttcaggattcatattaagttcaataagggcatcatgtaatcccatgaaataagccatttctttttctgtctgattacctggtctgcttcctaaatcttctgatactggtgaaattataaatactagctttctatttttattaagatagtctgcgtaagcacaggctactgagcacattgttttacctgtaccggcttgactctcattccaaagtatttcaacattatcattaaagaaatcctcacagaaatctaactgctcggttgtagctttttcaaggaattcattaaagactagatgttctcccatgttgtatcttacattaggataatcttttaacttaaagtctaactcttttagttgtattgccatattttaaagttcccctatctataaatagttttactctcttttaatatagtactaatttccgatatattctcctgttgaagagcaataattactacattcacattcagggtagttatcacaaacatcttcatcttctacatcatcataaccaatatcataattattataattaaaatctacaatacaattttcactattacctttagataatcctgtataaataatatcatccacagaatcccaatcgttatctgccaagtaatttacactatctagtactgattcattatcaggtaaataaatactaccgtctgaaaatttaattaaaatatcaccttgaggtaaggtatcattaattaaatcaatctctgtttcttcttcaatagtgaatacagttccttctaatctttccggtgtagtatgtgttaaatgttttacagtatcccctgattcttcatagaatcctactgcattcatatctttattatattttgcaataaatttaccattgtcacttaccaaatattgactagttgcattatagtcgtttgcgtcatctactgtcatgcaagggttataatctttaacataataactaattttcctaacatctgctgtttgtactttcttaccttcacctttaattactgaattaattttcttcataatattttctcctttttatatatcaattgatttttttgcaagattatcggcatagtcattccatttgtcatttgaatggctctttacttttacaaagtttatatctattactttttggtattctcgtatcatattaatatatgttttacttagaatatttcttgcagaccaagtaccttcataccaatgtattaaaccaatataatctatataaactattgcctgattgtatcctagttttatagcctcttcaataccataacaacaagccaatatttcacctgcaacattattatactttattaatcctggtttgtcaacacttttactaatttccgatattatatttccttctttacttaccaagacagcacctgagcctactttacctttattatatgaggagctaccgtctgtgtatatatttacactatcctgcatacttataatcctccataaattgagggaattcacaatctgaatagacttctctgcaaaaagatactgagatatagttaaaatcaaaacatttgaaacagtgttcttgaacttcttttttatctttagcaatcacattaaatttaaaaccatcagctattactgtaaatactccttttttcataaaacaaatacctccactaattttattttaaattaataactaactcaataaatgatttaatagttttatttttaccttcatcaatatctgaaaagaaattaattaaactgtcatcctcatcaaataaatcttcaacatcatcaaatttatttaatatgtctgtaacactgtaaccctcttctgatatatactcatgtaagtcttctccatcttctgacagtgttgcttctattttaccatttttactttcaattaaatataaagtatttaacactttaacagaatctacaactacactgtagttactaatagtaggatactctgtataaagtatttctacattagtattcatataactatcaattacagagttaactgtatctctttttagctcagatacattatgttttcgtatagtaggaaattcttcatcatattctactaattcttttctatctgtattcaataacttgtctaaagaggacaacaatactattttatattggttatcaggaagactgtctgtaatttccattattgttaaaaacgtatcttcacctagaactttgtttatatcttgtaattcaaatgaatctaccatttcaatagtatcatctatatcatctgtagtcattaaaaaattaactaaattattattctccatcgtcttcctccaattctttaaataactcttttcctggagtatttaacgctttctctaaccgcattaaattagcacttcttggtttcttttttccatactcccaataagatataagagagtaatgaacacctatctcagaagctagacttcttaacgtatgtcctttttctactctaattttttgaaggtttagaggtttactttcctttttttcatccataattatttctcctctacttttaaaaatttaaaatcctcagatgcttttgcattttttagtatatactcttgtgatttatttcttgcctctgccttacttttagcatataactctatatgaaatacatgaggtttttttaaagacggtgactcgtatctccaataaactttaaaaagtagtgtttctttttttaaaacattaattcgaaaccatcttttaaatttattcattcattatcctcctctatttatttgttaaactaattatagcatagttaacttatgaagtcaactataatatacaaaaaaagactaagaaattaatcttagtcttaatatattaataactattatgtgcgttgtggtatgcaagagctcctgatgttgaaccgtaacggtcaatcatatattgttttgcacctttagtttgttctgctatagaaccaccactccatgatttacctaatccttggaataatccttgagctcctgatgatgcattaacagcattagggttcattgtagattcacgcatagcaatttcaatcattgcctcgtctccacctgcttgtctaatctgttctgctacagagcctcctgtagaactagttgattgtgtaggttgtttagtttccttttgaactggtgctgatgttgtttgtacttcttttttagtatcttgtttattttgagtatcaaattgtgcttgttgttggtctactttttgttcaggtgtttgttcttctcctgctaatctagatactgtattatctacttgagttgagcctgaatggtattcataaccaaagttaccattataattatagaaatgataagtaaattcaccatcactaaaagagaaatcataattacctgcttgaattggttttgtatttacttctgctgaatttgatttagcttgttctgctaacttattataatcaatttcgtctgcactagcttcatttgtagcaatacctccaaaagtaatagctgtacctaatgctaatgttgcaaaaattgttttcttcataaatttaaaactccttaaataattttttagaattgtttatttgtaaaccgacataagtaatcataacatatatctttaaataacgcaagtataatatagcactaattagtgtaatattattaaggttttattacaaacattacagttatcagataattaaatacaaaaaaagagaggtaattatatttaccactctccagtttcattatatttatttattacattatctcttaattctatagcctcttctaaagattttgcactaaaatatttaactttattttttcttacaatttgtatattatatacattatttgattttttgtatatattatgtgtacttttctttcttatattagttgagttttcagacctagtagaccacttaacattaccaggttcataattaccatcattatttattctatctatttgataattttcattcggagggtctcccatatagtcgtagaattttttaaaatcattcttccattcttcacatatttctataccccttcctccatagtttttataatttatagcgttaacatcgtaacaacgctgtttcatacctaaccatctttggtacattgggttactggataatccatgggtagtattcctttctctcattaactcactatgaattttattactctcacacccacaagattttattttaccttgccttaaagtggagcttctaactattattacttctccgcattcacataaacattcatacattttacatcttgatttatccctcttactagactcttttataacttttagtttattaatagtttctccaatcataatatctcctcctataataaaactatagcataaaaaaccacctatgtcaataggtggttaaacatattatttatttaagtttgtaataacactatctgaacctatactaataggttgttttccattccatttttcaattaattgttgttgtaaaacttcctctgttaaggattcacttctaatgtcattggctttcttttcaccttttgcttcaatttctttctttttagcgttttcttctgctatttgcttatcaactttagtgcgctctagttcttggtttgcttttactctctcatcaattgctttttgagtatttttatctgcagttgggcttgataatgcaatatcatcaataataaaaccttgcttttctaaattatcattaagtttatttaaagtatcttgtttaatttctcctgtttttacaccaaaagcatcaattacagagtacttagaaattgcttgtctaacattatcttgtactctagaacgaagataccctttttcaagttcttctatgtcagcacttccgaatctattaaaaaggtttactgccttagttgcatctactttataagatacatcaatgtctaatttaatatttttaccatctgaagttgctacatttaaatctttatatttatgtgtttgtgttttagttgggtatttatttaccttatcaaaaggtgctgttaaatgccaacccggtgatttagtatcttccttaacaccatttactgagtatacaactccaacatgaccttgtggaatcttagtaatacacattaataaaataataaaccctataattgctaaaaaccctaatactcctgaaataactactgactttctcattacatttctcctttttctatttcttttattaagctatttaaagctttttcctcttggtctatttcttgtttatcggctctagttacaattgattgtctacggtcatttaagaattgttttttatactttacatattgttctaaaccgtattcatctaatgtaccttgcctaactaattccctgtattgttttcttatgttactcttcttctctttcattgaaagaaaatcaaataaataacttataccaaaacctacaaggactagaaaaacaataaaaatagcaaaatatgttaaaagtagtgccatgtaattcctcctttatttgattacatatataactatacactatgtatttaattttgtcaacacttttttgcaaaaaaaatagacggattttaaatccgtctaaatttatattctatttgaatactccccaggcaacgccaggtatttgattaggtggaacaccttgacaagttctaacagggcaatatactctgttaccgttgtaagcattataacctatccaaatgtgacctgcttggatacaaacttcgtcatatacaattgtagcccctgccggtaagttaccgcctactggagcatttaagaatggagaacctattctagttactataggttggttaccattgacaaatgttgcattttccggtttataccaagttccgtactggttctttttccaagaacctgtaactggtctagttgccggtgtacttgcgctacttgttttaccatctttaactactgtagaacttgaagttcctttatccatgtagtttttaatttgtttaatgaaataatcttttaatttattcattattgcttgtgatggtcttccttgtgttactggattaaatcctgtatgaagaaccatagaacggtgaggacaggcagttggtacaaattccatatgcaatcttacagttttacggttaggagtaagaccccattctttaaatttctctgctgtaaattggaatactgcttgttcatttttaaggaattgagcatcactagcactcattgattgacagacttcaatacctgcaaatctaaagttacctgagtttgctcctgttccatccgattattcaataaaagacgtaactcctttaccggttctcttatgaactccttatagtttcctataagactagactatatcttcaccctaattttgttaggggttctccatttcgatttaagggattctcacccactccattaacttgagccctactcctattgacgatttatttttattggcactcgtccgagggatagtcgttgaaccttaatattgtttccaaatgtatccgtaggctgtttttctctctcctttgatacatcttcctatattaccacttcttttattagaaatacctaagaactctaaagcctcttgagctgtgtaaaagacattcaataaattaccttctaagtcatactgagctatattataatttatttcattttttaatcctgtctttattgcatgttctctgttctcggaattagaaacccattctaaatttcctacactattatcatttttaccatttaaatggttaacttgttctttattatcaggattaggtataaaagccatagcaactaaacgatgtattttaggtgaatggtatcgtaaccttacaaacaagtaacccttgttatttttttgaagttttaactttttaggctctttacctttataagatattacttctcctttatcagtaatagtgtaattttcatatatttctaatccaggtatttcatttaatttcttttccataataacatctcctttacttaagtatataggaaagttattatgttgtcaagtagttttttaaaacaatattcttggatgctgattagcatagcttgatagccttagcctcccagtcaattaaaagaactttcattatacattactgtataacagggcaatttatttacccgtgtgccaagcaatttgattcttagcatctattgcttcccatacataaccttcagagccgtagtaatgagcaataccattagcgtatctagcataacctgcattagctaatgaattctcgtattgttgtcctgaagaacgacctgcatcgttgtgtattaccattccttcaggttttttaccacgtttatccattgtatagttaatgtgattcttagaaacttttagtgttgctttctttttaggtgcaggcgttttacttgcgcttttcttagctgtttcttttttaacagtagttcctgcttttacaggtatttcaatgaagtgagttaatccgtaataattatctacacgttttgtaggttttttattagcataaccattccagttttgctctaaaatagtaaatgtagaagtattacctccatcatatacaatacctatgtgaccccactgttcataactaccggatgtaaataccgcaatccaaccttttttaggtacagtagaaggtttattttcatgtattttaaatccagtaccataactctgtttaatttggtctttagcattaccccaagttctaactttattatctgttaaccataaaacatagtctgtaataaggtcttgacactgagcgtgatagtaaccatctgcatcaatggctcctgcttccattacaccaaatgatgggtcataacttgtagcttttttaactctgtaagggctatctactgttccttttgcataagcatctaaacgtttatttatttctgcttgagtcttagccattacttaacttcctcctctgcaaatactttaccatgttcctcggtatcttcttcatcttgagaaggtgctgaaccaccatcaatttcatcttcaatagcaggtacttcatcactatcatctgtgtcaggttctgcattgttttcgtagctgtctatctcaaaagtactagtgttatttgcatttgcttgccattgaacgaattcattagggtctttactatcacgaggtttaagatagtctgtttgaacaatatcactatctttaagacctttagtattattatcaacaataatacctaaacctgctaatagtgttagtatagaacctacaatatttacaccttgctcaatttgagctgagtagtctaaaccgaaagcacctataatttggttagcaaataatgctactgctgatataattgctacccaaaatgttttgctcttagttcttgtgctaaggtttattcctccaacaactttaggttgtttagtttcattagccattaaaaaaccgacctttctattatatttatttctaacaataatataacagtaggtcggtcatgtttatctatattaatttaacacttactcattaatttggtttagttttttgataacttcagacatttgtttgttatctaaatcttctaatttagtttcaggaagtagctctaacttatcccaaacttcttctttattagatactttattattaataattgccttaccaactaaactttccgtataatataattgttttgctgatgccatttgtatctctccttttaaatatgtaaagtatatagctagtatcgtatcctaggaacaaacacttgcgctatatactcaatgaaatcctaccctcattcgaggacacagcaaaccggttcgtcaaccgcacatatgaattctcagatttcatttatgtaaaacacaccctctttgatttgcacaaagactaagggttttggagacccttgtactactaattatactaagggtgtttattatggtttctattggatttgaaccaatgacacctagagcttcaatctagtgctctaccatctgagctaagaaaccttaaaacgacccatacgagactcgaactcgtactctctgccgtgacagggcagtgtgttaaccagttacaccaatgagccaaaattataatgctataccctaaccttaccttaatgtatagcaggtttttatataagctcgaagcaacgattattaccactcataacaactatatattaagtgaaaggaggtgaaatgaacaaaacgtggtaattggtacttatataggaaatatgtataatctacaaggagtaagttattggttcataaaggagtgtgaacaataaatacatgaaagagtgaaagtttactccctgtagattctttttttaattatcaatcaaaggaggaaactgataattgttaataataaactataaagaggaaaatatttatagtcacattctgatataatgcaactaaatatccaagcataacccgtctcacgaggaacctacctataagacctgttattaagtgaatcactacgattgactctattaaggagctaccttaagtccatctcacgcaatttaaaagggacttacaaaccgtaaaacggtaataagtttattaaataatgtgatattaacatattagttaataactttcacatggtcgaagaaaagtaaatttatttgattaccaaattatttttatcaaatatagctcttttgaacctgtagatttatgctacttatactgataacctctattatctaacacatttctgtgctccaactacagttagtcgttacagcgtatctttctaggattccgctaagaccctaaaaagaaattaaaccctagccgttatcatactctacagaccttataagtaagtaccaagtataccaatcgtatttaacaatactaatgacgacccatcctaccgatatatctccgataggttttgattcgtttgattatcttgtaccttatgactaccaaatcattattcagtcactatgctcagatatttagttgtattatttatatattaattataacataatttttattacttgtcaagttaatttcaaaaaaattatagaagtagggacgtttacctacttctatttaatttacacaaggatgataacattgttattgttttatactggaaaacaatgtaataaaaacagtgatgtgtaaggtatttgttttattgttaattatattatagcatatactgatacctttgtcaagttaatttaatactttttttaaaacattagttatcttttgttagttcctcctgaatagcatcccatcttctttctgcttcactacgattatcttctatatgttttgtagttttacaacatttgatacaatatatatctttgatatgaccttcttctcttttatttgctctttttcttggtactttgaatacatttccacattctttacatattaaacttgagtaaaacattttttgtcttttcataattaatcaattccttttctcttttatttgataatttaactatatactatattgataaataagtcaacagttttctaaaaataatttaaattattttgaagaatactttaatatcaagggttacaagagaaaaagtacgtatttagaaaataaggagtactcctattatatataattatattctgatatagagtaataaataatattaaatatataattataattaataaggttgggaaaattgatataaacataactgatactgcttatagatactcagtataaaagtaaaatcccttagtatcagtacttacaggcaaaaaagtacgtatttagaaaataaggagctctcctattatagttatatatatttattactattattaattactatttaaatatataattataattaacaatgttagaaagtcaacaatagtataaataaaaaagtgactacttaaagtcactcaataattagaatactattttaaaagattctattctgtttggattaatatatacttgaggtgaagttatagcactttcagtatatacttttatagaggtttcatccattcctcttaacatataatctatatcttgcctattgtaactcttttcatcagtagatactaaaaagtatttagctccacttgacattgttatttcaatatgttttgacatctacaatctctcctatgcaaatttgttaaagacaaaggataatatagctcctagaacaagtaaaagaaccttctcagttgtatcctttttcttagtatccttagtttttgtactttcagcaagttctgaaatcttttcatcaagtctttctaattggacgtaaattgctgattgtttttcactattgacagctacatctttatctatactaactatcatttttcttagttcagctacctcaacttctaaatctttgaaagttcctctatctatataattaccttcttgtatcttagacttaatagtttctacttgagaaacaaggttgtttatctccttatccaactagaatcacctctaaggtctaaccgtttcagattcagaatggatatcataattttctaagaaatcattgataatctccatataattatccgtaacgacttttccgtaagatgtttttgtatcaatttcaaacctaagcttaccaaaactttggaggtctaattcttttattacaatattagggtcatcagaaggaaggtaataatagtcgaagtatataattgagccatttattaatactctgtctattctatagacgtggaaatagcgtctgtctcttttaaaatgggctagtgcatctttaaactctaacttaaggatatccttatatttagtcaaagtggtaacctccttactattaatttttaaatttacttattttgtggtataatagttatgataaaggcagttattataattatattaagaataatgataataattattttttctgagaaaataagccaaatactaaaaacagataaagcatagatagctgatagatatactatattaagagttaccttacttttatcttttctatagatagaataacctaaagacgttgtaacaccactaagtataaaataatagaaacaaaaaagaggtatagacagaaaaaaagatacgataatcattgttaaacacctatttctttttgacctattatttctagaacttttagattacaccactaatataacattaaaagccagtcataaaagtcaattgttagattaataatataataaaaaaagacaataggaggttaaagtggttgaataataacatagctatattcatattcaaaacactggttatcattatattcttactactaattttgtctgttattaattccttgtcccttatttactcaataagaccgagtgtagttatgacatactttatctttggtggtattgtttctaatgtcgcacttactgtaacagataagttcttactgaagaaagaagaccccctacctgaatatgttcttaaaaaagtagagataaatgataaagaaataagaataatcaagaaaataatagaaagtaattatggtataacagcagaagagataaaagttagggctaaagcacaaagaagagtagaggaagatagtaaaaaggaagattacaatgaaaacaaagaaagaaattaaagaacaaaggaaagaacttaaggatggtgctacatctgtttctttagtaaaaaagggagataagagaatagctagccctagtagaatttgtagtctatgtggtcagcagttatcaggtatgaattacactaaaggaaaagcattatcaaaagttaatcattttcatttacagtattctaagtatatttattttgatatttgcgcagatatcaacaattgttataaaaatttaagaaaacgaggtgaaatggattgagtgcagaaaatattagagatataattaacaagaaaaagttagaagaagaggatacaagaaaatatatagctgatgggtttatgaatggtatcggtaaattaatgtacgaatttaataagaaagtagataacaaagaaatagaagttaaagacccgaatgatttatacaaattatttgtgatattctctcaaatgcaaaatatggtcaatgaaacttctgaaggaggagcaatacctcaactatctagacctcaacaggaattatttgatgagattacaacagaagatagtaatggagaatctacagttgatttacagaagatatcagaaatgtcagcggaagatattacagcaatgatttctgaaaaggaaaaagtaatgaatgaggaaaattcagaaacattctaaggagaaagatataaatggatggaaaagaactaattaagatagcacaagaaacatttcaaactgaaaaaataacaagagaacagatagaccatataatcaatatgctaaatccttctacctatatgcttaagtatcatacactgagagggcatcctataacttttagtattcctaatagagatagaagtaaagcacaggctcatagaccttggcaaactaggattgtaaatgatactcatcctaataaggctgtaataaaatcacgtcagttaggtcttagtgaaatgggtgtaatggaaatggttcattttgcagatatgcatagttatgctaatgcaaagtgtctgtatacattccctacaaacgaacaaatgaaaaaatttgttcagtcacgtttgaaccctgttttagagaaagaatattttagagacattgttgattgggataaagactcgttaggttttaaaaagataagaaactctagtttattctttagaacaagttctaaagcaagtactgtagagggtgtggatattgactatttatctttagatgagtatgacagggtaaacttattagcagaatcgtctgcattagaatcaatgtcttcatcaccttttaagattgtgagaagatggagcacaccttctgtacctgggatgggtatacacaaattataccaacaatcagaccagtggtattacggtcatagatgtcaacattgtgattacttaaatgaaatgagttataatgattacaaccctgataatcttgaagaaagtggaaatatgttatgtgttaatcctgaaggtgtagatgagcaagctaaaacagtacagaatggcagttaccaatttgtttgtcaaaaatgtggtaaaccactagatagatggtataatggtgagtggcattgtaagtaccctgagcgtacaaaaggtaataaaggggtacgaggatacctaataacacaaatgaacgctgtatggatttctgctgatgaattaaaagagaaagaaatgaatacagaatctaagcaagcattctacaactatattttaggttatccttttgaagatgttaaacttagagttaatgaagaagatgtttatggtaacaaatcacctattgcagaaacacaattaatgaaacgagatagatattctcatatagctattggtatagattggggaaatactcactggataactgttcatggtatgttacctaatggtaaggtagacttaatacgattattctctgttaaaaaaatgacaagacctgatttagttgaagcagatttagaaaaaataatttgggaaatatctaagtacgaccctgatattataattgcagataacggggactcaggtaataatgttttaaaactcattaatcattttggaaaagataaagtatttggatgtacttataaatcttctcctaaatctaccggacaattaagacctgaatttaatgagaacaataatagggttacagtggataaattaatgcagaataaaagatatgtacaagcacttaagacaaaggatataagtgtttatagtacagtagatgatgatttaaaaactttcttaaaacattggcagaatgttgttattatggatgaagaagatgaaaaaactggagaaatgtaccaagttatcaaacgtaaaggtgacgaccactatgcacaagcaagtgtctacgcctatataggattaacaagaataaaagaacttcttaaagaaggaaacggtacaagctttggttctacatttgtttctactgattacaatcaagaaggaaataaacaattctactttgatgaatagaggtgaaatagacttgacagataaattattttatggtacaattagtaatgaagaaattaataaaagtgtattgaatttgttattgggtgaggaattatccttagattatgtttctaaaaatagtgatactttagatgttaaatatgaacatgtttataaatctctaggattcgataatttctttgattgttttttatatgctaatagagagcctgaaatagtccataaaggtggagataaaaatcttggtggactaaataaggttaaacgtactgttattcgtaatggtaaagaaatggaaatgacagtttacgaagatggtaataaagagaacgatagtaaagaaaaacaagaaggaaaagaagaagttagtagaagtgcagtaggagcaagggctatttctaatggtgaagaaggaaaggtaaaccctaaaaaggtagcaaattcattatctaatttaagtaaaaaaggtgtagatgtatcacatattaatacaaactcatcattgtataaagagtttgttgatgataacggtgatacattaggaattacatcttttaaacgaactgaaaatgatataatattagaatcttatgcaagttcacatgattcagatggtgtaggagcaagagctattatggaattattacgtttaagtattaaggaaaataaaaatgcagttgtgtatgatatagaattacctgaagcagtagagtatttaaaaactttaggatttaaacctaataaagatggatacatcttaagaaaaaaagatgtaaaacaattcttaggtgattatagtgattttatttagcactatagtcatctattctattgtatttattctatatattgtattaaaaacaatttatataaagtctaatatgagtagaatagataacacaactgaattattaaaaatattacaggaagatattgaaggtaagataaaaaaggaaggaagaaataaatgactttagaagaaaataaattaacattagaagaatcaataactccacttagcaaagaggagaaagaagatagtattaaagaatttagcagtttattatgtgaaatggtaaatagactatataagtcttataatgtatttagacaagaccctatggatgaaactcaacgtctagatggctctttaatggtctttcaaagtagattaaatgaccctttaacaggagatttacatgataagatgtataaacttgctttttcaaaacgtattgatattttcgaagctaataagcaatttagaaaagatgtagaagcaggtaaagcaattgagttaggtgatgtagctattatagatacagcattaagtaacatcctttcaggcaatgagttccaaggaagtatttcatttatgcttagaaaagactttgaagaaaaagaacgaattagaaaagaagaagaagagaaacttaataacttataaaagggaagaattatgagactatataaaatgaggtatcataattgaaaaagaaaccacaaggcaatgaggtaatcataaccataataacggttatgatagcagtatttgtagtcattatgaccatattttttaataaatatcaagatgctaaagaagataaagatagatatcaaagattagtagagatttataaaaaagcagatgataatgatggtgagactaaaaagaaatatgttaaaagattaaataaggctgaagaagaacttaaaaaagtaaaaaaagaaacaaattataaagattataataagaagtcaagtaaagaaagacaaaaagaagataaagaaactagagagaaaatatatgatgtaactggtgatgatgacttaatattagtaaaaaataatattgagtttagtgataaagtagacaagcccgaaatacttattagtgaagatggaattggtacgataactgttcctgtagatagtgggtatgaaaaacaaacagtaggttctattattactagtgtattaggttctcctttcctatcacctggttcaaatagtatagatggtttaagtgttattaacgataatgtttatccaaatacagtagatagcatagtagaagatacaaaaccttctattaacttaccaacggataatcctattataacaaatccagttgaaccaactataccttcagatattatacctcctattgataatccttcagttccgatatctcctgagaacccaggagataataatcaaggaaatacagataatccaaatcctccccctccagggtacacagatgaagatggtggaagaggctccggtggtggaggaaattctgaaccaccatcaacggaagaaccttcggataatggtaacaccggaggaggagattgggaagaaaaacctgacccaggagaagaaccttcagataatggtaatacaggaggaaatggtggagaagttacgcctgaacctgaacctgaacctgaacctgaacctgaacctgaacctgaacctgaaccatctgaaccgtctgacaatcctgatgaaaatggaggatgggaaactgaaccaactgaacctgagtcaccttcagagccggacgataaagtggacgaagaagataaaaatgaagatactacagatgataaacagcccactgaacaaccggacgataacaacatagataatgaagataaaactgaagaggagtaattactcctcttttttgtttgctatattaaataagagctaaatataaaaaaattgaacattacggtggtgaaaactttgttaggaatgaatattataacgtcactatcagtagtatttacttgtttaagtcttttaactttaatgatttttgttcatagtaagttctctagtaaaaacgtttttgttttgtatgtaatttatgctataataggaataggtacatacatagttttaactatgtttcaaacaacatctgtacttattaagaatgatgtaatagattccatagaaaatactgaacattatattggattcaatgaccctataattatatttactataagttttataggtgcaatacttggaggaatttggtacaagatgatgaaaattattaaaaagagtaactttaaagataaaaaataaaaaagacggtgaataggttgatattctctaaagataaaaaatgggatgaagcaaaagatttcatcaaaggtcaaggtatgcaagataattggatagagattgtagattattatagacagataggtggaaaacacgtagctgtttttattgctttaaacaaagtaaaatacatgattctagaagcaacaaaagacaataaggtaatattagtagataaagataataatatactattagaagattatgatattgttatggaaagtaagaagatgttttattacattgaagaaccgttcgaggttaaaataaatatccctcaacatattagagatgtaacttataataatactgttgtattaactacagtaagagggagtagaggtgactagtaattggcagatttatttaagcaattcagattaggtaaagactatggtaataatagtaccattgctcaagttcctattgatgaaggattacaagctaacattaaaaaaatagaacaagacaataaagagtatcaagatttaactaagtctttatacggacagcaacaggcttatgcagagccatttatagaaatgatggatacgaatcctgaatttagagataagagaagttacatgaagaacgaacataacttacatgatgttttgaaaaagtttggtaataaccctatccttaatgctatcatacttacacgttcaaatcaagtagctatgtattgtcaacctgcaagatattcagagaaaggtttaggttttgaggtaagattaagagacctagatgcggaacccggtagaaaagaaaaagaagaaatgaaacgtatagaagattttattgttaatacaggtaaagataaagatgtagatagagattcatttcaaactttctgtaagaaaattgttagagatacttacatctatgaccaagttaactttgaaaaagtatttaataagaataataaaactaaattagaaaaattcatagcagtagacccttctactattttttatgcaacagataaaaaaggtaaaattattaagggtggtaagagatttgttcaagtagtagataaaagagtagtagctagttttacttctagagagttagctatgggtataagaaaccctagaactgaattatcttcttcaggatatggattatcagaagtagagatagctatgaaagagtttattgcctacaataacactgaatcatttaatgatagattcttctcccacggtggtactactagaggtattttacagatacgttcagaccaacaacaatcacaacatgcattagagaactttaagcgtgaatggaaatctagtttatcaggtatcaacggttcatggcaaataccagtggtaatggcagatgatattaaatttgtcaatatgacaccaactgctaatgatatgcaatttgagaaatggttaaattaccttatcaatattatatctgctttatatggtattgaccctgcagaaattggtttccctaatagaggaggagctacaggttctaaaggtggttctactttaaatgaggctgacccgggtaaaaaacaacaacaatctcaaaataaaggtttacaacctttacttagatttattgaagacttagttaatagacatattatatcagaatatggagataagtatacattccaattcgtaggtggagatactaagagtgctactgataaacttaatattcttaaactagagactcaaatatttaaaacagttaatgaggctagagaagagcaaggtaagaaacctattgaaggtggagacattattctagatgcttcattcttacaaggaacagcccaattacaacaagataaacaatataatgatggtaaacaaaaagaacgtttacaaatgatgatgagtttactagaaggagacaatgatgattctgaagaagggcaatcaacagattctagtaatgatgataaagagataggaacagatgcacaaataaaaggtgacgataatgtttatcgtactcaaacatctaataaaggtcaaggaagaaaaggagaaaaatcttctgactttaaacattaattaataagcctagaataaatctaggctttgtttatttttttcgtaatttaattttgataaatgtaataactatgatatactatatgtaattgatattaatacataaaaaatattaatatttcacttacaagttattattgttatattattaacgtaaaagtaaataaaataacaagtggaggtgtagacacctttggaagaaataaaatttaatgcttttgtacctatggatttgaagaaatctgtatcaacagcttctgatactaatgagtattctatcgtttcaggatgggctagtactccaagtatggatttacagaatgatatagttaatcctaaaggaatagatatagagtattttaagtcacaagggtacattaattatgagcatcaaagtgataaagttgtaggtatacctacagagaattgctatgtggatatagaaaaaggtttatttattgaagcaaagctatggaagaatgacgaaaatgttgttaagatgcttgatttagctgagaaattagaaaaatcaggtagtggaagacgtttaggtttttctattgaaggtgcagttaaaaaacgtaatataaatgacaatcgagttattgatgaagttatgataaccggagttgcattagttaaaaaccctgctaatcctgaagcaacatgggaaagctttatgaaatcatttttaactggtcatggtacatcacctgacactcaagttgatgcaggagctttaagaaaagaagaaatagcatctagcattacaaatttagcttacgtcactaagattaaagatttaaaagagtttaatgatgtatggaatggcgttgttgaagatttgagtaaatctaatagtatgggatatgaggaatcagtccttacgttacaactagctaaaggtttatctcgtaaagatgcagaactagcagtaatggatataaacaaacaaaaactagaataggtaaggagaatacattctatgagtaaagaaatgcaaaatattttagaagagtatgataagttaaatgctcaagaggcagtttcgaaatctgtagaagatgatgaaaagaatacagtagaatctaccgaagagcaagtagcagaaacaactgaagaacctgctaaagaacctgaaaaagtatctgaggaagatgctaaagaagcacaagagcaaggtgaaaaagttgaatctgaagaggtagcagaaggcaatgaagatgaggaagttgaaaaatcagctaaagaatcaaaagaccctgtagaccaaaaagatactaagacagaaaataaagacaacgagaaacgtaaaaataaaaaagataaaaaagaagattctgacgatgaagataaagatactgacgatgataaagataagaaagaagataagaaggaaaaaacttctaaatcaatttctgatgaagatatcacaacagtatttaaatctatcttaacatcttttgaaaacttaaataaagagaaagaaaactttgctactaaagaagatttaagtgaagttagtaaatctattaatgagttatcagcaaaaatttctgaaatccaagctgaagatgtttctaaatcagtagacactgatgaagaagctgtagaaaaatcagtaacatctacaaacggagagcaagaaaaagtagaaggttacgtttctaaatcagtagacactgaagaacaagctgaaactggtgaagcaaaatcagaagaagctgaagaagtacaagaagataacacatttaaaggattaagtcaagaagaacgaactaagttcatggattcttacaaagcacaagctaaagaccctagagcttctaaacatgacttacaatcagcttaccaatcttacttgaacattaacactgaccctactaatgcatcagagaaagatattaaaactgtaaaagactttgcacaaatttaattaatgcacaaagttgtgttatattatacggtgtaactaaagaatataaatagggtacattttactgtaccctacataaaataaaaagaacacaaatgaaaggtgataaatttatatgactatcgaaaagaacctgtcagacgttcaacaaaagtacgctgaccaattccaagaagacgtagtaaagtcattccaaactggttatggaatcactcctgatacacaaattgacgcaggagctttacgtagagaaattttagatgaccaaatcacaatgttaacatggactaatgaagacttaatcttctatcgtgatatctcacgccgtcctgctcaatctacagtagtaaaatacgaccaatatttacgtcatggtaacgtaggtcactctcgtttcgttaaagaaatcggagtagcaccagtatctgacccaaatatccgtcaaaaaactgtatcaatgaaatacgtttctgatactaaaaatatgtcaattgcatcaggtttagtaaataacattgctgacccatcacaaatccttacagaagatgctatcgcagttgttgcaaaaacaattgagtgggcttcattctacggtgacgcttcattaacttctgaagttgaaggtgaaggtctagagtttgatggtttagctaaattaattgacaaaaataacgtaattaacgctaaaggtaatcaattaactgagaaacacttaaatgaggcggcggtacgtatcggtaaaggtttcggtacagctacagatgcttacatgcctatcggtgtacacgcagacttcgttaactcaatcttaggtcgtcaaatgcaattaatgcaagacaacagcggtaacgttaacactggttacagcgtaaatggtttctactcatctcgtggattcattaaattacatggttctacagtaatggaaaatgaactaatcttagatgaatcattacaaccattaccaaatgctccacaacctgctaaagttacagctactgttgaaactaagcaaaaaggtgcttttgaaaatgaagaagaccgtgcaggattatcatataaagtagtagttaactcagatgacgctcaatcagctccttctgaagaagtaacagctacagtatctaacgtagacgatggtgttaaactttcaattaatgttaacgctatgtaccaacaacaaccacaattcgtttctatctaccgtcaaggtaaagaaacaggtatgtacttcctaatcaaacgtgtaccagttaaagatgcacaagaagacggaacaatcgtattcgtagataagaacgaaacattgcctgaaacagcagacgtatttgttggtgaaatgtcaccacaagtagttcacttattcgaattacttccaatgatgaaattaccattagctcaaattaatgcttctattacatttgcagtattatggtatggtgcattagcattacgtgctcctaaaaaatgggctcgtattaaaaacgttcgttatatcgcagtttaatagaataagaaaaactgaatacaagagaatagggataaacttagggtttatcccttttttattaaaataaacttgaagggatttaataaatatgttatactataagaaactattagataaaaaaatggctactgtttatggtacagtggagattgacaaagatggagtagtcaaaggattaactaaagaacaagaaaaagaatttgccaatgttccaggttttgaatttgaagaagaaaagaaaactactagaaaacaatcagcttctactagtaaagaagaagagcctaaggaagaggaaaagaaagcctctactagaaaaactacaaatactactagaaaatctacagcacgtaaaacaacagccaaaaaagatgaaaataagtaaagggtgaattaaatggttaactcaatgtttggaggggacttagacccttatgaaaaatcattaaactatgaatatccttatcatcctagtggtaatcctaaacacatagatgtaagtgagatagataatttaacattagctgattatggatggtcaccggatgcagttaaagcatatatgttcggtattgtagttcaaaatcctgatacaggacagcctatgggtgacgagttctataaccatatattggaaagagcggtaggtaaagctgaaagagcattagatatatctatactacctgacactcaacatgagatgagagattatcatgagacagagtttaatagttacatgtttgtacatgcttacagaaaacctatattacaggtagagaacttacagctacagtttaatggtagacctatatataaataccctgctaactggtggaaagtagagcatctagcaggacatgttcaattattccctacagcacttatgcaaacaggacaatcaatgtcatacgatgcagtattcaatggataccctcaattagcaggtgtatacccaccatcaggagcaacatttgcacctcaaatgatacgattagaatatgtatcaggtatgcttccacgtaaaaaagcaggaagaaataaaccttgggaaatgccccctgagttagaacagttagttataaaatatgcattgaaagaaatataccaagtatggggtaacttaattattggtgccggtattgctaataaaacattagaagtagacggtattacagagacaataggtactactcaatcagctatgtatggtggagctagtgctcagatacttcaaataaatgaagatataaaagaactattagatggtttaagagcttactttggatataatatgataggattataaggagggttagaaaatggaaaaaccgtatatgataggagctaactctaaccctaatgttattaataagtcaacaacatatactactacaacacaagcagatgaacaagataaacctaagtatactactagactagagtttgatacgattgacatgattaggtttattaatgaccgaggtataaaagtactatgggaagaagcatatttctgtccttgtcttaatcctgatacaggacatcctagagtagattgccctagatgtcatggtaaaggtattgcatatctacctcctaaagagacgataatggcaatacagtctcaagagaaaggaactaaccagttagatataggtatattagatacaggtactgcaataggtaccactcaattagaaaagagaatttcctatagagacaggtttactgttcctgaggtattgatgccccaacaaatgatttattttgtgaataaagatagaattaaaaaaggtatacctttatactacgatgtaaaagaaataacttatatagccactcaagacggtacagtctatgaagaagattatgaaatcaagaataatagattgtatttaaatgaaaaatatgagaatcatacagtaactttaaagatacttatgactttaagatatgtagtatcagatatactaaaagaaagtcgttatcaatatactaagtttaatcaacctaaatcaaaatttgaaaacttacctcaaaaattacttcttaaaagggaagatgtcattgtactacaagacccttataaagttaatgatggtatagaagaagacctagaaattcaagtagatgaccctaaggcttcggcatctaatcctagtaatttaggtggattcttcggaggtgcatttaaataatgccagttcatggaaagagacctaatttatttaaaaataaaaactataagcaggtaggtaagagaacaattgatggtatgcgttcagaagttcttgataaattacaagcaacagcacagcaagtagagaatactagtattaaacgtatgcctacttatctacaaataacagagaaaaagcttgaaaaagaaggagtagtagaccttaaaaaagcttttgctcactcatctaaaaagaaaactagtaaagatggcggatggtatttaactgtaccaatccgcatcaaaactagtagaatgaataacagtacttatcaagatatgagaactttaaaagtagataaaggaacaggttcagtttcgaagataactgattacctagaaggacgtaggaagaatgtaagccacccttcaatgaagcctgaacctatgactcataatatgactaaagttaaaagaggaaagcaatcttcttactttatatttagaactgtttctagtaagtcacctgctagttcttggatacttaacagagataaagttaatgaagataacttctctaaaacaactctaaaaactgttaagcaattaatgaactggaagatgaaaaatttaaattaagaggagggttagtattaaatggcaataacatcagttgattcatatttattatcagaaataaagcctagacttaacactgtgctagagaattgttatattatagatgaagttttaaaagactttgattatcaaactagagagagctttaaagaagctttctgtggtaagaatgcacaacatgaagtaacggtaggatttaacttcccaaaatttaaaaataactatgaagctcattacttgatacaattaggtcaaggacaagagacaaaaaactctttagggagtattcagtcatcttactttgaggcaacaggagatactttagtcgaatcttctacagcaataagagaagatgataagttagtttttactgtttctaaaccaataggagagttaataaaggtagaagatatagagtttgctaaatacgataatcttcaggttgaaggtaataaggtatcatttaagtatcaaacaaatgaagattatgagaactacaatgctaacattatatttaccgaaaagaaaaatgattctaaaggtttagtaaaaggattcacagttgaagaacaagtaacagttgtaggtctttcatttaatgtagacgttgcaagatgtttggatgctgtactgaaaatgattttaatatctatgagagatagtatagaagagcaacaaacattccaattacagaatttgtcttttggtgatattgcaccaataatagaagatggtgactcaatgatttttggtagaccaacaattattaagtacacaagttctctagatttggattatactattacacaagatattaataaactaacttttaaagaaagaaaggattggaagtaggatggctagaaaaaagacacctgaaaataacactcctaaatttaatggttatgttcatatagatacattccttgatactgcaaaaaccctttttaatatgagggattcacaagtagcaggatttaaagcttatatggaaggtagtcattatttgtttagtgagcaagaattcttaccatcattagagaagtatctaggtaggaaattagatatataataacattcagataaggagaattaaatatggcagtagaaccattcccaagaagacctattacccgtcctcatgcatctattgaagtagatacttcaggtatcggtggctcagcaggttcaagtgaaaaagtattttgcttaatcggtcaggctgaaggcggagaaccaaatacagtttatgaattacgtaactattcacaagctaaacgtttattccgttcaggagaattacttgatgcaatagaattagcatggggttctaaccctaactatacagcaggacgtattttagctatgcgtatagaagatgctaaacctgcttcagcggaaattggcggattaaaaataacatctaaaatctacggtaatgttgctaacaacattcaagtaggattagaaaagaatacactaagtgattcattacgtttaagagtaatattccaagatgaccgtttcaatgaggtttatgataatatcggtaatatcttcacaatcaagtacaaaggagaagaagctaacgcaactttctctgtagaacatgatgaagaaactcaaaaagcaagtcgtttagtattaaaagttggagaccaagaagttaagtcatatgatttaactggtggagcttatgactacactaatgctattattacagacattaatcaattacctgatttcgaagctaaattatcacctttcggagataagaacttagaatctagcaaattagataaaattgaaaatgcaaatataaaagataaagctgtatatgtaaaagcagtttttggtgacttagaaaaacaaacagcttacaatggtatcgtatctttcgagcaacttaatgcagaaggagaagtaccaagtaatgtagaggttgaagcaggagaagaatcagctacagtaactgctacttcacctattaaaactattgaaccgtttgagttaactaagttaaaaggcggtactaatggtgaaccacctgctacatgggcagacaagttagataaatttgcacatgaaggcggatactacattgttccattatcatctaaacaatcagttcatgcagaggtagcttcttttgttaaagaacgttctgatgcaggagaaccaatgagagctattgttggtggaggattcaatgaatctaaagaacaattgttcggtagacaagcatcattatctaatccacgagtatcattagtagctaactcaggtacttttgttatggatgatggacgtaaaaaccacgtacctgcttacatggtagccgtagctctaggtggtcttgcaagtggtttagaaatcggtgaatcaatcacattcaaaccactacgtgtaagttcattagaccaaatctatgagtcaatagacttagatgaattaaatgaaaatggtattattagtatagagtttgttcgtaaccgtactaatacattcttcagaatcgttgacgatgtaactacattcaacgataaatcagacccagttaaggctgaaatggctgttggggaagctaatgacttcttagtaagtgagcttaaagttcaacttgaagaccagtttattggtactcgtactattaatacaagtgcttcaatcattaaagactttatccaatcttacttgggtcgtaagaaacgtgataatgaaattcaagacttccctgctgaagacgtacaagttattgttgaaggtaacgaagcaagaatttcaatgacagtttacccaatcagaagcttcaagaaaatctctgttagcttggtttacaagcaacagacattacaagcctagtctaggtgacggagtacctggattaggtactcctattaatataatttgaatactttaggagagtgaatacagatggcatcagaagctaaacaaaccgtccatactggtaataccgtcctacttatgattaaaggtaaaccggtaggaagagcacaatcagcatcaggtcaacgtgaatacggtacaactggtgtatacgaaatcggttctatcatgcctcaagaacacgtatacttacgttatgaaggtacaattacagtagaacgtttacgtatgaaaaaagaaaactttgcagatttaggatatgcttcacttggtgaagaaattcttaagaaagacatcattgatattttagtagtagataacttaacgaaacaagttattatctcatatcatggttgctctgcaaataactacaatgaaacttggcagacaaatgaaattgtaacagaagaaatcgagtttagttacttaacagcaagtgacaaagcacgtacttaatagattagaccaactaaaaagttggtctttttttattgacactttaaaatttatatgttattataaatataataatttaaacaggagatgtactagatggcaaataagagaaaaacaataggaaaaatgagtaacacaagagcaacatggaatattaatccggtaactaaagttaaaaaagataaaacaaaatattctagaaaaaataaacataaaggtcttgacaattataattaactaaggtatattattagtataacaaaaaaaggagatgggtatatgagtacattttggtcagaaagaagaacaactaataaagataggcaagttaaaaaacattatactcaaatgagtatgtatgaaagaaagaaatgtgtagagttattacaagagacaattactgaaaatagaattattaattttacacgacatagtgcaaaaaaggttaaaggtaaaccaacaacaaatatacctaaattaataggttttatttttaaaaataagtttgcctacgaaaatatcatagagtacaataacacagattataatggtaatattgagaggagaattgttgttaaacatcctaaagttataactgtagaaggaaaacctagctatcagtttttgacaattagtcttgaagatgctagagttattacggtgtggtataacagtgtagatgatacacatagaacactagatttaaattattatagtaaagacttgacaattcaataaggaggtattataatgggtataacaatagtaaatagttattttattctgtctagcattttcctcatcatattaaccatattaaatggtaagggtacagttacaagggaatcattaactatgagtaaaatattagtagtaataacatcaattcaatttttagcatgtttaattattaatggtatttattggtcactaaaatttatgtagtagaactagaataaaagtattgacaaattaaaactaataaattataataaaggtataacaaattaaaggagaagatataaaatgtcacaagataaattaagagcaatttacacagaaatgaaagtagaattacacaaatttcctaaagaggtagatataacaagtaaatcaactgcaattgcaatcaatcagattttagataaattcaaaacattaacagaacaagcaggaaagattactagaaaatatttagaaggtcaagaaatattaactattgattatgagtattatgattcattacaagaatactatatttacctacttagaaatagtgaaaagattgaacaaagtttacaagaaattactaagcgtacaggtgaatatgtaaagtaattttgatttaaaaacaaaatatgatatactatgtttaaagtagtaagcctacactagtccgtgttatattaatattgaatcggataagcgtaggctttattaatatttaaaaaaggaaggtatatcatattatggcagaagaaattaaaaaggaacaagatgtacaagaaacaactaaagaagaaaaaaaagatgttagtaaaatgacaccggaagaaatagataaattaaaatatcaagacaaacaagaaaaagaacaagttattaacaaagttattaaaggcgttaatgatacttgggaaaaagaatataactttgaagaactagacttaagatttaaagttaagattaaattacctaatgcacgagaacaaggtaatatctttgcgttacgttctgcttacttaggtggtatggatatgtaccaaacagaccaagtgattagagcatatcaaatgttagctaccttacaggaagtaggtattgaagttcctaaggaattccaagaccctgacgatatttataacttatatcctttaactgttatgtatgaagattggttaggattcttaaactcctttcgttactaatagtatagaaacattagataaagatatagaacgattgggcggtatggaatcaattgttaaacaacctttatctagaaatctatgggctattatgaaagagtttaatgttttacctaccgagcaaagatttaaggacttagacgattatcagatagagtttattattgggaatatgaatagagatgtttatgaacataataaacaacttaaacaagctcaaaaaggtggaaaattcgatagtcaattcgaagatgatgatagtagttggtggaatgaatctcatgaagactttgacccagtacctgatttcttagatgctgatgatttagcacaacagatggaagctaaattatccgatagagataaggaagaaagagctaagagaaacgatgcagagttaaatgatgaaacagaaggacttactacacaacatctagctatgatggaatacatcagacagaaacaacaagaattagatgatgaagtaggaaatggtaagactagtgaagatgacgctactatatcacaagatagcgttaataaagcactagaagacctagatgatgactggtatatgtaaagggtggtaggtgatactaccatccttatttttttaaaatggatggtgaataatgatggcaatgaatgacgattatagattggtcttgtccggtgatagttcggatttagagaatagtctaaaggcaatagaactttatatggattctttagagtctaagaatattgatgctcctttagataatttcttaaaaaaattaaaagtaattgctaaagaagttaaaaatgtacagaacgcaatggataaacaagatggtaaatctgttatatcttctaaagacatggatgaatctattaaatccactcaatctgctacaaagaatataaatgaattaaagaaagctttagatgaccttcaaaaagagaatatatctaaaggtattgcacctgaccctgaagttgaaaaagcatatgctaagatgggtaaagttgtagatgaaactcaagaaaaacttgagaaaatgtcttcacaaaaaataggttctgatgctagtattcaaaatagaattaaggaaatgaaaaccttaaatcaagtaactgaagaatacaataaaataagtaaagattctagcgcaactaaagattatacaaaacgattaagagctaatcgtaatatgactagaggttacatggagcgttcagaaggaacaggacgtttgacatatgaccaaggtgcacgagttagaagtgaactaggtaaagtaagttcttatgagagccaaagaaaacaaaaccaacgtaatttggaacaagcaagagaacaatatagcaactatagaaaccaacaacaagacttgactaaacgtagagctagcggtcaaattaataaggcacaatatgaacaagagttagcttctattaaacaggaaatgaaagctagagaagaacttatatctaactatgagaaattaggagcagaacttgataaaacagttcagtattataagggttcagttcaaaaggatttccaatctagagacgtagaccaacaaagaggaacatttggtagaatggttcaagaacgtttgccatctattggttctcatgctatgatgggtactacagctatggctacaggtttatacatgaagggtgcctcactaagtgaaactaatagacctatggttacatcattaggtcaaaattccgataatatggatatagattctgtaagaaatgcatatggagacttgtcaattgataacaaattaggttataatagtactgacatgttgaaaatggctacttcatatgaagcatcagtaggacataaaagtgatgaggacacaatggcaggaactaaacagcttgctattggaggacgttctttaggcattaaagaccaagaagcttatcaagagtctatgggtcaaatcatgcataccggtggagtaaattctgataacatgaaggaaatgcaagatgcattcttaggtggtattaaacaatcaggcatggttggtcgtcaagatgaacaacttaaagcactaggttctatagcggaacaatcaggagaaggaagaactctaactaaagaccaaatgagtaaccttactgccatgcaatctacttttgcagagtcaggaagtaaaggattacaaggtgaacaaggtgccaatgctattaacagtatagaccaaggacttaaaaatggtatgaatagttcttatgctcgtatagcaatgggatggggaacgcaataccaaggtcttgaaggtggatatgatttacaaaaacgtatggatgaaggtatatctaatcctgaaaacttgacagatatggctgatatagctactcaaatgggtggcagtgaaaaagaacaaaaatacctatttaatagaagtatgaaagaaataggcgctaacctaactatggagcaatctgatgaaatatttaaggactctaaagaaggaaaactgtctaaagaagagttagctaagaaagctaagaaaatggaaaaagaaggtaaaaaagaaggagaagataacgccactgattataaagaatctaaatcaggaaaaaatgaccaaaataaatctaagactgatgataaagcagaagatacttatgatatggctcaaccactaagagatgctcatagtgctttagcaggtcttcctgcccctatatatttagctattggtgctataggagcatttacagcttcactaattgcatctgcaagtcaatttggagcaggtcacttaattggtaaaggagccaaaggacttagaaataaatttggtagaaataaaggcggtagctccggtggtaaccctatggcaggtggaatgcctagtggtggtggttcacctaagggtggaggctcacctaaaggtgggggcactcgttctactggaggaaaaatacttgatagcgctaaaggtcttggaggattcctagtaggtggcgcaggatggaaaggtatgtttggcggggagtctaaaggtaaaggctttaaacaaacatctaaagaagcctggtcaggtactagaaaagtatttaatagagataatggtagaaaagccatggataaatctaaagatatagctaaaggtaccggtagtggtcttaaagatatctataatgatagtatatttggtaaagaaagaagacaaaacctaggagaaaaagctaaaggttttggtggcaaagctaagggtctctatggtaagtttgctgataagtttggtgacggaggtaaaaatggtattctttcacaatcaccaaaagcaggtggaagtggcatagggaaacttggaaaacttgcaggtggacttggaaaaggagccggagttttaggtgttgctacgtctgccttatcattaatacctgctttagcttccggagatagtaaagctatcggcggaggaataggctctatgggtggaggaatggcaggtgcatcagcaggagcttctataggagctttatttggtggtgtaggtgcaatacctggagctttaataggtggagctataggttccttcggaggaggagctgttggtgaaaaagtcggagacatggctaaaaaagctaacactaaagaaggatggaacctaggatggactaacggagataaggatggtaagaataaattccaagattctttattaggaaaacctatatctaaagcatggagcggtataacaggtctctttgataatgacgctgaagcatccgaagaagatagtaaagataagaaaaaaggtgttaaaggcgttaaaggagatactaagaagaaagaaaaaatgacagcagaacaacttagagaaaagaataaccaatctgaaactaagaatcttaaaatctatagtgatttacttgacagagctcagaaaattattgagagtgctaaaggtattaatatagatggaggaacttctgatagtggttctgatagtggaggctctgcatctgatgtaggtggagaaggcgcagagaagatgtacaagttccttaaaggaaaaggactatctgataatcaggtaggagctgttatggggaacttacaacaagaatctaatcttgaccctaatgctaagaatgcttctagtggagcatttggtattgctcagtggttaggggctagaaaaacaggattagaaaattttgctaaatctaaaggtaaaaaatctagtgacatggatgttcaattagattacctatggaaagaaatgcagtctgattatgaaagcaataatcttaaaaatgcaggttggagcaaaggtggaagcttagagcagaatacaaaagcatttgctactggatttgaacgtatgggagcaaacgaggctatgatgggtactcgtgttaacaatgctaaggaattcaagaagaaatacggaggctccggtggcggaggtggtggaggagccctatcctctacttaccaagaagctatgagtaatcctgtattaactactggttctaattataggggctctaatgatgcttctaatgcttctacaactaacagaataaccgtcaatgttaatgttcaaggtggaaataatcctgaagaaactggagacattatcggaggaagaattagagaagttctagatagtaacatggatatctttgcaaatgaacataagagaagttattagtaattttgtattgacacaagagtagtatcatagtatactactcttatacatataaaaaataaaaggaagtatgtgtatatgcgtagaataagaagacctaaggtaagaatagaaatagttacagatgataatacatttacattgagatttgaagatacacgagactataatggtgatgagtttggagctaaacttttaggattccaaactaaaaactctatggaagatgatagttcagttttccaaataaatatggcaggagatacttattgggataagctagttatggctaatgatatcataagaatatttattacacctaatgatgaccctaacgataaagaaggaaaacaagaacgacttatccaggtaggtatggtttctcaagtatcaaaagtaggtagttacggtaatgaccaaactcaatttagaataacaggtcaatcttttgtaaaaccttttatgaaatttggattaggcgttattcaggaagttcaagctgtattacctgaagtaggttggcttattgatggtgatggagataatgaagtaaaatttactggtagctcagctcatgaagtaatgactggtattatacgtagatttataccttatatgaaatataactatactgaaaaaacatataatacaattgataactatcttgattatgatgatttaagtagttgggatgagtttgaaaaacttacagaagtttcagcctttactaattttgatgggtcattaaaacagttaatggatatggtaacagctagaccttttaatgagttattcttcaaaaattcagaaaaaacacctggaaaggctcaacttgtattaagaaagaccccttttaatcctactgagtggagagctttagatatgattaaagtacctactgaggattttatagaagaggatgtaggtaaaagtgatgtagagacatattctatatttacagcaacacctgcaggtatgttgaaagagcttaacggtgatgtattttctaaaccacaattccaccctgaattaactgatagatatggttatactaaatttgaagtagaaaatatttatcttagtacaaaatcaggttcagctactgaggattcagattcttcaggtgatgataatggcacagaacgaggaacttattctaaaattatgaaagatttaagtaactatggaagagataatatatctaaaggtatagataagtatacaagtaaattatcttcaaaatataaaaacttaaaaaaagcccaagctaaaaaaattatagagaagtttgttaaagaaggaaaagtaacagaaaaagaatatgaaaaaataacaggtaataaggtagatgatgaattaacatcagataacagaccgaagttgacaaaagataaattaaagagtatactaaaagagaagtttaaaacacaagatgattttaataattctaagaaaaagaaaaaagctaagacagatgcacttaaagaattgacaactaaatatcgttttggtaataaaacacatgctacaactttattagatgaatatattaaatataaaggagagccacctaacgatgaggcttttgataaatatcttaaagctattgaaggtgttagtaatgtagctacagacacaggttcagatgcaagtgatagccctttagttatgttttctagaatgctatttaattggtatcatggtaaccctaacttctatgcaggagatattattgttttaggagaccctaagtatgacctaggtaaaagattatttattgaagataagcaacgaggagacacttgggagttctatattgaatctgtagaacataaattcgattataaacaggggtattatacaactgtaggagtaactagaggtttaaaagacgctattctagaagatggtaaaggtagtccgcatagatttgcaggattatggaatcaatcatcagacttcatgggaggtcttatgggtgaagatacttctaaagaacttaaagaaaaaggtgtagcagagaaacaaagtagtggagataaagatggtggttctgatagtggtggagctcaagatggtggctctttagattcacttaaaaaatataacggcaaacttcctaagcatgacccaagttttgttcaacctggtaaccgacattataagtatcagtgtacatggtatgcttataatagaagaggtcaattaggcatacctgtgcctttatggggggacgccgccgactggataggtggtgctaaaggagcaggttatggtgtaggtagaacacctaaacaaggtgcttgtgttatatggcaaagaggagttcaaggaggtagcccacaatatggtcacgtagcgtttgtagagaaagtattagatggaggtaaaaaaatatttatctctgaacataactatgctacccctaatggatatggtactagaacgatagatatgagttcagccataggtaagaatgcacaattcatttacgataagaaataaaggaggatagtctatggcaacagataaagaagctaaagatgttattgataaatttatagacaatgtatttaattttgatgtacttacaaaagaaagaataaaagaaaaagatgaagaaattaaaaaaataactacagatgatatgtatgaaaaggttgtgtatatacgaccttatgttggagtaatacaaagccttaaccctcagcatgttcagtatgaatcattttctaataatggttatgatatagaggcagaattaagtttcaggaaagtaagttatttagttgataaagggtctatacctacagattctttatctactttaacagttcatttagtagaacgaaatcaagaactattaatagattactttgatgagatacaagatgtgttgtatggagaatatatggaagaagaatatgtatttgatgaagatgtaccattaagtacgatactagcattagacttaaatgataatcttaaatccttatcaaatataaagtatatgttcaaaggtgctcctaaagagaatccatttggaacagataaagatgtttatatagatacttataacttattatactggttatatttaggtgaagatgaagagttagcatatcctatgaatattaactacttctttacagagggaagattctttactatattcggtaaaggacataagtataaggtagatgttagtaaatttatagttggagatatattattctttggtagaagtgatactaatataggtatttatgtaggagatggggagtttatatctatgatgggtaaattccctaaagatgaaacacctataggaaaatataaacttgatgattactggaatgaatttaacggaagagttatgagattcgatgaagaggtgtatatttaatggtagtaagattccaatcttccatggggagaagtttaaaaagagtagattcggatgatttaaatgtaaaaggattagttttagctacagttagtaaaattaattataaatatcaatcagtagaagttaaagttaacaatttaactctaggaagccgtataggtgatgatggtagcttagctgtaccttatcctaaatctttcataggaagaacacctgaaggaagcgtattcggtacaaaacctcttattactgaaggttctgtagtattaatagggtttctaaatgatgatataaatagtcctattattttaagtgtttatggtgataatgaacaaaataaaatgattaataccaatcctctagatggaggtaagtttgatacagaaagtgtttataaatatagtagttcactatatgaaattttaccatctttaaattataaatatgatgatggagaaggaacaagtattaggacttataatggtaaatcatttttctctatgacatcaggtgaagaagagaaacctcaggcaacagatttttatactggaactgagtatcaagatttatttacttcttattatggtaataagacattaattgagcctagaatacaaaaggctcctaatatgttatttaaacatcaaggagttttttatgatgatggcacgccggataatcatataactactttatttatatctgaaagaggggatataagagcctcagttttaaatacagaaacacagaaaagaactacacaggaaatgtcaagtgatgggtcttatagagttatcaaacaagatgacgatttaatgttggatgaagctcaagtttggattgagtatggtattagtgaagataataaattttatattaaaaatgacaagcataaatttgaatttactgatgagggaatctatatagatgataaacctatgttagaaaacttagatgagagtatagcagaggctatgaagaatttgaatgaaatacaaaaagaactcgatgatataaactaccttctcaagggtgtaggtaaagacaatttagaagaattaatagagtctacaaaagagtctatagaagcttctaaaaaagcaacttcagatgtcaatagacttacaactcagatagcagaagtgagtggtagaactgaaggtattataacacagttccaaaaatttagagatgagacttttaaagatttttatgaagatgcttctactgttattaatgaagtaaatcagaatttccctactatgaaaacagatgttaagaccttaaagactaaagttgataacctagagaaaactgaaataccaaatattaaaactagattaacagaactagagaacaataataacaatgctgataaaataatctcagatagaggagaacatataggtgctatgatacagttagaggaaaatgtcacagtacctatgagaaaatatatgccaataccatggagcaaagttacttataataatgcagagttttgggattctaataatcctactcgattagtagtacctaaaggaataacaaaagtaagagttgcaggtaatgttttgtgggactctaacgccacaggacaacgtatgttgagaatattgaaaaatggtacttatagtataggattaccttatacaagagatgtagctatatctacagcacctcagaatggtactagtggagttattcctgttaaagaaggagattactttgagtttgaagctttccaagactcagaaggtgacagacaattcagagcagacccttatacatggtttagtattgaagctatagaattagaaactgaaactatggagaaagactttatgcttataggacatagaggagcaaccggatacacagatgagcacacgataaaaggatatcaaatggctttagataaaggtgcagattatatagaattggatttacaattaacaaaagataataagttattgtgtatgcatgattctactatagacagaacaacaacaggaacaggtaaggtaggagatatgaccttatcttatatacaaactaactttacatctctcaatggtgagccgataccatctcttgatgatgtactaaatcattttggaacaaaagttaaatattatatagaaactaaacgtccgtttgatgctaatatggatagagaattattaactcaattaaaagcaaaaggattaataggaataggttcagagagattccaagtaattattcaatcatttgctagagaatctttaattaatattcataatcaattctctaatatacctttagcttacctaacaagtacattttctgaaagtgaaatggatgattgtttaagttatggtttttatgctattgcgcctaaatatacaactataactaaagaattagtagatttagctcatagtaaagggcttaaagtccatgcatggacggtaaacacaaaagaagaaatgcaaagcttaatacaaatgggtgtagatggattctttacaaactacctagatgaatataaaaagatttaatattaaagacctattaatttaggtctttttttagttgtaatttaaactagttcgtgatatattagtagtatgagatttatatacatactgaaaaggagaggataaaatgccacaatcagatggaataagtaatcttcatagaatagctttacgcttccctaaagaaggcggtggttatgatatgtatagatttaaagttaaccctgagaactacacaatagattcaccacaacgtacgacagcaattaaaacaaaatcagatattgtaatagaagattatggtaaagacatagaagttattaacttcacaggtacaactggttttagacctgttagagaagcagatggattaaaaacaggtaagcagaaaatggaagagttacaaagtagagttagtgaatatgctatgcaaggtggcagtggtaatgtaagtggttcttacttacaattttttaactttacagatgatagttattataaagttcatttagctcctcaggggttaaagataactaggtctaaagatgaaccattactttttagatatgaaataacattagtagttattggttcattaacagaagcagatagaagtgctgtaacaacagaagagtttggtaacgttaaacctaatgcttctcaaagagtagatgagggtataaaagaattagataaaaatgctcgtaaaacgagagatagaaacaatcaagaaatatctagaagagaaaatacaatacctaaatctacaggagataatacgaacgagggtaatagacttaagcaaagcttccctagtagttctatatataatcctagacaatctactaacggattaaaaggtaatattgacaatatggcgctgataataggttacggtgatggaggtgtatctagctaatgaataattttataccacaacctcaaggtctacttagatttttaaataccctagatacagatttaacttcttctcatatgaatttactggatgaagaggtatcatttgtatctaaattttatacaccacagctacaattaagtgaattagcaaaaaaagtattgacaaatataaagacagatgatatacctgtattagaaagagaatttaatgataatacaattatccataaagctaacgatacattactaaaagtacaggctccaagaatgtatatgattctacagtcgattgtacttgaagcatatgctattgttaattgctttgtagaaaatccgagctctttaaaatacttaactgaagaagatgttagtataacacgggaaaatttaaattatgtagctgactacttaggtaactatgatgactataatagtgttgtcttagacttaagagatttagacttatgttttagtgctatagaattacaattacctctaatcaaaaaggaggctaacgtataatgagatttaagaagcacgtagttcaacatgaagaaacgatgcaagcaatagcacagagatactatggtgatgtgagttattggatagacctagtagagcataataatttaaagtacccctatttagtagaaactgatgaagaaaaaatgaaagaccctgaacgattggcttctacaggtgatacactgattatacctatagaatctgatttaacagatgtatcagcaaaagaaattaattctagagataaagatgtactagttgaattagctttaggaagagatttaaatattactgcagatgaaaagtattttaatgaacatggtactagtgataatatactagcattcagcacaaatggtaatggagatttagatactgtaaaaggcatagataatatgaaacagcaattacaggcacgtttattaactcctagaggttctttaatgctacatcctaattacggttcagatttgcataatttatttggtcttaatatacctgaacaagctacattaatagaaatggaagtattgagaacattaacatcagataatagagtaaaatctgctaatctaattgattggaaaattcaaggtaatgtttattcaggtcaattttcagtggaaataaaatctgttgaagaatcaataaattttgtcttaggacaagatgaggaaggaatttttgctttatttgaataggaaaggattaaattatgaaaactagaaaattaactaacatactatcaaaattaatagataagacaatggcaggtacaagcaagataacagactttactcctggttcagcttctcgttcattattagaagctgtatcattagagatagagcaattctatattctaacaaaagaaaatattgattggggtatacaagaaggtatcattgaagcttttgattttcaaaaaagacaatctaaaagagcttatggtgatgttactattcaattctaccaacccttagatatgagaatgtatatacccgcaggaacaacttttacttcaacacgacaagaataccctcagcaatttgaaacattagttgattattatgcagagcctgattctactgagattgttgttgaagtttattgtaaagaaacaggggttgcaggtaatgttcctgaaggaacaattaatactatagcatcaggttctagtttgattagaagtgttaataacgagtattcttttaatacaggaactaaagaagagagccaagaagactttaagcgcagattccactcttttgtagaatctagaggtagagcaactaataaatcagtaagatatggtgcactgcagatacctgatgtagaaggtgtttatgtttatgaagaaacaggacatattacagtatttgctcatgatagaaacggtaatttatcagataccttaaaagaagatataattgatgctttacaagactatagaccaagtggtataatgttagatgttacaggtgtagaaaaagaagaagttaatgtttctgctacagtaactatatctaataaatctagaattggtgatacattacaaaaacatatcgaaagtgttattagaagctatttaaataatttaaaaacttctgatgacctaataattacagaccttattcaagctataatgaatattgatgatgtattaatatatgatgtgtcatttgataacctagatgagaacattatagtaccgccacaaggaattattagagcaggagaaataaaagtagaactaaagtaaagagaggtgaaacttaagtcgtggctaattttttaaagaatcttcatccattattaagaagagatagaaataaaaaagataatcaagaccctaactttgctctgatagatgcactcaatgaagagatgaatcaagtagagaaagatgctatagaaagtaagttacaatcttctctaaagacatctaccagtgaatatttagataagtttggggattggttcggagtttatcgtaagaccgatgagaaagatgatgtttatagagcaagaattataaaatatttactcttgaaaagaggaactaataatgctataatagatgctataaaagattatttaggtagagatgatattgatgtaagtgtatatgaaccttttacaaatattttctatactaacaaatcacatttaaatggtgaagaccacttaatgggatactattatagatttgctgttattaatgtctctataggtgattatttccctgtagagattatagatgtaattaatgaattcaaacctgcaggtgtaactctatatgtcacttatgatggggcttctactattagaggtggagcaattattaagtggttagatgggttacctaaaatagaaacataccaagagtttgatagatttacaggttatgatgatacattctatggtcatattaatatgaatcaaagtaaagatactgataacagttcatcagatatttttaaaacaaaccatagcttaattaatagtttagatgttttaacaggttcatctagtgtagggagacagtatattaactacggatatgtaacatcatatgtttataatccaggtatgacatcttctgtaaatcaaataagcgctagtacagaaggtagaggtcaagaagtacctactgactattatatgtatactagtactaagaataacaatacagtagaacttagtatgcaaactacttccggtgtgtcttatttatataataactttaattttagggactatatgagtaaatatagacctcaagtagatttacaatctgatgaggctagaagaattgtatctgattatataaaagaattaagtattgattactatcttagtgctgtgatacctcctgatgaaagtatagaaattaaactacaagtttatgatttttctattaatagatggcttacagtatcaattaataatttatctttctatgaaaaaaatatcgggagcaatataggatatataaaagattatctaaacagtgaattaaatatgtttactaggttagagataaatgcaggtaaaagagattcagtagatattaaagttaattacttagatttaatgttttattactatgaacgaggtatttatacaataaaaccgtataaagcattaatagaaaattatttagatatatctagagagacttatgtagaagcatttaaaatagcatcattatctaatggagatattataactaaaacaggttttcagcctatagggtatttaaaactagttggtaattatgaaaatacaatacctagcacaataaatatagtagctaaagatacagataataaccctatagaatctaatgaattagatgtatataatacagtagagaatagaaatttattacaatcttataaaggtgtaaatacgatagctagagaaataacttctacaaaagagtttactgtatcaggatgggctaaagagatatactcaactaattatctttctaaagtattaaaaccaggtaaagtgtatacgttatcttttgatatggaaataacaggtaatgacccaactcttaaatcttattctgataatcatggtatatatttatacagtaatactaagggaattgttgttaatggtgttaaatctatggaacgtactataggtaacaaagtatccgtaactcaaacttttacagcccctactattactgaccatagattactaatatatactggaagatatacatctgatggtaaagcatcaactcctccagtgttctttaatacagttaaaattacggaattaaaattgactgagggttcttctaagctagagtactcacctgctccggaagataaacctaacgtaatagaaaaaggaattaaatttaataatatcctaactaatatacagactttaagtattaattcggatactatcttaaaaaatgtaactttatattattcttactatggtgatagttgggtagaactaaagactctaggaaatattagtactggagaaacaacagaaaccaataacttaatagatttatatggattacagacagtagattattctaatataaatccaatgtctaaagtatcattacgttccatttggaatgttaagctaggtgaattgaacaatcaagaaggttctttatctaatatgcctaatgattactttaatgctgtatggcaggatatagataaattatcagatattgagctaggttctatgagaatggttaaagacactgagggcggagtattcgatggagctacaggtgaaattattaaggctactctatttaatgtcggtgcttatactgatttagatatgttagcctatactttgactaattatactgaaccgttaacgttaggctctagtcgattaataagtgagctaaaagaagaactactaacatcagaatcatttaatgtcgataatagaattaaagtaattgactcaatatatgaggagttaccaaatacaagcattattaaaaatggatttgttgaaagagaagttacaggttctaaatatttagattacggtttatatgagcctatagaagatggtactagatataaacttattgtcgaaggagaatttaaagataatatagaatttatatctttatacaattctaaccctaactttaatgaaacatttatatatccatcagagataattaatggagttgctgaaaaagaatttattgcaaaaccatctactgaagacaaaccaaggttaaatacagatgttagaatatatatacgaccttatgattcaactatctctaaagtaagaagagtagaattaaggaaagtttaataaataagttgacagaaagttaataatatggtatacttataaagtaatatttagtgggtataccatgttatattaataaagaaaacaacagatgaaaggaattaaaaaatatggcaattgcaacgtataattctcatgttgagttagcaaaatatctagttagtaaagctgattcagtttacttaacaattggaaagagcacaccgtggtctaatgaaacaaacccaccgcaacctgatgaaaatgcaacagtattacaggaggttattggatataaaaaagctactaaagttactttagttagaccttctaaatcacctgaagatgataataagaatttaatttcttatggtaataaatcgtgggtagaagtaacacctgaaaatgctaaagctgaaggagctaaatgggtttacttagaaagtagtattgttggtgacgaactacctcttggaacatatagacaagtaggatttgttatggacttagtagcaaaaagtggtattagtaaatttaacttagtacctagtgaagtagaatcaactggaacattattattctttgataataaacaattccaaaatagaagtgagcagacaactgctaaagaaagatttattgtagaagtttaaagaaagggagataattctaaatggcaattaattttaaaggttcaccttatttagatagatttgacccgtctaaagatagaacaaaagtattatttaatcctgatagacctctacaacaggcagaattaaatgaaatgcagtctatagaccaatattatttaaaaaatctaggagacgctatttttaaagacggagataaacaatcagggcttggattcacattgtctgaagataatgtattgacagtaaatcctggttatgtatatatcaatggtaaaataagatattacgataatgacgattcagttaaaataactggcgtaggtaaagaaactattggtattaaattaacagaacgtattgttacacctgatgaagatgctagcctattagaccaaactagtggagtaccaagttacttctctaaaggtgcagatagattagaagaaaagatgtcattaacagttaatgacccgacatcagcaactatttatactttcatggatggggatttatatattcaatcaactaatgctgagatggataaaatcaacaaagtattagctgaacgtacttatgatgagtcaggttcatataaagtaaatggttttgaactattttcagaaggtaatgctgaagatgatgaccacgtttctgtagttgtagatgcaggtaaagcctatgtaaaaggttttaaagtagacaaacccgtatcaacaagaattagtgtacctaaatcttatgacttaggaacagcagaaaatgaaagtactatctttaataagtctaataactctattagtttagctaatagccctgtaaaagaaattagacgtgttacaggtcaagtacttattgaaaaagaacgagttacaagaggagctcaaggtgatggtcaagattttctttcaaataatacagcatttgaaattgtaaaagtttggactgaaacaagccctggagttactacaaaagagtataaacaaggagaagacttcagattaacagatggtcaaacaattgattggtcacctcaaggtcaagaaccttcaggaggtacttcatactacgtttcttataaatataacaaacgtatggaagccggtaaggattatgaagtaacaactcaaggtgaagggttaagtaagaaatggtacattaactttacaccttcaaatggtgctaaacctattgaccaaacagtagtattagtagactatacttactacttggctcgtaaagattcagtgtttattaataagtatggtgatattgcaatattacctggtgaacctaatattatgagattagttacaccaccattaaacacagaccctgagaatttacaattaggtacagttacagtattacctgattcagatgaagccgtatgtatttcatttgcaatcactagattgtctatggaagacttacagaaagttaaaacaagagtagataacttagagtataaccaagcagtaaatgctctagatgatggtgctatggaaggacagaaccctctaacattacgttcagtattcagtgaaggtttcattagtcttgacaaagcagacattacacatcctgacttcggaattgtatttagttttgaagatgcagaagctactctagcttatacagaagcagttaaccaacctaagattattccaggagatacaacagctcatatttggggtagattaatttcagcaccatttactgaggaacgtacaatctaccaaggtcaagcatcagaaacattaaatgttaacccttataatattcctaacaaacaaggtgtgttaaaattaacacctagtgaggataactggattgatactgaaaatgttacaatcactgaacaaaaaactaaaaaagtaactatgaaacgattttggagacataatgaaagttactatggtgagactgagcattacttgtattctaacttacagttagatgcaggacaaaagtggaaaggtgaaacttacgcttatgatagagagcatggtcgtaccggtactttattggaatcaggaggacaacgtactctagaagaaatgattgaattcattagaatcagagatgtatccttcgaagttaaaggactaaaccctaatgataataatttatatttattatttgatggagtaagatgtgctataacacctgcaactggctatagaaaaggctctgaagatggtacgataatgacagatgctaaaggaacagctaaaggtaagtttactattcctgcaggtattcgttgtggtaaccgagaagttacacttaagaatgctaactctacaagtgctacaacttacacagcccaaggacgtaaaaaaaccgctcaagatattattatcagaactcgtgtaacagtaaacttagtagacccgttagcacaatcattccaatatgatgagaatagaactatatcatcattaggattatactttgcttctaaaggtgataaacaatctaatgttgttatccaaattagaggtatgggtgaccaaggttatcctaataaaacaatctatgcagaaacagttatgaatgctgatgatattaaagtatctaataatgctagtgctgaaactagagtatactttgatgaccctatgatggctgaaggcggtaaggagtacgctattgttattattactgagaacagtgattatacaatgtgggtaggtactagaactaagcctaaaattgataaacctaatgaggttatttcaggtaatccatacctacaaggtgtattattcagttcatcaaacgcatcaacatggactcctcaccaaaactctgaccttaaatttggtatttatacttctaaatttaatgagacagcaacgattgaattcgaaccaattaaagatgtatcagcggatagaatagttcttatgtctacgtacttaactcctgagagaacaggatgtacgtgggaaatgaaattaattctagatgatatggcatcttctacaacattcgaccaattgaaatgggagcctatcggtaactatcaagacttagatgttttaggtctagcaagacaagttaagttaagagcaactttcgaatctaatagatatatctcaccattaatgagctctagtgatttaacattcactacattcttaacagagttaacaggttcatatgttggtagagctattgatatgacagaggctccttacaatacagtaagatttagttatgaagctttcttacctaaaggtactaaagttgttcctaagtattctgcggatgatggaaaaacttggaaaacatttactaaatcccctacaactactagagccaataatgagtttacacgctatgtcattgacgagaaagtaaaatcatcaggaacaaatactaaactacaagttagattagatttatcaactgaaaatagctttttacgtcctcgtgttcgtagacttatggttactactagggatgaataaactagaggggttgattgacccctctttatttaataaggagagatttatatgcctagagaagttagagacccttattctcaagctaaattatttatacctacagttgaggaaaaatcaattaaggaattagaaaaaacatacaaagaaaaaattgatgaagctactaagttaatcaatgaattaaagaaagagagaggagaaaaatagatggcatttaactacacgcctcttactgaaacacagaagttaaaagatatgtatcctaaagttaatgatataggtaactttttaaaaacagaagttaaccttagtgatgtaaaacagatatcacaacccgactttaataatattttagcatctatacctgatagtggtaactattatgtaactaattcaaaaggtgctcctagtggagaagctacagcaggatttgtaagattggataaaagaaatgtaaattattataaaatttactattcaccatatagcagtaacaaaatgtatatcaagacttatgctaatggtactgtatatgattggattagttttaaattagatgaaggtagcttatacaatgaaggtaatactttgaatgtaaaggaacttactgaatccacaactcaatatgcaacactagttaatcctccaaaagagaacttaaatacaggttgggttaattacaaagaaagtaaaaatggtgtttcttctttagtagaatttaacccggttaactccacttcaacttttaagatgataagaaagttaccagtacaagaacaaaagcctaacttattgaaagatagtttatttgtttatcctgaaactagctattctaatattaaaacagataactgggatacgcctccattttggggatattcttctaatagtggtcgttcaggagttagatttagaggagagaatacagtacagatagatgatgggtctgatacgtacccttcagtagtttctaataggtttaaaatgggtaaagaactttctgtaggtgatactgtaacggtatcagtatatgctaaaattaatgaccctgctttacttaaagataacttagtttactttgaattagcaggatacgatactgtagatgatactagtaaaaatccttatacaggaggacgtagagaaataacagcaagtgagataacaactgagtggaaaaaatactctttcacattcactatacctgaaaatacaatcggagcatcaggcgttaaagttaattacgtatctttactactaagaatgaattgttcatctagtaaaggtaatggtgctgtagtatactatgccttacctaaattagaaaaatcatctaaagttacaccatttattacacatgaaaatgatgttcgtaaatatgatgagatttggtctaattggcaagaagttattagtaaagatgaattaaaaggtcactcccctgtagatattgaatataatgattattttaaatatcagtggtggaaatctgaagttaatgaaaagagtttaaaagatttagctatgacagtacctcaaggatatcatacattttattgtcaaggctctattgccgggacgcctaagggacgttctattagaggaaccattcaggtagattatgacaaaggtgacccatatagagctaataagtttgttaaattattgtttactgacacagagggtattccttacacattatattatggtggttataaccagggttggaaacccttaaagcaatcagaaacttctactttactatggaaaggtattttagattttgggtctacggaagctgttaacttaaatgactcattagataattacgatttaattgaggtaacttattggactcgttcagcaggacatttttctacaaaaagattagatataaaaaatacatcaaatttactgtatattagagattttaatatttcaaatgatagtacaggttctagtgtagacttttttgaagggtattgcacttttcctactagaacatcagtacaacctggtatggtaaaatctataactttagacgggtctacaaatacaacaaaagtagcatcatggaatgaaaaggaacgtataaaggtatacaatattatgggaattaatagaggataaagaaaggtggaataaaaaaactatggctgttaaatatgatataggtaataatgagatagtattacatttaagagaaggtaaatatataacagggtttacaacagtaggagggtatgataaggagttaggacaagtaaaagttaatagagaaatcttacctgcttacttctttgataattttgcctatgaaagatatttgtattatagtaaacctgaagaggttatagaaaataaaaactatgtaccaccacaaatcaatgatgatgatgaggaatcccaacaaattactgtacctaaagaacaatatgatagtttaaaagaagaactagagcttatgagaaaacaacaagaagctatgatggaaatgcttcaaaagctcttaggtcaaaaggggtaattataaatggcattaaattttactacaataacggaaaacaatgttattagagacctgactactcaggtcaataacattggagaagaattaacaaaagaaagaaatatatttgacattaccgatgatttagtttataattttaataaatcacagaaaattaaactaactgatgataaaggattaactaaatcttatggaaacataacagcccttagagatataaaagaacctggttattactatataggtgctagaacattagcaacattattagatagacctgatatggaatctcttgatgttgttttacatgtagtacctcttgatacttctagtaaggtagttcaacatttatatacactatctactaacaataaccaaattaaaatgttatatagatttgtctcaggaaactctagttcagaatggcaatttattcaaggattacctagtaataaaaatgctgttatatcaggaactaatattttagatatagcttcaccaggtgtttactttgttatgggaatgacaggaggaatgcctagtggagtaagctccggatttttagacttaagtgtagatgctaatgataatagattagctagactaactgatgctgaaaccggtaaagaatatactagcattaagaaacctacaggaacatacacagcctggaaaaaagaatttgagccaaaagatatggagaaatatctactaagtagtattagagacgatggtagtgcatcattcccactcctagtttatactagtgatagtaaaacatttcaacaagctattatagaccatatagatagaacaggtcaaacaacctttactttctatgttcaaggcggtgtatccggttcccctatgtcgaatagttgtcgagggttattcatgtcagacacacctaatacttctagtttacatggtgtttacaatgctataggtacagatggtagaaatgtaacaggttcagtggtaggtagtaattggacttcaccaaaaacatccccttctcataaagaattatggacaggagcacaatcattcttatctacaggaactactaagaatttatcagatgatattagtaactactcttatgtagaagtttatactacacataagacaacagagaagactaaaggtaatgacaatacaggaactatatgtcataagttttatttagatggtagtggaacttacgtttgttcaggtacatttgtttccggggatagaaccgatacaaaaccccctatcacggagttttatagagtaggtgtatcttttaaaggttctacatggactcttgtagatagtgcagtacaaaatagtaaaactcaatacgttacaagaattataggtattaatatgccatagactaggagaaatttcctagtctttttttttcttgacttgaaaaggattctgtggtatactataactcgtgtaaggatataaggagattaaaatgagattaagaattaagaacttatatacctatgtagaatttgaggaggatgataaatacttaaaagatatatttttaaagagagtccatacgactataggagcaagacaagaaggatttcagtacagccctgcgtacaaaagaggtagttgggatggttatgtagatttttatgtttatgaggaagataaattccccactggacttttatttaaaattgagttattattaggtgagctacaatcaaggtataatttccagtttgaaacaattgatgagcgtgatgaaagtttcttatctgaagaagatattgatgatgagataacattgcttgataataatgtcggtcaaattaccttaagagattaccaatatgaagcagtgtacaatagcttaacattttacaatggtattgctcacttagctactaatggtggtaaaactgaggttgctagtggtattatagaccaattattacctcaattagaaaaaggtgagagagtagcattcttcacaggctctacggagatattccatcagtctgcagataggctccaagagcgtttaaatattcctattggtaaagtaggtgcaggtaagtttgatgttaagcaggttacagttgtaatgatacctactttaaatgcaaaccttaaagacccaacacaaggggtaaaggttacgcctaaacaaaatattagtaaaaagattgctcaagagatattacctaaatttgaaggtggaacaaatcaaaagaaattactaaaagtattacttgataacacaacacctaaaacaaaagtagaacaaaatgtattaagtgccttagagataatttaccaaaatagtaagacagatgcagaagttttattaaacttaagaaatcataatgcacattttcaaaaaattgttagagaaaagaacgaaaagaaatatgataaatatcaagatatgagagattttttagactcagttacagttatgatagttgatgaggcacaccattctaaatctgattcttggtacaataatttaatgacatgtgaaaaagctttatatcgaattgcattaacagggtctatagataaaaaagatgaattactttggatgagattgcaggcgctattcggtaatgttattgcacgaactactaataagtttttaattgatgaaggtcattctgctagaccaacaataaatattatacctgtagctaatcctaatgacatagatagaattgatgattatagggaagcttacgataaaggtataacaaataatgattttaggaataaacttattgcaaaactaacagaaaagtggtataatcaagataaaggtacattgattattgtaaacttcattgaacatggagacacaatatcagaaatgttaaatgatttagatgtagagcattacttcttacatggagaaatagactctgaaactaggagagaaaaattaaacgatatgagaagtggtaagcttaaagtaatgatagctacatcacttattgatgagggtgtagatatatcaggtattaatgcactaatattaggtgcaggaggtaagtcattaagacaaacattgcaacgtattggtcgtgctttacgtaagaaaaaagacgataatacaacacaaatatttgattttaatgatatgacaaatagatttttatatactcatgctaatgagcgtaggaaaatttatgaagaggaagattttgaaataaaagacttaggaaaataggagggtaagagatggcaacaaaaacacaaagaaagctataccaatatctagaggaaaatgctacagaaaataaatttcatatttctactaagaaagagctagcagattctctaggtgtttccatctctgctttatccaataaccttaaaaagttagaagaagaaaataaagtcgttactgtttctaaaagaggaaaaaacggcggggtaataataactttagttagagagtatgacacagaagaattgaaagaattcaataattctacagataatattattacttccgatttacagtatgctaaggcattaagagaaaagcacttcccttcttatagatatgagagaaaagaacaacgtagacgtactaagatagaaatggcacaatacaatgccattaaggatgagaagagaagaattatagcagatatgaatttctattcagaaggtcttccttatccttctaaagatatttttaatatgtcctatgacccggaagggttttataaagcgtacatcttatgtaagttatacgaccaatatgctatttctcatatggatgctaaacatacaagtcatcttaaagcaatgagtaaggcaacaactaaagatgaatacgactaccatcaacatatgtctgaatactatagaaataaaatgattcaaaatttacctagaaatagcgttagtgataatttctttggtagtaaaatgtttaatactttttataatttttatttaaaaataaaagataaaaatattaatgtatttaagtatatgcaaaatgtatttaaaaatgtaacattttattacgagaacggtatgcaacctaatccaataccttctcctaacttctttagctcagataagtattttaaaaactataataattatattaaaggaataaaaaaaggtgttaacagtactaatagacacctaggtgatacagacagcatcattaattcatcagactatgtgaaaaaccctgctgtattacatctacaccaactatatactacaggattaaattctactttacatgatattgatactatgtttgaacaagccttagaccttgaaaatgcctcctatggattatttggagatatgaaacatattattttactacagtataattctatgattgaagaagaaattaagaatttacctagagaagaaaaggatattattaataaatatgtaaaacaatgcataattaatgattattcaccaacaagtatttcaccttctgcaaggttatcaatgtttactatgcagaaagagcatatagtttacaataagcagttaaataaaggaatcaagagagaggatttattaccattaagtctaggaggtatagtgaataaagatttattgagtggtatggatatacaaaacttagaacagaatggtaatgaatacctatatatgagacaacatacttcaacttattatatattaagaatgtttggtgactatttagggtatgaggtaaacttaagagaagtaaaatatattgtagagaaatataatttaattgataaaataccattgacaaaagagggtatgttggattataataaacttatacatttagtagaggaagaggttaataactatgagtaagaagataaaggagcttatccttcataaatcaatgaaggatatacattttgcaagagaagtattagataacttacctaagaatctattttcagcagagtctgaggacatgggttacttatttacagctataaagagaacagcacatatttccgataagatgtcaaatgaagcattagcaattaaagtagaacagcttatgggtaataataaggaagatgaagagaaagtaaccaagacattaacttacttagaagatttatataaagtagacgttaatgaaaaagatgaatctgttaattatgaaatagagaagtatattaaaacagaaatgtcaaaagaagttttagttaaatttattgcagaaaataaacaagaagactctgataatctacatgaacttgtagacaaactaaagcaaatagaagtaagtgacatctcaggaggtaatggggagtttattgacttcttcgaagatacagaaaagaaacaagaactattgagtaatttagctacaaataaattctctactggatttacttctattgacaaccatattgaaggtggtatagcaagaggagaggttggattaatcatagctcctaccggtagaggtaaatcattaatggcttcaaacttagctaagaattatgttaaaagtggattaagtgttttatatattgccttagaggaaaaaatggatagaatggttttgcgtgctgagcaacaaatggcaggagcagaaaagagtcaaattgtaaatcaggatatgtctttaaataataaagtttatgatgcaatacaaaatcattatcagaagaatagaaagttattaggtgacttttatatttctaaacatatgccaggtgaagttacaccaaaccaattagaacaaattattgtcaatacaacaattaagaaggataaaaatattgatgttgttattattgactatcctcacttaatgagaaatccttatgctaaatatcattcagaatcagatgcaggagggaaattgtttgaagatattcgtagattatcacagcaatatggatttgtttgttggacgttagctcaaactaaccgtggtgcttatggttcagatgttattacaagtgagcatgtagaaggttctcgtaagattgtcaatgctgttgaggtgtctttagcagtaaaccaaaaagatgaagaattcaagagcggtttcttaagattgtatttagataaaattcgtaatagctctaacacaggagaacgatttgttaatcttaaagtagaaccaactaagatgattgtaagagatgaaacacctgaagaaaaacaagagcatatacaattgctatcagataatggaaaagaagacacaagtaaatttcaaaataaagataataaaatagaagctataaataacacattcggaggattaccgggagtttaattttttaaaatataccacttgacattttatatgttaggtggtataattattttataaagaataaaggagagattaataatgaaatttgtattctttacagatagccactttcacttatttactaactatgctaaacctgatgagcagtatgtgaatgatagatttagagaacagatacaagctttacagaaaatgtttgatattgcaagagaagaggatgcaacagttatatttggtggggatttattccacaaacgtaacgcagtagatactagagtatataataaggtatttgaaacattccaacttaatagagatatagaagtactaatgttaagaggtaatcatgattcagttacaaatagtttatatacagattctagtatagaacctttcggttacttacctaatgtagaggtttgtaaaaaccttgatactttagggtttttaggagaagaacaggatattaatattgttatggctccttatggagacgagactgaagaaattaaagagtttattaaaaataaatatgtagaagatagagtaaatatcttagtaggtcatttaggtgtagaaggctctttgactggaaaagggtctcatagattagaaggggcatttggataccaggatttattacctgataaatatgatttcattttactaggtcattatcaccgtagacaatatttccaaaatccgaatcatttttatggtggttcattaatgcaacaatcattttctgatgagcaagaagctaatggtgttcatttaatagatacagaaaaaatgactacagaattcatcccaatccatacacgtagatttattactattcaaggagaagatattcctgagaactttgaacagctaatcgaggaagataattttattagggttatcggtacagcaaatcatgctaaggttttagaaatggatgacagtatgaaagataagaatgttgaagttcaaattaaaaaagagtatactgtagagaaacgtattgatagtgatgtgtctgatgaccctttaacaattgctagtacctatgctaaacaatactcacctgaatcagaacaagaaatacttgagtgtttgaaggaggttttataatgaaaaaatatagagaatatctaaataagacagatgcagaaaatttagcagaggattgggagaaagtaaccgaagatttatggaaagtgtttaaagatatgaaacctaaaattaatacattagatatcagtaatgtagtaagtaaagacttagataaaagtaaacctattttacaattccaagattcagatggagtaatagagaatatttgtaatgttgaaggtttagaagatggtctaagtaaaatgaaaaagatttttgatgatagtaattttgaaaagcattattacaatagagtagtagaccatgatgagtattactggattgattatggctctcatcattgtttctttagagttacgaaaggggataagtaatggttgtatttaaacaagtagaagttaataattttttagcaattaaagaagctacgctagagttagacaatagaggattaattctaattgaaggtgagaataaatctaatgagtcatttcattcaaacggctcaggaaaatcaactttaatatctgccattacttacgctttatatggtaaaactgaaaaaggactaaaagcagatgatgtagtaaataatattgagaagaaaaatacatctgttaaacttaagtttgatattggggaagatagttatttaattgaacgttatcgtaaagataaagagaataagaataaagtaaaattattcgttaatgaaaaagagattacaggttcaacaaatgacgttaccgataaacaaatacaagatttatttggtattgagtttaatacttacgttaatgccatcatgtatggtcaaggagatatccctatgttctctcaagcaacagataaaggtaagaaagaaattcttgaatctattactaagacagacgtatataaacaagcacaagatgtagcaaaagagaaagttaaagaagtggaagaacaacaaaataacataagacaggaaatctataaactaggttatcagttatcgacaaaagatgagtactttcaaagagaaatagagcagtacaatcaatataaagaacaattggttcagatagaaaacagtaataaggaaaaagatagattaagagaacaagaggagaagcaaatagaagctcaaatagagcaactagcttcacagataccaacaatacctgaagatgaatttaagcactcagaggagtataataaagcctctcaaagcctagatttactttctaataaattaacggagttaaatcaagtttactcagagtataataccaaagaacaagtactaaaatctgaaatagctacattaagcaatagtctaaatcagttagatacaaatgaccattgtcctgtttgtggctcccctatagataattctcataaattaaaagaacaggaaaatatcaataatcagattgagaataagaaacaagagattactagtgtattagaaatgaaagatacgtataaagaagctattgataaagtaaaagataaatcacaagaaattaaagataaaatgtcacaggaagaccaacaagaacgagagcacaataataagattaacagcataattcaagaggcttctaggattaaatcagacattagttcattagagaataataaaacgtatttaaaagttaaatatcaacatcaatctgttcaaggattagagagagaagaaccaagtaaagaaaaacatgaggaagataagaaagaattacaagaatctattgacaaacatgaagagaatatagtacaattagaaactaagaaaggtaaatatcagcaagctgtagatgcttttagtaataaaggtatacgttcagtagtgttagactttattacaccattcttaaatgaaaaagcaaatgagtaccttcaaactttatcaggttcagatattgaaatagagttccaaactcaagtgaagaatgctaaaggagaactaaaagataagtttgatgttattgttaagaatagcaagggcggaggttcgtacaaatccaattcagcaggagaacaaaaacgtattgatttagcaattagttttgcaattcaggatttaattatgagtaaagatgagatatctacgaatattgcactttacgatgagtgttttgatggattagatactatcggttgtgaaaacgtgattaaattattaaaagatagacttaatacagtaggaacaatatttgtaattactcataataccgagcttaaaccactgtttgaacaaacaattaaaatcgtaaaagaaaatggagtatcaaaactggaggaaaaataatgaaattaaagattttagataaagataatgcaacacttaatgtgtttcatcgtaataaggagcacaaaacaatagataatgtaccaactgctaacttagttgattggtaccctctaagtaatgcttatgagtacaagttaagtagaaacggggaatacttagaattaaaaagattacgttctactttaccttcatcttatggtttagatgataataaccaagatattattagagataataaccatagatgtaaaataggttattggtacaaccctgcagtacgcaaagataatttaaagattatagagaaagctaaacaatatggattacctattataacagaagaatatgatgctaatactgtagagcaaggatttagagatattggagttatattccaaagtcttaaaactattgttgttactagatacctagaaggtaaaacagaagaagaattaagaatatttaacatgaaatcagaagagtcacaactgaatgaagcacttaaagagagtgatttttctgtagatttaacttatagtgacttaggacaaatttataatatgttgttattaatgaaaaaaattagtaaatagtaaggaaggatattatgaggtttgaagactttttaacccaagaattaggagaaccaaaagaaaatactataggtgagctaagatactgttgtccgttttgtggagaaaaaagttataagttctatgttaagcaagccctagactctagtaatggtcagtatcattgtaaaaaatgtgatgaatcaggtaaccctattacatttatgaagacttattataacattacaggtaaacaagcttttgatttattagagtctaagaatatagatatagagagagcccctttacttacgaccaataataaggatttgacagaatcagagaaacttatattaatgcttagaggtgtgcaccaagataaaggaaatactagtattaaacctcctagattacctgaagggtataaattattaaaagataacttaaataataaagagattataccctttttaaaatacttaaaaggtagaggtataactttagaacaaatcattaataacaatataggttatgttattaatgggagcttttataaagttgacggggaatccaaagtatcattaaggaatagtattatattttttacttatgataatgatggaaactaccagtactggaatacaagaagtatagagaagaacccttatattaaatctattaatgctcctgctaaacaagatgaagtagggagaaaagatgtcatatttaatttgaatatagcaagaaagaaaaagttcttagttataactgagggtgtatttgatgctttaaccttccatgagtatggagtagcaacattaggtaaacaagtaaccgagaatcaaataaaaaaaataattgattatgttagtatagatacatcaatatatattatgttagacactgatgcactagataataatatagacttagcttataagttaaaaacacattttaataaagtttactttgtacctcatggtgatgaagatgcaaatgatatggggacaaggaaagcctttgagttattaaaacagaaccgggtgttagtaacacctgaaagtatacagagttacaaaatacaacaaaaacttaaactttaggcttgaccttagagaagttttatgttatactagtaattaagtaattaataaaggagaaaaaaaataatgtcaaataataaaaaagatattttagaatttgtagatgaatacattacagctttaagagttggtaatgagcaacgacaacatcaattagaagaaatgggtaaagaagaaacagcaacattaacagatgtagctaaagctattactaaccttatgttaggtgttaatgagcagatgacagacttagaatataataatgagttaaacttaaatattttaattgacgctttatataaagcagagcttattaatgaagatgtattagactacattcaagaatcaattgataaatcacaagaagaacctaaaaatgaagaagaaaaaggagaacaagaataatggaaaaaaatattagcacacacacaaaaggtattagtcaagcagacatggagaaatggattgaagctgcagtacaaggaactgttgatggtaaacaagttgatgagaaaacagctaaacaattagatagaattggttcacgtagtgtttctttagaagaagcaactcgtattgctaaagttcttaatgctgtaacagctcaagaggttacaggagactttaatgatgcatttaatgcaattgacttaatgatgattatcatggaagatgagttaggagtaactcaagaaaaagtagggaaagctaaagataaactaaatgaaaaacgagaagcttacctaaaagagaaacaagaagaattacgtcaaaaacaacaagaagaggcacagaaaaaaactgaatctgacagcaatgaaaaagtaattcagttgaagaaaaatgacgaacagtaagaaaaaaggggatacattcgaacgtaaaatagctaaagaattaacttcttggtggggataccaattcaataggtctcctcaatcaggtggtgcttcatggggtaaagataataatgctgtcggagatatagtagtacctcaggaagctaattttcctttagtagtagaatgtaaacatagagaagaatggactatagataatgttcttctaaacaacagagagccacatacatggtgggagcaagtcattaatgatagtagcaaggtgaataagacaccttgcttaatatttactagaaatagagctcagagttatgttgctttaccttatgatgaaaaagtatatgaagatttaagaaataatgaataccctgtcatgagaacagattttattattgataatattagaaaagataaatttttttatgatgtccttataactaccatgaatgggttgacctcatttacaccttcttatattatatcttgctacgacaaaaaagatataaaaccatacaagaaggtcgagtctaatttatctgaggtaagtaagcatgaagatgaattgattaatgaccttcttagtgatatataaggaaggtaagataagtatgacaagtaaagaaagaccattaatcgtatatttttcaggtacaggacaaacagaaagattagtaaacaaaattaatattaataattcatttgaaacatttagggttaagagtggaaaagaaaaagtaaataaaccttttatactaataacacctacttataagaaaggtgcaatacctaaacaaatagaaagattcctagaaattaatgggagccctaaagaagttattggtacaggaaataaacaatggggctctaatttctgtggagcaagtaaaaagatttcagagatgtttaagattcctttaattgctaaagtagagcaatcaggacactttaacgagatacaaccaatattagaacactttagtaataaatataaagtagcgtaaaggatgagagatatatggcaacatatggaaaatggattgagttaaataatgaaataactcaattagatgacaatggaaaaaataaactctataaagaccaagaagctttagatgagtatttaaaatatattgaagacaatacaagaaagtttaatagtgaagtagaaagaattagagtattgacaaaagaaggaacatatgataaaatatttgacaacgttcctgacactattattgatgaaatgactaagttagcttacagttttaattttaaattccctagtttcatggcagggcaaaagttttatgaatcttacgcatcaaaacagtatgatgaaaacaaaaaacctatttttgttgaagactatgaacaacataatgttcgagtagctttatatttatttcaaaatgactatgtaaaggctagagaattactagtacaacttatggagcaaacattccaaccatctacacctacgtataacaactcaggacaagctaatagaggtgaactaagctcatgttatctatttgtagtagatgattcaattgagtctttaaactttgttgaagatagtgtagctaatgctagttctaatggtggtggagttgcaattgatttaactagaattagacctaaaggagctccagtacgtaatagacctaattcaagtaaaggtgttattgcttttgctaaagctattgaacataaagttagtatttatgaccagggtggtgtaagacagggtagtggtgctgtttacctaaatatattccacaatgatatcttggatttattaagctctaagaaaatcaatgccagtgagtctgttagactagataaattatctattggtgttacaatccctaacaaatttatggagttagttaaagaaggtaaacctttctatacttttgatacttacgacattaataaaatgtacggtaagtatttagatgagctaaacattgatgaatggtatgataagttactaaataatgatagtatcggtaaagtaaaacatgatgctagagaagttatgacagacattgctaaaacacaattagaatcagggtacccttatgtattctatattgataatgctaatgataatcacccattgaaaaacctaggtaaagttaaaatgagtaacttatgtacagaaatttcacaattacaagaggtatcagaaatttatccgtattcttacagtaatcagaatgttattaatagagatgttgtttgcacattaggttctcttaacttggttaatgtagttgaaaaaggtttattgaatgaatctgtagatattggtacaagagcattaacaaaagttactgatattatggatttaccttacttacctagtgttcaaaaagcaaatgatgatattagagctatcggtttaggttcaatgaatttacatggacttttagctaagaatatgattagttatggttctagagaagcattagacctagtaaacagtttatatagtgctattaacttccaatctattaagacatctatgttaatggctaaagaaacaggaaaaccatttaaaggctttgagaagtccgattacgctacaggtgaatactttgtaagatacattagagaatccaatcaacctaagacagataaagctaagaaagtcttagataaggtttatattccaacacaagatgattgggatgaattagctaaagcagtgaaagtacatggtttgtataatggttaccgaaaagcagaagcacctactcaatctatatcttatgtacagaatgctacaagttctattatgccagtacctagtgctatagagaatagacaatatggagatatggagacatattacccaatgccttacctaagtcctataactcagttcttctacgaaggagaaacagcttataagattgacaataaacgtattattaatacaagcgcagttgttcagaaacatacagaccaagcagtgtctacaatactttatgtagagtcagaaatacctactaataaactagtatcattatactattatgcttgggaacaaggattaaaatcattatactatacacgttcacgtaaactttctgttattgaatgtgaaacatgttcggtttagaaaggaaatagatatggatattacacaaaaagtaaaacaacataataaaaatgctgtattaaaagcaacaaactggaatattgaagatgacgggatgtctgatatttattgggagcaaggaatctcccaattttggactcctgaagagtttgatgtatcaagagatttaagttcttggaatagtttaactgaaagtgaaaagaacacttataagaaagtccttgcagggctcacagggctcgatacaaagcaaggaggagaaggtatgaacttagtatcctaccacgaaccaagacctaaataccaagctgtatttgcgtttatgggtggtatggaagagatacatgctaaatcgtatagtcatatctttacaacattactaagtaataaagaaacaagttatttattagatacttgggtagaagaaaacgactttttaaaagtaaaagctcagtttatcggatattactacgaccaactattaaaacctaatcctactatatttgatagatacatggctaaagtagctagtgcctttttagaaagtgcattattctactcaggattttattatcctttacttcttgcaggaagaggtcagatgacacaatcaggagctattatttataaaattactcaagatgaagcttaccatggttcggcagtaggattaacagctcaatatgattataatcttctaacagaagaagagaaaaaacaagcagataaagaaacttatgaattattagatattctttacactaatgaagtagcgtatacacatagtctatatgacccactagaattaagtgaagacgtaattaactatgttcagtataattttaatagagctcttcaaaaccttggaagagaggactattttaatcctgaaccttataaccctattgtagaaaatcaaactaatgtagacagattacgaaatgttgatttctttagtggtaaagcagactatgaaaaatctacaaatatcaaagatattaaagatgaagatttctcattcttagatagtaaagaatacagtactgccaaggaattcctataaaaaggagaaaagatattatggatagaaaagaagcaatggatttactaagtaaagcagaaatattatttaaaaaacatgatgagttttcatgtgtaagtgatatcaatgaccctatgaagttattcagtaactctaaggatgctaaagctgatgatacgtctaattcttttcagctagagtttatgcatgatatgaccatgtatactttatcttatggctcaggacagctaaaacttattgatttagcagaaggttatgaagcacaaaaagctacaatagttaactcatttcccgaaattattaaaacattagaaaaggatgattcagaagatggaaaaaatgaatagtttagtagatttaaatacagcaattagacaaaagaaagatgttattgtcatgattacacaagataattgtggtaagtgtgagattttaaaaagtgtaatccctatgtttcaagagtcaggtgacattaaaaaacctatcttaacattaaatctagatgctgaagatgtagatagagaaaaagctgttaagttattcgatatcatgagtacaccagtattaattgggtataaagatggtcagttagttaaaaagtatgaagaccaagttacacctatgcaattacaagaattagagtcactttaatttggaatttcctactatctgtgctatactataatagtacaaggtagtaggattttttaatggaaggaagatgacatatcgcaaagaataaaacattaacgatatataatagtgatagatattttaatatacacacaaaagataaagataaaattaatgaggctattaaagtaacacacggtaatgaagaagaaattgaaaagaatatggatgaattaatatctaagtctagaagatatatcatgagggatgaaaagcattacatgctatttaatgagaagtacaataatgataggcttatagaaaaagtatgtaaacacggtggtaaagttacatactatactgattcagtattaccttactatgttttaaaagacttatctagtcaccctgactcagaagttgtttatcgtatgcgcaacggttttactgcaaaagaagtagataatatagctttatcattcatgggtacaaaagttattattgatatttctgtagtatttccttatgtaaacccttatgatattattagaagtttacatgatattaaaacaaatgtagatgaagttcatttatcatttccacgaatattaggggtagatgaaaaacaagaaaagttttatttctttgatggtgaagcttatgatttaaaacccgaatataaagtcgattttgcagataaaattagagtatctttatcagtatggaaaatgtatatctatatcttaacaagtagtcgtgattttgaggatgtagacaatgtaattacgaaattaaaacaacaacgaaagattaagatataaggtgattatatgagtacagcaaatagaagagatatagcaagaaagatatcagagaatacaggttactatatccaagatgtagaggaaatactaagtgcagagacagatgctatttctgacttgctagaagaagggtatactaaagtaaagaatcataaatttatgcaaatagaagttattgaaagaaaaggtaaaaaagcgtgggatggtctgaataaagaatacttccatttacctaatagaaaagctataaaattcaaaccactaaaagaactagaagaggttattgatagacttaatgaagaagagaaataattctcttctttttttattgacaaggtttaaaatatatggtatagtattattaagttaaaaaaggagaggaattaaatgaaagtattaatcttatttgaccacattagagaagagcatttttctgtaagtaaagatgggagtgtgaaatctaatgtactaaatacacctaacggaaaaacacttaagaaattacttgagaagtgttctaacttaaagagagataaaacaaacagagattatgatattgattttctctacaatgcagtacctacacctattagaaatgactacggtaaaatcattaaataccaagatgttaaacaagcagaagtaaagccatactatgagagaatgaataatattattattgataattcttatgatatggtaattcctgtaggtaaactaggtgttaaatacctattaaatgttacagctattggtaaagtaagaggtgtaccaagtaaagtaactattgaaaatggaacatcttctcatgatgtgtgggtattacctacttatagcattgaatatactaatgtaaataaaaatagtgaacgtcatgtagtatcagatttacaaacagttggtaagtttgtagagcaaggagaagaggcatttaaacctaaggaagtatcttacgagttggtagataacattgaaagagtaagagaaatattcaataaggaagtaaagaatgataattatgatggggtagatattaccgcatgggacttagagactaactcattaaaacctgataaagaaggaagtaaacctttagtactatctctatcatggagaaatggtcaaggtgtaactatacccttatacaaatcagactttaactgggaaaacggtcaagatgatattgatgaagtcttagaattgcttaagaattggttagctagtaaagaagatattaaagtagcacataacggtaaatgatttgctgttgtaaaatccctctcatatcgggcatagctttaagtagctgataagagaacctaagtcctgtaataaggatagtggtaatcccgagcttacattattggtgacaatagatggggtgtagagactgagccgaggttttgtagaccaaggtgagacatagtgtatcgacttaatagaggtggtacagtgaaaaaagattatatgacatcagttaaaaataacaaaaaagtatgtagaagatgcaacgaagaattagatttatctaactttaaaacatataagaagaatgataaaacttattatcaaagtatgtgtataccttgtcggaaggaatataataagttagataaaactaaaaatactattaaaaaatgttatgagaaaaacggagataaatatagaagacaaagtaatgagtataatacttctgacagaggtagagagcttaataaaaataggtctaggaaatacagagaaaacaattctttaaaatcgaaagctagaagctctgtaagaaccgcattaagaaatggttctctcataagacctgataagtgttcagagtgtaataaagattgcatacctgaagctcaccatcctgattatactaaacctttagaaataaaatggttatgtaaatcctgtcatgaagatactcatcataaaaaataatcacactatgtaaatgagggacatcaagcccatttaggtaactacaaacaaacctaatggtaagggcttatgaaggtatagtccgttctatatagaaatatataggctaaaacgaaatatgatattaagttcttaatgagtactgaaaactttaaagattttgagagtattcaggatactaaagtaggttggtacctagctgttacccaagaagttaaagaatctttaagattatctgatttagcttatgaggttacagatgtcggaggctatgataaaccattagaagactttaaattatggtttgttactaagttattaagattcttctcagataaaattaaagagatacagaaagaaaataaaaagattgctaagaaagagtatgatgttaaagctcctgaatataaagaatggttagagaataaattaaatgaaacagtagtagaactagatgatactgagaaaaaatttagagttagtgaattagagaaaaagtatattcaactaggtctttcacctgaaattgtaaatatgaatttagttatggataatgatgaattcataaatattgcagaacaatcacctgagtacatggggttatctgactacgctaagtcttacacgttaaatactgcaattaatttaattaatgagtatagagatgtaaaagatgtagttaatgatattgacggaggtaactttaattatgattggttccctattgagttaatgcatccatacgcatcaggagatactgatgtatgtagaagaattcattgtgatgtaattaagaaacttaaagaacaagatagacctaagtcaatgcatttattagaagttaattacccaagacttactaagtctttagctagaattgaatcaaatggtttatattgtgacttagattatatgaaagaaaatgatgagtcatacgagtctgagatggctaagaaccatgctacaatgagagagcactgggctgttaaagaatttgaagaataccaatacaatctttaccaaatggcgttagaagaacatgagaaaaagccaaaagatagagataaagatatccatcagtacagagataaatttaaagatggtaaatggatgttttccccaagttccggagaccataaaggtagagtaatttatgatattctaggaattcaattaccttatgataaagaatatgtcaaggaaaaaccatttaatgctaatgttaaagaagcagaccttacttggcaggactataaaacagacaagaaagctattggttatgcgttagataatttagaattaaaagatgatgttaaagagcttcttgaattacttaaatatcatgctagtatgcagacaaaacgtaattcatttactaagaaattacttaatatgattaataaacaaaaacgaacattacatggttctttttctgagacaggcacagagacatcaagactaagtagtagtaacccttaaattggggttgtaaaactttgttaactgcgggaagagactcgttaggtcttaactactaacttataatggaaacatatataagggcaaacagtaacgtgtttgatatagtaaaaaggttaagaatagagagaatccgcatccaagaccctgaaagtatataaaagtatgggtaaggttcaacgactaggtgttgagacaatacaatcaatacacacccacgaaagcaaaggtattatttctgtggtagggaataataaggagagttatatgaaagagatttggaagaaagtagtaggatttgaaaactacgaggtaagtaataaaggaaaagtaaggaatataaaaactaactatattttaaagccgtggataataaattccggatatgagcaagtatctataggtattgctaatgtattagtacatagattagtggctatgacatttatacctaccgacagctatagtatagttaaccatattgataataataaattaaataactgtgttgaaaatttagaatgggtaagttacaaaggtaatagtgctcacgctaataagcaaggaagattgaatacttatagtgcaagagaaaaacttagttctgtatctaagaaagccatttatcaaaaagatatggaaggtaacatcattaagttatgggattcaccaagtgaagctgaaaaagaatctaatgggtactttaaaagtactaagattagttccgttgctcacggtaaacgtaagcatcatagaagttatacttgggaatacgtatataaggattcaaagagaagtttaaataagtctattaatatgtatgatttaaataataatttattatatgaagatttgacaatgaataaaattatgggtatactagaaatgaataatcataaaacattaagagataaactaagaaatacagatgactttgttgaatacagaggatataaatttaaaaataataattaaaacctaccacagaaatgatatatgatatagtctactcaatagtgagagctattgtgttacctaaacagtaacagattgtaaactaaaaagcttacaaattatagaatttacaaaacttacctgcacacacatcagatgtaaataagtttgattacaaacatccaattaaacgttcatttgtttctagatttgaaaatggagtactgctagggtccgactatagcgccctagagatgcgtattatcggattatttactaaagaccctgatatgctacaatcattcttaaatggggaagatatccataaggctactgcaagtattgtttacaataaaccagtagaagaagtaactaaagaagaacgacaagcaactaaagcagttaacttcggattagcctttggtgaatcacccttctcatttgcaggtaaaaataatatggaagtaagtgaagcagaagaaatatttgaaaagtatttccaaacaaaaccaagtgtaaaaacttctattgacaatgtacatgagtttgtgcaacaatatggttatgttgatacaatgcacggacatagaagatttatccgttcagcccaatcaacagataaaaagataaaaaatgaaggtctaagacagtcatttaacactatcatccaaggttcaggtagtttcttaacaaacatgtctttaacttacttagatgattttattcaatctcgtaatttaaaatcaaaagttattgccacagtacatgatagtatcttaattgattgtcctcctgaagaagctaaaattatggctaaagtgacaattcatattatggaaaacttaccatttgatttcttaaaagcagaaattgatggaaaagaagtacaatatcctattgaagccgatatggaaattgggttaaactataatgatatggttgaatatgatgaggaagaaatagatacatttaattcttaccaaggttatattaagtatatgatgaatttacagaccttagaagattataaagagtcaggtaaactaacagatgaacaatttgaaaaggctactaatgttgttaaaagtgaaaaacatatttatcaagaaatttaataaaagtattgacaatatagttaacttatgttatactatataagtaataaatataaggaggaaaaagagtgaatacaggggagattagatttaatcgttctatggatgaatggattataacaagcatgtaccaggatgagctaggtgggatgaatattgttgttacattctataatagagaagaaaataaacatggttctacagttttaccaacagagtcatctactggagaagtaacagaggaattggcaagtcttgaagaagaatatcctttagctttacctttaagtagtatctcagttaatatttaaaaggaggaactgataaatggaaatacacattgattccctagattttacaaactttactattaaagatagaaatgggaactcacaagagtttgatattacagatgagttaagaattacagagtatacaatacaagaggattttatgcaacaatcagctaaatatgctttttgggcttctatattagagaaggtaagagcatattctgaaatggaacaaagaaacctagaaacaattggtagtaagctaaaccttacaattagacaagagtacgaacaacaaggtaaaaagcctactaaagatatgattgaatctagtgtttatattcacgattcttatcaacaacaacttaaagttgttgaggcttggaattataaagttaaacaacttcaatatgttgtaaaagcttttgagacaagaagagatatgatgattcaattaggtgcagaattacgacaaacaaataaaaatggtggaattactaatccattttcacattaaaaaataaagtaaagaatataattgacaaatataaaaaactatgttataataaataagtaaattaattaaaaggagaaaagataattatggatttcaatcaatttattaacaatgaggcaagcaaattagaaagcaataacagttcttttaacaataatgtagagagctacaaacctaaaaaccctgtactacgtttaggtaatattaaagatgcaaacggaaataaggttgttaaagaaaatgcttttgtacgagtattacctcctgcacaaggaacaaatgttttctttaaagaatttagaacaacaggtattaactattctaagaaagatggttctcagggattcacagggttaacattacctgcagaagagggttcatctgtccttgacccatacattcaggattggataacaaatggtgttcagttcagtagattccctaataaaccaggagtacgctattacattcatgttattgaatactttaataacaatggtcaaattcaaccaaaaacggatgctcaaggaaatgtaatgattcaacctatggaattatctaatacaggatataaagaattattagctaacttaaaagacactatgttaaaaccatcacctaatgcacctcatagctttatctcagcaactgaagcattcctagttaatattgttaaagctaagaaaggtgaaatgtcatggaaagtaagtgtttatcctaatgcccctttaggtgcgttacctcaaggttgggaacaacaattatctgacttagaccaattagcaaaaccaacagaagaacaaaatcctaattttgttaacttcttaatcaataacgttaataacacagagttaagtcatgataactttaaatttaaccgtgaaacaaatgtcttaggtgaagaaccttcagagcctaaacaagcacccacacaacaagatgtagatagtcaaatgccaagtaatatgggaggacaacctaatcagcctcagcaaggtcaagtaggtcagtatgcacaacaaggtcaaagtaatggtcaaggacagcagttacaaggtacacaacaacctatcaataacactcaatttggtcaaggaactccttcaggacaacaaccaagtaacacaggttctgttgattgggataacttagcgcaacaacaatcacaacctgattcaaacccattcaatgattttgatgttagcagtgttgatgattcacaggtaccttttgagacacaacctcaaaatacacaacaagcacctgaaccacaacaaactacacaagagcctccaaaacaaaaacaaacgcaaagtattgacgatgtattaggtggtctagacttagataacctataagatatagagtgccttagagcactcttttatttgagatataattactaggaggatattaaatggcaagagcaaaaaaaggtaaagaagtcgatttaacagatttaaatacaattgatttaggtaaagaattaggattaacattgctatcagatacaaacagagcagatattaaaaacgttatacctacaatggttcctcagtatgactatattttaggtggaggtattccattaggtcggttaacagaagtttacggtttaactggcagtggtaaatctacttttgcagttcacttatctcgaattgcaacacaattaggtgttatcactatttggattgatattgaaggaacagcagataacaatcgtatggagcaacttggtgtagatgtttcaaaactattctctattcaatcaggagaaggtagacttaaaaatacagtagaattatctgtagagcaagtaggtaaagaattagagtactggattgacactttcaatgaaaagattccgggagtacctattgtatttatttgggactcattaggggctacaagaactcagaaagagattgatggcggtattgatgagaagcaaatgggtcttaaggcatcagctacccaaaaagtaattaatgcagtaacacctaaactaaatgatacaaacacagggttaattgttattaaccaagcccgtgatgatatgaatgcaggtatgtatggtgaccctattaaatctacaggtggtagagcttttgaacatagtgctagtttacgtattaaggttcataaagcatctcagttaaaacagaaaagtgagttaactggtaaagatgaataccatggtcacattatgcgtattgaaactaagaaatctaaactatcacgaccagggcaaaaagctgaagcagacttactatctgattatatggtaggtaaagaagatgaccctatcttattaaatggtatcgacttagaacatactgtatataaagaagcagttgaaagaggtttaattaccaaaggagcatggagaaactatgttacattgaatggtgaagaaattaaacttagagatgctgaatgggttcctgtacttaaagataataaagagttatatctagaattgtttagtagagtttatggagaacacttccctaatggttactcaccattacttaataacaaagtaatcgtaactcaattagaagagtatcaagctcttgaaaactactataaagaatgggctacagataataaacaagaggaacaagaggaagaactaaaaggagaatctcaagaaaaggattctgaataatagatggataatttaatagataaaaacatgaatcaggtaaaagaatctttggggaatgcaaattcctcagatgttcttcctttaccttataaagatatagcaaagaaatttgaagaagtaaaagaaaaaggtgaatcaattatcattgaagaaggtggattcccttatacagattctacagtgatgtatatagaacatgtaacagatagatgggcaggaggatattccttaattagacatgaaggtgaagaagttaaagtacctaagactatccatttctctgatatatatgttaaagataaatcacacaaagtaagaataatcttcgagggggctaatccttatgaagaaagctaataatggtaatagatatgtaatagatatagatggtatacctgttgattttgaaagggatttagatagtttacttaataggtataaaaaccttagatggtcgttatatcataggtacgcagggattttatctaatgattttgaaagacaagaactaagagaatatattgatgagcaatttattaaattagttaaagaatataatattagaagtaaagtggattttcctggatatattaaagctaaactaactttaagagttcaaaatagttatgttaagaagaatgaaaaatataaacgtactgaaattatcggtaaaaaagattatacagtagagtctttaacagaagatttaaatgaagacttcgaggataatcaaattatgagttatgtatttgatgatatagaatttacagaggttcaaagtgagttacttaaagaattacttattaaccctgaaagagaagatgatgcctttatcgtttctcaagtagcggaaaagtttgatatgaaaagaaaagaagtagcaagtgagttgacagaactcagagactatgttagatttaaaataaatgcataccatgagtactatgctaagaaagaattaaataaccatagagttaatactgaaaatcatatttgggaaaactagttacagtgccttccttgtgttatattattatcgagaattcaataataaagcatagggaaggcttttttctatgtcttatagaatgctttaaaatagattactaaaataaagattggagattaagcttatggctaaaaagaatgttaatgatgtattacaacaagaatctgttacagtagcagataagtatttacaagttaaagttaaccgtgacggttatactcgtacacatgaaggacaatatgcgtacaaagtagtttcagagggagaagaattattcttataccctgtacaaacagatggtaaaggtacattaaatgtaatgaagaaatcacctattgcttacactgatggagacaatatccatttcgtagtaaacacagtagtagacccttataatcactcatttatccgtactgaagatattaaaggattagataaaggtaaacaacttattcaagctttcttagctttcgttgaagaccgtttcaaatttggtgtttataacgtatttgttgcaaacaacaaagaggatgtattatctattgtagaccctacagataatgatgcagatgaagttaaagatagtttagagcacgcacatgaagatgtaattgcggatttccctgctagccctgctcgtaaggacgttaaaggcgtagattcaggagaaggtcaaggagacacttcagaaccatcagcacctaagaacgttcaagttactcctaaggaagacggagcagacgtatcagcagaataatatatagataaggatggtaaatttggctaagttaaatttatacaaaggtaatgagttactaaacagcgtagaaaaaacagaaggaaaatcaacaatcacgattgagaatttagatgctaatacggattaccctaaaggtacttttaaagtatcattctcaaatgattcaggagagtcagagaaggtcgatgttcctcagtttaagacaaaagcaattaaagttatttcagttacccttgacgttgatagtttagaccttacagttggagatactcaccaactatcaacaactatcacgcctagtgaagcatctaacaaaaatgtgtcatttgaatcagacaaatcaggtgttgctagcgtaacatcagaaggcttaattgaagcagttagtgcaggaacagctaatgttactgtaactactgaagatggtagtcacactgatattgttgctgtaacagttaaggaacctattcctgaagcacctgcagacgtaacagttgaacctggtgaaaatagcgcagatattactgtataggaggacaataaagaatggaaaagacattaaaagtttatagtaatggtgaagttgtgggctctcaagtagctaataacgatggagctactacagtatctattacaggcttagaagccggaaaaacttatgctaaaggagattttaaagtagcatttgctaatgattcaggtgaatcagaaaaagtagatgttcctgaatttacaactaaaactcctactgaagaaccttcaggagacgcataataattaagaccaactaaaaagttggtctttttttattgacaatttataatatctatgatacactatataagaattaagaaaaggaggggaaagtaatggatattccaacaatattatttagaaatccatatgattatacgaaagtaaaaaaattaatggaaaacaaagagcagtatattgtagtaaagtttgattctgtttctgttcataatttaaatgttcaaggtatgatgaatgtcatccaagattacctacacatctatggttacagagttaaagagtacggacaagaaaattcttctaaagatgatgaaagagacgttaaaggctacttatatgaaagagtaggtgagtagggtatgggaattatagtaaactccaaccatattcaatcagacactttatatgagtatgatagcttttttgatattgagaaagtagatacatttgaagaaggattgctttcaatacaggatgagccaactgttttagcaggattcatctatgatgatatcacatttaataaggtcattaattctaattcagatattgatgattatattaagaataatgatatttattatgtctctgatataggattacttcctgatacttttatcactgttgattctgatagaaaatattattcattattacaacagataactgagttaagtaaagacccttttcctaaatgggtagaggatgatgcaaaaggtttaactaagtattataactttcaagattttgaagatgtatttgatttaaatagtttttacaaaaaagaagttgacatggtaagagaaaagtgctataataatggtaatgtatatttattatatgaggttctgcctgattataaattacctctagcttatagtttactttcaaacaaggagcatggtattgttattatcggttcacagacacgttctaataatgatatactgactttttatgttaaaggtatggatgctaaggcaatagctagtatgttcaatgtagaacatgattatgattctaatattttccatacatttgtaaacagtcacattaatattttaggaaatcaaataactaagtttataagagagaaaggaagcagttatgagtaactataaaacaatagaagaagtacaagcagttattattggggtattatttaaagatgaaggtaaaattgtaacatctaagtttaataaaattactaaagagtttggtttagatagaatcggtaaagatgaccttaaagaaattgtagaggatattagacaagacgcttatctaaatgaacttaaaaacaaagcaattaaaggtaaagtaacgttaggtgatttaaaagatgttgcagataaccaagtattcgaaggtaataactaccatgaagaagtatctacttatgtagtagctaaagaaaaagaattgtctcacttaagagaacagcgtaagcacaataggcatactgcataccctcaaattatgtttgatgaacttaaagaacatatggttaaggaattacaaggggaaacattagtagaacatcacggaagtaaagctaatattaatgatacagagctaattgtgttactatcagatttccatattggaagtattgtatctgatatgactaatggtaaatatgattttgaagttcttaaatcaagattaaatcattttattaatacaacagttaaagaaattgaagatagggaaatttctaatgtaactgtttactttgttggggacttagtagaacatattaatatgagagatgttaaccaagcatttgaaacagagtttactttagcagaacaaatctctaaaggtactcgattacttattgatatcctaaatgtactatctaatgtagtttcaggagaactaagatttggtattattggtggtaaccatgaccgtatgcaaggtaacaagaatcagaagatttataatgataacattgcttatgtagtgttagattctttattgttattccaagaacaagggctattaaatggtgtagatattattgataatcgtgaagatatttatactattagagatacctttggcggtaaatctattatcattaaccacggagatgggttaaaaggtaaaggtaatcatatcaataaatttatcttagatagtcatattgacttattaattacaggtcatgtacatcatttctcagtaaaacaagaagattttaatagaatgcacatcgtagcttcatctccgatgggatataataactatgctaaagagttacatttatcaaaaactaaaccttcacagcagttattatttgtaaataaggaaaataaagatattgatattaaaacagtatttttagattaaggatggttaataaatggatacaatttttattataggtgtagcgtttataacttttgcaacatttaacatagtctttagattatttgatttatggactacagagaaaaaaatggtaagtcaaggacaacctccactaagtaactttgagtactatcatgtgatagtaccttacttagtaggtgttattgttattatactgagtattatttttagggattccttgtattccgcacaatcagggttcggtgttattattacaagctttatttacatgctagtttatgttataattggtcttgtagggtcatttgtacttacaatattccaagctagaaaagctagacagtatcaaacacaggaggataataatgaagttcaatgatatttatgagcaattaattaaaaatgatacagtacaaaacattcatgagtctcaagatgacaaaggaaatatttatacaatacagtttgataaaggtaatgataagtatttatttaatgttattaatgatggattcttgaaagaaatgacaaatggtatggtagaccatcctgaaggtcagccatattcagtaagtttaatcaataaagaaacacctagtatgtcagtgaaacaatatttaacagatgtagaagatattgtacctactattagaaaaatggaaaaggatttcttatagagtcaagtctttacttgactctttttactatatatggtatattaatatagaggtgacttaaaaatggattttaattttagtgcttttgataatagctcattagcaatgagaattagtgagggtgtatactatttcaatgatacgccttattactttattgagcatgtagaagaagaaatgtctgagtatgttattgtatatgacatacatgacagagaggaaaaagaaaatcctcagaagaaatatagaatagaaccttaccaacgtacaataccgggaggaacacctcttagtaatttaattaagagtatgatgcctcaacgtaagtatcctaagaaggttacagaagaccctatatttgtagctaatgttattcctttaggaacagatacagtaacaggtaaaaccggtaaaggattttttgaaagagataaggatagaactatctattctcaaaaggaaccaactaaagtcgttcatggtcaatacacaggtgtttttataggtctaacaagtgttaagtggaatagaacatatacccccttagaaagtgttgttgagtactacaaaagggttaaaggagataggttaaatgtctaatgatgtagttaagttctatgaaaaagatattaaagaccttatcagaactaaaaaacacatgttcaaagacgatgaaataactagtgatataaacgatatacgaatcttcaatgagaaagtcatttgtcaaggtaaatgtagaacagattgtttagtgttagaccgtaatggtacagtaatgggtatagagataaaaacagaacgagactctacacaaagattaaataaccaattaaaatattatagtctagtatgtaagtatgtatatgtaatgtgccatgacaaacatgtacctaaagtagaacaaatacttaaaaggtacaaacataatcatgtaggtataatgagttacattagttttaaaggcaaacctgttgtaggtaaatacaaagatgctacaccatcaccacatagaagcccttatcatacaatgaatatattatggaagacaaacttaatgacaatacttagattgattagagaccctcatacgtatagaacagggtatagctataatgctagtggtagatatagtggaggggaaggtaatttctcccaaacaactcaaagtaaaagaatgaaaaaacctgctattattaaccaaataattcattatgtaggggtagataatacttataaactctttacaagaggtgttatctatggttataataataggtgggaagttatagaagaagatttctttaatactatgaagaatggggtaagagtaattaatgagcaaagacaaaccaaatagacgtaaagagatacagcatcaacctgttaactttgcccctacgaatactttaacaggagctaataatagtttctttgctaaaaagccttcagagcctaaagatgcaacatctgttattgaatatcgtatactatttattaaaagatttgataacgtaacaagtacagatgtgaaattacagaaaaagtatgcactaaatcttattagtgaagcacttgatgttaaagaaacttacttgtctcttaagcaaaaaggaaaaaaaacagaatctattttgcatacagatagagtttattatgttcatagaggtaaaaaacttattggaaagtgtagtatcagagaacaaagaacatttaagggtaaacatttgatatttatattcaaaacaagacatagagttaaagcagaaaggaaagataaataatgttaaaaggattttcagaacatgtagacaaacctacaactattaagaccttatacaagaccttaacaagtggtaaagtagaattactaggtgtatcttacgatagtgattacttcccttcaggtgttacagtacaatcttacattgaggatataggtaatgaagatgagggtctacagtttgttaataaggtaaatgtagtagaatcaatgaaacaggctgtagtaggtatgaataatcaattaggttcttcaggtcttggctatgtgagaactgaacaacttaaaaaagagttggaagagactggactaatgacagatttacttgctagaggtactaacttaacctctactaagaaagtagatattgtaagtacttttattgagcctgaggtaacataccaaaatattactatagctaaagatattaaactacgtttgtataaagtagaagaagaatcaccattaaatggttacactcatattgtatacttacttactacagaaaaactatatgatggtcaaacactcttcggtatgctctctaaaaaagataagttatctaaaggagatactgataaattattagcattcttcagaaacaatagtttaataagtaaaagtgtattttgtgttaagttattaagtaaagactactactttaatttatataatacacatgagacagggatattctttttagaagacacagatgttattactattgcttgtggtcagtcatatgttaaagttaacactaaagatattaagtctagttatgttaaaattgaagataagactcataaattaactgagctagtaattaacctaaagggtgacgacacattaactattttattctaggaaaatgttataaatatgtgataattaagtataaatatacgttatataagaagttttcataatgtttttaatacagaaactagttaagttttttctacttgctctagtttctgtgaaattatatttatgaaaagttaaaatatcttttaggtaaaggctttgtaaatagttaaaaaatatattaaaattttatacaaagtagttaataaaattatattacatttatatattatgaaataataacagaaattgtgatatattatatagtgtaaccttgaaacagttgatgttgtagggtttgtttatgttcgttaaactggtttcagaacatcagttaccataaataaatgacagttaaggagagctatataatggctagaaaaaagaatttaagaaataaaaacagtgatataaaagttgttcctgataaagaaaaagaaagtatattatctaagttataccataataagttactacgttcaaaggtagataatgcattagatgaagatatgagttatgatgatattatagaattatgtaaagaatatgatttagaattgtctaaatcagctattacaagatataaaagtaaaagaaaagaagctattgaaaatggttgggatttaggagaattaattgataaacgtaaaaaaacaagtgtaaaagatattaaggaaaaagaaactcctatattagaagaggagcaactttctccattcgaacaatcaaaacatcacacacaaacaatttatgatgatattcaagtactagatatgattatttctaaaggtgcaaaaggattagagtttgtggaaactttagaccctgctttaatgatacgtgcaatggaaactaaagataagattaccggaaatcaattaaaaggtatgtcatttattggacttagagaattacaattaaaacaaacagctcaagatacagctatgagtgaagtattattagaatttatacctgaagagaaacatgaagaggtattacaacgattagaagaactacaaaatgaattctacaaaaatctagatttagatgaggaaagtagaaaattaaaagaagctcttgatagagtaggctatacaatttagatagtgaggttagagtaatggcagatgagattagtttaaatccaatacaagatgctaagccaattgacgatatagtagatatcatgacatacttaaaaaacgggaaagtactgagagttaaacaagacaaccaaggagatatccttgttagaatgagtccagggaaacacaaatttactgaagtatctagagacttagataaagaatcattctactataaaaggcattgggttctctataatgtatctgttaactctcttataacatttgatgtttatctagatgaagaatattcagaaacaactaaggttaagtatcctaaagatactattgtagaatatacaagagaagaccaagaaaaagatgttgctatgattaaagaaatacttacagataataatggtaattatttctatgcacttataggggaaacaatgctctttgatgaaaataaattaaataaagttaaagattagggttgacagctcctatagtttatgatatagtatatgtatactaaaagtaaaggagctaacaattatgtttatttcattaaatcaagaagagaaagaattattaactaaagaggaaagtaaatacacaccattagaaacatcaagagagtttaacacacctaaagaagaattcattgtaacaagctataatgaaggtaaacctttagattacattgcaaaagaagctaaggtaagtatgggattaatttacacagttctaaactactataaagtaggtaagcgtaataagaaatcacctgtagaagaaagaattgcacatatcttaaaagataaaaacttagtcaaagagattattaaggattaccaatatatgaatttacaggacatttatagtaaatataatcttcataagaatggtttatattacatcttagatttataccatgtggagagaaaatctgaacttaaggacaaagcattagaagaggataatattgtcgttgagtaagtaaagaggttataatatgagaaataaaaaatcatttcaagagcagttaaatgacatgcgtaataaagagaaatgggtatctgaagaggagttcactgaagaagtggctcctcctgaagaacctgaagtagaagaagaaaaactatatactttaaatgagttaaaagagagcttactagatgctcaaggattaaaagatgttgtagctgattttcctgcatctaaagatttatatgaacctaataagttatatatctgtacaatacctaaaggatatcagtctaccgaagtacaaccaggacaatatattggtattagtactggattattatcagagtcagaagacttcagccatttaagaggtcaaatgcctagaaacttatatgaaacttctcatgttttaaaacctttgatacgtattaataatacaaatattgaataccaacaacatgagttacttgaagacattaaggatgacaaaaagatatatgatgtagagttagaagacttaagattagcaacaggagaagaagtttctcatttagaaattgttgataataagttttttgaaagtcgtattaatgaagttcttgaccgatacactgaactaacggattccaatgatttacttaagtactatagtaaattacgagaattagtaggtagtgacaaaatgatttattgttcactcctcgataaatgtgttaaaattatagattaatagtagtctcctcttatattataattgtaagaggggacatttttgtatagaggtgttaattatgtcaagaaaagcaagtatattctatatactagtggttattgttttggctttctctatctcatcttattatatatcttctttcatgtatcacgacaaagcaaaaaatgaagtctctactgagttatcgaacacaggaaagattaaagaagaaaagaacgtagaatttgtcggtgactacacattgaaaaaagtggaagataataaagcttattttatggaaacattacctacttacctaccaggtagaacaggagataacagcatagatatgaggtactacaaaacaagtagatttaaggaaggggtaaatttcaagcttattagggtatatactgaagatggagaagataatccaattcataagtataggtttgaagcagtaccaaccaaaaagtaataaggaggtgacttaaatgacaacattaattgtcgtcatctttattgctatcatttattacttatggaacagtgattgagtcaagttaattcttgactctctttttgttttatggtatattaatatatagaaaggagagattaactatggaaatggcagatttagaaagatttgatgcatttgtaagactaatttcagatgatgagctttcggaggaaagaatactggagttaagcgtagacttactaaacccgatactagaaggaggtacagcttacaaggctaaaaaacgtattaagagtaaatttggtaagttagaagcaaaaaattttaaacgaaactataaattcttacttaagtcgatagctcaaatagaccaaaggagataggacaatgacagaaagggaaaaattaattaaagatattgaagaggctaatagagacatacagttacagttaaaagaagtagataattataaggacagcatacgttctaaaggaacaagaaattatatttctacaaaggtattagattctattatggttggtttcatagttagttttttaatactcattataatgcgtgtacttgaatattttgtaacaggtaatgctgtttactcacctttagcgcctgcagttattattatgtttgttttagcactaggtacatggaaagtaagtaagatgaacaaaatagtatcttatagaggaactattaagatgtactgggaactaagtaatgctgagcaaaaacaagctaaggtatttaagtatcctaatgatgaagtagatattgtatcaaaacataacttaagacaaataacttttagtgagattaatatacttcatcttaaatatatgagatataataaggcagtagaacaacatactaaattatctaaagaactttttaaaaaagataaagaaactgttgacaagaacaaataagtgtagtatagtattactaaaggaggagagatattatggttatacctagtattaaagcacaaaacaaattcaagaatgagttagagtattataaacaaggtcacattagtgaaagtaaaatgttagaattagcttttgattacatccaagaattagaacaaaataacgaatacgttactaacttgctagaagaggagagatatggtgagtaaatttatcggagtgtacttatttaatttactaatagctattattttaactttaaccttaataggtactattactgactcaattgagagtactttagcccaaataatcgtagggatgttcataatcattactatatatggaatcctatcagcgttaatacctattctagttcataaagctgtatcaccgggatggagctatactgaatggaatgaatcctattacatcagattacctggagaagagaactacaagtactatagtaaatggtatttagatttattaggagttaaagaattttactataagagagacaatggagaagaagtaaaagaaaaaaatatatcatgggcttttcaagctgaagtaaaaagacctgaagatgttaaccactggaaaaaccaattgcttactaatagacctttaacaattttagaatataaaaaattaaagaaattagataaggaaagtgaaattaggaaacaagaagatttagaagaatataaacaatacaatagtaattaaagaggtggaaagcaatgataagctcatttgatagtatactacttgtcatatacattattatagcttttgcagtagctatggcaattatctacttagtatttaaaggtatgactattctactagataagctaatgatgttattattaagtaaaactacattagatgtagaagcttgctctatgataatggcagtcatcagtacaattgtgtttggaattattgtacttttaatatggctagcagtaaataatattttactataaggagatttactatggattttaatgactttataaacagtgaatcggatagggtaggtaagcctaaacaaaagaagaaggtagagaataagctaccttcttctactcctattgaagataaggaaaagaaattaaaagagataagaaagaaatcattatatattgatttaaggagaaaaagaaatgactaaagaaacaaatgtactttacaaagataagtatagagattatactatagttgtaagattagcagggaatattattgttactgaagtagataagaaacataaaacagcatttacacctattatatttgacaatggtgtagaaggcgtagagcttgtaatgcgtataggttctgtagagcttaacatgacagatttacgtgagttcacaaaagaagtatctacggctcagaaagctttagaatattttaataaaaaactttacattaaaggcttgacagatgaagcattttaatatatactaaaagtataaataaaataaagaaaagaggaatgattattatgttattaggaattttatggtttatatggggatttgtatcatactttgtattgatgtttggaattgagttttggaaagatagatggatgccaggtgttatcggagcaggagccttactactattcttattttggattatgaaatctatccataatgctatgacagtagtatacttgtattaggaggttgtatagatggatatactaattattcattataaagaaacaaataaacgggttttaaaagaaacaatacaaacaatacaaaatcatttaaatgatgaacatggtttggttaagatgacagcaacaaaacttagcagagagaatatagagaaaagatttaataactataatatagtcattgcagaagatgaccctgataattcttatcattacggtgaagctgtagaagacgcagattttattatagacataccaatttcatatttagatatacatgcaggaatagaatgggatgttgataatcctgtagatatgctagataggaatcctgattttatagaagctgtaaataaactaaatgaagacttaatgttataaggaggaaatagaatgctaaatgaaaaactaaaaaacctggaagatacaaaagtatacatgattaatagtattgcaagtttactaagcgcaagtacaggaaaatcaagtaaagtattttttgatgaagggactattaaaattgtaagtggtgaaacaaaagcagtagaagtcattgataacttagttcaccctcactcaggacgtttacctattaaaacaacagaacgtattgcgctaggtagattaacagattctttacagtttgttatttcagaaatagaagtagttaaagaccaaattatagatgaagaaaatgaagcttacattgattttgtgatggaagactggaactgggattaatgcctatggacttattaactattgcttctgttgcttttatagctgtagtcattattgatttgattaatgatgatatgagctatatgcttactggtactgcaatcttaataaatatttgggcaggattttatggatggtttttcttactgcaagcaggaatgttacttttcttactattagctaggaaagttaaagatgataaggagtcaatactatattccagtgcttcattaatatgtgcactaggaatgataataaatcttctttcattttcttaaaaataagtattgacacctttgtacttttgtattatacttagtatataacaagtacaggagatgattactatgagtaaagaaacaattagaagacaattttcaaatgcaattgagattatggcaacaactaaagaatggtggaacttccctaaaagttttgatacgaataaagaatttaaaattaaaacttttaaaaatgatacacttgtatttgaagtcagagaaggcagtagaaatttaggaagctttgtagtttttacaaacattgattttgattatgataaactagaaggaacttcaacacaatatatgattaattactttgctaagaaattaactaaagatatgtttaactatcataaattacaattatagtaggaggtggaaagatgagagaagagttaaaaccttttaataggaaacaagttaatgttaagggttacttagatgatgttaagtattcaaagcgtagaagacataaaggtaatcaacatgggtgtgttaaaatcacagttactgatgtaaagattaatggtatacctattgaccacgttaacattgaagttggtatctctttctatgaaaaactaaaggagcttcaaggaaagagaattcaatttgtaggtactgtttataagtatgttaaacatgctagggggcgcaaaggtagaattaaaggattttataaagaggattatagcgtaactttagataagaagttacaaaaggaggaaaaataatgattaaaagaagaaaacatttagaccactcattacagcctgagaaaggatggagaacagtaccttttaatgggtattatgaagcgcatcctacgggtttaattagaaataaagtaacgaaaaagttaattaaaggtacacagacaagaaagaaccatcctaagtggactgctcatgagattgtatacctaattaaccctaagaaaacaagttattctaggggagtagttattgcacatacattccctgaaatgattagtcaatcacgaggagaccttaagaacggtcatgtgtgttttaaagatggtgaccgaagtaattgtcatgtagacaatatgtttattggtaaaggtaatgttaacaaaaatatctataaattaaatgattcttatttaactagaaaagatattgaagaggatgttaataatttagttaatgaaagattattctctcaattagaattattgattaagaaaaatgaaccggaaagaattacacctagtaatcactttattaaaagagataataatgtgttcagtatcacagatttatctaaaaactcactagtagagtttgagttagaaatcaagaatattaagtaaggtggttatataaatgaatgagtggtatgctttatgttattataacaaaataggtaaaaagaaaatacctagacaaattaaagctcacagggatgtatctgtattagaggatttaaaagatagattagaagaacaaaatcctaaagaagaatacaagattaaaacaacaaaagaatttgataaggaaagataattaatgttaacacctcaacaaaaggattcattaaaagagcaacaaaaaaaattaagtaaaaagaagaaataagtcttgacaattgagtatacataggttatacttaagttaacaaataaagaggaggtatgacctatgttattcgtaatttttatattggcagtactgtttgtacttggatttatgaatggatggaactcagaagactagataaggagtggttgtaatgaagttagaagataaagtgttagagagaattgattctcttggaaataaagcaggtaacttaagtaatcaagtaatggagtcattagtaaagtatcaaattacgtacggtattatagatattgttgtaagtattttagttattgcactaacaatatttttaggtaaggtttaccttaaagaatataagaaggttaaaatggatttaaaagaaagcttattgtatgatgattacgatgacttaagtggtatcggatggtgttacacaattctattaatactattaacgttattctctctttacgcaatagttgcaggtatcccaactgatattatgagattaattaatccggaagtttatgcagtaaaagatttaattgagcaagttaaaggaggaaattaaaatgaaacaaagagactttgaatttgaagaggattttgtattaacttatgagtgtgaggattgtaagcatttcgaagactggggtcatgatgaagagcctgaagaatgtagtgaatgtggaagtagtgatttaatcaataatacaagtcatgaagatactgagtgtgatatgtgtcgagggtatattgatatgtggcaagatggatatagatatatgggagataataaagagtatattgaaaaagaggaatcaggtttgatttgtgaagattgttatgagaaattagatatttaataaggaggaaattaatatgaataaagcagtagaacaagcaagtaatgcattaggtcaaggattttcagctatggtatggcatcaagtattagtagggttagggtttattttattaggattggtattatctttactggtttgggtattagtaaaaaaattccatgtaccttttaatcacccgacagcttttgtagtgtactcaattatgttagtgagtattgttgctagttttatttggggcggtttacatgtaattaaccctgagtattatgctattttagaacttaaaggttttataaagtaggaggaattctatgactaaagaagagttagagcaaaaagtaaaagaacttgaagcagagaataaagagcttaaaaaacaaatagaacgttttgaagacgaaggaggaaaaacaaaagatgaacagtagagaaaagaaaattttaacactaacagtaaataattttttaatgttagctttagatattgtagcacttgttaggtacaagaaaggtaaaattaagcaagagaattataacacaggtcaaatttcaagaactatagttacaacagccaactcattaggtattctttacctagaagagcaagaacgtaaagaaaaaaaatctgttaaaataggtactcttgaaagtggtactctaagagggtttaaaaataaataaaaaagtttaagaaacctattgacattaggtttcttttattatatactaagagtataagaaataaggaggttaatttatgaatggtattattgtattttacaaagaagaaaataaacatattctaaagaagctcttagaattcattaacacaacatcaaaaggattaacttacaccttagagggtacattagttgataatgatatcgttttacttaaagataacaacatatgtgattataacctaagacagtttagtaagacaaatgatggcttagttataggtctattgagtgaatcatataatgatgttcattactatgaaaaaggagatgcctacggtatagaaagattaactatgtatttagaggagatgagtcaataatggtaattgcattttttattttaggtctattattagtattatgtttagttggtcttgctattattaaagtactagaagcaaggctaaagaaaaaagaactaaggagagaaagagaatcttttggtattcctgaaccagggagaaaattagggcacataggatatgtcgaaagtaaatatgctttaattcataaagaatcaagtaagataataataagcgcagagaaaagtaaagttgtagaaacattacagaaaatgtataatttagagttaacatctgtagatgtttctagtgtaacgggactctctcccttaggtacaggacgtatggagaatatggtattgctttcatacaaactagaaagagaatgactttataaattaattgaacttaataaggagtgatacaatggaatttatagataaaaataatgtaattaaagcttatgatataccgaatgtttacttaaaaggttacgtattgtatgcatgtgataaaaatggagacattacaacctatgatgggtatgaccaaatacactataaagatggtagagtattaacattcccttttgataagccgttaagaaagataaatgtactttcaggatattacaaactatttaagaaggaggacatactatgatttattttgttagtgatttacattttggtcatgataatattagagaatttgaagcacctacaagaagtcattggaattcagtagaagaaatgactgaaggtttgattgaactatggaataacacaattacaaataatgatattgtttataatatcggagacttcttctttaatatgaaaccatctaaagtagaagaaatacttaatagactaaattacaaagagatgacattgattgcaggtaatcatgaccataaaaagcttgttaagctatatgaacgtaatggtatcacagtgaagtatgctgatatgcttaagaaggaaggtaaaagattttatctgagtcactaccctacacttattggaagaaagaatatgtttaatattcatggtcatatacattcacaactaatggatactgattaccacattaatgtaggttatgatgtagaaggaaagattgcatatagttttgatgatattctaagtatagcaggtgagtatagtggagaaattcaaaggtaaagacttatataagactagaattagaaaacaaacaattaaaaatttaattattaaagtagagaaattacataataaacatagcccttatcaacctattggtcatgtttattattaccctaaaacaaaagagtttactttatctaaacccgagcaaaagatatttatagagtatatgaaagaattaggttttaatgtaaaacacaagagacgtaagaaaacacttattatttataagaatgcattcactgaatacattagtaggtatcatgaagcaatagagcagattgaaggagggaaataatggaatatttatttttatttataggtattggcatgataatttggggtttcatagcaccttatcttgcatttgtagtttactataaacatgtaagagaaaatcataatggattcagtgatgaggaatctctagaagaggctatagtatttggtatgggattcatgtttatagcatttattcctataggtatactagttgtaattgaagaaattaagattttattcttttaaatgttgacagctacaatatagtgtgttacagtatagaaaaggaggttaattaatgaagcattttattttaattttaggaattgtaatactagttattgcattaggtattgttttaccggcatggattttacagttagtactatctgcattcggagttaaagtaagtatttgggtatgtatcggaatatttattttaatcagtgcaataggaagtatgtttagcagaaattaaaggaggaactataaatggcaaaatatgaatcaaatattaatggagagaattatattgcaacaccgtcacaagctttaagagaggcactagcaaaattaataactgaagaaaagagctttgcggagtaccaaactaaaggtgaggagcagtatgaatcacagttacaactaagacactttgatacaatgatatctcagtatgaggaagctattagagtactagaagataaatatagacctcagatttttattccgaaagataataaggaggaaaattaattatgaaagcagaatcaatagcaagattttttaatgacaaagtactacaaatagagggttataaagtaagattcttacaggctagttcatcgtatattttagatatagatactatagatgaatcagtattgtttttagaagctcaagtatctacactttcaggtaaacatttattagatacagctattacaattgagagacctgaaacattaagtgctaaagagctatatacagaaattagtaataaactacaagctattgtaggagaccaaactaaaacaactatagaactatcaagatattttaaggaggaaaaataagtgtctaataaaaccattacaaattatttattaaatttagaaggaataaaaggagaaacgtatagtattattgctcatatcaataaacaaactggttggggtgataaaggggattattttgaaataagcataagttataaagctgataaagaccctagaacaacgagatatattacaactgaaatttttgttgattatggtagtaataatccaaaagaaattttattacaattaagagataagattttttctattgtagaagaacaggtagagactgacaatgattttattgaatctattaaagaaattaattcaactaaagaattagaaaaactaaagccttatatcaataatgaatattattcaatgtttaaatcttctattgaaaaggaaatacctgtagctttatcttctgaagtactcaatagatgtacaggtaaaacaagcacattagcttatttagcactagaaaaggatttacccttagtagtatcaaatgaacctatgagaaaaatgcttaaaaataaattccctcatcttagagtagcttctgctgaagattattcaaattatgatattaaaggtgaaattgttctaatagatgaagtagatattgaccagttatatagtgctgataaagtatctgttgatgcacttttagtgggtatcattaaaaattaaataaatttataaatacctgttgacagcctgttgacagcaggtattttttatagtatactttagatataaagaaaaaggaggtaatataatgatacccgtaatagttatacttattggactcatattatttttatctagcggttataagttggtattgggtaagtattatgatgatgtagatttaaaaatactatttaccatatttggtgttgggattgcattactacttggaggatttatattataaagcaggagctattttattttaaggagaggtaaatatgaattatagagattttattacagattgtattagcggtggttacaacgtacacatcagtgttacagaaaaacgagtacacattatttctgagatgacatcagcatcttaccctaaaaaggaaattaacttagatgaactacaagcttatgtgtactatatgaataattttggaagtcaaattacaacggaggggttataaatggaattggttattaatattgtagcagtattggttggtatgtatgctatttatttctatgttacaaagtttagtactggcttatcaggtattttaattgttttagggatggctattggtctttacttctacttagactatttaaatgtcagagaaaatgttattcgattagtttcagtaatgttcggagctttcttatttagtattgaaatgatttataataaaattatgttcgaaattaaaaaaagcaatgttcagaagactgttagagtgtatgataaagagcagtaatgattttaccataagagtacctaaattactttaagtgctctctatggtaccttaaagtagcttagaattgaaattaaggagatgaacaattatgtatcctgaaatagatgtggaagaattagcgtataagctaaaaagtacaagagagtatttagagagcattacaacaaaagaagtagaaatttatgaaatctatcatcttaaaacaggtaagttagtttttaaaggtgaatacattgaggtaaaagaattactgaggaaaatgtataaagaaaatttaacacttgtagatgtagatacaatgttaagcattggtaaaggatttattgatgtaattaagaatatatcggcagaaaatgtattccaaataacatataaaaaggagctatcaacaaaatgattaaaatattttcagaagtagataaagaatacaaacctattattactgaaaagtttcctaatggtgagattaattttaaatatgatgatttaaagtatttagtagaagaggacttaagatttgatgttttctttaaatgggaaaatgacgcagacttaatgcatttgtatatgtttactaagtatttagagcaactaggtattaaagataaagctgaatttttagagattgcatatctaccttatagcagaatggatagagtagaagaaggacataataatatgttcagtcttaaatacattacagaatttattaataaccttaattataaatcggtatgggtagcagaacctcatagccctgtaacagaagaattacttactaattcttttgctattgatgttacacttaaattattaaatcagtatattgaaatgtccgaagagcctgtaacaatagtactacctgataaaggggcatacgatagatatctatttgatgtagaacgtatcttaatggaatctaatattgaatcatattcaattgtatatggtgagaagaaacgagattttgaaacaggtaagattaaaggtattaaaataattaaagataaaaatactttatatgataattgtattatactagatgacttaacaagttacggtgggacatttgtcggttgtaaaaaagcccttgacaaacttaaggtaagtagtgtatcattaatattgactcatgcagaacgagcttttgcagaaggagcattacttagctcaggatttaaagatattattgtaacagactctatgttccctaaaaataattgggaaaaagctattgctaaacatagagctagaatcaacggaactgaattacaaataaaagatatcgaaagatatttataaaaggagaaaaataaattatgctaaatccaactttaatgtgtgacttctataaactaagtcacagagaacaataccctgaaggtacagaaattgtatatagtacactagtacctagaagtaataaatattatgaacacagtgataatattgtagtatttggtattcaatcacttgttaaaaaatattttattgatatgtttaataaagagttctttaacagacctaaagaggaagttattaatgaatacaaacgtacagttaaatttacactaggacaagaaaatcctgatgctaaacacttagaacaattacatgacttaggttatttacctattgatgtaagagctttaaaagaaggtactgttgttcatcctaacacacctgttatgacaattgaaaatactcactcagatttcttttggttaactaattacctagaaactattattagtactcaaacatggcaagcaatgactagtgctacactagcatatgatatgcgtaaaatgctagataaatatgcaatggaaacagtaggtaatattgaagcagtagatttccagggtcatgactttagtatgcgtggtatgagttctttagaaacagctcaattaagttcagcaggtcatgcaattagttttaaaggtagtgatacagtacctgtagtggatttcttagaatcatattacaatgcagacgtagagaaggaaatggttgttgcttctatccctgctactgagcactcagtaatgtgtgcaaatggtaattatgaaaccatggatgagtatgaaacatataaacgtatgttaacagaaatatatccaacaggcattttctctattgtgtctgatacttgggacttttggggtaatatgactaaaactttacctagattaaaggatattattatggaacgtaatggtaaagtagtaatcagacctgatagtggagaccctgttaaaattatttgcggagaccctgatgcagacactgaatatgaacgtaaaggtgcagtagaagtgctttgggatacatttggaggtactgaaactgaaaaagggtacaaagtattagatgaacatgtaggattaatttatggagactctattaactatgaacgtgctcaacaaatttgtgaaggattaaaagaaaaaggttttgcaagtattaatgttgtattaggtgtaggtagtttctcttaccaatttaatactcgtgatacccacgggtttgcaatcaaagcaacgtatgctaagattaaaaatgaagaaaaacttatctataaaaatcctaaaacagatagtggtaaacgttcacataaaggtcgagtagctgtatataaagacggttcatgggaagataacttaaccttacatcaatggctaaacaaacaaaatgttaatcaattagaaagagtatttgaagatggtaaactttatagagaccagtcgttaagtgaaattagagaaataattaaaaataattaataaatatttaaactccctattgacaaagggagttttttattatatagtagggctatagtaaataaaggagtgaaagaaatgatttataaaatatcaaaacataattactatagtaggtttgaatattcatcttatttacctgatgaaggatttgcatatatagattatgtagatgtcattcttataggtgtagataatccaaagaagagaaaagttattactttaaaagcagatgagtttaatcctagtgattttaaggttggtcataaatataatattataaaaatactatggtttgagaaatgggaatggttacagccatagggaggagaggtatacaatgattatagataaattaaatggagttaaattagagattggcggtcatgttgtatcatttagtgtaagtaagtttaaaacgattaatggtgaaagacaattacttgattaccaccatatcaaaagaggtaaacagagatattttagaactactgaggaattctataatgagtacaaagaaataaaaccggataagaatgagatagatgaaatgtttgaatctttaggttacgtaaatactgaattagaagatgtagtaagaaaccaagagaaagtgacagagatattaggagttagtgaacagtatttaaaccaattgtcttataaggctatagaggaatatgtagaaaaaatagttatcttagaaattaaagaattaaaaggagagatacaatgataaacattaatgtaactgaaaaagaaaaattagttgtaggtgatttagtaaaatcaagagaagatggtacatggggtattgtagtagaagataagcaagacttaaatgtagttgtgttaaatgacgagccctggttattttataaatcaggaattaaaagagtagaagggcaattagaagaggactttaaatttattaaaaacagagaagagtatgatatagatgtagttaattcttcttataaataaattcacatctacctattgacttaggtagatacttattatataatagtatacaaggagatgaagtatgatgaatggaaaacaaatttatgtatttttaagtgaccaatacagtaaagatatactcagtttacaattaggtcttattaaggaatggtctaggagagaactaacttattcagatgatgtcggttcagatgcagatgttgttatttgtactgatatagtaagagatgatttcgtaaaaaaactaagtaaaaataatagcaatgcattatttgtatttattagttctagttattggataggttataaaggcggagaattctttgttgcagttcaagactatgtgaaaggtatgtaagatatgaaaaaattattaatattatttacattagctagcactttactattagcaggatgtacaccggataatcatgaaggaaaagttttaggaacaggagaatatagagagccaactacttatatcaagtcaggaagtgttactgtaccagttattggtgaaatgaaatactatgtagacttagaaacagataaaggtgaagaccgtgtttatcttaatagggaagtttatcgtaaatttgataaaggtgatgatttctctaatgtaggtaaaaaagtatataaaaatgatgaattaatatataaaggggactaattagtatgaaacaatttatacatgataaaaaagatagttataatagtacaaatcgtaattttgatattcaatattataaaggtatacctttacaacaaattgatagggggtatggtcaagcaagagctaggagatttacaataaataatacgaaccaaaatatatggatacctatgacatatttaaaacctaatggtactcttaaaaataacattgatatagattggatacttgttaaagaaaaatgtagtttaaagaaagcaggattagtaataaaaatagaaattacaggagatgtattataatgtatatattagaaagaacaattagaggttttgcaggtcaaacagaagatattttaccttaaaggagatgaattaataatggagtatgaaaaaatgattagagaaataatggtaaactctaaagaaatgtcactagaagataaaaaacatttaatgagtttattgatgagtgcttatggtgacttatcaatactagtagcttttgaagaagaaaacacagcacatatgtatgaagaaataaaacagtatgatactaaaaagttactgaaaccaagtatggtaagtaaagataattatatgaaataatatgtaaccaatcaggaggaataactaatgataaatatagaacatgattatacaataagaactgtagataatagaaagtatacttactatagtaaacatgaatccccagttactttatataaaaatattataagtaaagattgtattgaagtaactaaatatgggaaagataaaaaagttattatagctactaaatatattgtatctattgaacgatggtaattacaaggaggagtagttatgaatgctagggaagcacgtaaaaacactaaaaactataaggactctaatgtagtaactaaagagcaacacttaacttatatctataataagataaactacttgattgcaaatagtagtagtcagggtaagacatatgtggcaatgaatctaagaacagattatcctgatgagttttctttatctaaattaaaatatctaaaagaaattaaacagcactataaagacctaggatttaatgtgaaaacgcaagtaagaaaggcaaagtggtcagagaaaagtgtaatcaggtactactttaacttaggctatatagacagcgtgttagtacctattatacacattagttggtaattacaaggaggaatagttatgttttttaaaaagaagaagttaagcaatgtagagaaacaaataagacaaaaccgtaataaagaagacaaagaaagaaaagaacatcaagataagttaaatacagatatgtataaaacgtatgaattagataaaattgtagaagaacatttaagaaaattagacaatatatcccttgaaggattagaactaacttcagtgtgtttagggacaagacttgtttattattattcaataggcaaggattgggataaacaagtatatagtttaaacgaattagaatatatgaagaagaaatttaagaaactaggatttgaaactcagataacaaacgaagatataggatttcaaccttatatttatttaagattattatgggatgcataagtaattattattagaggaggaatagttggtgttgcacagttaattacggattatcatgacggacattaagtattgaatattgttgactaataataagaagaaaatattattactactaagtacctttgttatgtactactattactactactaagtacctttgttatgtactactattactactactaagtacctgggaattcttttacctctctcactcagcctattacttattaccgacttccctaactacttattctatagttataatattcatttattatacaatacttaaactatagtattctactgttaatctatgctgaagcggtcttaatctatggttattatataataatcttatataatggtacattaatctagtatattacattagaatcattctaatctaggattttaatctttagaccctaggaaaagtggtactaaaatataaaaccctataggtatgggattcttatttttaaaattactaaaaagtattaggttttccctagggcaaagttttaatgtacttaaaatagtaagtagctacttatcatttagggttctataattgagaatattgagagataatccgcttcaattgtaattaattgttgacaactatgaagcgggtatgctataattaggtatagtcaaatttaggagatgaaatagatgattgatatatacttaggagaaggttataataaagaatacttgtctaaagcactcagattaatcaatgaccatgctcctagggagttaagttatgattttaataatgtagaagcggatgttaatattcacacaatgttatatgttaaacctgaagatagatttatatataaggatatatcctatgacttcccgggtgatttaattatttgtatagttgatgatgatgctattgtataccaccaaggtgagcagatttcaggtattagtattttaagaatactagaagagatattttaaggaggataagtaatcatgataggaataacaatattaattacgataatgagtatatcaactatctctatgtatatttattttttagtagacttgattcagtcaatcagatataatagttttgataaggtaattaacgtcataacatttgtacttatgacagttataatagcatcaggtattttagctatacttggaatatagagctcatttaagaagcggttaagtagttagaggggatttgtcctaaaatagtataccgcttctatatggaaggctgagaggtcttagaattgaaaggagagatataatgattcatatatttttaactgatagttatgataataaagttttaaatactgtactcagatatattaatactactagtgatagagagcttagttacttaatgggtaaaggtgaagcggatgtatgtatagaaaagggagtatttagtaatatagaagatgttaaaattgactctgagtttattgatagaggtaacttatgtatacttataaatgaagatggattagtatgtagttactacagaggagaatcatgtaatgttggttcctttgtaaaggagaggttataatgatagaaattaggttaactgaagattataatgacttgagtcttaaggcattactaaaacgtattaaaagggtagctcctagggaattaacttatggtttagaagcggatatggatactacagatgttaatattggagattcagttccttctagaggtttatatgtagagtactcagaacgttttactagggacttatggataattgtacacccttcaggttatgatgcttattatcaaggagagaaatatggtggagagtctttagatgagattatacatgatatgttttatgattatgcagacccttttgacttagattattagaaaggagagattataatgatagagatataccttagtgaaaattatgataagaatttactaaaagcagaattaaaatggattaaagagaccgcttcaagagaactaacttatgatattaataggaaacctggattggatgtttatgttaatccctataggtgtactaaagacgaagttgaagaatggagtacacttcctccatttgaagatgatatacttgtatttatagcggagacgtggatacatgaatatcttaagggtgaatcaataggtgtagatagtatggaagagtatgtaaaggagatgtaactaatgtttaaggtatattatacagtctaccatagaggtagtatgaaaactattaaggataagctagatagaagtagtttaatatacttcttgtatgatacttggtataaagatattagtaacgtattccctaatcactataataaagagtttgggagtaagagtgatgatatagatatagataaacttattgaagcggttaatgaggaaggtatattacttatcaatagaggtaattatgttacaataagagaatggtaggataggataaacttaggatagaaaataatttaggatgagttacaataggataggataggatagggggttaagttaggatggatactttaacatacactattattcataaagaatctgatagggtaatagctagcggtttaaatgagacagaaactatgaacttagttcaaaggatgataaatactaatctagttactgatatatcattagatgattatatacgcagaccacatggaaagatagatgtagtcaatttactagtagatattagaagacaaggcgtatttgatttcaatcacatttggcacgtaggataggagggataggatgatagttatatatacagatgtttctaaggattatttaaaagacgagttcttaccttggcttaatgaaagggatagatacttagaatactataaagatgaattacctgaggatatagattcctcttatattgtatcagttgtatactgtaaggatatggaaggtctattagaaagaaaagacattgttcttgataatagttataatgaacctgtagctttattaggtgttcctgagttttttggtaattatagtaattatttctattatagaggagaaagtattagtaaacatgacctaggagaaattgttaggttaaaagcttggcaacgtatgggtggggattgactaagtagctctccctaatttcactaagtagctccctaggaattgcctaagtagctcggtatgattttaccctaagtagctccctctgttttctactagtttattttaaccgcttcaggtgtctatatatatatagacggttggaataatatcagaccgcaaaaataaatacactaggatattattcccagtgtattatataatttttttatagaatatttataacattgtattcaaattcatttacttcatgttgtgatttaattaaatttttaattaatccgttttgtgttttatactcttttattagtttttcattttctataattaaattattaaattcttcttttgttgtttcctcatctacataaaatttactttcatatatttcataatattttttatctgttccgccatctaaatcatctgatatttgataatttttgaatataatttcttttgtttctaattcatttactaataattgtgattttgcatattgtaatacatcttcattgtcccacattggaatatagtttattttcatttaaatcaaatccttttcttataatttttttatataatatttgtagaagcggttggggtttgtcccttgccttactacactttatatattacagtatagttattcagaagtcaatacttttgagtaactttttttaaattcttttttcttctatataatagtagtttttagccctaaaaatgtttttaaaagaatttgcattttcttattgactttattatcatatggtagtaatataaaggtacagcaagggaacagcaacaagatattagaattatataaaaaaattatttaatttgagatgatttaaatggatgtaaaagaaattgcaaatactataatggagttgtggcaaatggacggctacagatgtgcagaacctccattatatgaaagcacactaaaccacacacgcacacacacggcgttaattgtttctattaatggaaactatgacacagtgcagatgttccgcaaaacgcctataatgagcatgagagggcaaagccaaccggctagcatgttagttaatgtgattgacgatgtaattataatcgtatatgaaaatgtagtgtacggagttcaaaacaaagaaataaaatttattgaagaaatttaaaaataggggttgcaatcctcaagcatctatagtaatataataggtgtaggggatagcaacacacctcaaaaaactttttaaaaaagttaaagaaaagtgttgacaccttacaagatacatgttattattaagataacaaataagacaagccacctagcaaataacgaaattaaataaaaaaattatagaataggatttgattattatgacaaacaaaaattacttatacgaagaaactcacacagtacaagggcaagacattacggctttcagaattccaaatgacgcaaacggcaacccacgttatgtagtgaatttcagggacttaggtatcggattatgggactatgacaacatcaataaactttacggatttaataaatatcgtgccaaatggtttggcggtggtgtagtattccaaagctataatatagaagatacattaaattttgcactagataatgttaaagaaatagaagcggttaagaattaaaaccgcttctgaattaaataaaaaatttatataaaaaggatatgataatatgaaatttaaaatagaaaaaaataatagtgatataaaaactttatggaatttagcaaaaaatggatatatgagctatcaaactgtacacaatatatttaaaaatgaatcagatgaatttattatatttaacagtaaacaaacttataataaatttatgaaattaagatataatagaagtgcaatacaataatataaaaaaaattatacaattccctaggattagatttctagggatttttatttattttaatttatataaaaaaattatttaataaataagttagtgtaaaattgactattgacaaggttgtattttttatggtataatgaagtgaagaccttttttagtataaaaaaattattatataaaaaatttatattaaatgattttagaaacgctctttcccggaacctcttctcttatatagcggacacgtaggctccttaccgctttcttactatacttatagtatactatagaaaaaagaaaaggtcaacccttttctttaatcttttttatatttttttataattcttctaacggttatttcacttatattatattcctttgctaacataggttgagtgtactttctaggcttgtatttgcttgctatggcttttctttcatcttctgttaatgcataacccctattatacctattgaaagtattgtctttatggtgccttttattgtgttctgacgggctaatacattctaggttatcaatacaattattttgtttgttaccgtctatatggtgtacatggtttgatttaataaaatcattgccaaaatattcatatacaagtctatgcaccatgtgttttttattattgacccttacggttaaatatccgtcacggtcttgatgtactttactcaacctatcatttatttttaccctacctaaattagaaacataaaaattatatttattaaagtattctttttttataggtttccaaatttcattaattttattttcaaccatttttttactcctccttttttggtatcacttccattatataataattcggtcttaatgtcaatagataaatgtaaaaaagttttttaaattaatttcattaaatctattgactttaatatcattatagtttaatataaaggtataccaaatgaaagggattgaacaaaatgataaaattcaaatggaaaaacaaaacaattaaatcaactcaaaaaacggataacattctattacttattataggtggtttagttgcaacaatcacacctaaacttgtaaactggtttttactactacaagataatataaatatttttttaagataactattgacaacctagaaacaacatgttaatattaagataacaaataaatcaataaaggaaatgataaaaatgaaaaaaatcacaacaactttaaacttaatcggcatgaaaaataatgaaaggtttacagaagagttaaaaaactaccgtcaagatgttactttcttgaaagcaaataaaattgtaaaatattcaaaataaggcttgacaacttaaacactacatgttattattaaggtacaaggtaagggaagcggtcaaccgcttccaacctaaataaaaaaagtttaaaaaaaactattgacagtcacttgaaaccatgatattattaagataacaaaaaacaaacagaaaaggaattgattataatgaaatttatcaaaactatcgaaaacttattaactaaagcagaaaacaaagggcaagcaattttaaacggtcgttattatgacggatatagaaacggtgagcttgaggaaaaatatgcaatcgaaattgatggcaacaaattaattatgcgtcattggggtacacaaacaattgagattgacttaagcataaatgaaattgtttcatactatggtgaaagcaattcagaccgtgacagtttaaacacacttgtatattgcttaggaattgcgccaaactttagatacttaccaagcaaagacttgttcatttacgaaaattaattaaataaagggcttgactttcaagccctaccatgttattattaaattgtaaggtaatcaagcacaacgacaaaataaactgaaaaggaattgatacaaatgaaattaatcaatagagataatgaaatcgtaattagcatagcaacacttgagagtgtaaaacaagccctaatttgggagtacatcgaccacttagataataacatcctagacaaagaaatacatgaccaggaagcggttgttattacttcagacactttgcaatcactcaaatttgcggacactatggaagaactagaagaatatgtaaacgacatcggttggaaattagtttagaaaaggtattgacatcctaacatatagatggtaatataagagtatagaaaaaataaaaaaagaaaaggatttgattattatgacaaatacaatacaagcatttttacaaggacaagaagcaagcacagttaaggacgtagcaactcatggagtacaaagcggagcaattggcaaattaatctacacatcagacgtagtaaacttctttgatagttacgagcaggacattgaagcggtcatcactgaatacattgaagaggttacaggacaacaatattatgacttattgaactatgagcttatgagagacctcgagaattatgcaaatgtagaatttgaagacgaagacgaatataataacattcaatttgacctagcagaaaacattgcttctgatgaggttgaaggatttgaagacatggacgaagcagaccgggcggaagcaatctatgaggctatggatgatgttgaattagaactacaagaaactgacaaggttcaatatgttaatctagcggttgagattgtagctcaaagaatggcactatagaaagcacacagagaagcttaaccgcttctctaatacaattaatcaggagatgttgaagatgaatacaagacgggtaaacagagcgttaaacgaagcagttagattattagatgaacaaatagcagatactcaaaagactatgcaggagttgaataaacaactagagaagcaaataaaggctaagcaagagctaatggtattagttgatgttatgaatggtgatgatgagtaatgaacattagagaggttcacaatgtcgttaagagtgctaagagcaaactcctgcaggagcaggctcacccaacggataacctcatagagcagtacatcaatgatgagctacacagacgcacacagagaagcggaacaatacagatgaacaataatactacttcatatagtaatagcccatatggtagcttagaagagcttagagaagcttatgacctatcgtcattatctactggtgagattaaagaactaatacaaacatttgtttaaattattttatcaaaacgctttacaatcttttagtttgtatgatataatgaacttaacaaattaaaagaaaaggaaatgatgaacatgacaaacttacaagaaagaaaacaagaattgaaaacgttactatttaacttagctttagagaagaacaaagcaactgatgagacactgcttagcgtattagagcaagcacatcaagaggtaggaaatcaattaagaaaagtaagaaaagaaatagaaattttagtagaagaaaaagaaagggaattttggaatgatatcgaatttaatggattagactaagagggaataaaatccctcttttatttttatcctattatataatttttttatattatacgggggcaggggtaaaatgccactcaatgggggtgggtctatatacccctatggtctacc

(19) United States
(12) Patent Application Publication
(10) Pub. No.: US 2025/0352592 A1
(43) Pub. Date: Nov. 20, 2025

(54) POLYVALENT BACTERIOPHAGE COMPOSITIONS AND METHODS FOR TREATMENT OF BACTERIAL INFECTIONS

(71) Applicant: Baylor College of Medicine, Houston, TX (US)

(72) Inventors: Austen TERWILLIGER, Houston, TX (US); Haroldo Hernandez SANTOS, Houston, TX (US); Anthony MARESSO, Houston, TX (US)

(21) Appl. No.: 18/872,518

(22) PCT Filed: Jun. 9, 2023
(86) PCT No.: PCT/US2023/068222

§ 371 Date: Dec. 6, 2024

Related U.S. Application Data
(60) Provisional application No. 63/351,252, filed on Jun. 10, 2022.

(51) Int. Cl. A61K 35/76; A61P 31/04; C12N 7/00

(52) U.S. Cl. CPC A61K 35/76; A61P 31/04; C12N 7/00; C12N 2795/10021; C12N 2795/10032; C12N 2795/10071

(57) ABSTRACT

Disclosed are compositions, devices, kits, and methods for prevention and/or treatment of Staphylococcus infection. In specific embodiments, Staphylococcus are S. pseudintermedius and/or S. aureus. Aspects of the present disclosure can be directed to bacteriophage compositions comprising one or more of vB_SpsM-DH2, vB_SpsS-DH5, and/or vB_SpsM-DS10. Further disclosed are devices and kits comprising such compositions, and methods for use of such compositions in treatment and prevention of pathogenic Staphylococcus infection.

### CROSS-REFERENCE TO RELATED APPLICATIONS

This application claims priority to U.S. Provisional Patent Application Ser. No. 63/351,252, filed Jun. 10, 2022, which is incorporated by reference herein in its entirety.

### SEQUENCE LISTING

The instant application contains a Sequence Listing which has been submitted in ST26 format and is hereby incorporated by reference in its entirety. Said ST26 compliant copy, created on May 31, 2023, is named BAYM_P0370WO_Sequence_Listing.xml and is 397,263 bytes in size.

### BACKGROUND

### I. Field of the Invention

Aspects of this invention relate to at least the fields of microbiology, veterinary medicine, medicine, and virology.

### II. Background

Multidrug-resistant (MDR) bacteria infect millions of people around the world, yearly. Many of these infections caused by these bacteria have become untreatable due to resistance to last resort antibiotics. Exacerbating this crisis, the pipeline for antibiotic development is slow and resistant strains rapidly develop in the wake of new drugs. The genus Staphylococcus, including the species Staphylococcus pseudintermedius presents a growing antibiotic resistant threat to human and companion pet populations. S. pseudintermedius is an opportunistic pathogen that colonizes the skin and mucosal membranes in dogs. Although its clinical significance was unknown when first classified separately from Staphylococcus intermedius ¹, the field now recognizes its unique pathogenesis and virulence mechanisms in canine pyoderma, otitis externa, urinary tract infections, respiratory tract infections, and reproductive tract infections ²⁻⁵. Traditional penicillinase-stable-β-lactam antibiotics quickly became ineffective against S. pseudintermedius as multidrug resistance spreads ^{4,6-8}. As such, the European Food Safety Authority (EFSA) now classifies S. pseudintermedius as the third most important MDR pathogen in the European Union, behind only Escherichia coli and Pseudomonas aeruginosa ⁹.

A promising response to MDR infections is bacteriophage (used interchangeably herein with the term “phage”) therapy. Viruses which infect bacteria, phages are environmentally ubiquitous, host-specific, and effective at infecting MDR bacterial strains. Importantly, they have been shown to be safe and effective in animal and compassionate-use human trials. Because they use the replication machinery of their bacterial host, phage mutation rates are directly influenced by those of that host; as such, phages may rapidly adapt to target strains of bacteria. However, due to the cognate rates of evolution between a phage and its host, a mixed population of phages and bacteria will result in an evolutionary arms race. Consequently, phage-resistant bacteria are likely to develop.

The rise of MDR pathogens coupled with the decline of antibiotic development highlights the need for alternative antimicrobials like bacteriophage therapy. Consensus phage therapy guidelines dictate the use of strictly lytic phages without undesirable elements like antibiotic resistance, integrases, toxins, or other virulence factors ^{10,11} However, as reported in literature and congruent with the inventors experience, discovery of lytic S. aureus or S. pseudintermedius phages is difficult ⁵. Nonetheless, lytic phages from other members of the Staphylococcus genus have been isolated and characterized including for S. aureus and S. epidermidis ¹². In 2022, the PhageAI database contained 26 S. pseudintermedius phage genomes while Zeman et al. reported 19 such genomes in the NCBI database, none of which were lytic ^{13,14} The reported paucity of S. pseudintermedius phages—whether virulent or temperate—demonstrates the need for discovery of new phages for potential clinical and/or veterinary applications.

There exists a need for new and improved methods for identification of lytic phages, and compositions for treatment of bacterial infections, including Staphylococcus infections such as Staphylococcus pseudintermedius and/or Staphylococcus aureus infections.

### SUMMARY

Embodiments of the disclosure include bacteriophages, bacteriophage compositions, kits, devices, medical devices, therapeutic devices, polynucleotides, methods for preparing a device, methods for treatment of a Staphylococcus infection, and methods for prevention of a Staphylococcus infection.

In certain embodiments, disclosed herein is a composition comprising an isolated bacteriophage vB_SpsM-DH2 (Accession No. OM373548). In some embodiments, comprising about 10⁸ to about 10¹¹ plaque forming units (PFU) per milliliter (PFU/ml) of isolated bacteriophage vB_SpsM-DH2. In certain embodiments, disclosed herein is a composition comprising an isolated bacteriophage vB_SpsS-DH5 (Accession No. OM373549). In some embodiments, comprising about 10⁸ to about 10¹¹ PFU/ml of isolated bacteriophage vB_SpsS-DH5. In certain embodiments, disclosed herein is a composition comprising an isolated bacteriophage vB_SpsM-DS10 (Accession No. OM373557). In some embodiments, comprising about 10⁸ to about 10¹¹ PFU/ml of isolated bacteriophage vB_SpsM-DS10.

In certain embodiments, disclosed herein is a composition comprising two or more of an isolated bacteriophage vB_SpsM-DH2, an isolated bacteriophage vB_SpsS-DH5, and an isolated bacteriophage vB_SpsM-DS10. In some embodiments, a composition comprises isolated bacteriophage vB_SpsM-DH2 and isolated bacteriophage vB_SpsS-DH5. In some embodiments, a composition comprises about 10⁸ to about 10¹¹ PFU/ml isolated bacteriophage vB_SpsM-DH2 and about 10⁸ to about 10¹¹ PFU/ml vB_SpsS-DH5. In some embodiments, a composition consists essentially of isolated bacteriophage vB_SpsM-DH2 and isolated bacteriophage vB_SpsS-DH5. In some embodiments, a composition comprises isolated bacteriophage vB_SpsM-DH2 and isolated bacteriophage vB_SpsM-DS10. In some embodiments, a composition comprises about 10⁸ to about 10¹¹ PFU/ml isolated bacteriophage vB_SpsM-DH2 and about 10⁸ to about 10¹¹, PFU/ml vB_SpsM-DS10. In some embodiments, a composition consists essentially of isolated bacteriophage vB_SpsM-DH2 and isolated bacteriophage vB_SpsM-DS10. In some embodiments, a composition comprises isolated bacteriophage vB_SpsS-DH5 and isolated bacteriophage vB_SpsM-DS10. In some embodiments, a composition comprises about 10⁸ to about 10¹¹ PFU/ml isolated bacteriophage vB_SpsS-DH5 and about 10⁸ to about 10¹¹ PFU/ml vB_SpsM-DS10. In some embodiments, a composition consists essentially of isolated bacteriophage vB_SpsS-DH5 and isolated bacteriophage vB_SpsM-DS10. In some embodiments, a composition comprises a combination of isolated bacteriophages vB_SpsM-DH2, vB_SpsS-DH5, and vB_SpsM-DS10. In some embodiments, a composition comprises about 10⁸ to about 10¹¹ PFU/ml of isolated bacteriophage vB_SpsM-DH2, about 10⁸ to about 10¹¹ PFU/ml of isolated bacteriophage vB_SpsS-DH5, and about 10⁸ to about 10¹¹ PFU/ml of isolated bacteriophage vB_SpsM-DS10. In some embodiments, a composition consists essentially of a combination of isolated bacteriophages vB_SpsM-DH2, vB_SpsS-DH5, and vB_SpsM-DS10. In some embodiments, a composition consists essentially of about 10⁸ to about 10¹¹ PFU/ml of isolated bacteriophage vB_SpsM-DH2, about 10⁸ to about 10¹¹ PFU/ml of isolated bacteriophage vB_SpsS-DH5, and about 10⁸ to about 10¹¹ PFU/ml of isolated bacteriophage vB_SpsM-DS10. In some embodiments, a composition comprises an isolated bacteriophage with greater than 95%, 96%, 97%, 98%, 99%, 99.1%, 99.2%, 99.3%, 99.4%, 99.5%, 99.6%, 99.7%, 99.8%, 99.9%, or 99.95% genomic sequence identity to the genomic sequences of vB_SpsM-DH2 (SEQ ID NO: 1), vB_SpsS-DH5 (SEQ ID NO: 2), and/or vB_SpsM-DS10 (SEQ ID NO: 3). In some embodiments, one or more direct terminal repeats (DTRs) may or may not be included in the calculation of sequence identity.

In some embodiments, isolated bacteriophage are not lysogenic, do not comprise antibiotic resistance coding sequences, do not comprise bacterial virulence coding sequences, and/or have lytic capacity. In some embodiments, isolated bacteriophage are evolved to improve lytic capacity, reduce lysogenic capacity, reduce antibiotic resistance gene obtainment capacity, reduce bacterial virulence gene obtainment capacity, and/or increase the number of bacterial strains subject to lysis by the isolated bacteriophage lytic. In some embodiments, the amount of the bacteriophages in the composition are substantially the same. In some embodiments, the amount of the bacteriophages in the composition are not substantially the same.

In some embodiments, a composition is a lotion, cream, body butter, mask, scrub, wash, gel, serum, emulsion (e.g., oil-in-water, water-in-oil, silicone-in-water, water-in-silicone, water-in-oil-in-water, oil-in-water-in-oil, oil-in-water-in-silicone, etc.), solution (e.g., aqueous or hydro-alcoholic solutions), anhydrous bases (e.g., stick or a powder), ointment, milk, paste, aerosol, solid form, jelly, and/or powdered form (e.g., dried, lyophilized, particulate, etc). In some embodiments, a composition is a solution, lotion, and/or cream. In some embodiments, a composition is shelf-stable. In some embodiments, a composition is formulated for topical, oral, aural, nasal, and/or ophthalmic application. In some embodiments, a composition is formulated for application more than once a day, once a day, twice a day, once a week, twice a week, once a month, or twice a month during use. In some embodiments, a composition is housed in a delivery apparatus. In some embodiments, a composition is comprised in a suitable container. In some embodiments, a composition is comprised in a kit.

Also disclosed herein are methods of treating and/or preventing a Staphylococcus infection in a subject, comprising administering to the subject one or more compositions described herein. In some embodiments, a Staphylococcus infection comprises a Staphylococcus pseudintermedius and/or Staphylococcus aureus infection. In some embodiments, an infection is a urinary tract, blood, gut, abdomen, stomach, lungs, skin, ear, eye, nose, oral, kidney, prostate, bladder, brain, vaginal tract, heart, liver, spleen, tendons, or wound (cuts, burns, sores, etc.) infection, or a combination thereof. In some embodiments, an infection is a wound, skin, ear, eye, nose, mucosal, and/or oral infection. In some embodiments, an infection is a catheter-associated infection. In some embodiments, a Staphylococcus is of pulse-field gel electrophoresis (PFGE) type USA200, USA300, and/or USA400. In some embodiments, a Staphylococcus is of clonal complex CC1, CC12, CC121, CC15, CC22, CC25, CC30, CC45, CC5, CC59, CC8, CC80, and/or CC97, CC398. In some embodiments, a staphylococcus is MW2, No. 10, NP66, CI/BAC/25/13/W, XQ, 046, ST20130941, UP_620, HO50960412, H-EMRSA-15, 014S_SA, NCTC8317, MRSA252, FORC_001, LA-MRSA ST398, 08BA02176, CA-347, USA600, Mu3, N315, SA40, HZW450, USA300_FPR3757, COL, GR2, 11819-97, MOK063, CC1153-MRSA, TCH1516, LMB2, MN8, CDC587, MNWH, MNPE, FRIl169, Newman, LAC, MNLevy, c99-529, JH1, JH9, FPR3757, RF122, and/or ST228. In some embodiments, a Staphylococcus is MW2, No. 10, NP66, CI/BAC/25/13/W, XQ, 046, ST20130941, UP_620, HO50960412, H-EMRSA-15, 014S_SA, NCTC8317, MRSA252, FORC_001, LA-MRSA ST398, 08BA02176, CA-347, USA600, Mu3, N315, SA40, HZW450, USA300_FPR3757, COL, GR2, 11819-97, MOK063, CC1153-MRSA, TCH1516, LMB2, MN8, CDC587, MNWH, MNPE, FRI1169, Newman, LAC, MNLevy, c99-529, JH1, JH9, FPR3757, RF122, ST228, AZM21, AZM24, AZM28, AZM29, AZM34, AZM35, AZM36, ATCC 49051, AZM1, AZM2, AZM19, AZM20, AZM22, AZM23, AZM25, AZM26, AZM27, AZM30, AZM31, AZM32, AZM37, and/or AZM38. In some embodiments, a Staphylococcus is drug-resistant. In some embodiments, a Staphylococcus is multidrug-resistant.

In some embodiments, a subject has a urinary tract infection, neonatal meningitis, a blood-stream infection, pneumonia, sepsis, a surgical wound infection, a wound infection, a skin infection, an eye infection, an ear infection, an oral infection, a prostate infection, meningitis, a vaginal infection, or a combination thereof. In some embodiments, a subject is immunosuppressed. In some embodiments, a subject has an immune cell defect, asplenia, impaired splenic function, nephrotic syndrome, or an autoimmune condition. In some embodiments, a subject is administered the composition prior to a medical procedure or regimen. In some embodiments, a subject will be subject to immunosuppressive conditions. In some embodiments, a subject is taking or will be taking chemotherapy. In some embodiments, a subject is taking or will be taking an immunosuppressant. In some embodiments, an immunosuppressant is a glucocorticoid, a calcineurin inhibitor, an antimetabolite, or an antibody therapy.

In some embodiments, a source of the Staphylococcus was a beverage, comestible, an individual, or an environment. In some embodiments, an environment is ground or surface water, water used to irrigate crops, a public water system, a hospital, a school, a nursing home, a petting zoo, a daycare, a lodging, a cruise ship, a train, a razor, a towel, clothing, a gymnasium, or an airplane.

In some embodiments, a subject is a mammal. In some embodiments, a subject is a domestic animal. In some embodiments, a subject is a farm animal. In some embodiments, a subject is a zoo animal. In some embodiments, a subject is a dog or a cat. In some embodiments, a subject is a cow, a horse, a sheep, or a goat. In some embodiments, a subject is a human.

Also disclosed herein is a device, comprising, on, in, and/or around the device, isolated bacteriophages vB_SpsM-DH2, vB_SpsS-DH5, and/or vB_SpsM-DS10. In some embodiments, a device is a catheter, drive line, syringe, tube, implant, defibrillator, artificial joint, pacemaker, screw, rod, disc, intrauterine device, pin, plate, stent, dental device, eye lens, shunt, valve, neurological or neurosurgical device, gastrointestinal device, genitourinary device, catheter cuff, vascular access device, or wound drain. In some embodiments, a device is further defined as having a coating comprising the bacteriophages.

Also disclosed herein are methods of obtaining lytic bacteriophages comprising, a) obtaining hair and/or skin sample(s) from one or more subjects, b) isolating phages from the sample(s), and c) analyzing the genome of isolated phages to predict lytic capacity and/or directly determining plaque forming capacity of the phages by exposing a target bacteria to the isolated phages. In some embodiments, a hair and/or skin samples comprise at least a portion of a hair follicle. In some embodiments, a hair and/or skin samples comprise an upper portion of a hair follicle. In some embodiments, an isolated phage is analyzed to determine lysogenic capacity, and wherein the isolated phage is determined to be non-lysogenic. In some embodiments, an isolated bacteriophage has greater than 95%, 96%, 97%, 98%, 99%, 99.1%, 99.2%, 99.3%, 99.4%, 99.5%, 99.6%, 99.7%, 99.8%, 99.9%, or 99.95% genomic sequence identity to the genomic sequences of vB_SpsM-DH2 (SEQ ID NO: 1), vB_SpsS-DH5 (SEQ ID NO: 2), and/or vB_SpsM-DS10 (SEQ ID NO: 3). In some embodiments, isolated bacteriophage are not lysogenic, do not comprise antibiotic resistance coding sequences, do not comprise bacterial virulence coding sequences, and/or have lytic capacity. In some embodiments, isolated bacteriophage arc evolved to improve lytic capacity, reduce lysogenic capacity, reduce antibiotic resistance gene obtainment capacity, reduce bacterial virulence gene obtainment capacity, and/or increase the number of bacterial strains subject to lysis by the isolated bacteriophage.

The following aspects describe certain inventions disclosed herein.

Aspect 1 is a composition comprising an isolated bacteriophage vB_SpsM-DH2 (Accession No. OM373548).

Aspect 2 is the composition of aspect 1, comprising about 10⁸ to about 10¹¹ plaque forming units (PFU) per milliliter (PFU/ml) of isolated bacteriophage vB_SpsM-DH2.

Aspect 3 is a composition comprising an isolated bacteriophage vB_SpsS-DH5 (Accession No. OM373549).

Aspect 4 is the composition of aspect 3, comprising about 10⁸ to about 10¹¹ PFU/ml of isolated bacteriophage vB_SpsS-DH5.

Aspect 5 is a composition comprising an isolated bacteriophage vB_SpsM-DS10 (Accession No. OM373557).

Aspect 6 is the composition of aspect 5, comprising about 10⁸ to about 10¹¹ PFU/ml of isolated bacteriophage vB_SpsM-DS10.

Aspect 7 is a composition comprising two or more of an isolated bacteriophage vB_SpsM-DH2, an isolated bacteriophage vB_SpsS-DH5, and an isolated bacteriophage vB_SpsM-DS10.

Aspect 8 is the composition of aspect 7, comprising isolated bacteriophage vB_SpsM-DH2 and isolated bacteriophage vB_SpsS-DH5.

Aspect 9 is the composition of aspect 7 or 8, comprising about 10⁸ to about 10¹¹ PFU/ml isolated bacteriophage vB_SpsM-DH2 and about 10⁸ to about 10¹¹ PFU/ml vB_SpsS-DH5.

Aspect 10 is the composition of any one of aspect 7-9, consisting essentially of isolated bacteriophage vB_SpsM-DH2 and isolated bacteriophage vB_SpsS-DH5.

Aspect 11 is the composition of aspect 7, comprising isolated bacteriophage vB_SpsM-DH2 and isolated bacteriophage vB_SpsM-DS10.

Aspect 12 is the composition of aspects 7 or 11, comprising about 10⁸ to about 10¹¹ PFU/ml isolated bacteriophage vB_SpsM-DH2 and about 10⁸ to about 10¹¹ PFU/ml vB_SpsM-DS10.

Aspect 13 is the composition of any one of aspects 7, 11, or 12, consisting essentially of isolated bacteriophage vB_SpsM-DH2 and isolated bacteriophage vB_SpsM-DS10.

Aspect 14 is the composition of aspect 7, comprising isolated bacteriophage vB_SpsS-DH5 and isolated bacteriophage vB_SpsM-DS10.

Aspect 15 is the composition of aspects 7, or 14, comprising about 10⁸ to about 10¹¹, PFU/ml isolated bacteriophage vB_SpsS-DH5 and about 10⁸ to about 10¹1 PFU/ml vB_SpsM-DS10.

Aspect 16 is the composition of any one of aspects 7, 14, or 15, consisting essentially of isolated bacteriophage vB_SpsS-DH5 and isolated bacteriophage vB_SpsM-DS10.

Aspect 17 is the composition of aspect 7, comprising a combination of isolated bacteriophages vB_SpsM-DH2, vB_SpsS-DH5, and vB_SpsM-DS10.

Aspect 18 is the composition of aspect 7, or 17, comprising about 10⁸ to about 10¹¹ PFU/ml of isolated bacteriophage vB_SpsM-DH2, about 10⁸ to about 10¹¹ PFU/ml of isolated bacteriophage vB_SpsS-DH5, and about 10⁸ to about 10¹¹ PFU/ml of isolated bacteriophage vB_SpsM-DS10.

Aspect 19 is the composition of any one of aspects 7, 17, or 18, consisting essentially of a combination of isolated bacteriophages vB_SpsM-DH2, vB_SpsS-DH5, and vB_SpsM-DS10.

Aspect 20 is the composition of any one of aspects 7, or 17-19, consisting essentially of about 10⁸ to about 10¹¹ PFU/ml of isolated bacteriophage vB_SpsM-DH2, about 10⁸ to about 10¹¹ PFU/ml of isolated bacteriophage vB_SpsS-DH5, and about 10⁸ to about 10¹¹ PFU/ml of isolated bacteriophage vB_SpsM-DS10.

Aspect 21 is a composition comprising an isolated bacteriophage with greater than 95%, 96%, 97%, 98%, 99%, 99.1%, 99.2%, 99.3%, 99.4%, 99.5%, 99.6%, 99.7%, 99.8%, 99.9%, or 99.95% genomic sequence identity to the genomic sequences of vB_SpsM-DH2 (SEQ ID NO: 1), vB_SpsS-DH5 (SEQ ID NO: 2), and/or vB_SpsM-DS10 (SEQ ID NO: 3).

Aspect 22 is the composition of any one of aspects 1-21, wherein the isolated bacteriophage are not lysogenic, do not comprise antibiotic resistance coding sequences, do not comprise bacterial virulence coding sequences, and/or have lytic capacity.

Aspect 23 is the composition of any one of aspects 1-22, wherein the isolated bacteriophage are evolved to improve lytic capacity, reduce lysogenic capacity, reduce antibiotic resistance gene obtainment capacity, reduce bacterial virulence gene obtainment capacity, and/or increase the number of bacterial strains subject to lysis by the isolated bacteriophage lytic.

Aspect 24 is the composition of any one of aspects 7-23, wherein the amount of the bacteriophages in the composition are substantially the same.

Aspect 25 is the composition of any one of aspects 7-24, wherein the amount of the bacteriophages in the composition are not substantially the same.

Aspect 26 is the composition of any one of aspects 1-25, wherein the composition is a lotion, cream, body butter, mask, scrub, wash, gel, serum, emulsion (e.g., oil-in-water, water-in-oil, silicone-in-water, water-in-silicone, water-in-oil-in-water, oil-in-water-in-oil, oil-in-water-in-silicone, etc.), solution (e.g., aqueous or hydro-alcoholic solutions), anhydrous bases (e.g., stick or a powder), ointment, milk, paste, aerosol, solid form, jelly, and/or powdered form (e.g., dried, lyophilized, particulate, etc).

Aspect 27 is the composition of any one of aspects 1-26, wherein the composition is a solution, lotion, and/or cream.

Aspect 28 is the composition of any one of aspects 1-27, wherein the composition is shelf-stable.

Aspect 29 is the composition of any one of aspects 1-28, wherein the composition is formulated for topical, oral, aural, nasal, and/or ophthalmic application.

Aspect 30 is the composition of any one of aspects 1-29, wherein the composition is formulated for application more than once a day, once a day, twice a day, once a week, twice a week, once a month, or twice a month during use.

Aspect 31 is the composition of any one of aspects 1-30, wherein the composition is housed in a delivery apparatus.

Aspect 32 is the composition of any one of aspects 1-31, wherein the composition is comprised in a suitable container.

Aspect 33 is the composition of any one of aspects 1-32, wherein the composition is comprised in a kit.

Aspect 34 is a method of treating and/or preventing a Staphylococcus infection in a subject, comprising administering to the subject the composition of any one of aspects 1-33.

Aspect 35 is the method of aspect 34, wherein the Staphylococcus infection comprises a Staphylococcus pseudintermedius and/or Staphylococcus aureus infection.

Aspect 36 is the method of aspect 34 or 35, wherein the infection is a urinary tract, blood, gut, abdomen, stomach, lungs, skin, ear, eye, nose, oral, kidney, prostate, bladder, brain, vaginal tract, heart, liver, spleen, tendons, or wound (cuts, burns, sores, etc.) infection, or a combination thereof.

Aspect 37 is the method of any one of aspects 34-36, wherein the infection is a wound, skin, ear, eye, nose, mucosal, and/or oral infection.

Aspect 38 is the method of any one of aspects 34-36, wherein the infection is a catheter-associated infection.

Aspect 39 is the method of any one of aspects 34-38, wherein the Staphylococcus is of pulse-field gel electrophoresis (PFGE) type USA200, USA300, and/or USA400.

Aspect 40 is the method of any one of aspects 34-39, wherein the Staphylococcus is of clonal complex CC1, CC12, CC121, CC15, CC22, CC25, CC30, CC45, CC5, CC59, CC8, CC80, and/or CC97, CC398.

Aspect 41 is the method of any one of aspects 34-40, wherein the Staphylococcus is MW2, No. 10, NP66, CIBAC/25/13/W, XQ, 046, ST20130941, UP_620, HO50960412, H-EMRSA-15, 014S_SA, NCTC8317, MRSA252, FORC_001, LA-MRSA ST398, 08BA02176, CA-347, USA600, Mu3, N315, SA40, HZW450, USA300_FPR3757, COL, GR2, 11819-97, MOK063, CC1153-MRSA, TCH1516, LMB2, MN8, CDC587, MNWH, MNPE, FRI1169, Newman, LAC, MNLevy, c99-529, JH1, JH9, FPR3757, RF122, ST228, AZM21, AZM24, AZM28, AZM29, AZM34, AZM35, AZM36, ATCC 49051, AZM1, AZM2, AZM19, AZM20, AZM22, AZM23, AZM25, AZM26, AZM27, AZM30, AZM31, AZM32, AZM37, and/or AZM38.

Aspect 42 is the method of any one of aspects 34-41, wherein the Staphylococcus is drug-resistant.

Aspect 43 is the method of any one of aspects 34-42, wherein the Staphylococcus is multidrug-resistant.

Aspect 44 is the method of any one of aspects 34-43, wherein the subject has a urinary tract infection, neonatal meningitis, a blood-stream infection, pneumonia, sepsis, a surgical wound infection, a wound infection, a skin infection, an eye infection, an ear infection, an oral infection, a prostate infection, meningitis, a vaginal infection, or a combination thereof.

Aspect 45 is the method of any one of aspects 34-44, wherein the subject is immunosuppressed.

Aspect 46 is the method of any one of aspects 34-45, wherein the subject has an immune cell defect, asplenia, impaired splenic function, nephrotic syndrome, or an autoimmune condition.

Aspect 47 is the method of any one of aspects 34-46, wherein the subject is administered the composition prior to a medical procedure or regimen.

Aspect 48 is the method of any one of aspects 34-47, wherein the subject will be subject to immunosuppressive conditions.

Aspect 49 is the method of any one of aspects 34-48, wherein the subject is taking or will be taking chemotherapy.

Aspect 50 is the method of any one of aspects 34-49, wherein the subject is taking or will be taking an immunosuppressant.

Aspect 51 is the method of aspect 50, wherein the immunosuppressant is a glucocorticoid, a calcineurin inhibitor, an antimetabolite, or an antibody therapy.

Aspect 52 is the method of any one of aspects 34-51, wherein the source of the Staphylococcus was from a beverage, comestible, an individual, or an environment.

Aspect 53 is the method of aspect 52, wherein the environment is ground or surface water, water used to irrigate crops, a public water system, a hospital, a school, a nursing home, a petting zoo, a daycare, a lodging, a cruise ship, a train, a razor, a towel, clothing, a gymnasium, or an airplane.

Aspect 54 is the method of any one of aspects 34-53, wherein the subject is a mammal.

Aspect 55 is the method of any one of aspects 34-54, wherein the subject is a domestic animal.

Aspect 56 is the method of any one of aspects 34-55, wherein the subject is a farm animal.

Aspect 57 is the method of any one of aspects 34-56, wherein the subject is a dog or a cat.

Aspect 58 is the method of any one of aspects 34-56, wherein the subject is a cow, a horse, a sheep, or a goat.

Aspect 59 is the method of any one of aspects 34-54, wherein the subject is a human.

Aspect 60 is a device, comprising, on, in, and/or around the device, isolated bacteriophages vB_SpsM-DH2, vB_SpsS-DH5, and/or vB_SpsM-DS10.

Aspect 61 is the device of aspect 60, wherein the device is a catheter, drive line, syringe, tube, implant, defibrillator, artificial joint, pacemaker, screw, rod, disc, intrauterine device, pin, plate, stent, dental device, eye lens, shunt, valve, neurological or neurosurgical device, gastrointestinal device, genitourinary device, catheter cuff, vascular access device, or wound drain.

Aspect 62 is the device of aspect 60 or 61, further defined as having a coating comprising the bacteriophages.

Aspect 63 is a method of obtaining lytic bacteriophages comprising, a) obtaining hair and/or skin sample(s) from one or more subjects, b) isolating phages from the sample(s), and c) analyzing the genome of isolated phages to predict lytic capacity and/or directly determining plaque forming capacity of the phages by exposing a target bacteria to the isolated phages.

Aspect 64 is the method of aspect 63, wherein the hair and/or skin samples comprise at least a portion of a hair follicle.

Aspect 65 is the method of aspect 63 or 64, wherein the hair and/or skin samples comprise an upper portion of a hair follicle.

Aspect 66 is the method of any one of aspects 63-65, wherein the isolated phage is analyzed to determine lysogenic capacity, and wherein the isolated phage is determined to be non-lysogenic.

Aspect 67 is the method of any one of aspects 63-66, wherein the isolated bacteriophage has greater than 95%, 96%, 97%, 98%, 99%, 99.1%, 99.2%, 99.3%, 99.4%, 99.5%, 99.6%, 99.7%, 99.8%, 99.9%, or 99.95% genomic sequence identity to the genomic sequences of vB_SpsM-DH2 (SEQ ID NO: 1), vB_SpsS-DH5 (SEQ ID NO: 2), and/or vB_SpsM-DS10 (SEQ ID NO: 3).

Aspect 68 is the method of any one of aspects 63-67, wherein the isolated bacteriophage are not lysogenic, do not comprise antibiotic resistance coding sequences, do not comprise bacterial virulence coding sequences, and/or have lytic capacity.

Aspect 69 is the method of any one of aspects 63-68, wherein the isolated bacteriophage are evolved to improve lytic capacity, reduce lysogenic capacity, reduce antibiotic resistance gene obtainment capacity, reduce bacterial virulence gene obtainment capacity, and/or increase the number of bacterial strains subject to lysis by the isolated bacteriophage.

Throughout this application, the term “about” is used to indicate that a value includes the inherent variation of error for the measurement or quantitation method.

The use of the word “a” or “an” when used in conjunction with the term “comprising” may mean “one,” but it is also consistent with the meaning of “one or more,” “at least one,” and “one or more than one.”

The phrase “and/or” means “and” or “or”. To illustrate, A, B, and/or C includes: A alone, B alone, C alone, a combination of A and B, a combination of A and C, a combination of B and C, or a combination of A, B, and C. In other words, “and/or” operates as an inclusive or.

The words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

The compositions and methods for their use can “comprise,” “consist essentially of,” or “consist of” any of the ingredients or steps disclosed throughout the specification. Compositions and methods “consisting essentially of” any of the ingredients or steps disclosed limits the scope of the claim to the specified materials or steps which do not materially affect the basic and novel characteristic of the claimed invention.

The term “effective,” as that term is used in the specification and/or claims, means adequate to accomplish a desired, expected, or intended result.

“Individual, “subject,” and “patient” are used interchangeably and can refer to a human or non-human.

The terms “inhibiting” or “reducing” or “preventing” or “avoiding” or any variation of these terms, when used in the claims and/or the specification, includes any measurable decrease or complete inhibition to achieve a desired result.

Any method in the context of a therapeutic, diagnostic, or physiologic purpose or effect may also be described in “use” claim language such as “Use of” any compound, composition, or agent discussed herein for achieving or implementing a described therapeutic, diagnostic, or physiologic purpose or effect.

It is specifically contemplated that any limitation discussed with respect to one embodiment of the invention may apply to any other embodiment of the invention. Furthermore, any composition of the invention may be used in any method of the invention, and any method of the invention may be used to produce or to utilize any composition of the invention. Any embodiment discussed with respect to one embodiment of the disclosure applies to other embodiments of the disclosure as well and vice versa. For example, any step in a method described herein can apply to any other method. Moreover, any method described herein may have an exclusion of any step or combination of steps. Embodiments set forth in the Examples are also embodiments that may be implemented in the context of embodiments discussed elsewhere in a different Example or elsewhere in the application, such as in the Summary, Detailed Description, Claims, Abstract, and Brief Description of the Drawings.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1: Bacterial and Phage Isolation from Random Canine Samples. Sample pellets were streaked onto Mannitol salt agar (MSA) plates, with isolates sequenced based on catalase positivity. Identified Staphylococcal and Gram-positive isolates are shown. A phage hunt against S. aureus and S. pseudintermedius isolates was performed using a double agar overlay spot assay with the concentrated filtrates. This figure was created with BioRender.com.

FIG. 2 A-B: Morphology and host range of predicted lytic S. pseudintermedius phages. (A) Top—Phages imaged by TEM (scale bar represents 100 nm). Bottom—plaque size and morphology on strains AZM1 (DH2), AZM37 (DH5), and TCH1516 (DS10) respectively. Each tick (bottom line) represents 1 mm. (B) Bacterial host range determined via efficiency of plating (EOP) experiments on ten S. aureus and fifteen S. pseudintermedius strains. This figure was created with BioRender.com.

FIG. 3: Killing of S. pseudintermedius strain AZM22 on mouse skin. When both bacteria (AZM22, a S. pseudintermedius strain) and bacteriophage (e.g., DH2, also known as vB_SpsM-DH2) were placed on a bare patch of skin, DH2 reduced the bacterial burden of AZM22 by 3 logs when compared to AZM22 on skin alone. These data indicated that Staphylococcal phages can kill their bacterial hosts on animal skin.

### DETAILED DESCRIPTION

The present disclosure is based, at least in part, on the discovery and development of novel bacteriophages and bacteriophage combinations for treatment and prevention of Staphylococcus infection, including multidrug-resistant (MDR) Staphylococcus infection. Accordingly, disclosed herein, in some embodiments, are bacteriophage compositions comprising one or more of vB_SpsM-DH2, vB_SpsS-DH5, and/or vB_SpsM-DS10 (also referred to herein as DH2, DH5, and DS10, respectively). Further disclosed are devices and kits comprising such compositions and methods for use of such compositions in treatment and/or prevention of pathogenic Staphylococcus infection.

The inventors hypothesized that lytic phages may be found on companion pets like dogs, due to the prevalence of Staphylococcal species on these animals. For instance, in superficial bacterial folliculitis—a type of canine pyoderma—the upper portion of the hair follicle is commonly infected with S. pseudintermedius 15. As such, the inventors searched for S. pseudintermedius phages using canine feces, hair, and skin swabs. Described herein is the isolation and characterization of fourteen phages from canine hair and skin swabs, of which three (DH2, DH5, DS10, also referred to herein as vB_SpsM-DH2, vB_SpsS-DH5, and vB_SpsM-DS10, respectively) were predicted lytic. Mitomycin C (MMC) induction supported this designation, while DH2 and DS10—a phage K variant—were polyvalent against dozens of S. pseudintermedius and S. aureus strains. Described herein is the discovery and characterization of the first verified lytic S. pseudintermedius phages. the associated study design provides insights and guidance to facilitate discovery of additional lytic Staphylococcal phages.

The inventors opted for collecting feces, hair, and skin swabs from dogs, as methicillin-resistant Staphylococcus aureus (MRSA) and methicillin-resistant Staphylococcus pseudintermedius (MRSP) have been isolated from such samples 35. The Houston World Series of Dog Shows offered a unique opportunity to collect different samples from a variety of dog breeds, harboring a broad range of commensals and pathobionts. While S. aureus and S. pseudintermedius strains were isolated from the samples, phages were only found on S. pseudintermedius lawns. The preponderance of swab-derived phages is consistent with skin virome analysis and Staphylococci phage discovery ^{36,37}, suggesting that the source rather than the method determines phage recovery.

Two phages described herein, DSP1 and DSP2, were likely prophages from the bacterial hosts, and this explains their presence across several unique phage lysates. Three predicted lytic phages originated from concentrated hair and skin filtrates. These phages—DH2, DH5, and DS10—also targeted S. aureus strains as assessed by EOP assays. The phage K variant, DS10, plaqued on all S. aureus isolates tested. Interestingly, the DS10 gp146 mutation T523I is in a putative carbohydrate binding domain and not the annotated receptor binding protein of phage K, gp144. While not being bound by theory, it is possible that gp146 is also important for receptor recognition, for which small mutations may confer host range diversity. This rationale was supported by studies of Kayvirus mutants ^{38,39}. As a Twortvirus, DH2 represented a unique and promising candidate for phage therapy. While Kayviruses can expand their host range to S. pseudintermedius with limited lytic activity and infectivity, such ability is previously undocumented for Twortviruses ^{38,40,41}. While DH2 does possess a putative anti-repressor, the virulent lifestyle prediction agreed with the Twortvirus designation and results of the induction assay described herein (see Example 4).

Unlike myoviruses DH2 and DS10, siphovirus DH5 possessed elements of temperate phages, including an integrase and attachment sites. As shown herein, DH5 plaqued on S. pseudintermedius and S. aureus isolates but its EOP was very low (<0.001), indicating lysis-from-without rather than productive infection. As shown herein, Mitomycin C induced isolates produced no plaques, supporting the virulent lifestyle prediction (see Example 4). While not being limited by theory, a possible explanation for this is an effete integrase. Lysogeny modules are common amongst Staphylococcal phages with lytic variants arising due to mutations ^{33,42} DH5 may be one of these lytic variants, in support of this literature and the discoveries shown herein, where mostly temperate phages where discovered.

This project was undertaken, in part, to isolate and characterize lytic phages against S. pseudintermedius, given their virtual absence in literature ⁵. Moodley et. al. reported discovery of four phages from canine feces that appeared lytic against S. pseudintermedius and Staphylococcus schleiferi, however genomic analysis revealed a lysogeny model in all four ⁴³. To the best of the inventors knowledge, this is the first report of verified S. pseudintermedius lytic phages. Using canine hair and skin as novel sources, the inventors discovered three lytic phages: DH5, DH2, and DS10, with the latter two capable of infecting both S. pseudintermedius and S. aureus strains. While skin is a known repository for phages, hair follicles harbor a rich microbiome including S. pseudintermedius during diseased states ^{15,44}. While not being limited by theory, this microenvironment might favor the evolution of lytic phages due to the proximity and density of potential hosts and explain why 2 novel lytic phages were found from dog hair clippings. Based on EOP analysis, a phage cocktail of DH2, DH5, and/or DS10 may plaque on all S. aureus and S. pseudintermedius strains tested.

### I. Bacteriophage Compositions

Embodiments of the disclosure are directed to compositions comprising one or more bacteriophage. As used herein, a “bacteriophage composition” describes any composition comprising one or more bacteriophage (also “phage”). A bacteriophage of the present disclosure may be a lytic phage. A bacteriophage of the present disclosure may be a phage capable of infecting one or more pathogenic bacteria. A bacteriophage of the present disclosure may be a phage capable of infecting one or more bacteria of the genus Staphylococcus. In some embodiments, a bacteriophage of the present disclosure may be polyvalent. In some embodiments, a phage of the present disclosure is capable of infecting one or more bacteria of the family Staphylococcus pseudintermedius and/or Staphylococcus aureus. A bacteriophage of the present disclosure may be a polyvalent phage that infections greater than 2, 3, 4, 5, or more than 5 members of the genus Staphylococcus.

In some embodiments, a bacteriophage of the disclosure is a phage capable of infecting Staphylococcus, such as a drug resistant and/or multidrug-resistant (MDR) Staphylococcus. In some embodiments, a bacteriophage of the disclosure is a phage capable of infecting Staphylococcus pseudintermedius, such as a drug resistant and/or MDR Staphylococcus pseudintermedius. In some embodiments, a bacteriophage of the disclosure is a phage capable of infecting Staphylococcus aureus, such as a drug resistant and/or MDR Staphylococcus aureus. A bacteriophage composition may comprise, for example, abacteriophage generated using a method or system described in PCT Publication No. WO2020/264096, titled “Systems And Methods For Generating Bacteriophages Adapted To Infect A Target Bacterial Strain”, incorporated herein by reference in its entirety.

A bacteriophage composition of the disclosure may comprise, consist essentially of, or consist of, for example, one or more bacteriophage capable of lysing a pathogenic bacteria. In some embodiments, a bacteriophage is a polyvalent phage. In some embodiments, a bacteriophage is a monovalent phage. A bacteriophage composition of the present disclosure may comprise, consist essentially of, consist of, at least, at most, or exactly 1, 2, 3, 4, 5, 6, 7, 8, 9, or 10 different bacteriophages, or more different bacteriophages. In some embodiments, a bacteriophage composition of the disclosure comprises one or more of vB_SpsM-DH2 (Accession No. OM373548; also referred to herein as “DH2”), vB_SpsS-DH5 (Accession No. OM373549; also referred to herein as “DH5”), and/or vB_SpsM-DS10 (Accession No. OM373557; also referred to herein as “DS10”). In some embodiments, a bacteriophage composition of the disclosure comprises, consists essentially of, or consists of vB_SpsM-DH2. In some embodiments, a bacteriophage composition of the disclosure comprises, consists essentially of, or consists of vB_SpsS-DH5. In some embodiments, a bacteriophage composition of the disclosure comprises, consists essentially of, or consists of vB_SpsM-DS10. In some embodiments, a bacteriophage composition of the disclosure comprises, consists essentially of, or consists of vB_SpsM-DH2 and vB_SpsS-DH5. In some embodiments, a bacteriophage composition of the disclosure comprises, consists essentially of, or consists of vB_SpsM-DH2 and vB_SpsM-DS10. In some embodiments, a bacteriophage composition of the disclosure comprises, consists essentially of, or consists of vB_SpsS-DH5 and vB_SpsM-DS10.

A bacteriophage composition of the disclosure may comprise, in addition to one or more of vB_SpsM-DH2, vB_SpsS-DH5, and/or vB_SpsM-DS10, one or more additional bacteriophages (e.g., additional bacteriophages capable of lysing a pathogenic bacteria, e.g., an E. coli, a Staphylococcus, a Pseudomonas, etc.). Additional bacteriophages contemplated herein include, but are not limited to, bacteriophages EC1, CF2, ES12, ES21, ES26, HP3, and ES17 which bacteriophages are described in, for example, Gibson S B, Green S I, Liu C G, et al. Constructing and Characterizing Bacteriophage Libraries for Phage Therapy of Human Infections. Front Microbiol. 2019; 10:2537; U.S. 63/241,209 filed Sep. 7, 2021; and U.S. 63/274,815 filed Nov. 2, 2021; each of which are incorporated herein by reference in their entirety.

In some embodiments, a bacteriophage composition of the disclosure may comprise, in addition to one or more bacteriophages, one or more metals. The one or more metals may include, for example, calcium, magnesium, iron, sodium, and/or potassium.

In some embodiments, a bacteriophage composition comprising two or more different bacteriophages may comprise various amounts of each bacteriophage. For example, a composition may comprise substantially the same amount of each bacteriophage. Alternatively, a composition may comprise substantially different amounts of each bacteriophage.

A bacteriophage composition may comprise, consist essentially of, or consist of, at least, at most, or about 10³, 10⁴, 10⁵, 10⁶, 10⁷, 10⁸, 10⁹, 10¹⁰, 10¹¹, 10¹², or 10¹³ plaque forming units (PFU) per milliliter (ml) (PFU/ml) of each of the one or more bacteriophage in the composition, or more. In some embodiments, a bacteriophage composition comprises, consists essentially of, or consists of, at least, at most, or about 10³, 10⁴, 10⁵, 10⁶, 10⁷, 10⁸, 10⁹, 10¹⁰, 10¹¹, 10¹², or 10¹³ PFU/ml of vB_SpsM-DH2. In some embodiments, a bacteriophage composition comprises, consists essentially of, or consists of, at least, at most, or about 10³, 10⁴, 10⁵, 10⁶, 10⁷, 10⁸, 10⁹, 10¹⁰, 10¹¹, 10¹², or 10¹³ PFU/ml of vB_SpsS-DH5. In some embodiments, a bacteriophage composition comprises, consists essentially of, or consists of, at least, at most, or about 10³, 10⁴, 10⁵, 10⁶, 10⁷, 10¹, 10⁹, 10¹⁰, 10¹¹, 10¹², or 10¹³ PFU/ml of vB_SpsM-DS10.

In some embodiments, a composition may comprise an isolated bacteriophage with greater than 95%, 96%, 97%, 98%, 99%, 99.1%, 99.2%, 99.3%, 99.4%, 99.5%, 99.6%, 99.7%, 99.8%, 99.9%, or 99.95% genomic sequence identity to the genomic sequences of vB_SpsM-DH2 (SEQ ID NO: 1), vB_SpsS-DH5 (SEQ ID NO: 2), and/or vB_SpsM-DS10 (SEQ ID NO: 3). In some embodiments, a composition may comprise an isolated bacteriophage identified in Table 1.

In some embodiments, a composition may comprise an isolated bacteriophage, wherein the isolated bacteriophage are not lysogenic, do not comprise antibiotic resistance coding sequences, do not comprise bacterial virulence coding sequences, and/or have lytic capacity.

In some embodiments, a composition may comprise an isolated bacteriophage, wherein the bacteriophage were evolved to have improved lytic capacity, reduced lysogenic capacity, reduced antibiotic resistance gene obtainment capacity, reduced bacterial virulence gene obtainment capacity, and/or increase the number of bacterial strains subject to lysis by the isolated bacteriophage lytic. In certain embodiments, an isolated bacteriophage has been evolved to have mutations in the tail protein.

A. vB_SpsM-DH2

Embodiments of the present disclosure can comprise, consist essentially of, or consist of bacteriophage vB_SpsM-DH2 (also “Staphylococcus phage DH2,” or “DH2,” used synonymously herein), compositions, kits, and devices comprising vB_SpsM-DH2, and methods for use thereof. An vB_SpsM-DH2 genome is described by GenBank accession number OM373548, which is noted as 153,381 bp in length, and the sequence of which is incorporated herein by reference (SEQ ID NO: 1). The sequence represented by SEQ ID NO: 1 comprises direct terminal repeals (DTR), which in some embodiments may be considered superfluous when considering sequence identity, and thus can be excluded. In certain embodiments, a sequence identity relative to SEQ ID NO: 1 is calculated without taking into consideration the DTRs.

B. vB_SpsS-DH5

Embodiments of the present disclosure can comprise, consist essentially of, or consist of bacteriophage vB_SpsS-DH5 (also “Staphylococcus phage DH5,” or “DH5,” used synonymously herein), compositions, kits, and devices comprising vB_SpsS-DH5, and methods for use thereof. An vB_SpsS-DH5 genome is described by GenBank accession number OM373549, which is noted as 92,077 bp in length, and the sequence of which is incorporated herein by reference (SEQ ID NO: 2).

C. vB_SpsM-DS10

Embodiments of the present disclosure can comprise bacteriophage vB_SpsM-DS10 (also “Staphylococcus phage DS10,” or “DS10,” used synonymously herein), compositions, kits, and devices comprising vB_SpsM-DS10, and methods for use. An vB_SpsM-DS10 genome is described by GenBank accession number OM373557, which is noted as 148,313 bp in length, and the sequence of which is incorporated herein by reference (SEQ ID NO: 3). The sequence represented by SEQ ID NO: 3 comprises DTRs, which in some embodiments may be considered superfluous when considering sequence identity, and thus can be excluded. In certain embodiments, a sequence identity relative to SEQ ID NO: 3 is calculated without taking into consideration the DTRs.

### II. Treatment and Prevention of Bacterial Infection

Embodiments of the present disclosure can be directed to methods for treatment and prevention of a bacterial infection in an individual. In some embodiments, disclosed are methods for treatment or prevention of a Staphylococcus infection in an individual. In some embodiments, disclosed are methods for treatment or prevention of a Staphylococcus pseudintermedius infection in an individual. In some embodiments, disclosed are methods for treatment or prevention of a Staphylococcus aureus infection in an individual. In particular embodiments, the present disclosure provides methods for treatment or prevention of a Staphylococcus infection in an individual comprising, consisting essentially of, or consisting of administering to the individual an effective amount of one or more bacteriophage compositions disclosed herein, including bacteriophages disclosed herein. Bacteriophages useful for such treatment methods include those described herein, for example vB_SpsM-DH2, vB_SpsS-DH5, and/or vB_SpsM-DS10. Accordingly, in some embodiments, disclosed herein is a method for treatment or prevention of an Staphylococcus infection in an individual comprising administering to the individual an effective amount of vB_SpsM-DH2, vB_SpsS-DH5, and/or vB_SpsM-DS10.

In some embodiments, multiple bacteriophage (e.g., two or more vB_SpsM-DH2, vB_SpsS-DH5, and vB_SpsM-DS10) are administered to an individual in the same formulation. Alternatively, multiple bacteriophage may be administered to an individual in different formulations (e.g., 2, 3, or more formulations). Multiple bacteriophage may be administered to an individual at the same time or may be administered at different times.

Multiple bacteriophage may be administered to an individual substantially simultaneously, for example via a single composition. For example, vB_SpsM-DH2, vB_SpsS-DH5, and/or vB_SpsM-DS10 may be administered to an individual having a pathogenic Staphylococcus infection at the same time, as a single composition. As another example, vB_SpsM-DH2, vB_SpsS-DH5, and/or vB_SpsM-DS10 may be administered to an individual at risk of contracting a pathogenic Staphylococcus infection at the same time, as a single composition.

Multiple bacteriophage may be administered to an individual sequentially in any order. For example, an individual having a pathogenic Staphylococcus infection may be administered vB_SpsM-DH2, followed by vB_SpsS-DH5, followed by vB_SpsM-DS10; or in the opposite order, or with DH5 being administered first or last, or in any alternative order.

Bacteriophages of the disclosure may be administered to an individual once, or may be administered multiple times (e.g., 2, 3, 4, 5, 6, 7, 8, 9, 10, 11, 12, 13, 14, 15, or more times). Bacteriophages may be administered via an appropriate treatment regimen for an appropriate length of time, e.g., for effective treatment or prevention of a pathogenic Staphylococcus infection. For example, a bacteriophage composition may be administered to an individual 1, 2, 3, or 4 times per day (or more); 1, 2, 3, 4, 5, 6, or 7 times per week (or more); or 1, 2, 3, 4, 5, 6, 7, 8, 9, 10, 11, 12, 13, 14, 15, 16, 17, 18, 19, or 20 times per month (or more). A bacteriophage composition may be administered for at least, at most, or exactly 1, 2, 3, 4, 5, 6, 7, 8, 9, 10, 11, 12, 13, or 14 days (or more); 1, 2, 3, 4, 5, 6, 7, 8, 9, 10, 11, 12, 13, 14, 15, 16, 17, 18, 19, 20, or 21 weeks (or more); or 1, 2, 3, 4, 5, 6, 7, 8, 9, 10, 11, or 12 months (or more). In some embodiments, a bacteriophage is administered twice per week for between 6 and 12 weeks.

An individual receiving a treatment of the disclosure may have an infection in, for example, one or more of a urinary tract, blood, gut, abdomen, stomach, lungs, skin, wound (e.g., bums, scratches, surgical wounds, etc.) eyes, ears, mouth, nose, kidneys, prostate, bladder, brain, vaginal tract, heart, liver, and spleen. In some embodiments, an individual has a skin infection. In some embodiments, an individual has a wound infection. In some embodiments, an individual has a blood infection. In some embodiments, an individual has developed sepsis. In some embodiments, an individual has pyoderma. In some embodiments, an individual has otitis (e.g., otitis externa). In some embodiments, an individual has a respiratory tract infection. In some embodiments, an individual has a reproductive tract infection. In some embodiments, an individual has a urinary tract infection. In some embodiments, an individual has a catheter-associated urinary tract infection. An individual may have one or more of a urinary tract infection, neonatal meningitis, a blood-stream infection, pneumonia, sepsis, a surgical wound infection, a skin infection, a prostate infection, meningitis, and a vaginal infection. An individual may have one or more symptoms of a pathogenic infection, for example boils, carbuncles, blisters, sores, rashes, inflammation, swelling, diarrhea, stomach cramping, nausea, and/or vomiting. In some embodiments, an individual does not have any symptoms of a pathogenic infection.

In some embodiments, an individual receiving a treatment of the disclosure has a Staphylococcus infection. In some embodiments, an individual has been diagnosed with a Staphylococcus infection. In some embodiments, an individual has one or more symptoms of a Staphylococcus infection (including, e.g., boils, carbuncles, blisters, sores, rashes, inflammation, swelling, diarrhea, stomach cramping, nausea, and/or vomiting). In some embodiments, the individual does not have any symptoms of a Staphylococcus infection. In some embodiments, the Staphylococcus is multidrug-resistant (i.e. is a multidrug-resistant Staphylococcus).

An individual may have received a Staphylococcus infection from various sources. For example, in some embodiments, the Staphylococcus was from a beverage, comestible (e.g., undercooked meat, unpasteurized milk, apple juice or cider; or soft cheese made from raw milk), another individual, or an environment (e.g., towels, razors, daycare, boarding, gymnasiums, ground or surface water, water used to irrigate crops, a public water system, a hospital, a school, a nursing home, a petting zoo, a cruise ship, a train, an airplane, etc.).

An individual receiving a treatment of the disclosure may be an immunosuppressed individual. An immunosuppressed individual may be an individual having, for example, an immune cell defect, asplenia, impaired splenic function, nephrotic syndrome, or an autoimmune condition. An immunosuppressed individual may be an individual subject to immunosuppressive conditions, for example chemotherapeutic agents or immunosuppressant agents (e.g., a glucocorticoid, a calcineurin inhibitor, an antimetabolite, a medication to reduce stomach acid such as a proton pump inhibitor, or an antibody therapy). Accordingly, in certain embodiments, bacteriophages of the disclosure may be administered to an individual before, during, and/or after subjecting the individual to immunosuppressive conditions. For example, bacteriophages of the disclosure (e.g., vB_SpsM-DH2, vB_SpsS-DH5, and/or vB_SpsM-DS10) may be administered to an individual having cancer during treatment with chemotherapy, thereby treating and/or preventing a pathogenic Staphylococcus infection.

In some embodiments an individual is a mammal. In some embodiments, an individual is a domestic animal. In some embodiments, an individual is a farm animal. In some embodiments, an individual is a zoo animal. In some embodiments, an individual is a human. In some embodiments, an individual is a dog. In some embodiments, an individual is a cat. In some embodiments, an individual is a cow. In some embodiments, an individual is a goat. In some embodiments, an individual is a horse. In some embodiments, an individual can be but is not limited to, a dog, cat, ferret, rabbit, cow, duck, pig, goat, chicken, horse, llama, camel, ostrich, deer, turkey, dove, sheep, goose, oxen, and/or reindeer.

In certain embodiments, compositions and/or methods described herein are utilized to reduce Staphylococcus carriage by an individual and/or in an environment. In certain embodiments, reduction of Staphylococcus carriage is an infection preventative measure.

### III. Devices

Also contemplated herein, in some embodiments, are devices (e.g., medical devices) comprising one or more bacteriophages or bacteriophage compositions of the present disclosure. A device of the disclosure may comprise one or more of vB_SpsM-DH2, vB_SpsS-DH5, and/or vB_SpsM-DS10. A device is described herein as “comprising” a bacteriophage or bacteriophage composition where the device has in, on, or around it, or is attached to, the bacteriophage or bacteriophage composition. In some embodiments, a device of the disclosure is a medical device. In such cases, it may be desirable for a medical device to comprise bacteriophages capable of treating or preventing a pathogenic infection, such as an Staphylococcus infection. Various medical devices are recognized in the art and contemplated herein. Examples of devices contemplated herein include, but are not limited to, a catheter, drive line, syringe, tube, implant, defibrillator, artificial joint, pacemaker, screw, rod, disc, intrauterine device, pin, plate, stent, dental device, eye lens, shunt, valve, neurological or neurosurgical device, gastrointestinal device, genitourinary device, catheter cuff, vascular access device, and wound drain. In some embodiments, the device is a stent. In some embodiments, the device is a catheter. In some embodiments, the device is an implant.

Embodiments of the disclosure can comprise methods for preparing a device comprising subjecting a device to a bacteriophage composition of the disclosure (e.g., a bacteriophage composition comprising vB_SpsM-DH2, vB_SpsS-DH5, and/or vB_SpsM-DS10). Disclosed are methods comprising placing a bacteriophage composition on a device, placing a bacteriophage composition around a device, placing a bacteriophage composition in a device, and coating a device with a bacteriophage composition (e.g., coating one or more surfaces of a device). Aspects further comprise delivering the device to an individual following such preparation.

### IV. Pharmaceutical Compositions

In certain embodiments, the compositions or agents for use in the disclosed methods, such as bacteriophage(s) (e.g., vB_SpsM-DH2, vB_SpsS-DH5, and/or vB_SpsM-DS10), are suitably contained in a pharmaceutically acceptable carrier. In some embodiments, the carrier is non-toxic, biocompatible and is selected so as not to detrimentally affect the biological activity of the agent. The agents in some embodiments of the disclosure may be formulated into preparations for local delivery (i.e. to a specific location of the body) or systemic delivery, in solid, semi-solid, gel, liquid or gaseous forms such as tablets, capsules, powders, granules, ointments, solutions, depositories, inhalants and injections allowing for oral, parenteral or surgical administration. Certain embodiments of the disclosure also contemplate local administration of the compositions by coating medical devices and the like.

Suitable carriers for parenteral delivery via injectable, infusion or irrigation and topical delivery include, but are not limited to, distilled water, saline, physiological phosphate-buffered saline, normal or lactated Ringer's solutions, dextrose solution, Hank's solution, or propanediol. In certain embodiments, a suitable carrier is saline. In addition, sterile, fixed oils may be employed as a solvent or suspending medium. For this purpose any biocompatible oil may be employed including synthetic mono- or diglycerides. In addition, fatty acids such as oleic acid find use in the preparation of injectables. The carrier and agent may be compounded as a liquid, suspension, polymerizable or non-polymerizable gel, paste or salve.

The carrier may also comprise a delivery vehicle to sustain (i.e., extend, delay or regulate) the delivery of the agent(s) or to enhance the delivery, uptake, stability or pharmacokinetics of the therapeutic agent(s). Such a delivery vehicle may include, by way of non-limiting examples, microparticles, microspheres, nanospheres or nanoparticles composed of proteins, liposomes, carbohydrates, synthetic organic compounds, inorganic compounds, polymeric or copolymeric hydrogels and polymeric micelles.

In certain embodiments, the actual dosage amount of a composition administered to a patient or subject can be determined by physical and physiological factors such as body weight, severity of condition, the type of disease being treated, previous or concurrent therapeutic interventions, idiopathy of the patient and on the route of administration. The practitioner responsible for administration will, in any event, determine the concentration of active ingredient(s) in a composition and appropriate dose(s) for the individual subject.

Solutions of pharmaceutical compositions can be prepared in water suitably mixed with a surfactant, such as hydroxypropylcellulose. Dispersions also can be prepared in glycerol, liquid polyethylene glycols, mixtures thereof and in oils. Under ordinary conditions of storage and use, these preparations contain a preservative to prevent the growth of microorganisms.

In certain embodiments, the pharmaceutical compositions are advantageously administered in the form of injectable compositions either as liquid solutions or suspensions; solid forms suitable or solution in, or suspension in, liquid prior to injection may also be prepared. These preparations also may be emulsified. A typical composition for such purpose comprises a pharmaceutically acceptable carrier. For instance, the composition may contain 10 mg or less, 25 mg, 50 mg or up to about 100 mg of human serum albumin per milliliter of phosphate buffered saline. Other pharmaceutically acceptable carriers include aqueous solutions, non-toxic excipients, including salts, preservatives, buffers and the like.

Examples of non-aqueous solvents are propylene glycol, polyethylene glycol, vegetable oil and injectable organic esters such as ethyloleate. Aqueous carriers include water, alcoholic/aqueous solutions, saline solutions, parenteral vehicles such as sodium chloride, Ringer's dextrose, etc. Intravenous vehicles include fluid and nutrient replenishers. Preservatives include antimicrobial agents, antifungal agents, anti-oxidants, chelating agents and inert gases. The pH and exact concentration of the various components the pharmaceutical composition are adjusted according to well-known parameters.

Additional formulations are suitable for oral administration. Oral formulations include such typical excipients as, for example, pharmaceutical grades of mannitol, lactose, starch, magnesium stearate, sodium saccharine, cellulose, magnesium carbonate and the like. The compositions take the form of solutions, suspensions, tablets, pills, capsules, sustained release formulations or powders.

In further embodiments, the pharmaceutical compositions may include classic pharmaceutical preparations. Administration of pharmaceutical compositions according to certain embodiments may be via any common route so long as the target tissue is available via that route. This may include oral, nasal, aural, ophthalmic, buccal, rectal, vaginal or topical. Alternatively, administration may be by orthotopic, intradermal, subcutaneous, intramuscular, intraperitoneal or intravenous injection. Such compositions would normally be administered as pharmaceutically acceptable compositions that include physiologically acceptable carriers, buffers or other excipients. For treatment of conditions of the lungs, aerosol delivery can be used. Volume of the aerosol may be between about 0.01 ml and 0.5 ml, for example.

An effective amount of the pharmaceutical composition is determined based on the intended goal. The term “unit dose” or “dosage” refers to physically discrete units suitable for use in a subject, each unit containing a predetermined-quantity of the pharmaceutical composition calculated to produce the desired responses discussed above in association with its administration, i.e., the appropriate route and treatment regimen. The quantity to be administered, both according to number of treatments and unit dose, depends on the protection or effect desired.

Precise amounts of the pharmaceutical composition also depend on the judgment of the practitioner and are peculiar to each individual. Factors affecting the dose include the physical and clinical state of the patient, the route of administration, the intended goal of treatment (e.g., alleviation of symptoms versus cure) and the potency, stability and toxicity of the particular therapeutic substance.

### A. Other Agents

It is contemplated that other agents may be used in combination with certain embodiments of the present embodiments to improve the therapeutic efficacy of treatment. These additional agents include agents having antibacterial properties (e.g., antibiotics). For example, one or more therapeutic phage of the disclosure may be used in combination with one or more antibiotics. Various antibiotics are recognized in the art and contemplated herein including, for example, ceftazidime, ciprofloxacin, kanamycin, colistin (polymyxin E), trimethoprim, cefepime, sulfamethoxazole, levofloxacin, and polymyxin B.

### V. Kits

Embodiments of the present disclosure can also concern kits containing compositions of the disclosure or compositions to implement methods disclosed herein. In some embodiments, kits can be used to evaluate one or more biomarkers. In certain embodiments, a kit contains, contains at least or contains at most 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 100, 500, 1,000 or more probes, primers or primer sets, synthetic molecules or inhibitors, or any value or range and combination derivable therein. In some embodiments, there are kits for evaluating biomarker activity in a cell.

Kits may comprise components, which may be individually packaged or placed in a container, such as a tube, bottle, vial, syringe, or other suitable container means.

Individual components may also be provided in a kit in concentrated amounts; in some embodiments, a component is provided individually in the same concentration as it would be in a solution with other components. Concentrations of components may be provided as 1×, 2×, 5×, 10×, or 20× or more.

In some embodiments, kits of the disclosure comprise one or more bacteriophages housed in a suitable container. A kit may comprise, comprise at least, or comprise at most 1, 2, 3, 4, 5, 6, 7, 8, 9, or 10 different bacteriophages, or more. In some embodiments, a kit of the disclosure comprises 1, 2, or 3, of vB_SpsM-DH2, vB_SpsS-DH5, and/or vB_SpsM-DS10, and optionally 1, 2, 3, 4, 5, 6, or more additional bacteriophages. One or more phage may be specifically excluded from certain embodiments. Phage may be housed in a suitable container. A kit of the disclosure may further comprise a device. A kit may comprise a device having one or more bacteriophages of the disclosure on, in, and/or around the device. A kit may comprise a device separate from one or more bacteriophages.

### Examples

The following examples are included to demonstrate certain embodiments of the invention. It should be appreciated by those of skill in the art that the techniques disclosed in the examples which follow represent techniques discovered by the inventor to function well in the practice of the invention, and thus can be considered to constitute certain modes for its practice. However, those of skill in the art should, in light of the present disclosure, appreciate that many changes can be made in the specific embodiments which are disclosed and still obtain a like or similar result without departing from the spirit and scope of the invention.

### Materials and Methods

Unless otherwise noted, the experiments and procedures described herein were conducted as follows.

### Sample Collection and Processing

Fecal samples were collected using sterile gloves and placed in individual 1.5 mL centrifuge tubes; hair clippings from various dogs were collected in individual Ziploc bags; skin swabs were collected from canine underbellies (where there is little hair) using Amies swabs soaked in 1.5 mL saline.

Each fecal and/or hair sample was mixed thoroughly with either 1 or 5 mL respectively of phage buffer (6.7 mM Tris-HCl, 3.2 mM Tris-Base, 100 mM NaCl, 10 mM MgSO₄0.7H₂O, pH 8.0, filter sterilized). No phage buffer was added to the skin swabs in saline solution. For the fecal and skin swab samples, 1 mL was transferred to a new, sterile 1.5 mL tube and centrifuged at 17,000×g for 10 minutes at room temperature with supernatants filtered using a 0.22 μM syringe-filter (Millipore, SLGVR33RS). The hair samples were pooled, and the mixture filtered through a 0.22 μM bottle top filter (Millipore, SCGPS02RE). The pellets and the filtered supernatants were saved for bacterial and phage isolation, respectively, as described herein.

### Bacterial Isolation

Mannitol salt agar (MSA) (Thermo Scientific™, R01581) plates were used to select for possible S. aureus/S. pseudintermedius isolates. Pellets for each sample type were resuspended with 500 μL of lysogeny broth (LB) and titered (serially diluted in LB media). Next, 20 μL slants of each dilution were plated onto each plate and incubated overnight at 37° C. A single colony from each plate was picked and streaked onto a new plate to ensure purity and incubated overnight at 37° C. Lastly, 10% glycerol stocks were made from log phase LB cultures and stored at −80° C. Isolates that grew on the MSA plates were further tested for catalase activity for possible identification as S. pseudintermedius or S. aureus. Bacterial cultures were submitted to the Center of Metagenomics and Microbiome Research at Baylor College of Medicine for 16S rRNA sequencing and species identification. Bacterial strains isolated through this method were labeled AZM19—32. Additional S. aureus/S. pseudintermedius isolates (AZM1-2, 33-38) were received from veterinary clinics through Anizome LLC. Other S. aureus isolates from human origin (LMB2, MW2 and TCH1516) were obtained from TAILDR Labs' bacterial isolate library.

### Phage Isolation

Supernatants from each sample type were pooled and transferred to a 100 kDa centrifugal filter (Millipore, ACS510024) and centrifuged at 3,000×g until the hold-up volume was concentrated by a factor of 100. The concentrated filtrate was transferred to a new, sterile 1.5 mL tube and stored at 4° C. until phage isolation. Phages were isolated on S. pseudintermedius lawns by spotting the concentrated hair, skin or fecal filtrates via the double agar overlay method ¹⁶.

### Host Range and Efficiency of Plating

Host range and efficiency of plating (EOP) were determined as previously described ¹⁷ using 15 S. pseudintermedius and 10 S. aureus isolates. Phage plate lysates were titered (serially diluted in phage buffer) with 5 μL of each dilution spotted onto lawns of S. aureus or S. pseudintermedius in a double agar overlay assay and incubated overnight at 37° C. Resulting individual plaques were counted to determine the titer (PFU/mL).

### Transmission Electron Microscopy

Bacteriophage samples were imaged at the Baylor College of Medicine Cryo-Electron Microscopy Advanced Technology Core Facility (Texas Medical Center, Houston, TX). Quantifoil 2/2 200 Cu+2 nm ThinC (Quantifoil Micro Tools GmbH, Jena, Germany) grids were used for the negative stain preparation and imaging. A Pelco EasiGlow (Ted Pella, Inc) was used to glow discharge the grids and create a hydrophilic surface for the sample and stain adsorption. The glow discharge process was done for 10 seconds with a current value of 15 uA and a vacuum of −200 psig. Bacteriophage aliquots (5 μl) were applied to the grids and incubated for 1-3 minutes depending on the phage concentration. Whatman 541 (Whatman, Inc) filter paper was used to absorb any excess buffer, water, and 2% Uranyl Acetate (Sigma Aldrich) during staining. The final stain required 1 minute incubation to assure adequate contrast and stain quality in the images. Stained grids were dried overnight in a desiccator and imaged the following day.

Negative stained grids were imaged with either a JEOL1230 operating at 80 kV or a 200 kV JEOL 2100FS Electron Microscope (JEOL Ltd, Japan). The JEOL 1230 is outfitted with a 4k×4k Gatan Ultrascan CCD Camera (Gatan, AMETEK) and the JEOL 2100 is outfitted with a DE-12 3k×4k direct detecting camera (Direct Electron, San Diego, CA). To minimize astigmatism or other beam aberrations prior to imaging, each microscope was aligned on a neutral carbon grid. Gain and dark reference images were automatically applied by the cameras prior to saving the final images. Images were collected at various magnifications ranging from 5,000× to 60,000× on each scope to assess the bacteriophage particle concentration, distribution, size, and structure. Further image visualization and correlative studies were performed using FIJI ImageJ software.

### Genomic Sequencing and Bioinformatic Analysis

Genomic DNA purified from plate lysates via E.Z.N.A.® Universal Pathogen Kit (Omega Bio-Tek) was submitted to Novogene (Sacramento, CA, USA) for microbial whole genome sequencing using Illumina NovaSeq 6000-PE150 platform and 350 bp insert DNA library preparation (Q30 ≥85%) with a resolution of 1 Gb of raw data per sample.

Data were analyzed with software using default settings unless otherwise specified. Raw short-reads were trimmed to a quality score of 30 and reads shorter than 50 bps in length were filtered out using BBDuk (version 38.84) ¹⁸. After trimming, 3% of reads were randomly subsampled (resulting in ˜100,000-150,000 reads per sample) and assembled using Geneious assembler in Geneious 2022.0.1. To look for potential rare species in the samples, trimmed reads were also normalized to a target coverage level of 200× and minimum depth of 10× using BBNorm (version 38.84) ¹⁸ and then assembled using Geneious assembler. Assemblies from both normalized and subsampled reads were then compared using progressive Mauve (version 2015-02-26) ¹⁹ to verify that they matched. Assemblies were validated by mapping all trimmed reads from each corresponding sample. Final assemblies were then compared using progressive Mauve (version 2015-02-26) and unique contigs kept for further analysis. Assemblies were annotated using RASTtk 20-22

Annotated assemblies were screened for antibiotic resistance and virulence genes by using BLAST ²³ (version 2.8.1) to align assemblies to the Comprehensive Antibiotic Resistance Database (CARD; database version 3.0.7) ²⁴, the Virulence Factor Database (VFDB; database version 2021-09-24) ²⁵, the Victor V F database (downloaded 2020-11-18) ²⁶, and the PATRIC VF database (downloaded 2020-11-18) ²⁷. Genus was predicted using BLASTn (version 2.8.1) and the nr/nt database to determine the most closely related sequenced genomes and predicted the potential genus based on >70% nucleotide identity over >60% of the query. Phage lifestyles were predicted with PhageAI ¹⁴ by parsing annotated features for “integrase”. Assemblies were also analyzed using PHASTER ²⁸ to search for integrases and attachment sites using BLAST annotation software. PHASTER was used to predict prophage content in S. pseudintermedius ATCC 49051.

### Induction Assays

An overnight culture of the host strain was subcultured 1:100 in 20 mL of fresh LB media and incubated at 37° C. with shaking until OD 600 nm=0.4. Mitomycin C (MilliporeSigma, 47582010MG) was added to 10 μg/mL, covered with foil, and incubated at 37° C. with continuous shaking for 2 hours followed by addition of 2% chloroform for 10 min. The culture was centrifuged for 10 minutes at 4,400 rpm and the supernatant transferred to a new 15 mL conical tube. 50 μL of this induced lysate supernatant was mixed with 50 μL of the fresh host culture in 3 mL of 0.75% top agar, spread over an LB agar plate and incubated at 37° C. overnight. Plates were checked for plaque formation and served as the background lysogen plaque formation for that host strain.

Each phage was mixed with its host at a multiplicity of infection (MOI) of 10, plated, and incubated overnight at 37° C. At least five resulting resistant colonies were restreaked twice onto a fresh LB agar plate and incubated 37° C. overnight to remove lytic phage. After the second streak, cultures from each colony were induced as described above. Resulting plates were checked for plaque formation and compared to the host baseline.

### Example 1 —S. Pseudintermedius Strains and Phages Isolated from Canine Hair and Skin

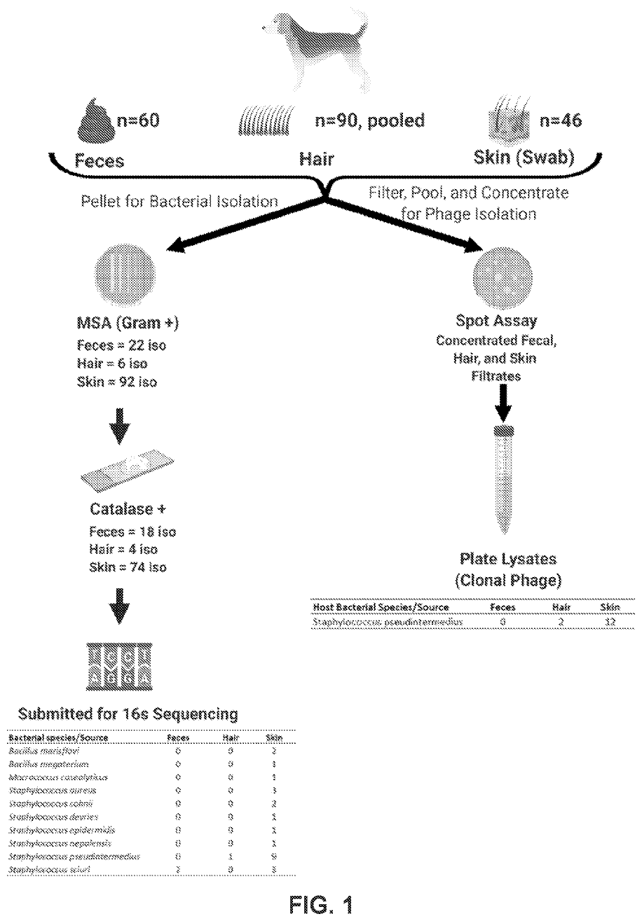

The inventors collected 60 fecal samples, 90 hair samples, and 46 skin swabs from dogs at the 2019 Houston World Series of Dogs for isolation of S. aureus and S. pseudintermedius strains and phages. 16S RNA sequencing of MSA-positive, catalase-positive isolates identified 12 as S. pseudintermedius with three identified as S. aureus. All strains were isolated from skin swabs except for one S. pseudintermedius isolate recovered from hair samples. Other recovered Staphylococcal and Gram-positive species and their origins are listed in FIG. 1. A total of fourteen phages against S. pseudintermedius were initially discovered. Twelve originated from the concentrated skin swab filtrates, but no phages against S. pseudintermedius were obtained from the concentrated fecal filtrates. Surprisingly, two phages were recovered from the concentrated hair filtrates. No S. aureus phages were initially recovered from any of the sources (FIG. 1).

### Example 2—Sequencing Analysis Predicted Three Lytic Phages and Co-Amplification of Two Prophages

The genomic analysis for all twelve phage plate lysates are summarized in Table 1. Genome sizes ranged from 35, 683-139,829 bp. Encoded ORFs ranged from 17-217, and tRNAs ranged from 0-5. Genera represented included Twortvirus, Sextaecvirus, Andhravirus, Fibralongavirus, Coventryvirus, Kayvirus, Biseptimavirus, and two unknown. None of the genomes contained antibiotic resistance coding sequences, while four phages (DS3, DS4, DS5, DS7) had at least one bacterial virulence coding sequence. Surprisingly, three phages were predicted to be lytic—a rarity for S. pseudintermedius phage. They were deposited and named via the Adriaenssens and Brister method as Staphylococcal phages vB_SpsM-DH2, vB_SpsS-DH5, and vB_SpsM-DS10 29. Further investigation revealed DS10 to be a phage K variant, with a SNP causing a T523I mutation in a putative carbohydrate binding domain (locus tag: CPT_phageK_gp146; protein ID: YP_009041342.1). The other phages were predicted to be temperate, containing putative integrases and/or attachment sites. Sequencing coverage depth revealed that multiple preparations from presumably clonal plaques contained more than one genome. For example, the DS1 preparation contained two contigs assembled into two different genomes, denoted DS1 and DSP1. Additionally, two phages—DS9 and DS10—were identical, but DS9 had an additional contig (DSP2); thus, only DS10 is reported here. Interestingly, these two predicted prophages appeared to have co-amplified with multiple phage isolates: DSP1 appeared in plate lysates of DS1 and DS4 through DS8, while DSP2 appeared in lysates of DS3 and DS4. In total, 14 unique phage genomes were identified in DNA preparations from 12 plate lysates (Table 1). Phage genomes were analyzed as described in the Materials and Methods section.

TABLE 1
Isolated phage genomes

| Bacteriophage | DH2 | DH5 | DS1* | DS3† |
|---|---|---|---|---|
| Accession Number | OM373548 | OM373549 | OM373550 | OM373551 |
| Host Strain | AZM22 | AZM37 | AZM22 | AZM25 |
| Source | Hair | Hair | Skin | Skin |
| Morphology | Myovirus | Siphovirus | — | — |
| Genome Size (Bp) | 138,526 | 92,077 | 17,344 | 38,198 |
| G + C (%) | 30.8 | 30.3 | 33.3 | 35 |
| ORFs | 205 | 134 | 23 | 56 |
| Hypothetical | 159 | 121 | 11 | 27 |
| Proteins | | | | |
| Proteins with | 46 | 13 | 12 | 29 |
| Functional | | | | |
| Assignment | | | | |
| tRNAs | 1 | 5 | 0 | 0 |
| Genus | Twortvirus | Sextaecvirus | Fibralongavirus | Coventryvirus |
| Antibiotic Resistance | 0 | 0 | 0 | 0 |
| CDS | | | | |
| Bacterial Virulence | 0 | 0 | 0 | 1 |
| CDS | | | | |
| Closest Sequenced | AY954970.1 | MW589261 | MK075001 | NC_055022 |
| Relative | | | | |
| Attachment Sites | 0 | 2 | 0 | 2 |
| Integrases | 0 | 1 | 0 | 1 |
| Lifestyle Prediction | Vir | Vir | Temp | Temp |

| Bacteriophage | DS4*† | DS5* | DS6* | DS7* |
|---|---|---|---|---|
| Accession Number | OM373552 | OM373553 | OM373554 | OM373555 |
| Host Strain | AZM26 | AZM30 | AZM32 | AZM22 |
| Source | Skin | Skin | Skin | Skin |
| Morphology | — | — | — | — |
| Genome Size (Bp) | 38,285 | 26,100 | 42,714 | 84,555 |
| G + C (%) | 30.2 | 37.8 | 35.4 | 37.3 |
| ORFs | 57 | 35 | 68 | 95 |
| Hypothetical | 28 | 15 | 43 | 32 |
| Proteins | | | | |
| Proteins with | 29 | 20 | 35 | 63 |
| Functional | | | | |
| Assignment | | | | |
| tRNAs | 0 | 0 | 0 | 1 |
| Genus | Coventryvirus | Coventryvirus | Fibralongavirus | Unknown |
| Antibiotic Resistance | 0 | 0 | 0 | 0 |
| CDS | | | | |
| Bacterial Virulence | 1 | 1 | 0 | 10 |
| CDS | | | | |
| Closest Sequenced | NC_055022 | NC_055022 | MK075001 | CP065925 |
| Relative | | | | |
| Attachment Sites | 2 | 2 | 2 | 0 |
| Integrases | 1 | 0 | 1 | 0 |
| Lifestyle Prediction | Temp | Temp | Temp | Temp |

| Bacteriophage | DS8* | DS10 | DS11 |
|---|---|---|---|
| Accession Number | OM373556 | OM373557 | OM373558 |
| Host Strain | AZM22 | AZM22 | AZM64 |
| Source | Skin | Skin | Skin |
| Morphology | — | Myovirus | — |
| Genome Size (Bp) | 35,683 | 139,829 | 38,381 |
| G + C (%) | 35.8 | 30.4 | 36.2 |
| ORFs | 58 | 217 | 58 |
| Hypothetical Proteins | 35 | 179 | 35 |
| Proteins with | 23 | 38 | 23 |
| Functional Assignment | | | |
| tRNAs | 0 | 4 | 0 |
| Genus | Fibralongavirus | Kayvirus | Biseptimavirus |
| Antibiotic Resistance | 0 | 0 | 0 |
| CDS | | | |
| Bacterial Virulence | 0 | 0 | 0 |
| CDS | | | |
| Closest Sequenced | MK075001 | KF766114 | KX827371 |
| Relative | | | |
| Attachment Sites | 0 | 0 | 2 |
| Integrases | 0 | 0 | 1 |
| Lifestyle Prediction | Temp | Vir | Temp |

| Bacteriophage | DS12 | DSP1 | DSP2 |
|---|---|---|---|
| Accession Number | OM373559 | OM373560 | OM373561 |
| Host Strain | AZM64 | AZM22 | AZM22 |
| Source | Skin | Skin | Skin |
| Morphology | — | — | — |
| Genome Size (Bp) | 43,755 | 16,901 | 41,107 |
| G + C (%) | 36.4 | 28.1 | 35.2 |
| ORFs | 61 | 17 | 60 |
| Hypothetical Proteins | 36 | 10 | 41 |
| Proteins with | 25 | 7 | 19 |
| Functional Assignment | | | |
| tRNAs | 0 | 0 | 0 |
| Genus | Unknown | Unknown | Fibralongavirus |
| Antibiotic Resistance | 0 | 0 | 0 |
| CDS | | | |
| Bacterial Virulence | 0 | 0 | 0 |
| CDS | | | |
| Closest Sequenced | NC_048192 | NC_047813 | MK075001 |
| Relative | | | |
| Attachment Sites | 2 | 0 | 2 |
| Integrases | 1 | 0 | 1 |
| Lifestyle Prediction | Temp | Temp | Temp |

Key:
*= contains DSP1;
†= contains DSP2;
Virulent = Vir; Temperate = Temp; CDS = coding sequence; Bp = base pair; G + C (%) = Guanine and Cytosine content percentage.

### Example 3—Phages DH2, DH5, and DS10 are Polyvalent S. pseudintermedius and S. aureus Phages

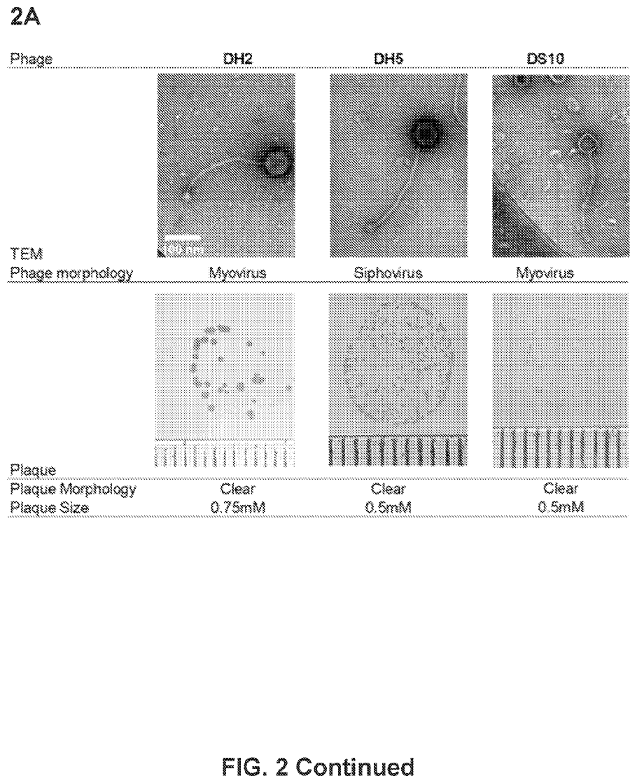

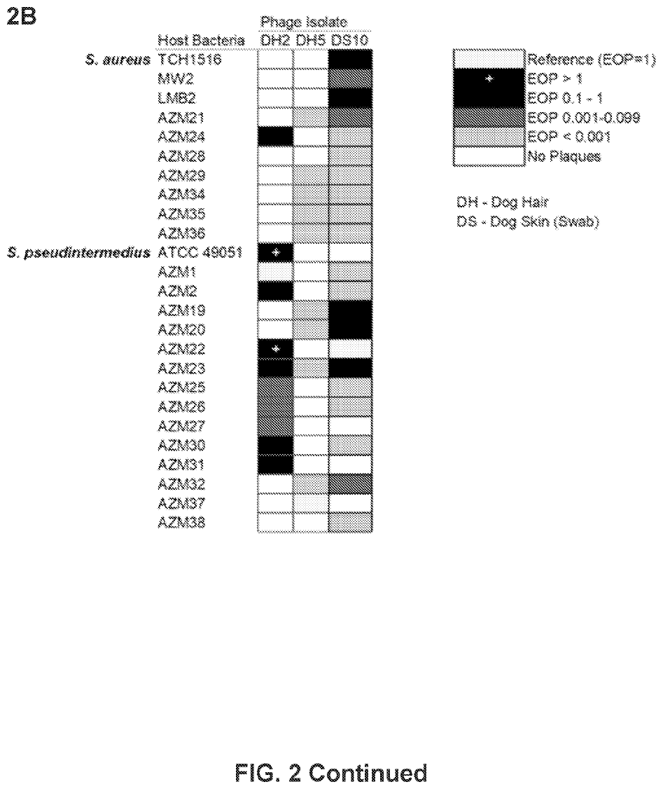

The absence of any confirmed lytic S. pseudintermedius phages in the literature compelled the inventors to further investigate the predicted lytic phages (DH2, DH5, and DS10). FIG. 2 displays TEM morphology, plaque morphology, host range, and efficiency of plating (EOP) for DH2, DH5, and DS10, respectively. As predicted from genomic analysis, DH2 and DS10 exhibited typical myovirus morphology with long contractile tails, while DH5 possessed a non-contractile tail with a large base plate (FIG. 2A). The plaques produced by the bacteriophages varied in size (0.5-0.75 mm), but each of the plaques were clear (FIG. 2A). A panel of 15 S. pseudintermedius (ATCC 49051, AZM1, AZM2, AZM19, AZM20, AZM22, AZM34, AZM25, AZM26, AZM27, AZM30, AZM31, AZM32, AZM37, and AZM38) and 10 S. aureus (TCH1516, MW2, LMB2, AZM21, AZM24, AZM28, AZM29, AZM34, AZM35, and AZM36) isolates were challenged with DH2, DH5, and DS10 to determine host range and EOP (FIG. 2B). DH2 exhibited species specificity, plaquing on 10/15 (66.7%) S. pseudintermedius isolates and only 1/10 (10%) S. aureus isolates. DH5 plaqued on 5/15 (33.3%) of S. pseudintermedius isolates and surprisingly, on 5/10 (50%) of the S. aureus isolates. However, the EOP was less than 0.001 on all strains, indicative of lysis-from-without. Lastly, DS10 plaqued on 11/15 (73.3%) S. pseudintermedius isolates, and all 10 S. aureus isolates—including the three clinical isolates TCH1516, MW2, and LMB2. TCH1516 and MW2 are USA 300 and 400 strains respectively, while LMB2 is an uncharacterized patient isolate of TAILΦR Labs ³⁰⁻³²

### Example 4—Mitomycin C Induction Supports DH2, DH5, and DS10 as the First Confirmed Lytic S. pseudintermedius Phages

DH2 is a Twortvirus with no antibiotic resistance or bacterial virulence coding sequences (CDS), no attachment sites or integrases, but does harbor a putative anti-repressor. DH5 is a Sextaecvirus without any antibiotic resistance or bacterial virulence CDS but contains putative attachment sites and an integrase. DS10 is a Kayvirus and like DH2, lacks antibiotic resistance and bacterial virulence CDS, attachment sites, and integrases. Given the ambiguity of 2 of the 3 genomes, the predicted lytic lifestyle was tested using mitomycin C induction assays (e.g., mitomycin C activates the SOS repair system, including the enzyme RecA, which cleaves a phage repressor and induces lysogenic phages to enter the lytic cycle).

DH5 was propagated on AZM37, the only newly isolated S. pseudintermedius strain that produced a productive infection. DH2 and DS10 were propagated on ATCC49051 and TCH1516 respectively. These S. pseudintermedius strains were chosen because they are sequenced, their prophage content is known ³⁰ or has been predicted, and the isolated bacteriophages are virulent on these strains as assessed by EOP assay (see FIG. 2 B). A phage K (e.g., a well-studied lytic phage ^{33,34}), and predicted temperate phage DS3, were also included as negative and positive controls cultured with TCH1516 (S. aureus) or AZM22 (S. pseudintermedius) respectively. Table 2 displayed the results of the mitomycin C induction assays. Surprisingly, DH5 did not exhibit plaque formation from 15 induced isolates tested. Likewise, DH2, DS10, and phage K did not exhibit plaque formation from 5 induced isolates tested for each. As predicted, the positive control DS3 formed plaques with 8 of 10 induced isolates. For Table 2, Mitomycin C (MMC) induction assays were performed with either host strain alone, or with phage as described in methods. Number of plates with plaques and number of biological isolates tested were reported. Predicted temperate phage DS3 and phage K were included as positive and negative controls for lysogeny respectively.

TABLE 2
Outcome of Mitomycin C (MMC) Induction Assays

| | | MMC Only | | MMC + Phage | |
|---|---|---|---|---|---|
| | Strain - Phage | Plaquing | Isolates | Plaquing | Isolates |
| Species | Challenge | Observed | Tested | Observed | Tested |
| S. aureus | TCH1516 - DS10, phage K | 0 | 5 | 0 | 5 each |
| S. pseudintermedius | ATCC49051 - DH2 | 0 | 5 | 0 | 5 |
| S. pseudintermedius | AZM37 - DH5 | 1 | 8 | 0 | 15 |
| S. pseudintermedius | AZM22 - DS3 | 0 | 8 | 8 | 10 |

### Example 5—Staphylococcus Phages in Murine Models of Pyoderma

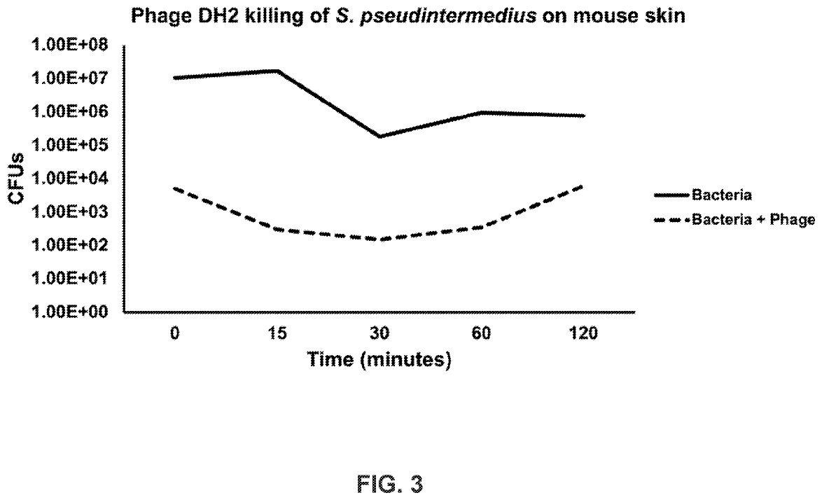

The ability of DH2 to kill S. pseudintermedius strain AZM22 in a physiologically-relevant environment: mouse skin, was tested. When both DH2 and S. pseudintermedius strain AZM22 were placed on a bare patch of mouse skin, DH2 reduced the bacterial burden of AZM22 by 3 logs compared to AZM22 on skin alone (FIG. 3). These data indicated that Staphylococcal phages may be able to kill their bacterial hosts on animal skin.

The ability of DH2, DH5, and/or DS10 to kill Staphylococcus strains are tested in physiologically relevant environments and/or models (e.g., mouse skin, urine, blood, sputum, or other medically relevant media). When DH2, DH5, and/or DS10 are applied in conjunction with a Staphylococcus strain, a reduction in bacterial burden is observed. These data indicate that cocktails of DH2, DH5, and/or DS10 are able to kill bacterial hosts on animal skin.

The ability of DH2, DH5, and/or DS10 to treat and/or prevent Staphylococcus infections (e.g., pyoderma, diabetic ulcers, sepsis, etc.) in animal models are tested. DH2, DH5, and/or DS10 treat and/or prevent Staphylococcus infections and/or symptoms associated with the same.

DH2, DH5, and/or DS10 are evolved to reduce bacterial resistance to phage. Phage are evolved in a manner similar to that as described in Keiko Salazer et al., Antiviral Resistance and Phage Counter Adaptation to Antibiotic-Resistant Extraintestinal Pathogenic Escherichia coli. mBio, 2021, PMID 33906920; which is incorporated herein by reference.

All of the methods disclosed and claimed herein can be made and executed without undue experimentation in light of the present disclosure. While the compositions and methods of this invention have been described in terms of certain embodiments, it will be apparent to those of skill in the art that variations may be applied to the methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.
1. Devriesc L A, Vancanneyt M, Bacle M, et al. Staphylococcus Pseudintermedius Sp. Nov., a Coagulase-Positive Species from Animals. Int J Syst Evol Microbiol 2005; 55(4):1569-1573; doi: 10.1099/ijs.0.63413-0.
2. Ross Fitzgerald J. The Staphylococcus Intermedius Group of Bacterial Pathogens: Species Re-Classification, Pathogenesis and the Emergence of Meticillin Resistance: Recent Studies of the Staphylococcus Intermedius Group. Vet Dermatol 2009; 20(5-6):490-495; doi: 10.1111/j.1365-3164.2009.00828.x.
3. Weese J S and van Duijkeren E. Methicillin-Resistant Staphylococcus Aureus and Staphylococcus Pseudintermedius in Veterinary Medicine. Vet Microbiol 2010; 140(3-4):418-429; doi: 10.1016/j.vetmic.2009.01.039.
4. van Duijkeren E, Catry B, Greko C, et al. Review on Methicillin-Resistant Staphylococcus Pseudintermedius. J Antimicrob Chemother 2011; 66(12):2705-2714; doi: 10.1093/jac/dkr367.
5. Lynch S A and Helbig K J. The Complex Diseases of Staphylococcus Pseudintermedius in Canines: Where to Next?Vet Sci 2021; 8(1):11; doi: 10.3390/vetsci8010011.
6. Moodley A, Damborg P and Nielsen S S. Antimicrobial Resistance in Methicillin Susceptible and Methicillin Resistant Staphylococcus Pseudintermedius of Canine Origin: Literature Review from 1980 to 2013. Vet Microbiol 2014; 171(3-4):337-341; doi: 10.1016/j.vetmic.2014.02.008.
7. Hartantyo S H P, Chau M L, Fillon L, et al. Sick Pets as Potential Reservoirs of Antibiotic-Resistant Bacteria in Singapore. Antimicrob Resist Infect Control 2018; 7(1):106; doi: 10.1186/s13756-018-0399-9.
8. Jantorn P, Heemmamad H, Soimala T, et al. Antibiotic Resistance Profile and Biofilm Production of Staphylococcus Pseudintermedius Isolated from Dogs in Thailand. Pharmaceuticals 2021; 14(6):592; doi: 10.3390/ph14060592.
9. EFSA Panel on Animal Health and Welfare (AHAW), Nielsen S S, Bicout D J, et al. Assessment of Animal Diseases Caused by Bacteria Resistant to Antimicrobials: Dogs and Cats. EFSA J 2021; 19(6); doi: 10.2903/j.efsa.2021.6680.
10. Principi N, Silvestri E and Esposito S. Advantages and Limitations of Bacteriophages for the Treatment of Bacterial Infections. Front Pharmacol 2019; 10:513; doi: 10.3389/fphar.2019.00513.
11. Valente L, Prazak J, Que Y-A, et al. Progress and Pitfalls of Bacteriophage Therapy in Critical Care: A Concise Definitive Review. Crit Care Explor 2021; 3(3):e0351; doi: 10.1097/CCE.0000000000000351.
12. Melo L D R, Sillankorva S, Ackermann H-W, et al. Isolation and Characterization of a New Staphylococcus Epidermidis Broad-Spectrum Bacteriophage. J Gen Virol 2014; 95(2):506-515; doi: 10.1099/vir.0.060590-0.
13. Zeman M, Birdy P, Vrbovski V, et al. New Genus Fibralongavirus in Siphoviridae Phages of Staphylococcus Pseudintermedius. Viruses 2019; 11(12):1143; doi: 10.3390/v11121143.
14. Tynecki P, Guzinski A, Kazimierczak J, et al. PhageAI—Bacteriophage Life Cycle Recognition with Machine Learning and Natural Language Processing. bioRxiv 2020; doi: 10.1101/2020.07.11.198606.
15. Patel A. Superficial Bacterial Folliculitis. Companion Anim 2018; 23(6):308-313; doi: 10.12968/coan.2018.23.6.308.
16. Terwilliger A L, Gu Liu C, Green S I, et al. Tailored Antibacterials and Innovative Laboratories for Phage (Φ) Research: Personalized Infectious Disease Medicine for the Most Vulnerable At-Risk Patients. PHAGE 2020; 1(2):66-74; doi: 10.1089/phage.2020.0007.
17. Gibson S B, Green S I, Liu C G, et al. Constructing and Characterizing Bacteriophage Libraries for Phage Therapy of Human Infections. Front Microbiol 2019; 10:2537; doi: 10.3389/fmicb.2019.02537.
18. Bushnell B. BBMap. 38.84. https://sourceforge.net/projects/bbmap/
19. Darling A C E. Mauve: Multiple Alignment of Conserved Genomic Sequence With Rearrangements. Genome Res 2004; 14(7):1394-1403; doi: 10.1101/gr.2289704.
20. Brettin T, Davis J J, Disz T, et al. RASTtk: A Modular and Extensible Implementation of the RAST Algorithm for Building Custom Annotation Pipelines and Annotating Batches of Genomes. Sci Rep 2015; 5(1):8365; doi: 10.1038/srep08365.
21. Aziz R K, Bartels D, Best A A, et al. The RAST Server: Rapid Annotations Using Subsystems Technology. BMC Genomics 2008; 9(1):75; doi: 10.1186/1471-2164-9-75.
22. Overbeek R, Olson R, Pusch G D, et al. The SEED and the Rapid Annotation of Microbial Genomes Using Subsystems Technology (RAST). Nucleic Acids Res 2014; 42(D1):D206-D214; doi: 10.1093/nar/gkt1226.
23. Johnson M, Zaretskaya I, Raytselis Y, et al. NCBI BLAST: A Better Web Interface. Nucleic Acids Res 2008; 36(Web Server):W5-W9; doi: 10.1093/nar/gkn201.
24. McArthur A G, Waglechner N. Nizam F, et al. The Comprehensive Antibiotic Resistance Database. Antimicrob Agents Chemother 2013; 57(7):3348-3357; doi: 10.1128/AAC.00419-13.
25. Chen L, Zheng D, Liu B, et al. VFDB 2016: Hierarchical and Refined Dataset for Big Data Analysis-10 Years On. Nucleic Acids Res 2016; 44(D1):D694-D697; doi: 10.1093/nar/gkv1239.
26. Sayers S, Li L, Ong E, et al. Victors: A Web-Based Knowledge Base of Virulence Factors in Human and Animal Pathogens. Nucleic Acids Res 2019; 47(D1):D693-D700; doi: 10.1093/nar/gky999.
27. Mao C, Abraham D, Wattam A R, et al. Curation, Integration and Visualization of Bacterial Virulence Factors in PATRIC. Bioinformatics 2015; 31(2):252-258; doi: 10.1093/bioinformatics/btu631.
28. Arndt D, Grant J R, Marcu A, et al. PHASTER: A Better, Faster Version of the PHAST Phage Search Tool. Nucleic Acids Res 2016; 44(W1):W16-W21; doi: 10.1093/nar/gkw387.
29. Adriaenssens E and Brister J R. How to Name and Classify Your Phage: An Informal Guide. Viruses 2017; 9(4):70; doi: 10.3390/v9040070.
30. Highlander S K, Hult6n KG, Qin X, et al. Subtle Genetic Changes Enhance Virulence of Methicillin Resistant and Sensitive Staphylococcus Aureus. BMC Microbiol 2007; 7(l):99; doi: 10.1186/1471-2180-7-99.
31. Centers for Disease Control and Prevention (CDC). Four pediatric deaths from community-acquired methicillin-resistant Staphylococcus aureus—Minnesota and North Dakota, 1997-1999. MMWR Morb Mortal Wkly Rep. 1999 Aug. 20; 48(32):707-10. PMID: 21033181.
32. Baba T, Takeuchi F, Kuroda M, et al. Genome and Virulence Determinants of High Virulence Community-Acquired MRSA. The Lancet 2002; 359(9320):1819-1827; doi: 10.1016/S0140-6736(02)08713-5.
33. O'Flaherty S, Coffey A, Edwards R, et al. Genome of Staphylococcal Phage K: A New Lineage of Myoviridac Infecting Gram-Positive Bacteria with a Low G2C Content. J BACTERIOL 2004; 186:10.
34. O'Flaherty S, Ross R P, Meaney W, et al. Potential of the Polyvalent Anti-Staphylococcus Bacteriophage K for Control of Antibiotic-Resistant Staphylococci from Hospitals. Appl Environ Microbiol 2005; 71(4):1836-1842; doi: 10.1128/AEM.71.4.1836-1842.2005.
35. Beck K M, Waisglass S E, Dick H L N, et al. Prevalence of Meticillin-Resistant Staphylococcus Pseudintermedius (MRSP) from Skin and Carriage Sites of Dogs after Treatment of Their Meticillin-Resistant or Meticillin-Sensitive Staphylococcal Pyoderma: MRSP in Canine Pyoderma. Vet Dermatol 2012; 23(4):369-e67; doi: 10.1111/j.1365-3164.2012.01035.x.
36. Hannigan G D, Meisel J S, Tyldsley A S, et al. The Human Skin Double-Stranded DNA Virome: Topographical and Temporal Diversity, Genetic Enrichment, and Dynamic Associations with the Host Microbiome. Heitman J. ed. mBio 2015; 6(5):e01578-15; doi: 10.1128/mBio.01578-15.
37. Valente L G, Pitton M, Furholz M, et al. Isolation and Characterization of Bacteriophages from the Human Skin Microbiome That Infect Staphylococcus Epidernidis. FEMS Microbes 2021; 2:xtab003; doi: 10.1093/femsmc/xtab003.
38. Azam A H, Kadoi K, Miyanaga K, et al. Analysis Host-Recognition Mechanism of Staphylococcal Kayvirus ϕSA039 Reveals a Novel Strategy That Protects Staphylococcus Aureus against Infection by Staphylococcus Pseudintermedius Siphoviridae Phages. Appl Microbiol Biotechnol 2019; 103(16):6809-6823; doi: 10.1007/s00253-019-09940-7.
39. Botka T, PantnMek R, Maslanovi I, et al. Lytic and Genomic Properties of Spontaneous Host-Range Kayvirus Mutants Prove Their Suitability for Upgrading Phage Therapeutics against Staphylococci. Sci Rep 2019; 9(1):5475; doi: 10.1038/s41598-019-41868-w.
40. Junjappa R P, Desai S N, Roy P, et al. Efficacy of Anti-Staphylococcal Protein P128 for the Treatment of Canine Pyoderma: Potential Applications. Vet Res Commun 2013; 37(3):217-228; doi: 10.1007/s11259-013-9565-y.
41. Lobocka M, Hejnowicz M S, Dabrowski K, et al. Genomics of Staphylococcal Twort-like Phages—Potential Therapeutics of the Post-Antibiotic Era. In: Advances in Virus Research Elsevier; 2012; pp. 143-216; doi: 10.1016/B978-0-12-394438-2.00005-0.
42. Deghorain M and Van Melderen L. The Staphylococci Phages Family: An Overview. Viruses 2012; 4(12):3316-3335; doi: 10.3390/v4123316.
43. Moodley A, Kot W, Nalgird S, et al. Isolation and Characterization of Bacteriophages Active against Methicillin-Resistant Staphylococcus Pseudintermedius. Res Vet Sci 2019; 122:81-85; doi: 10.1016/j.rvsc.2018.11.008.
44. Polak-Witka K, Rudnicka L, Blume-Peytavi U, et al. The Role of the Microbiome in Scalp Hair Follicle Biology and Disease. Exp Dermatol 2020; 29(3):286-294; doi: 10.1111/exd.13935.

What is claimed is:

1. A composition comprising an isolated bacteriophage vB_SpsM-DH2 (Accession No. OM373548).

2. The composition of claim 1, comprising about 10⁸ to about 10¹¹ plaque forming units (PFU) per milliliter (PFU/ml) of isolated bacteriophage vB_SpsM-DH2.

3. A composition comprising an isolated bacteriophage vB_SpsM-DS10 (Accession No. OM373557).

4. The composition of claim 3, comprising about 10⁸ to about 10¹¹ PFU/ml of isolated bacteriophage vB_SpsM-DS10.

5. A composition comprising isolated bacteriophage vB_SpsM-DH2 and an isolated bacteriophage vB_SpsM-DS10.

6. The composition of claim 5, comprising about 10⁸ to about 10¹¹ PFU/ml isolated bacteriophage vB_SpsM-DH2 and about 10⁸ to about 10¹¹ PFU/ml vB_SpsM-DS10.

7. The composition of any one of claim 5 or 6, consisting essentially of isolated bacteriophage vB_SpsM-DH2 and isolated bacteriophage vB_SpsM-DS10.

8. A composition comprising an isolated bacteriophage with greater than 95%, 96%, 97%, 98%, 99%, 99.1%, 99.2%, 99.3%, 99.4%, 99.5%, 99.6%, 99.7%, 99.8%, 99.9%, or 99.95% genomic sequence identity to the genomic sequences of vB_SpsM-DH2 (SEQ ID NO: 1), and/or vB_SpsM-DS10 (SEQ ID NO: 3).

9. The composition of any one of claims 1-8, wherein the isolated bacteriophage are not lysogenic, do not comprise antibiotic resistance coding sequences, do not comprise bacterial virulence coding sequences, and/or have lytic capacity.

10. The composition of any one of claims 1-9, wherein the isolated bacteriophage are evolved to improve lytic capacity, reduce lysogenic capacity, reduce antibiotic resistance gene obtainment capacity, reduce bacterial virulence gene obtainment capacity, and/or increase the number of bacterial strains subject to lysis by the isolated bacteriophage lytic.

11. The composition of any one of claims 5-10, wherein the amount of the bacteriophages in the composition are substantially the same.

12. The composition of any one of claims 5-11, wherein the amount of the bacteriophages in the composition are not substantially the same.

13. The composition of any one of claims 1-12, wherein the composition is a lotion, cream, body butter, mask, scrub, wash, gel, serum, emulsion (e.g., oil-in-water, water-in-oil, silicone-in-water, water-in-silicone, water-in-oil-in-water, oil-in-water-in-oil, oil-in-water-in-silicone, etc.), solution (e.g., aqueous or hydro-alcoholic solutions), anhydrous bases (e.g., stick or a powder), ointment, milk, paste, aerosol, solid form, jelly, and/or powdered form (e.g., dried, lyophilized, particulate, etc).

14. The composition of any one of claims 1-13, wherein the composition is a solution, lotion, and/or cream.

15. The composition of any one of claims 1-14, wherein the composition is shelf-stable.

16. The composition of any one of claims 1-15, wherein the composition is formulated for topical, oral, aural, nasal, and/or ophthalmic application.

17. The composition of any one of claims 1-16, wherein the composition is formulated for application more than once a day, once a day, twice a day, once a week, twice a week, once a month, or twice a month during use.

18. The composition of any one of claims 1-17, wherein the composition is housed in a delivery apparatus.

19. The composition of any one of claims 1-18, wherein the composition is comprised in a suitable container.

20. The composition of any one of claims 1-19, wherein the composition is comprised in a kit.

21. A method of treating and/or preventing a Staphylococcus infection in a subject, comprising administering to the subject the composition of any one of claims 1-20.

22. The method of claim 21, wherein the Staphylococcus infection comprises a Staphylococcus pseudintermedius and/or Staphylococcus aureus infection.

23. The method of claim 21 or 22, wherein the infection is a urinary tract, blood, gut, abdomen, stomach, lungs, skin, ear, eye, nose, oral, kidney, prostate, bladder, brain, vaginal tract, heart, liver, spleen, tendons, or wound (cuts, burns, sores, etc.) infection, or a combination thereof.

24. The method of any one of claims 21-23, wherein the infection is a wound, skin, ear, eye, nose, mucosal, and/or oral infection.

25. The method of any one of claims 21-23, wherein the infection is a catheter-associated infection.

26. The method of any one of claims 21-25, wherein the Staphylococcus is of pulse-field gel electrophoresis (PFGE) type USA200, USA300, and/or USA400.

27. The method of any one of claims 21-26, wherein the Staphylococcus is of clonal complex CC1, CC12, CC121, CC15, CC22, CC25, CC30, CC45, CC5, CC59, CC8, CC80, and/or CC97, CC398.

28. The method of any one of claims 21-27, wherein the Staphylococcus is MW2, No. 10, NP66, CI/BAC/25/13/W, XQ, 046, ST20130941, UP_620, 11050960412, H-EMRSA-15, 014S_SA, NCTC8317, MRSA252, FORC_001, LA-MRSA ST398, 08BA02176, CA-347, USA600, Mu3, N315, SA40, HZW450, USA300_FPR3757, COL, GR2, 11819-97, MOK063, CC1153-MRSA, TCH1516, LMB2, MN8, CDC587, MNWH, MNPE, FRI1169, Newman, LAC, MNLevy, c99-529, JH1, JH9, FPR3757, RF122, ST228, AZM21, AZM24, AZM28, AZM29, AZM34, AZM35, AZM36, ATCC 49051, AZM1, AZM2, AZM19, AZM20, AZM22, AZM23, AZM25, AZM26, AZM27, AZM30, AZM31, AZM32, AZM37, and/or AZM38.

29. The method of any one of claims 21-28, wherein the Staphylococcus is drug-resistant.

30. The method of any one of claims 21-29, wherein the Staphylococcus is multidrug-resistant.

31. The method of any one of claims 21-30, wherein the subject has a urinary tract infection, neonatal meningitis, a blood-stream infection, pneumonia, sepsis, a surgical wound infection, a wound infection, a skin infection, an eye infection, an ear infection, an oral infection, a prostate infection, meningitis, a vaginal infection, or a combination thereof.

32. The method of any one of claims 21-31, wherein the subject is immunosuppressed.

33. The method of any one of claims 21-32, wherein the subject has an immune cell defect, asplenia, impaired splenic function, nephrotic syndrome, or an autoimmune condition.

34. The method of any one of claims 21-33, wherein the subject is administered the composition prior to a medical procedure or regimen.

35. The method of any one of claims 21-34, wherein the subject will be subject to immunosuppressive conditions.

36. The method of any one of claims 21-35, wherein the subject is taking or will be taking chemotherapy.

37. The method of any one of claims 21-36, wherein the subject is taking or will be taking an immunosuppressant.

38. The method of claim 37, wherein the immunosuppressant is a glucocorticoid, a calcineurin inhibitor, an antimetabolite, or an antibody therapy.

39. The method of any one of claims 21-38, wherein the source of the Staphylococcus was from a beverage, comestible, an individual, or an environment.

40. The method of claim 39, wherein the environment is ground or surface water, water used to irrigate crops, a public water system, a hospital, a school, a nursing home, a petting zoo, a daycare, a lodging, a cruise ship, a train, a razor, a towel, clothing, a gymnasium, or an airplane.

41. The method of any one of claims 21-40, wherein the subject is a mammal.

42. The method of any one of claims 21-41, wherein the subject is a domestic animal.

43. The method of any one of claims 21-42, wherein the subject is a farm animal.

44. The method of any one of claims 21-43, wherein the subject is a dog or a cat.

45. The method of any one of claims 21-43, wherein the subject is a cow, a horse, a sheep, or a goat.

46. The method of any one of claims 21-41, wherein the subject is a human.

47. A device, comprising, on, in, and/or around the device, isolated bacteriophages vB_SpsM-DH2, and/or vB_SpsM-DS10.

48. The device of claim 47, wherein the device is a catheter, drive line, syringe, tube, implant, defibrillator, artificial joint, pacemaker, screw, rod, disc, intrauterine device, pin, plate, stent, dental device, eye lens, shunt, valve, neurological or neurosurgical device, gastrointestinal device, genitourinary device, catheter cuff, vascular access device, or wound drain.

49. The device of claim 47 or 48, further defined as having a coating comprising the bacteriophages.

50. A method of obtaining lytic bacteriophages comprising,
a) obtaining hair and/or skin sample(s) from one or more subjects,
b) isolating phages from the sample(s), and
c) analyzing the genome of isolated phages to predict lytic capacity and/or directly determining plaque forming capacity of the phages by exposing a target bacteria to the isolated phages.

51. The method of claim 50, wherein the hair and/or skin samples comprise at least a portion of a hair follicle.

52. The method of claim 50 or 51, wherein the hair and/or skin samples comprise an upper portion of a hair follicle.

53. The method of any one of claims 50-52, wherein the isolated phage is analyzed to determine lysogenic capacity, and wherein the isolated phage is determined to be non-lysogenic.

54. The method of any one of claims 50-53, wherein the isolated bacteriophage has greater than 95%, 96%, 97%, 98%, 99%, 99.1%, 99.2%, 99.3%, 99.4%, 99.5%, 99.6%, 99.7%, 99.8%, 99.9%, or 99.95% genomic sequence identity to the genomic sequences of vB_SpsM-DH2 (SEQ ID NO: 1), and/or vB_SpsM-DS10 (SEQ ID NO: 3).

55. The method of any one of claims 50-54, wherein the isolated bacteriophage are not lysogenic, do not comprise antibiotic resistance coding sequences, do not comprise bacterial virulence coding sequences, and/or have lytic capacity.

56. The method of any one of claims 50-55, wherein the isolated bacteriophage are evolved to improve lytic capacity, reduce lysogenic capacity, reduce antibiotic resistance gene obtainment capacity, reduce bacterial virulence gene obtainment capacity, and/or increase the number of bacterial strains subject to lysis by the isolated bacteriophage.
